(19) United States
(12) Patent Application Publication
(10) Pub. No.: US 2026/0234163 A1
(43) Pub. Date: Aug. 13, 2026

(54) NOVEL COMPOUNDS AND USES THEREOF

(71) Applicant: ACELINK THERAPEUTICS, INC., Newark, CA (US)

(72) Inventors: Yeyu CAO, Foster City, CA (US); Bing WANG, San Jose, CA (US)

(21) Appl. No.: 19/470,812

(22) PCT Filed: Apr. 2, 2024
(86) PCT No.: PCT/US2024/022567

§ 371 Date: Sep. 30, 2025

Related U.S. Application Data
(60) Provisional application No. 63/493,775, filed on Apr. 3, 2023.

(51) Int. Cl. C07D 515/18; A61K 31/4192; A61K 31/4748; C07D 487/08; C07D 487/18; C07D 487/22; C07D 498/08; C07D 498/18; C07D 498/22

(52) U.S. Cl. CPC C07D 515/18; A61K 31/4192; A61K 31/4748; C07D 487/08; C07D 487/18; C07D 487/22; C07D 498/08; C07D 498/18; C07D 498/22

(57) ABSTRACT

The present disclosure relates to novel compounds, and tautomers, stereoisomers or pharmaceutically acceptable salts thereof, which modulate the level or activity of Nrf2 protein. The present disclosure also relates to pharmaceutical compositions comprising one or more of the compounds and tautomers, stereoisomers, or pharmaceutically acceptable salts thereof as an active ingredient, and to the use of the compounds and tautomers, stereoisomers, or pharmaceutically acceptable salts thereof in the treatment of Nrf2-associated diseases, disorders or conditions, including ocular diseases or kidney diseases.

### TECHNICAL FIELD

The present disclosure provides novel compounds, and tautomers, stereoisomers or pharmaceutically acceptable salts thereof, which modulate the level or activity of nuclear factor erythroid 2-related factor (Nrf2) protein. The present disclosure also relates to pharmaceutical compositions comprising one or more of the compounds and tautomers, stereoisomers, or pharmaceutically acceptable salts thereof as an active ingredient, and to the use of the compounds and tautomers, stereoisomers, or pharmaceutically acceptable salts thereof in the treatment of Nrf2-associated diseases, disorders or conditions, including ocular diseases or kidney diseases.

### BACKGROUND OF THE INVENTION

Nuclear factor erythroid 2-related factor (also known as “Nrf2” or “NF-E2 related factor 2”) is a member of the cap-n-collar (“CNC”) family of transcription factors containing characteristic basic-leucine zipper motif. Under basal conditions, Nrf2 levels are tightly controlled by the cytosolic actin-bound repressor, Kelch-like ECH associating protein 1 (also known as “Keap1”), which binds to Nrf2 and targets it for ubiquitylation and proteasomal degradation via the Cul3-based E3-ubiquitin ligase complex. Under conditions of oxidative stress, modification of reactive cysteines on Keap1 produces a conformational change that alters Nrf2 binding and promotes Nrf2 stabilization. Thus, in normal conditions, the levels of Nrf2 in the cytosol are low, but the system is designed to respond immediately to oxidative stress by increasing Nrf2 activity.

Oxidative stress refers to a condition where oxidation and anti-oxidation is out of balance leading to excessive oxidation reactions, which can adversely affect organisms and contribute to various pathogeneses. When cells are experiencing oxidative stress, Nrf2 dissociates from Keap1, translocates from the cytoplasm to the nucleus, and binds to a transcriptional binding site called ARE (anti-oxidant response element). By binding the AREs present in the promoter regions of specific genes, Nrf2 regulates the transcription of approximately 250 genes, which together build a multifaceted network that integrates cellular activities including detoxification reactions, the maintenance of both redox and protein homeostasis, and energy metabolism (Hayes, J. D. et al., Trends Biochem. Sci. 2014, 39(4): 199-218). The activation of Nrf2 also leads to the suppression of NF-kB proinflammatory signaling. In recent years, therapies based on the activation of Nrf2 to combat both oxidative stress and inflammation have been proposed to be beneficial in chronic diseases (Cuadrado, A. et al., Pharmacol. Rev. 2018, 70, 348-383; Cuadrado, A. et al., Nat. Rev. Drug Discov. 2019, 18(4): 295-317; Lu, M. C. et al., Med. Res. Rev. 2016, 36(5): 924-963; Zhuang, C. et al., MedChemComm. 2017, 8(2): 286-294). Given its pivotal role in adjusting cellular defenses under stress conditions and during hormesis, Nrf2 has been proposed to hold great promise as a drug target for the prevention or treatment of a wide range of pathological and chronic conditions that are linked to increased oxidative stress and inflammation, impaired redox potential, impaired detoxification and deregulated metabolism (Calabrese, V. et al., Nat. Rev. Neurosci. 2007, 8(10): 766-775; Trovato Salinaro, A. et al., Immun. Ageing 2018, 15: 8; Calabrese, V. et al., Antioxid. Redox Signaling 2010, 13(11): 1763-1811).

Needs remain for additional drugs to treat Nrf2-associated diseases, disorders or conditions, including ocular diseases or kidney diseases.

### SUMMARY OF THE INVENTION

In one aspect, the present disclosure provides a compound represented by Formula (I) below:

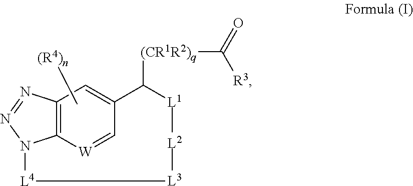

wherein
W is N or C;
L¹ is selected from the group consisting of

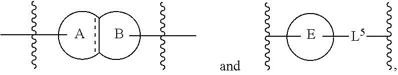

wherein
   Ring A is selected from the group consisting of cycloalkyl, heterocyclyl, aryl and heteroaryl, each of which is independently optionally substituted with one or more R^{a1};
   Ring B is selected from the group consisting of cycloalkyl, heterocyclyl, aryl and heteroaryl, each of which is independently optionally substituted with one or more R^{a2};
   --- is a bond via which Ring A is fused to Ring B;
   Ring E is selected from the group consisting of cycloalkyl, heterocyclyl, aryl and heteroaryl, each of which is independently optionally substituted with one or more R^{a3};
L² is selected from the group consisting of —C(O)—, —CR⁷R⁸—, —S(O)— and —S(O)₂—;
L³ is a bond or selected from the group consisting of cycloalkyl, heterocyclyl, aryl and heteroaryl, each of which is independently optionally substituted with one or more R^{a4}; provided that when W is C, L¹ is

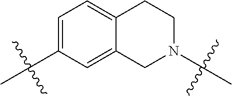 [SMILES: *c1ccc2c(c1)CN(*)CC2]

and L² is —C(O)—, L³ is not an aryl or heteroaryl;
L⁴ is selected from the group consisting of alkyl, alkylalkoxyl, alkenyl, alkynyl, heteroalkyl, heteroalkenyl and heteroalkynyl, wherein the alkyl, alkenyl and alkynyl, either alone or as part of another group, are independently optionally substituted with one or more R^{a5};
L⁵ is a bond or selected from the group consisting of alkyl, alkenyl, alkynyl, -alkyl-N(R^{b})—, heteroalkyl, heteroalkenyl and heteroalkynyl, wherein the alkyl, alkenyl, alkynyl and -alkyl-N(R^{b})—, either alone or as part of another group, are independently optionally substituted with one or more R^{a6};
each of R¹, R², R⁴, R⁷ and R⁸ is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl, wherein the alkyl, alkenyl and alkynyl, either alone or as part of another group, are independently optionally substituted with one or more R^{a7};
R³ is selected from the group consisting of hydrogen, halogen, hydroxyl, sulfhydryl, cyano, alkyl, alkenyl, alkynyl, heteroalkyl, heteroalkenyl, heteroalkynyl, haloalkyl, alkylalkoxyl, —OR^{c}, and —N(R^{d})₂, wherein the alkyl, alkenyl and alkynyl, either alone or as part of another group, are independently optionally substituted with one or more R^{a5};
each of R^{a1}, R^{a2}, R^{a3}, R^{a4}, R^{a5}, R^{a6}, R^{a7} and R^{a8} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl;
each of R^{b}, R^{c} and R^{d} is independently selected from the group consisting of hydrogen, alkyl, alkenyl, alkynyl, heteroalkyl, heteroalkenyl and heteroalkynyl, wherein the alkyl, alkenyl and alkynyl, either alone or as part of another group, are independently optionally substituted by one or more groups independently selected from the group consisting of hydrogen, halogen, hydroxyl, sulfhydryl, cyano, —NH₂ and —NO₂; or two R^{b} together with the nitrogen atom to which they are attached form a heterocyclyl; or two R^{d} together with the nitrogen atom to which they are attached form a heterocyclyl;
n is 0, 1, 2 or 3; and
q is 0, 1, 2 or 3,
or a tautomer, a stereoisomer, or a pharmaceutically acceptable salt thereof.

In another aspect, the present disclosure provides a compound, tautomer, stereoisomer, or pharmaceutically acceptable salt thereof, wherein the compound is represented by Formula (Ia) or Formula (Ib) below:

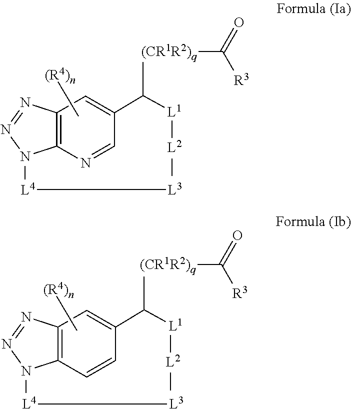

wherein each of R¹, R², R³, R⁴, L¹, L², L³ and L⁴, n and q are as defined above.

In another aspect, the present disclosure provides a compound, tautomer, stereoisomer, or pharmaceutically acceptable salt thereof, wherein the compound is represented by Formula (II), Formula (III) or Formula (IV) below:

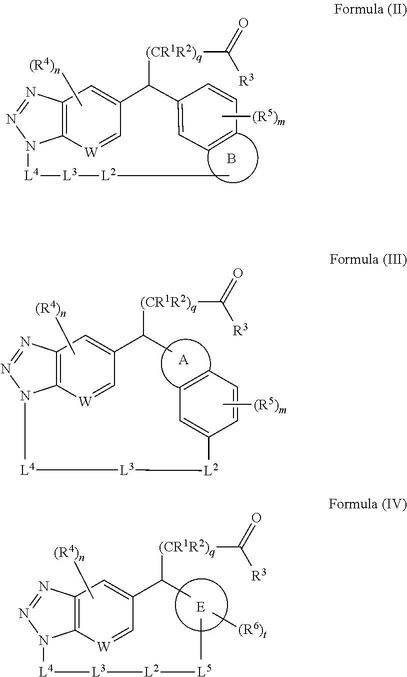

wherein,
each of R⁵ and R⁶ is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl, wherein the alkyl, alkenyl and alkynyl, either alone or as part of another group, are independently optionally substituted with one or more R^{a7};
m is 0, 1, 2 or 3;
t is 0, 1, 2 or 3; and
each of W, R¹, R², R³, R⁴, L², L³, L⁴, L⁵, Ring A, Ring B, Ring E, R^{a7}, n and q are as defined above.

In another aspect, the present disclosure provides a compound, tautomer, stereoisomer, or pharmaceutically acceptable salt thereof, wherein the compound is represented by Formula (IIa), Formula (IIb), Formula (IIIa), Formula (IIIb), Formula (IVa) or Formula (IVb) below:

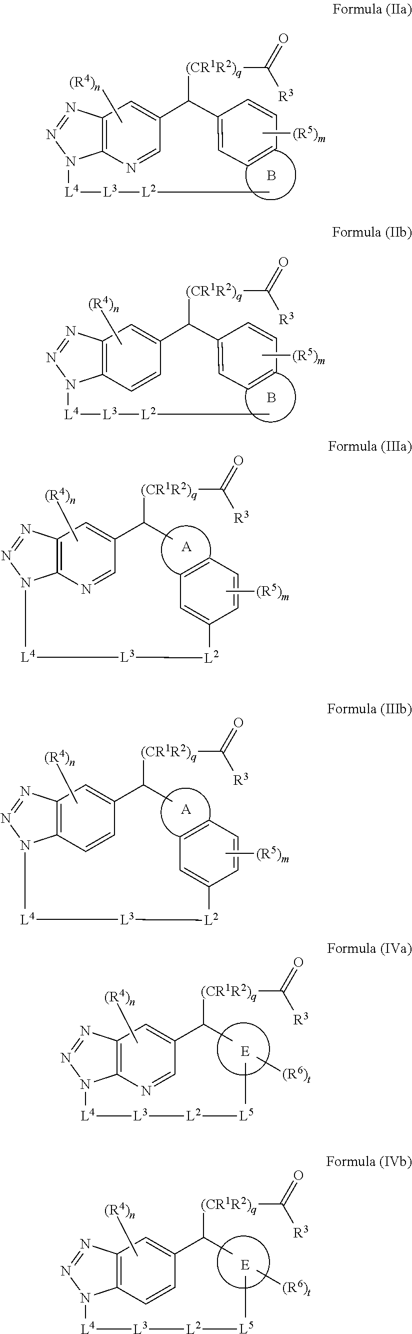

wherein, each of W, R¹, R², R³, R⁴, R⁵, R⁶, L², L³, L⁴, L⁵, Ring A, Ring B, Ring E, n, m, q and t are as defined above.

In another aspect, the present disclosure provides a compound, tautomer, stereoisomer, or pharmaceutically acceptable salt thereof, wherein the compound is represented by Formula (IIa1) or Formula (IIa2) below:

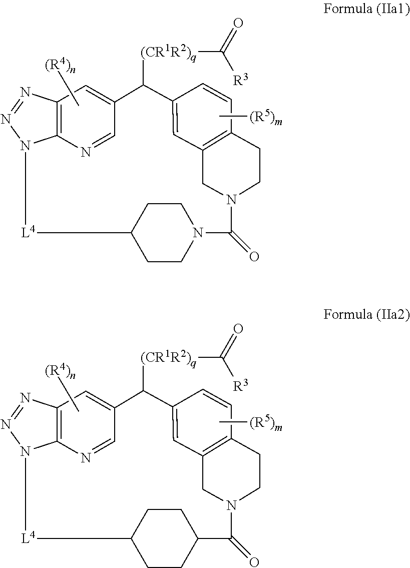

wherein,
L⁴ is C₃₋₈ alkyl or C₃₋₈ heteroalkyl;
R¹ is hydrogen, halogen or C₁₋₆ alkyl;
R² is hydrogen, halogen or C₁₋₆ alkyl;
R³ is —OR^{c}, R^{c} is hydrogen or C₁₋₆ alkyl;
R⁴ is hydrogen, halogen or C₁₋₆ alkyl;
R⁵ is hydrogen, halogen or C₁₋₆ alkyl;
n is 0 or 1;
m is 0 or 1; and
q is 1.

In another aspect, the present disclosure provides a compound, tautomer, stereoisomer, or pharmaceutically acceptable salt thereof, as shown in Table 1.1 and Table 1.2.

In another aspect, the present disclosure provides a pharmaceutical composition comprising the compound, tautomer, stereoisomer, or a pharmaceutically acceptable salt thereof described herein, and a pharmaceutically acceptable carrier or pharmaceutically acceptable excipient.

In another aspect, the present disclosure provides a method of increasing level or activity of Nrf2 in a cell, comprising exposing the cell to the compound, tautomer, stereoisomer, or a pharmaceutically acceptable salt thereof described herein, or the pharmaceutical composition described herein.

In another aspect, the present disclosure provides a method of increasing level or activity of Nrf2 in a subject in need thereof, comprising administering to the subject a therapeutically effective amount of the compound, tautomer, stereoisomer, or a pharmaceutically acceptable salt thereof described herein, or the pharmaceutical composition described herein.

In another aspect, the present disclosure provides a method of preventing, treating or alleviating a Nrf2-associated disease, disorder or condition in a subject in need thereof, comprising administering to the subject a therapeutically effective amount of the compound, tautomer, stereoisomer, or a pharmaceutically acceptable salt thereof described herein, or the pharmaceutical composition described herein.

### DETAILED DESCRIPTION OF THE INVENTION

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differ from or contradict this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination.

### Definitions

Unless defined otherwise, all technical and scientific terms used herein have the same meaning as commonly understood by one of ordinary skill in the art to which this invention belongs. As used herein, the following terms are intended to have the following meanings.

As used in the specification and claims, the singular forms “a,” “an,” and “the” and the like includes plural references unless the context clearly dictates otherwise. Thus, for example, reference to “a compound” includes both a single compound and a plurality of different compounds.

The term “approximately” or “about” as used herein intends to indicate that the values quoted are not to be construed as absolute, and measurement error, inter-batches variation and/or inter-apparatus variation should also be taken into account. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the values quoted unless otherwise stated or otherwise evident from the context (except where such number would exceed 100% of a possible value).

The terms “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

It is to be understood that the “compound” of present disclosure can exist in solvated as well as un-solvated forms, such as, for example, hydrated forms, solid forms, and the present disclosure is intended to encompass all such solvated and unsolvated forms. It is further to be understood that the “compound” of present disclosure can exist in forms of pharmaceutically acceptable salts. In some embodiments, the “compound” of present disclosure is an ionizable lipid. In some embodiments, the “compound” of present disclosure can exist as a cationic lipid at physiological pH.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula.

Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of R. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e., i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “C-j alkyl” refers to an alkyl having i to j carbon atoms. In some embodiments, alkyl groups contain 1 to 12 carbon atoms. In some embodiments, alkyl groups contain 1 to 11 carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

The alkyl groups can be further substituted by substituents which independently replace one or more hydrogen atoms on one or more carbons of the alkyl groups. Examples of such substituents can include, but are not limited to, acyl, alkyl, alkenyl, alkynyl, oxo, halogen, hydroxyl, alkoxyl, haloalkyl, haloalkoxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfmyl, sulfonate, sulfamoyl, sulfonamido, nitro, trifluoromethyl, cyano, nitro, azido, heterocyclyl, alkylaryl, or an aromatic or heteroaromatic moiety. Alkenyl, alkynyl, cycloalkyl, heterocyclyl, aryl and heteroaryl groups as described below may also be similarly substituted.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. In some embodiments, alkenyl groups contain one or more “Z” carbon-carbon double bond. Examples of alkenyl group include, but are not limited to, ethylenyl (or vinyl), propenyl, butenyl, pentenyl, 1-methyl-2 buten-1-yl, 5-hexenyl, and the like. In some embodiments, an alkenyl group has at least one carbon-carbon double bond. In some embodiments, an alkenyl group has at least two, at least three, at least four, at least five, at least six, at least seven, at least eight, at least nine, or at least ten carbon-carbon double bonds. In some embodiments, two or more carbon-carbon double bonds in an alkenyl group are conjugated. In some embodiments, two or more carbon-carbon double bonds in an alkenyl group are not conjugated. In some embodiments, two or more carbon-carbon double bonds in an alkenyl group are isolated, cumulated or conjugated.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkynyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like. In some embodiments, an alkynyl group has at least one carbon-carbon triple bond. In some embodiments, an alkynyl group has at least two, at least three, at least four, at least five, at least six, at least seven, at least eight, at least nine, or at least ten carbon-carbon triple bonds. In some embodiments, two or more carbon-carbon triple bonds in an alkynyl group are conjugated. In some embodiments, two or more carbon-carbon triple bonds in an alkynyl group are not conjugated.

As used herein, the term “alkoxyl”, whether as part of another term or used independently, refers to an alkyl group, as previously defined, attached to the parent molecule through an oxygen atom. The term “Cᵢ₋ⱼ alkoxyl” means that the alkyl moiety of the alkoxy group has i to j carbon atoms. In some embodiments, alkoxy groups contain 1 to 12 carbon atoms. In some embodiments, alkoxy groups contain 1 to 11 carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g., n-propoxy and isopropoxy), t-butoxy, neopentoxy, n-hexoxy, and the like.

As used herein, the term “alkylalkoxyl”, whether as part of another term or used independently, refers to an alkyl moiety substituted with one or more alkoxyl moiety. The “alkylalkoxyl” can be bonded to the parent molecular structure through the alkyl group or the alkoxyl group.

As used herein, the term “alkylcycloalkyl”, whether as part of another term or used independently, refers to an alkyl moiety substituted with one or more cycloalkyl moiety. The “alkylcycloalkyl” can be bonded to the parent molecular structure through the alkyl group or the cycloalkyl group.

As used herein, the term “amino” refers to —NH₂. In some embodiments, amino may be substituted by any possible substituents on nitrogen.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all the rings may be aromatic (e.g., quinoline). The second ring can also be fused, bridged or spiro. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 11 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 3 to 4 ring forming carbon atoms, 4 to 12 ring forming carbon atoms, 4 to 11 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system.

In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

In some embodiments, the cycloalkyl group may be saturated or partially unsaturated polycyclic (e.g., bicyclic and tricyclic) carbocyclic ring system, which can be arranged as a fused-, spiro- or bridged-ring system. As used herein, the term “fused-ring” refers to a ring system having two rings sharing two adjacent atoms, the term “spiro-ring” refers to a ring systems having two rings connected through one single common atom, and the term “bridged-ring” refers to a ring system with two rings sharing three or more atoms. Examples of fused carbocyclyl include, but are not limited to, naphthyl, benzopyrenyl, anthracenyl, acenaphthenyl, fluorenyl and the like. Examples of spiro carbocyclyl include, but are not limited to, spiro[5.5]undecanyl, spiro-pentadienyl, spiro[3.6]-decanyl, and the like. Examples of bridged carbocyclyl include, but are not limited to bicyclo[1,1,1]pentenyl, bicyclo[2,2,1]heptenyl, bicyclo[2.2.1]heptanyl, bicyclo[2.2.2]octanyl, bicyclo[3.3.1]nonanyl, bicyclo[3.3.3]undecanyl, and the like.

As used herein, the term “cyano” refers to —CN.

As used herein, the term “halogen” refers to an atom selected from fluorine (or fluoro), chlorine (or chloro), bromine (or bromo) and iodine (or iodo).

As used herein, the term “haloalkyl”, whether as part of another term or used independently, refers to an alkyl group having one or more halogen substituents. Examples of haloalkyl group include, but are not limited to, trifluoromethyl (—CF₃), pentafluoroethyl (—C₂F₅), difluoromethyl (—CHF₂), trichloromethyl (—CCl₃), dichloromethyl (—CHCl₂), pentachloroethyl (—C₂Cl₅), and the like.

As used herein, the term “haloalkoxyl”, whether as part of another term or used independently, refers to an alkoxyl group having one or more halogen substituents. As a result, the term “halo-Cᵢ₋ⱼ alkoxyl”, whether as part of another term or used independently, refers to a Cᵢ₋ⱼ alkoxyl group having one or more halogen substituents. Examples of haloalkoxyl include, but are not limited to, —O—CF₃, —O—C₂F₅, —O—CHF₂, —O—CCl₃, —O—CHCl₂, —O—C₂Cl₅, and the like.

As used herein, the term “heteroatom” refers to nitrogen (N), oxygen (O), sulfur (S), and includes any oxidized form of nitrogen or sulfur, and any quaternized form of a basic nitrogen (including N-oxides).

As used herein, the term “heteroalkyl”, “heteroalkenyl”, or “heteroalkynyl”, whether as part of another term or used independently, refers to an alkyl, alkenyl, or alkynyl group containing one or more heteroatoms. As a result, the term “hetero-Cᵢ₋ⱼ alkyl”, “hetero-Cᵢ₋ⱼ alkenyl”, “hetero-Cᵢ₋ⱼ alkynyl”, “Cᵢ₋ⱼ heteroalkyl”, “Cᵢ₋ⱼ heteroalkenyl” or “Cᵢ₋ⱼ heteroalkynyl”, whether as part of another term or used independently, refers to a Cᵢ₋ⱼ alkyl, Cᵢ₋ⱼ alkenyl, or Cᵢ₋ⱼ alkynyl containing one or more heteroatoms. For example, the term “hetero-C₁₋₆ alkyl” or “C₁₋₆ heteroalky”, whether as part of another term or used independently, refers to a C₁₋₆ alkyl containing one or more heteroatoms. In some embodiments, a heteroalkyl, heteroalkenyl or heteroalkynyl group contains at least one heteroatom. In some embodiments, a heteroalkyl, heteroalkenyl or heteroalkynyl group contains at least two, at least three, at least four, at least five, at least six, at least seven, at least eight, at least nine or at least ten heteroatoms. In some embodiments, two or more heteroatoms in a heteroalkyl, heteroalkenyl or heteroalkynyl group are the same. In some embodiments, two or more heteroatoms in a heteroalkyl, heteroalkenyl or heteroalkynyl group are different. In some embodiments, two or more heteroatoms in a heteroalkyl, heteroalkenyl or heteroalkynyl group are directly bonded. In some embodiments, two or more heteroatoms in a heteroalkyl, heteroalkenyl or heteroalkynyl group are not directly bonded.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, heteroaryl, cycloaliphatic, or heterocyclyl rings, where the radical or point of attachment is on the heteroaromatic ring or the other ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

Heterocyclyl group may be monocyclic. Examples of monocyclic heterocyclyl include, but are not limited to oxetanyl, 1,1-dioxothietanylpyrrolidyl, tetrahydrofuryl, tetrahydropyranyl, tetrahydrothienyl, azetidinyl, pyrrolyl, furanyl, thienyl, pyrazolyl, imidazolyl, triazolyl, oxazolyl, thiazolyl, piperidyl, piperazinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like.

Heterocyclyl group may be polycyclic, including the fused-, spiro- and bridged-ring systems. The fused heterocyclyl group includes radicals wherein the heterocyclyl radicals are fused with a saturated, partially unsaturated, or fully unsaturated (i.e., aromatic) carbocyclic or heterocyclic ring. Examples of fused heterocyclyl include, but are not limited to, phenyl fused-ring or pyridinyl fused-ring, such as quinolinyl, isoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, 5-aza-spiro[2.4]heptanyl, 6-aza-spiro[2.5]octanyl, 6-aza-spiro[3.4]octanyl, 2-oxa-6-aza-spiro[3.3]heptanyl, 2-oxa-6-aza-spiro[3.4]octanyl, 6-aza-spiro[3.5]nonanyl, 7-aza-spiro[3.5]nonanyl, 1-oxa-7-aza-spiro[3.5]nonanyl and the like. Examples of bridged heterocyclyl include, but are not limited to, 3-aza-bicyclo[3.1.0]hexanyl, 8-aza-bicyclo[3.2.1]octanyl, 1-aza-bicyclo[2.2.2]octanyl, 2-aza-bicyclo[2.2.1]heptanyl, 1,4-diazabicyclo[2.2.2]octanyl, and the like.

As used herein, the term “hydroxyl” or “hydroxy” refers to —OH.

As used herein, the term “sulfhydryl” refers to —SH.

As used herein, the term “sulfonyl” refers to —SO₂R′, wherein R′ is selected from hydrogen, alkyl, alkenyl, alkynyl, haloalkyl, cycloalkyl, heterocyclyl, aryl, or heteroaryl.

As used herein, the term “-Boc” refers to t-butoxyl carbonyl.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution”, “substituted by” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compounds

The present disclosure provides novel compounds or tautomers, stereoisomers, or pharmaceutically acceptable salts thereof, synthetic methods for making the compounds, pharmaceutical compositions containing them and various uses of the disclosed compounds.

In one aspect, the present disclosure provides a compound represented by Formula (I) below:

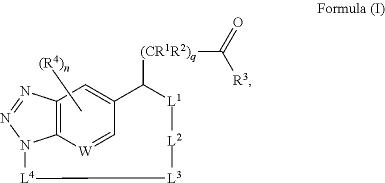

wherein
W is N or C;
L¹ is selected from the group consisting of

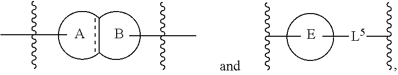

wherein
   Ring A is selected from the group consisting of cycloalkyl, heterocyclyl, aryl and heteroaryl, each of which is independently optionally substituted with one or more R^{a1};
   Ring B is selected from the group consisting of cycloalkyl, heterocyclyl, aryl and heteroaryl, each of which is independently optionally substituted with one or more R^{a2};
   --- is a bond via which Ring A is fused to Ring B;
   Ring E is selected from the group consisting of cycloalkyl, heterocyclyl, aryl and heteroaryl, each of which is independently optionally substituted with one or more R^{a3};
L² is selected from the group consisting of —C(O)—, —CR⁷R⁸—, —S(O)— and —S(O)₂—;
L³ is a bond or selected from the group consisting of cycloalkyl, heterocyclyl, aryl and heteroaryl, each of which is independently optionally substituted with one or more R^{a4}; provided that when W is C, L¹ is

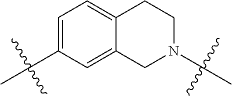 [SMILES: *c1ccc2c(c1)CN(*)CC2]

and L² is —C(O)—, L³ is not an aryl or heteroaryl;
L⁴ is selected from the group consisting of alkyl, alkylalkoxyl, alkenyl, alkynyl, heteroalkyl, heteroalkenyl and heteroalkynyl, wherein the alkyl, alkenyl and alkynyl, either alone or as part of another group, are independently optionally substituted with one or more R^{a5};
L⁵ is a bond or selected from the group consisting of alkyl, alkenyl, alkynyl, -alkyl-N(R^{b})—, heteroalkyl, heteroalkenyl and heteroalkynyl, wherein the alkyl, alkenyl, alkynyl and -alkyl-N(R^{b})—, either alone or as part of another group, are independently optionally substituted with one or more R^{a6};
each of R¹, R², R⁴, R⁷ and R⁸ is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl, wherein the alkyl, alkenyl and alkynyl, either alone or as part of another group, are independently optionally substituted with one or more R^{a7};
R³ is selected from the group consisting of hydrogen, halogen, hydroxyl, sulfhydryl, cyano, alkyl, alkenyl, alkynyl, heteroalkyl, heteroalkenyl, heteroalkynyl, haloalkyl, alkylalkoxyl, —OR^{c}, —NHR^{d} and —N(R^{d})₂, wherein the alkyl, alkenyl and alkynyl, either alone or as part of another group, are independently optionally substituted with one or more R^{a5};
each of R^{a1}, R^{a2}, R^{a3}, R^{a4}, R^{a5}, R^{a6}, R^{a7} and R^{a8} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl;
each of R^{b}, R^{c} and R^{d} is independently selected from the group consisting of hydrogen, alkyl, alkenyl, alkynyl, heteroalkyl, heteroalkenyl and heteroalkynyl, wherein the alkyl, alkenyl and alkynyl, either alone or as part of another group, are independently optionally substituted by one or more groups independently selected from the group consisting of hydrogen, halogen, hydroxyl sulfhydryl, cyano, —NH₂ and —NO₂; or two R^{b} together with the nitrogen atom to which they are attached form a heterocyclyl; or two R^{d} together with the nitrogen atom to which they are attached form a heterocyclyl;
n is 0, 1, 2 or 3; and
q is 0, 1, 2 or 3,
or a tautomer, a stereoisomer, or a pharmaceutically acceptable salt thereof.

In some embodiments, the compound is represented by Formula (Ia) or Formula (Ib) below.

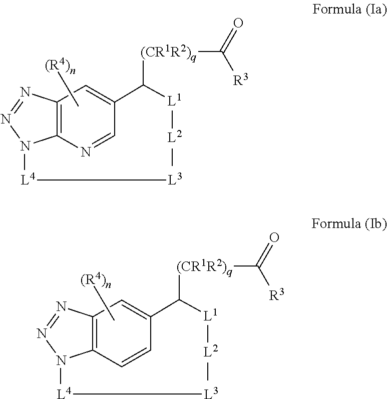

wherein each of R¹, R², R³, R⁴, L¹, L², L³ and L⁴, n and q are as defined above.

In some embodiments, L¹ is

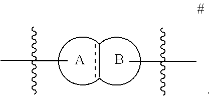

In some embodiments, L¹ is

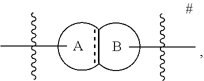

and # is linked to L².

In some embodiments, Ring A is aryl optionally substituted with one or more R^{a1} wherein each R^{a1} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. For example, Ring A is aryl optionally substituted with one, two, three, four, five, six, seven, eight, nine, ten, or more R^{a1}. In some embodiments, Ring A is a 3- to 12-membered aryl, 3- to 11-membered aryl, 3- to 10-membered aryl, 3- to 9-membered aryl, 3- to 8-membered aryl, 3- to 7-membered aryl, 3- to 6-membered aryl, 3- to 5-membered aryl, or 3- to 4-membered aryl, which is optionally substituted with one or more R^{a1}, wherein each R^{a1} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, Ring A is a monocyclic aryl optionally substituted with one or more R^{a1}. For example, Ring A is a monocyclic aryl optionally substituted with one, two, three, four, five, six, seven, eight, nine, ten, or more R^{a1}. In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic aryl optionally substituted with one or more (e.g., two, three, four, five, six, seven, eight, nine, ten or more) R^{a1}. In some embodiments, Ring A is a phenyl optionally substituted with one or more (e.g., two, three, four, five, six, seven, eight, nine, ten or more) R^{a1}, wherein each R^{a1} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. In some embodiments, Ring A is an unsubstituted phenyl. In some embodiments, Ring A is a phenyl substituted with a group selected from the group consisting of halogen, hydroxyl, sulfhydryl, cyano, —NH₂, —NO₂, C₁₋₆ alkoxyl, C₁₋₆ alkyl, C₁₋₆ alkenyl, C₁₋₆ alkynyl, C₁₋₆ heteroalkyl, C₁₋₆ heteroalkenyl, C₁₋₆ heteroalkynyl and halo-C₁₋₆ alkyl. In some embodiments, Ring A is a phenyl substituted with a C₁₋₆ alkyl (e.g., methyl, ethyl, propyl, butyl, pentyl, hexyl). In some embodiments, Ring A is a phenyl substituted with a methyl. In some embodiments, Ring A is

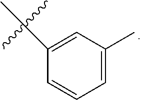 [SMILES: *c1cccc(C)c1]

In some embodiments, Ring A is

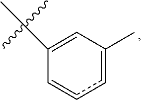

wherein --- is a bond via which Ring A is fused to Ring B.

In some embodiments, Ring A is a heterocyclyl optionally substituted with one or more R^{a1}, wherein each R^{a1} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. For example, Ring A is a heterocyclyl optionally substituted with one, two, three, four, five, six, seven, eight, nine, ten, or more R^{a1}. In some embodiments, Ring A is a 3- to 12-membered heterocyclyl, 3- to 11-membered heterocyclyl, 3- to 10-membered heterocyclyl, 3- to 9-membered heterocyclyl, 3- to 8-membered heterocyclyl, 3- to 7-membered heterocyclyl, 3- to 6-membered heterocyclyl, 3- to 5-membered heterocyclyl, or 3- to 4-membered heterocyclyl, which is optionally substituted with one or more R^{a1}, wherein each R^{a1} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, Ring A is a monocyclic heterocyclyl optionally substituted with one or more R^{a1}. For example, Ring A is a monocyclic heterocyclyl optionally substituted with one, two, three, four, five, six, seven, eight, nine, ten, or more R^{a1}. In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl optionally substituted with one or more (e.g., two, three, four, five, six, seven, eight, nine, ten or more) R^{a1}. In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or more (e.g., 1, 2, 3, 4 or more) heteroatoms (e.g., O, N, S), which is optionally substituted with one or more (e.g., two, three, four, five, six, seven, eight, nine, ten, or more) R^{a1} In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or more (e.g., 1, 2, 3, 4 or more) nitrogen atoms, which is optionally substituted with one or more (e.g., two, three, four, five, six, seven, eight, nine, ten, or more) R^{a1}. In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or two nitrogen atoms, which is optionally substituted with one or more (e.g., two, three, four, five, six, seven, eight, nine, ten, or more) R^{a1}.

In some embodiments, Ring A is a saturated monocyclic heterocyclyl optionally substituted with one or more (e.g., two, three, four, five, six, seven, eight, nine, ten, or more) R^{a1} In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl optionally substituted with one or more (e.g., two, three, four, five, six, seven, eight, nine, ten or more) R^{a1}. In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl containing one or more (e.g., 1, 2, 3, 4 or more) heteroatoms (e.g., O, N, S), which is optionally substituted with one or more (e.g., two, three, four, five, six, seven, eight, nine, ten, or more) R^{a1}. In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl containing one or more (e.g., 1, 2, 3, 4 or more) nitrogen atoms, which is optionally substituted with one or more (e.g., two, three, four, five, six, seven, eight, nine, ten, or more) R^{a1}. In some embodiments, Ring A is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl containing one or two nitrogen atoms, which is optionally substituted with one or more (e.g., two, three, four, five, six, seven, eight, nine, ten, or more) R^{a1}. In some embodiments, Ring A is piperidinyl optionally substituted with one or more (e.g., two, three, four, five, six, seven, eight, nine, ten, or more) R^{a1}. In some embodiments, Ring A is an unsubstituted piperidinyl. In some embodiments, Ring A is

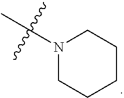 [SMILES: *N1CCCCC1]

In some embodiments, Ring A is

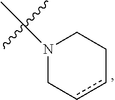

wherein --- is a bond via which Ring A is fused to Ring B.

In some embodiments, Ring B is heterocyclyl or aryl, each of which is independently optionally substituted with one or more (e.g., two, three, four, five, six, seven, eight, nine, ten or more) R^{a2}. In some embodiments, Ring A and Ring B are not heterocyclyl or aryl at the same time. For example, when Ring A is heterocyclyl, Ring B is aryl; when Ring A is aryl, Ring B is heterocyclyl.

In some embodiments, Ring B is a heterocyclyl optionally substituted with one or more R^{a2}, wherein each R^{a2} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. For example, Ring B is a heterocyclyl optionally substituted with one, two, three, four, five, six, seven, eight, nine, ten, or more R^{a2}. In some embodiments, Ring B is a 3- to 12-membered heterocyclyl, 3- to 11-membered heterocyclyl, 3- to 10-membered heterocyclyl, 3- to 9-membered heterocyclyl, 3- to 8-membered heterocyclyl, 3- to 7-membered heterocyclyl, 3- to 6-membered heterocyclyl, 3- to 5-membered heterocyclyl, or 3- to 4-membered heterocyclyl, which is optionally substituted with one or more R^{a2}, wherein each R^{a2} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, Ring B is a monocyclic heterocyclyl optionally substituted with one or more R^{a2}. For example, Ring B is a monocyclic heterocyclyl optionally substituted with one, two, three, four, five, six, seven, eight, nine, ten, or more R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl optionally substituted with one or more (e.g., two, three, four, five, six, seven, eight, nine, ten or more) R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or more (e.g., 1, 2, 3, 4 or more) heteroatoms (e.g., O, N, S), which is optionally substituted with one or more (e.g., two, three, four, five, six, seven, eight, nine, ten, or more) R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or more (e.g., 1, 2, 3, 4 or more) nitrogen atoms, which is optionally substituted with one or more (e.g., two, three, four, five, six, seven, eight, nine, ten, or more) R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or two nitrogen atoms, which is optionally substituted with one or more (e.g., two, three, four, five, six, seven, eight, nine, ten, or more) R^{a2}.

In some embodiments, Ring B is a saturated monocyclic heterocyclyl optionally substituted with one or more (e.g., two, three, four, five, six, seven, eight, nine, ten, or more) R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl optionally substituted with one or more (e.g., two, three, four, five, six, seven, eight, nine, ten or more) R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl containing one or more (e.g., 1, 2, 3, 4 or more) heteroatoms (e.g., O, N, S), which is optionally substituted with one or more (e.g., two, three, four, five, six, seven, eight, nine, ten, or more) R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl containing one or more (e.g., 1, 2, 3, 4 or more) nitrogen atoms, which is optionally substituted with one or more (e.g., two, three, four, five, six, seven, eight, nine, ten, or more) R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) saturated monocyclic heterocyclyl containing one or two nitrogen atoms, which is optionally substituted with one or more (e.g., two, three, four, five, six, seven, eight, nine, ten, or more) R^{a2}. In some embodiments, Ring B is piperidinyl optionally substituted with one or more (e.g., two, three, four, five, six, seven, eight, nine, ten, or more) R^{a2}. In some embodiments, Ring B is an unsubstituted piperidinyl. In some embodiments, Ring B is

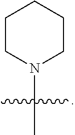 [SMILES: *N1CCCCC1]

In some embodiments, Ring B is

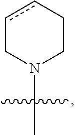

wherein --- is a bond via which Ring B is fused to Ring A.

In some embodiments, Ring B is aryl optionally substituted with one or more R^{a2}, wherein each R^{a2} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. For example, Ring B is aryl optionally substituted with one, two, three, four, five, six, seven, eight, nine, ten, or more R^{a2}. In some embodiments, Ring B is a 3- to 12-membered aryl, 3- to 11-membered aryl, 3- to 10-membered aryl, 3- to 9-membered aryl, 3- to 8-membered aryl, 3- to 7-membered aryl, 3- to 6-membered aryl, 3- to 5-membered aryl, or 3- to 4-membered aryl, which is optionally substituted with one or more R^{a2}, wherein each R^{a2} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, Ring B is a monocyclic aryl optionally substituted with one or more R^{a2}. For example, Ring B is a monocyclic aryl optionally substituted with one, two, three, four, five, six, seven, eight, nine, ten, or more R^{a2}. In some embodiments, Ring B is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic aryl optionally substituted with one or more (e.g., two, three, four, five, six, seven, eight, nine, ten or more) R^{a2}. In some embodiments, Ring B is a phenyl optionally substituted with one or more (e.g., two, three, four, five, six, seven, eight, nine, ten or more) R^{a2}, wherein each R^{a2} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. In some embodiments, Ring B is an unsubstituted phenyl. In some embodiments, Ring B is a phenyl substituted with a group selected from the group consisting of halogen, hydroxyl, sulfhydryl, cyano, —NH₂, —NO₂, C₁₋₆ alkoxyl, C₁₋₆ alkyl, C₁₋₆ alkenyl, C₁₋₆ alkynyl, C₁₋₆ heteroalkyl, C₁₋₆ heteroalkenyl, C₁₋₆ heteroalkynyl and halo-C₁₋₆ alkyl. In some embodiments, Ring B is a phenyl substituted with a C₁₋₆ alkyl (e.g., methyl, ethyl, propyl, butyl, pentyl, hexyl). In some embodiments, Ring B is a phenyl substituted with a methyl. In some embodiments, Ring B is

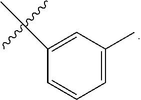 [SMILES: *c1cccc(C)c1]

In some embodiments, Ring B is

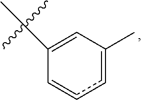

wherein --- is a bond via which Ring B is fused to Ring A.

In some embodiments, Ring A is a phenyl optionally substituted with one or more R^{a1} and Ring B is a piperidinyl optionally substituted with one or more R^{a2}. In some embodiments, Ring A is a phenyl substituted with a C₁₋₆ alkyl (e.g., methyl), and Ring B is an unsubstituted piperidinyl. In some embodiments, Ring A is an unsubstituted phenyl, and Ring B is an unsubstituted piperidinyl.

In some embodiments, Ring A is a piperidinyl optionally substituted with one or more R^{a1}, and Ring B is a phenyl optionally substituted with one or more R^{a2}. In some embodiments, Ring A is an unsubstituted piperidinyl, and Ring B is a phenyl substituted with a C₁₋₆ alkyl (e.g., methyl). In some embodiments, Ring A is an unsubstituted piperidinyl, and Ring B is an unsubstituted phenyl.

In some embodiments, Ring A is a phenyl optionally substituted with one or more R^{a1}, and Ring B is a phenyl optionally substituted with one or more R^{a2}. In some embodiments, both Ring A and Ring B are unsubstituted phenyl.

In some embodiments, L¹ is

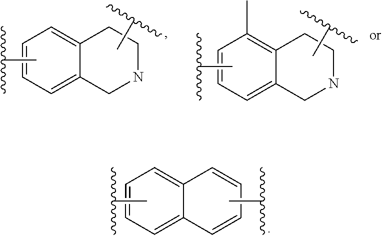

In some embodiments, L¹ is

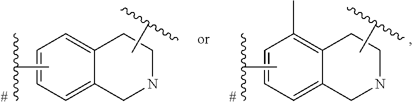

and # is linked to L². In some embodiments, L¹ is

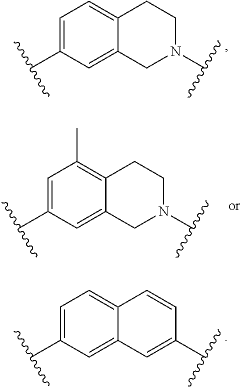 [SMILES: *c1ccc2c(c1)CN(*)CC2 ; *c1cc(C)c2c(c1)CN(*)CC2 ; *c1ccc2ccc(*)cc2c1]

In some embodiments, L¹ is

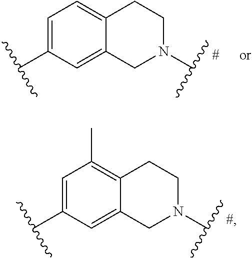 [SMILES: *c1ccc2c(c1)CN(*)CC2 ; *c1cc(C)c2c(c1)CN(*)CC2]

and # is linked to L². In some embodiments, L¹ is

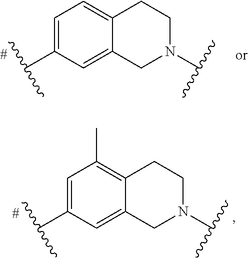 [SMILES: *c1ccc2c(c1)CN(*)CC2 ; *c1cc(C)c2c(c1)CN(*)CC2]

and # is linked to L². In some embodiments, L¹ is

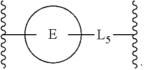

In some embodiments, Ring E is aryl optionally substituted with one or more R^{a3}, wherein each R^{a3} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. For example, Ring E is aryl optionally substituted with one, two, three, four, five, six, seven, eight, nine, ten, or more R^{a3}. In some embodiments, Ring E is a 3- to 12-membered aryl, 3- to 11-membered aryl, 3- to 10-membered aryl, 3- to 9-membered aryl, 3- to 8-membered aryl, 3- to 7-membered aryl, 3- to 6-membered aryl, 3- to 5-membered aryl, or 3- to 4-membered aryl, which is optionally substituted with one or more (e.g., two, three, four, five, six, seven, eight, nine, ten, or more) R^{a3}, wherein each R^{a3} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, Ring E is a monocyclic aryl optionally substituted with one or more R^{a3}. For example, Ring E is a monocyclic aryl optionally substituted with one, two, three, four, five, six, seven, eight, nine, ten, or more R^{a3}. In some embodiments, Ring E is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic aryl optionally substituted with one or more (e.g., two, three, four, five, six, seven, eight, nine, ten or more) R^{a3}. In some embodiments, Ring E is a phenyl optionally substituted with one or more (e.g., two, three, four, five, six, seven, eight, nine, ten or more) R^{a3}, wherein each R^{a3} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, Ring E is heteroaryl optionally substituted with one or more R wherein each R^{a3} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. For example, Ring E is heteroaryl optionally substituted with one, two, three, four, five, six, seven, eight, nine, ten, or more R^{a3}. In some embodiments, Ring E is a 3- to 12-membered heteroaryl, 3- to 11-membered heteroaryl, 3- to 10-membered heteroaryl, 3- to 9-membered heteroaryl, 3- to 8-membered heteroaryl, 3- to 7-membered heteroaryl, 3- to 6-membered heteroaryl, 3- to 5-membered heteroaryl, 3- to 4-membered heteroaryl, which is optionally substituted with one or more (e.g., two, three, four, five, six, seven, eight, nine, ten, or more) R^{a3}, wherein each R^{a3} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, Ring E is a monocyclic heteroaryl optionally substituted with one or more R^{a3}. For example, Ring E is a monocyclic heteroaryl optionally substituted with one, two, three, four, five, six, seven, eight, nine, ten, or more R^{a3}. In some embodiments, Ring E is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heteroaryl optionally substituted with one or more (e.g., two, three, four, five, six, seven, eight, nine, ten or more) R^{a3}. In some embodiments, Ring E is a monocyclic heteroaryl containing one or more (e.g., 1, 2, 3, 4 or more) heteroatoms (e.g., O, N, S), which is optionally substituted with one or more (e.g., two, three, four, five, six, seven, eight, nine, ten, or more) R^{a3}. In some embodiments, Ring E is a monocyclic heteroaryl containing one or more (e.g., 1, 2, 3, 4 or more) nitrogen atoms, which is optionally substituted with one or more (e.g., two, three, four, five, six, seven, eight, nine, ten, or more) R^{a3}. In some embodiments, Ring E is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heteroaryl containing one or two nitrogen atoms, which is optionally substituted with one or more (e.g., two, three, four, five, six, seven, eight, nine, ten, or more) R^{a3}. In some embodiments, Ring E is pyridinyl optionally substituted with one or more (e.g., two, three, four, five, six, seven, eight, nine, ten, or more) R^{a3}.

In some embodiments, L¹ is

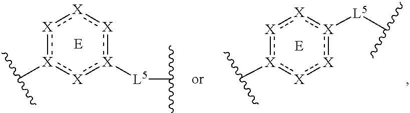

or each of X is independently selected from the group consisting of C, CR^{a3}, CH N, NR^{a3}, and NH.

In some embodiments, L¹ is

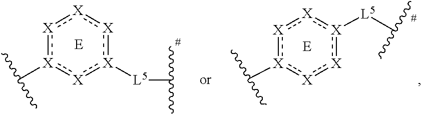

each of X is independently selected from the group consisting of C, CR^{a3}, CH, N, NR^{a3}, and NH, and # is linked to L².

In some embodiments, L⁵ is a heteroalkyl optionally substituted with one or more (e.g., two, three, four, five, six, seven, eight, nine, ten, or more) R^{a6}, wherein each R^{a6} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. In some embodiments, L⁵ is a heteroalkyl containing one or more (e.g., 1, 2, 3, 4 or more) heteroatoms (e.g., O, N, S), which is optionally substituted with one or more (e.g., two, three, four, five, six, seven, eight, nine, ten, or more) R^{a6}. In some embodiments, L⁵ is a heteroalkyl containing at least one (e.g., 1, 2, 3, 4, 5 or more) nitrogen atom and optionally substituted with one or more (e.g., two, three, four, five, six, seven, eight, nine, ten, or more) R^{a6} In some embodiments, L⁵ is a heteroalkyl containing one to six carbon atoms and at least one (e.g., 1, 2, 3, 4, 5 or more) nitrogen atom and optionally substituted with one or more (e.g., two, three, four, five, six, seven, eight, nine, ten, or more) R^{a6}. In some embodiments, L⁵ is selected from the group consisting of —CH₂—N(R^{a6})—, —(CH₂)₂—N(R^{a6})—, —(CH₂)₃—N(R^{a6})—, —(CH₂)₄—N(R^{a6})—, —(CH₂)₅—N(R^{a6})— and —(CH₂)₆—N(R^{a6})—. In some embodiments, R^{a6} is alkyl. In some embodiments, R^{a6} is C₁₋₆ alkyl (e.g., methyl, ethyl, propyl, butyl, pentyl, hexyl). In some embodiments, R^{a6} is methyl.

In some embodiments, L¹ is

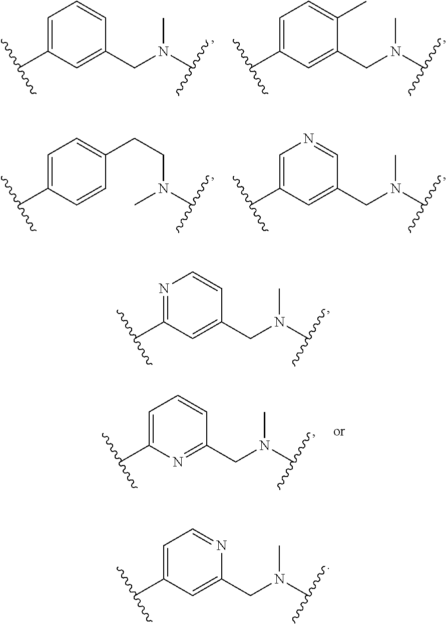

In some embodiments, L² is —C(O)— or —S(O)₂—.

In some embodiments, L³ is a bond. In some embodiments, L³ is a cycloalkyl. In some embodiments, L³ is a C₃₋₁₂ cycloalkyl (e.g., a cycloalkyl containing three, four, five, six, seven, eight, nine, ten, eleven or twelve carbon atoms). In some embodiments, L³ is a C₃₋₁₁ cycloalkyl, C₃₋₁₀ cycloalkyl, C₃₋₉ cycloalkyl, C₃₋₈ cycloalkyl, C₃₋₇ cycloalkyl, C₃₋₆ cycloalkyl, C₃₋₅ cycloalkyl or C₃₋₄ cycloalkyl. In some embodiments, L³ is an unsubstituted C₃₋₁₂ cycloalkyl (e.g., a cycloalkyl containing three, four, five, six, seven, eight, nine, ten, eleven or twelve carbon atoms). In some embodiments, L³ is a C₃₋₁₂ cycloalkyl (e.g., a cycloalkyl containing three, four, five, six, seven, eight, nine, ten, eleven or twelve carbon atoms) substituted with a group selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. In some embodiments, L³ is a cyclohexyl. In some embodiments, L³ is

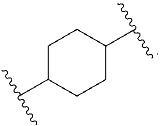 [SMILES: *C1CCC(*)CC1]

In some embodiments, L³ is a heterocyclyl. In some embodiments, L³ is a 3- to 12-membered heterocyclyl, 3- to 11-membered heterocyclyl, 3- to 10-membered heterocyclyl, 3- to 9-membered heterocyclyl, 3- to 8-membered heterocyclyl, 3- to 7-membered heterocyclyl, 3- to 6-membered heterocyclyl, 3- to 5-membered heterocyclyl, or 3- to 4-membered heterocyclyl. In some embodiments, L³ is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) heterocyclyl containing one or more (e.g., 1, 2, 3, 4 or more) heteroatoms (e.g., O, N, S). In some embodiments, L³ is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) heterocyclyl containing one or more (e.g., 1, 2, 3, 4 or more) nitrogen atoms. In some embodiments, the heterocyclyl is unsubstituted or substituted with a group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, L³ is a monocyclic heterocyclyl. In some embodiments, L³ is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl. In some embodiments, L³ is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or more (e.g., 1, 2, 3, 4 or more) heteroatoms (e.g., O, N, S). In some embodiments, L³ is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic heterocyclyl containing one or more (e.g., 1, 2, 3, 4 or more) nitrogen atoms. In some embodiments, the monocyclic heterocyclyl is unsubstituted or substituted with a group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. In some embodiments, L³ is piperidinyl or piperazinyl. In some embodiments, L³ is

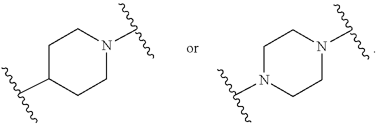 [SMILES: *C1CCN(*)CC1 ; *N1CCN(*)CC1]

In some embodiments, L³ is

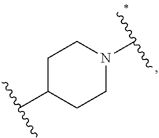 [SMILES: *C1CCN(*)CC1]

and * is linked to L².

In some embodiments, L³ is aryl. In some embodiments, L³ is a 3- to 12-membered aryl, 3- to 11-membered aryl, 3- to 10-membered aryl, 3- to 9-membered aryl, 3- to 8-membered aryl, 3- to 7-membered aryl, 3- to 6-membered aryl, 3- to 5-membered aryl, or 3- to 4-membered aryl. In some embodiments, the aryl is unsubstituted or substituted with a group selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, L³ is a monocyclic aryl. In some embodiments, L³ is a 3- to 12-membered (e.g., 3- to 11-membered, 3- to 10-membered, 3- to 9-membered, 3- to 8-membered, 3- to 7-membered, 3- to 6-membered, 3- to 5-membered, or 3- to 4-membered) monocyclic aryl. In some embodiments, L³ is phenyl. In some embodiments, the monocyclic aryl or phenyl is unsubstituted or substituted with a group selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl. In some embodiments, L³ is

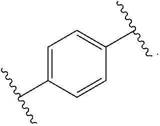 [SMILES: *c1ccc(*)cc1]

In some embodiments, L⁴ is selected from the group consisting of alkyl, alkenyl, heteroalkyl or heteroalkenyl, wherein the alkyl, alkenyl, heteroalkyl and heteroalkenyl are independently optionally substituted with one or more (e.g., two, three, four, five, six, seven, eight, nine, ten or more) R^{a5}, wherein each R^{a5} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, L⁴ is C₁₋₂ alkyl (e.g., alkyl containing one, two, three, four, five, six, seven, eight, nine, ten, eleven or twelve carbon atoms) or C₂₋₁₂ alkenyl (e.g., alkenyl containing two, three, four, five, six, seven, eight, nine, ten, eleven or twelve carbon atoms), which is optionally substituted with one or more (e.g., two, three, four, five, six, seven, eight, nine, ten or more) R^{a5}. In some embodiments, L⁴ is C₃₋₁₀ alkyl, C₃₋₉ alkyl, C₃₋₈ alkyl, C₃₋₇ alkyl, C₃₋₆ alkyl, C₃₋₅ alkyl, or C₃₋₄ alkyl. In some embodiments, L⁴ is

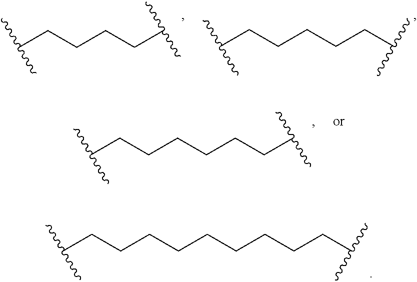 [SMILES: *CCCC* ; *CCCCC* ; *CCCCCC* ; *CCCCCCCCC*]

In some embodiments, L⁴ is a heteroalkyl containing one or more (e.g., 1, 2, 3, 4 or more) heteroatoms (e.g., O, N, S), which is optionally substituted with one or more (e.g., two, three, four, five, six, seven, eight, nine, ten or more) R^{a5}. In some embodiments, L⁴ is a heteroalkyl containing one or more (e.g., 1, 2, 3, 4 or more) oxygen or nitrogen atoms, which is optionally substituted with one or more (e.g., two, three, four, five, six, seven, eight, nine, ten or more) R^{a5}. In some embodiments, L⁴ is a heteroalkyl containing one to twelve (e.g., one, two, three, four, five, six, seven, eight, nine, ten, eleven or twelve) carbon atoms and at least one (e.g., 1, 2, 3, 4, 5 or more) oxygen atom, which is optionally substituted with one or more (e.g., two, three, four, five, six, seven, eight, nine, ten, or more) R^{a5}. In some embodiments, L⁴ is C₃₋₁₂ heteroalkyl (e.g., a heteroalkyl containing three, four, five, six, seven, eight, nine, ten, eleven or twelve carbon atoms) containing one or two oxygen atoms, and the oxygen atom is inserted into main chain of the C₃₋₁₂ heteroalkyl. In some embodiments, L⁴ is

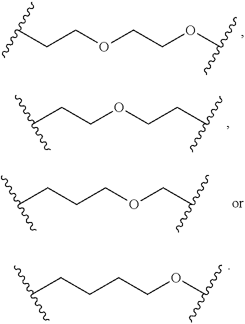 [SMILES: *CCOCCO* ; *CCOCC* ; *CCCOC* ; *CCCCO*]

In some embodiments, L⁴ is a heteroalkyl containing one to twelve (e.g., one, two, three, four, five, six, seven, eight, nine, ten, eleven or twelve) carbon atoms and at least one (e.g., 1, 2, 3, 4, 5 or more) nitrogen atom, which is optionally substituted with one or more (e.g., two, three, four, five, six, seven, eight, nine, ten, or more) R^{a5}. In some embodiments, L⁴ is C₃₋₁₂ heteroalkyl (e.g., a heteroalkyl containing three, four, five, six, seven, eight, nine, ten, eleven or twelve carbon atoms) containing one nitrogen atom, and the nitrogen atom is inserted into main chain of the C₃₋₁₂ heteroalkyl. In some embodiments, L⁴ is

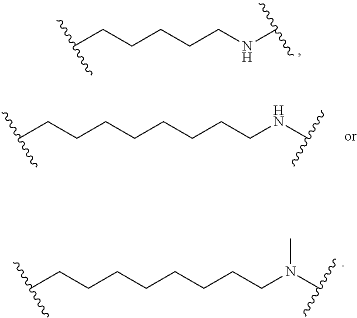 [SMILES: *CCCCCN* ; *CCCCCCCCN* ; *CCCCCCCCN(*)C]

In some embodiments, L⁴ is heteroalkenyl containing one or more (e.g., 1, 2, 3, 4 or more) heteroatoms (e.g., O, N, S), which is optionally substituted with one or more (e.g., two, three, four, five, six, seven, eight, nine, ten or more) R^{a5}. In some embodiments, L⁴ is a heteroalkenyl containing one or more (e.g., 1, 2, 3, 4 or more) oxygen or nitrogen atoms, which is optionally substituted with one or more (e.g., two, three, four, five, six, seven, eight, nine, ten or more) R^{a5}. In some embodiments, L⁴ is a heteroalkenyl containing one to twelve (e.g., one, two, three, four, five, six, seven, eight, nine, ten, eleven or twelve) carbon atoms and at least one (e.g., 1, 2, 3, 4, 5 or more) oxygen atom, which is optionally substituted with one or more (e.g., two, three, four, five, six, seven, eight, nine, ten, or more) R^{a5}. In some embodiments, L⁴ is C₂₋₁₂ heteroalkenyl (e.g., a heteroalkenyl containing two, three, four, five, six, seven, eight, nine, ten, eleven or twelve carbon atoms) containing one or two oxygen atoms, and the oxygen atom is inserted into main chain of the C₂₋₁₂ heteroalkenyl. In some embodiments, L⁴ is

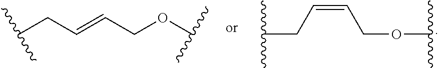 [SMILES: *C/C=C/CO* ; "or" ; *C/C=C\CO*]

In some embodiments, R^{a5} is alkyl. In some embodiments, R^{a5} is C₁₋₆ alkyl (e.g., methyl, ethyl, propyl, butyl, pentyl, hexyl). In some embodiments, R^{a5} is methyl.

In some embodiments, R¹ is hydrogen or C₁₋₆ alkyl. In some embodiments, R¹ is C₁₋₆ alkyl, e.g., methyl, ethyl, propyl, butyl, pentyl, or hexyl. In some embodiments, R¹ is C₁₋₆ alkyl, which is optionally substituted with one or more groups independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, R² is hydrogen or C₁₋₆ alkyl. In some embodiments, R² is C₁₋₆ alkyl, e.g., methyl, ethyl, propyl, butyl, pentyl, or hexyl. In some embodiments, R² is C₁₋₆ alkyl, which is optionally substituted with one or more groups independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, q is 0, 1, 2 or 3. In some embodiments, q is 1.

In some embodiments, R³ is —OR^{c}. In some embodiments, R^{c} is hydrogen or C₁₋₆ alkyl. In some embodiments, R^{c} is hydrogen. In some embodiments, R^{c} is C₁₋₆ alkyl, e.g., methyl, ethyl, propyl, butyl, pentyl, or hexyl, which is optionally substituted with one or more groups independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl.

In some embodiments, n is 0 or 1. In some embodiments, n is 1.

In some embodiments, R⁴ is alkyl. In some embodiments, R⁴ is C₁₋₆ alkyl, e.g., methyl, ethyl, propyl, butyl, pentyl, or hexyl. In some embodiments, R⁴ is methyl. In some embodiments, R⁴ is halogen (e.g., F, Cl, Br, I). In some embodiments, R⁴ is Cl.

In some embodiments, the compound provided herein is represented by Formula (II), Formula (III) or Formula (IV) below:

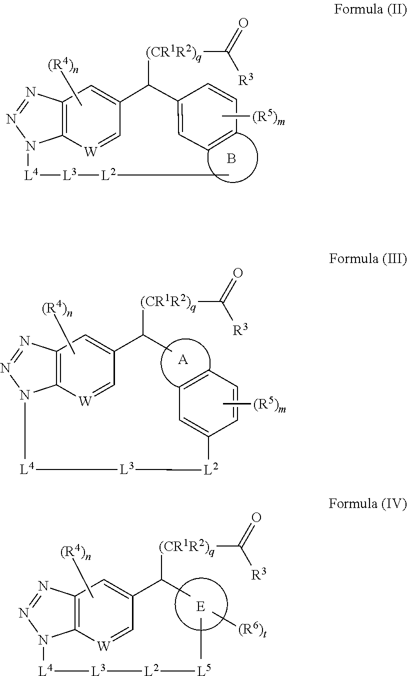

each of R⁵ and R⁶ is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl, wherein the alkyl, alkenyl and alkynyl, either alone or as part of another group, are independently optionally substituted with one or more R^{a7};
m is 0, 1, 2 or 3;
t is 0, 1, 2 or 3; and
each of W, R¹, R², R³, R⁴, L², L³, L⁴, L¹, Ring A, Ring B, Ring E, R^{a7}, n and q are as defined above.

In some embodiments, the compound provided herein is represented by Formula (IIa), Formula (IIb), Formula (IIIa), Formula (IIIb), Formula (IVa) or Formula (IVb) below:

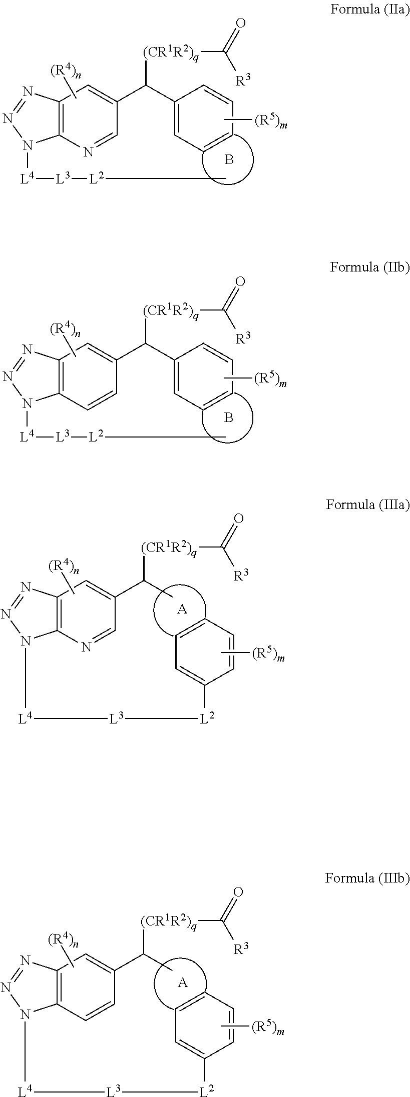

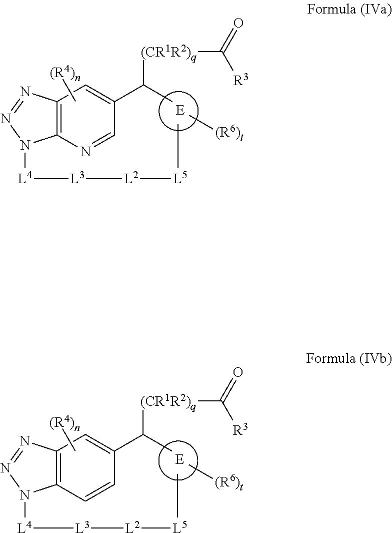

wherein, each of W, R¹, R², R³, R⁴, R⁵, R⁶, L², L³, L⁴, L⁵, Ring A, Ring B, Ring E, n, m, q and t are as defined above.

In some embodiments, the compound provided herein is represented by Formula (IIa1) or Formula (IIa2) below:

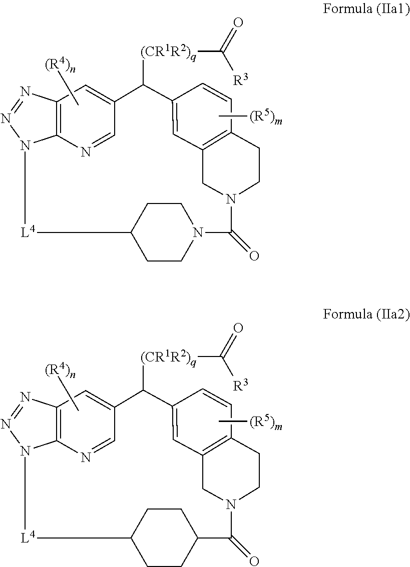

wherein,
L⁴ is C₃₋₈ alkyl or C₃₋₈ heteroalkyl;
R¹ is hydrogen, halogen or C₁₋₆ alkyl;
R² is hydrogen, halogen or C₁₋₆ alkyl;
R³ is —OR^{c}, R^{c} is hydrogen or C₁₋₆ alkyl;
R⁴ is hydrogen, halogen or C₁₋₆ alkyl;
R⁵ is hydrogen, halogen or C₁₋₆ alkyl;
n is 0 or 1;
m is 0 or 1; and
q is 1.

In some embodiments, the compound provided herein is represented by Formula (IIa1) or Formula (IIa2) above, wherein L⁴ is C₃₋₈ alkyl (e.g., methyl, ethyl, propyl, butyl, pentyl, hexyl, heptyl, octyl); R¹ is hydrogen; R² is hydrogen; R³ is —OH; R⁴ is hydrogen, halogen or C₁₋₆ alkyl (e.g., methyl, ethyl, propyl, butyl, pentyl, hexyl); R⁵ is hydrogen, halogen or C₁₋₆ alkyl (e.g., methyl, ethyl, propyl, butyl, pentyl, hexyl); n is 0 or 1; m is 0 or 1; and q is 1.

In some embodiments, the compound provided herein is represented by Formula (IIa1) or Formula (IIa2) above, wherein L⁴ is C₃₋₈ alkyl (e.g., methyl, ethyl, propyl, butyl, pentyl, hexyl, heptyl, octyl), R¹ is hydrogen, R² is hydrogen, R³ is —OH, n is 0, m is 0, and q is 1.

In some embodiments, the present disclosure provides novel compounds set forth in Table 1.1 or Table 1.2 below.

TABLE 1.1
Exemplary Compounds

| Compound No. | Chemical Structure |
|---|---|
| Compound 1 | 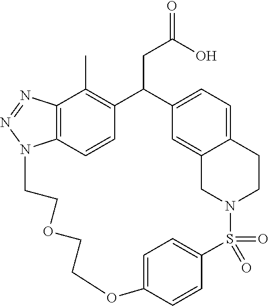 [SMILES: Cc1c2ccc3c1nnn3CCOCCOc1ccc(cc1)S(=O)(=O)N1CCc3ccc(cc3C1)C2CC(=O)O] |
| Compound 2 | 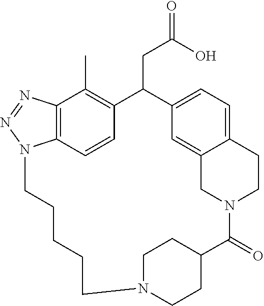 [SMILES: Cc1c2ccc3c1nnn3CCCCCN1CCC(CC1)C(=O)N1CCc3ccc(cc3C1)C2CC(=O)O] |
| Compound 3 | 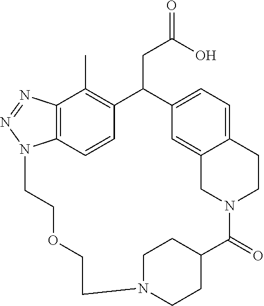 [SMILES: Cc1c2ccc3c1nnn3CCOCCN1CCC(CC1)C(=O)N1CCc3ccc(cc3C1)C2CC(=O)O] |
| Compound 4 | 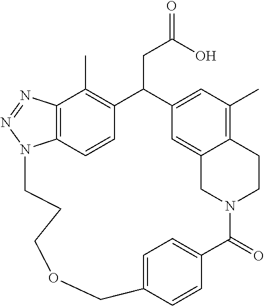 [SMILES: Cc1cc2cc3c1CCN(C3)C(=O)c1ccc(cc1)COCCCn1nnc3c(C)c(ccc31)C2CC(=O)O] |
| Compound 5 | 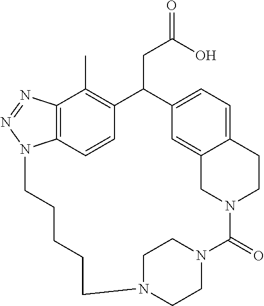 [SMILES: Cc1c2ccc3c1nnn3CCCCCN1CCN(CC1)C(=O)N1CCc3ccc(cc3C1)C2CC(=O)O] |
| Compound 6 | 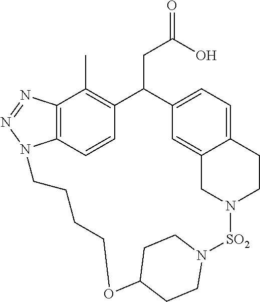 [SMILES: Cc1c2ccc3c1nnn3CCCCOC1CCN(CC1)S(=O)(=O)N1CCc3ccc(cc3C1)C2CC(=O)O] |
| Compound 7 | 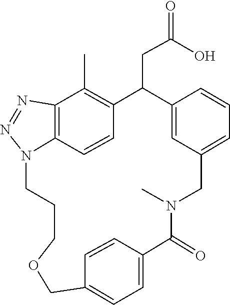 [SMILES: Cc1c2ccc3c1nnn3CCCOCc1ccc(cc1)C(=O)N(C)Cc1cccc(c1)C2CC(=O)O] |
| Compound 8 | 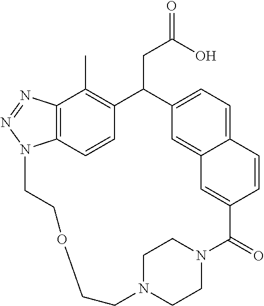 [SMILES: Cc1c2ccc3c1nnn3CCOCCN1CCN(CC1)C(=O)c1ccc3ccc(cc3c1)C2CC(=O)O] |
| Compound 9 | 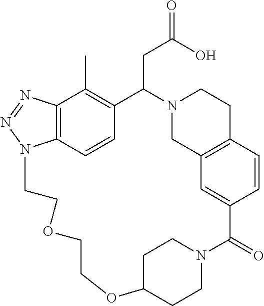 [SMILES: Cc1c2ccc3c1nnn3CCOCCOC1CCN(CC1)C(=O)c1ccc3c(c1)CN(CC3)C2CC(=O)O] |
| Compound 10 | 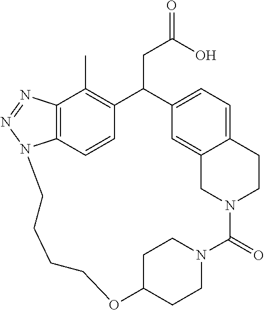 [SMILES: Cc1c2ccc3c1nnn3CCCCOC1CCN(CC1)C(=O)N1CCc3ccc(cc3C1)C2CC(=O)O] |
| Compound 11 | 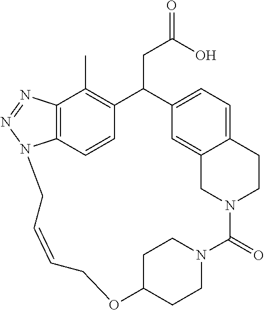 [SMILES: Cc1c2ccc3c1nnn3C/C=C\COC1CCN(CC1)C(=O)N1CCc3ccc(cc3C1)C2CC(=O)O] |
| Compound 12 | 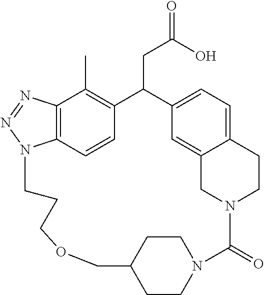 [SMILES: Cc1c2ccc3c1nnn3CCCOCC1CCN(CC1)C(=O)N1CCc3ccc(cc3C1)C2CC(=O)O] |
| Compound 13 | 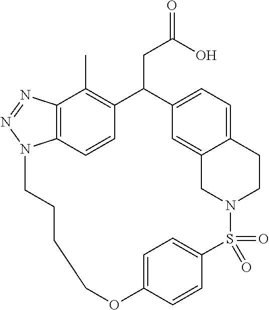 [SMILES: Cc1c2ccc3c1nnn3CCCCOc1ccc(cc1)S(=O)(=O)N1CCc3ccc(cc3C1)C2CC(=O)O] |
| Compound 14 | 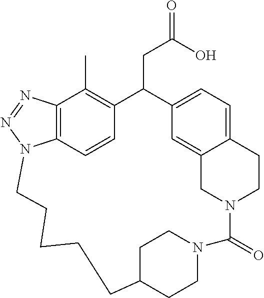 [SMILES: Cc1c2ccc3c1nnn3CCCCCC1CCN(CC1)C(=O)N1CCc3ccc(cc3C1)C2CC(=O)O] |
| Compound 15 | 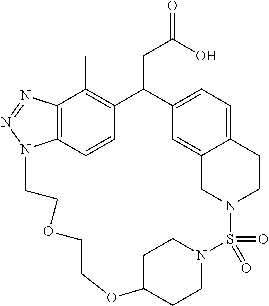 [SMILES: Cc1c2ccc3c1nnn3CCOCCOC1CCN(CC1)S(=O)(=O)N1CCc3ccc(cc3C1)C2CC(=O)O] |
| Compound 16 | 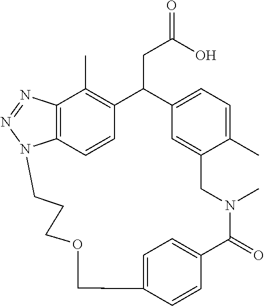 [SMILES: Cc1ccc2cc1CN(C)C(=O)c1ccc(cc1)COCCCn1nnc3c(C)c(ccc31)C2CC(=O)O] |
| | Isomer 1 |
| Compound 17 | 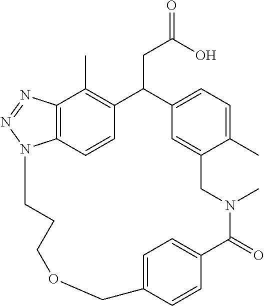 [SMILES: Cc1ccc2cc1CN(C)C(=O)c1ccc(cc1)COCCCn1nnc3c(C)c(ccc31)C2CC(=O)O] |
| | Isomer 2 |
| Compound 18 | 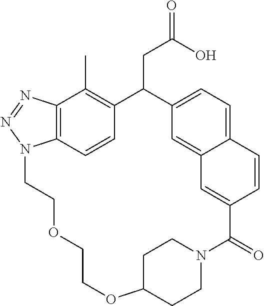 [SMILES: Cc1c2ccc3c1nnn3CCOCCOC1CCN(CC1)C(=O)c1ccc3ccc(cc3c1)C2CC(=O)O] |
| | Isomer 1 |
| Compound 19 | 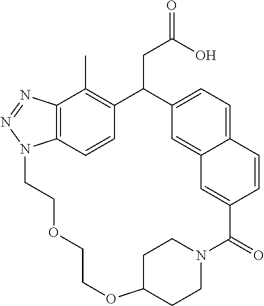 [SMILES: Cc1c2ccc3c1nnn3CCOCCOC1CCN(CC1)C(=O)c1ccc3ccc(cc3c1)C2CC(=O)O] |
| | Isomer 2 |
| Compound 20 | 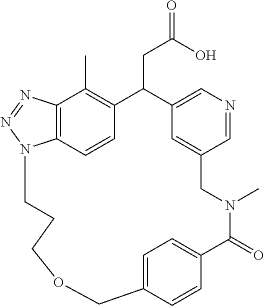 [SMILES: Cc1c2ccc3c1nnn3CCCOCc1ccc(cc1)C(=O)N(C)Cc1cncc(c1)C2CC(=O)O] |
| | Isomer 1 |
| Compound 21 | 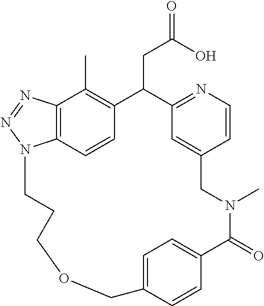 [SMILES: Cc1c2ccc3c1nnn3CCCOCc1ccc(cc1)C(=O)N(C)Cc1ccnc(c1)C2CC(=O)O] |
| Compound 22 | 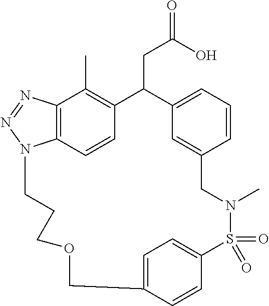 [SMILES: Cc1c2ccc3c1nnn3CCCOCc1ccc(cc1)S(=O)(=O)N(C)Cc1cccc(c1)C2CC(=O)O] |
| Compound 23 | 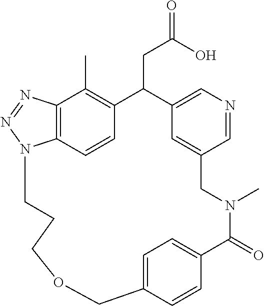 [SMILES: Cc1c2ccc3c1nnn3CCCOCc1ccc(cc1)C(=O)N(C)Cc1cncc(c1)C2CC(=O)O] |
| | Isomer 2 |
| Compound 24 | 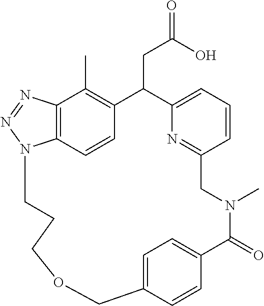 [SMILES: Cc1c2ccc3c1nnn3CCCOCc1ccc(cc1)C(=O)N(C)Cc1cccc(n1)C2CC(=O)O] |
| | Isomer 1 |
| Compound 25 | 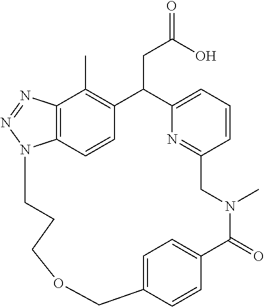 [SMILES: Cc1c2ccc3c1nnn3CCCOCc1ccc(cc1)C(=O)N(C)Cc1cccc(n1)C2CC(=O)O] |
| | Isomer 2 |
| Compound 26 | 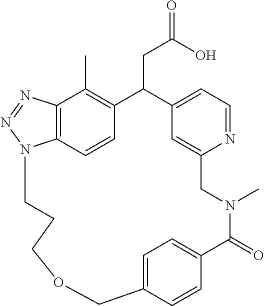 [SMILES: Cc1c2ccc3c1nnn3CCCOCc1ccc(cc1)C(=O)N(C)Cc1cc(ccn1)C2CC(=O)O] |
| | Isomer 1 |
| Compound 27 | 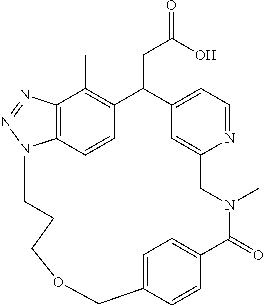 [SMILES: Cc1c2ccc3c1nnn3CCCOCc1ccc(cc1)C(=O)N(C)Cc1cc(ccn1)C2CC(=O)O] |
| | Isomer 2 |
| Compound 28 | 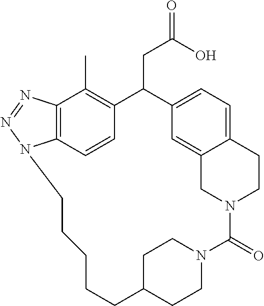 [SMILES: Cc1c2ccc3c1nnn3CCCCCC1CCN(CC1)C(=O)N1CCc3ccc(cc3C1)C2CC(=O)O] |
| | Isomer 1 of Compound 14 |
| Compound 29 | 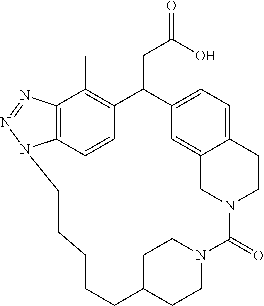 [SMILES: Cc1c2ccc3c1nnn3CCCCCC1CCN(CC1)C(=O)N1CCc3ccc(cc3C1)C2CC(=O)O] |
| | Isomer 2 of Compound 14 |
| Compound 30 | 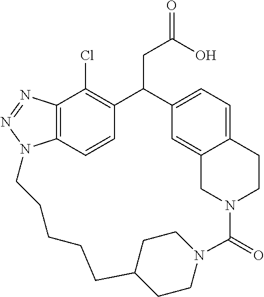 [SMILES: O=C(O)CC1c2ccc3c(c2)CN(CC3)C(=O)N2CCC(CCCCCn3nnc4c(Cl)c1ccc43)CC2] |
| Compound 31 | 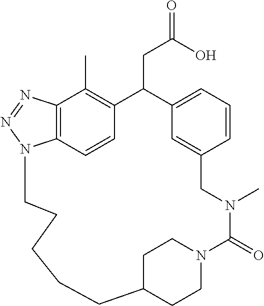 [SMILES: Cc1c2ccc3c1nnn3CCCCCC1CCN(CC1)C(=O)N(C)Cc1cccc(c1)C2CC(=O)O] |
| Compound 32 | 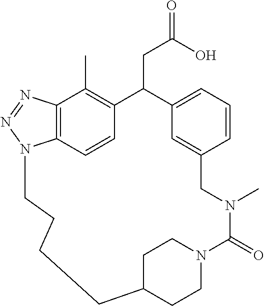 [SMILES: Cc1c2ccc3c1nnn3CCCCC1CCN(CC1)C(=O)N(C)Cc1cccc(c1)C2CC(=O)O] |
| Compound 34 | 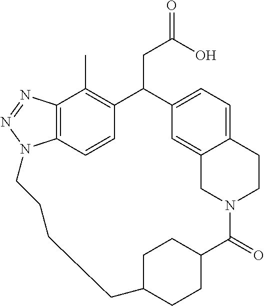 [SMILES: Cc1c2ccc3c1nnn3CCCCC1CCC(CC1)C(=O)N1CCc3ccc(cc3C1)C2CC(=O)O] |
| Compound 35 | 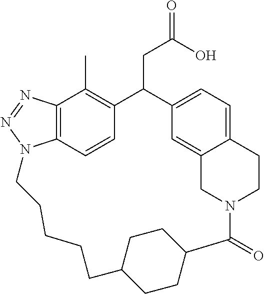 [SMILES: Cc1c2ccc3c1nnn3CCCCCC1CCC(CC1)C(=O)N1CCc3ccc(cc3C1)C2CC(=O)O] |
| Compound 36 | 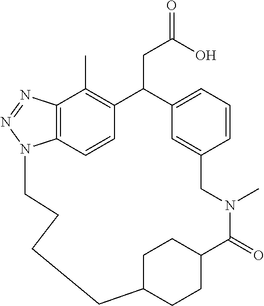 [SMILES: Cc1c2ccc3c1nnn3CCCCC1CCC(CC1)C(=O)N(C)Cc1cccc(c1)C2CC(=O)O] |
| Compound 37 | 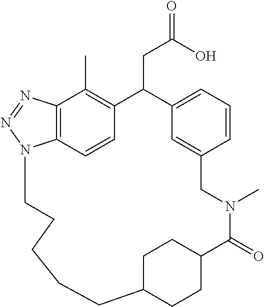 [SMILES: Cc1c2ccc3c1nnn3CCCCCC1CCC(CC1)C(=O)N(C)Cc1cccc(c1)C2CC(=O)O] |
| Compound 38 | 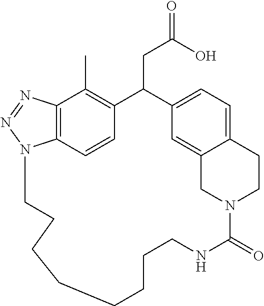 [SMILES: Cc1c2ccc3c1nnn3CCCCCCCCNC(=O)N1CCc3ccc(cc3C1)C2CC(=O)O] |
| Compound 40 | 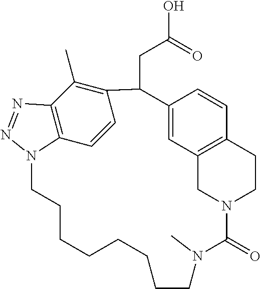 [SMILES: Cc1c2ccc3c1nnn3CCCCCCCCN(C)C(=O)N1CCc3ccc(cc3C1)C2CC(=O)O] |
| Compound 41 | 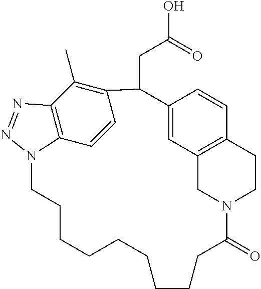 [SMILES: Cc1c2ccc3c1nnn3CCCCCCCCCC(=O)N1CCc3ccc(cc3C1)C2CC(=O)O] |
| Compound 42 | 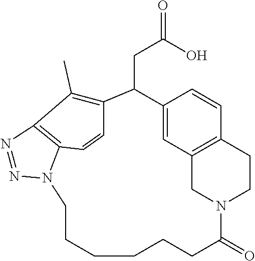 [SMILES: Cc1c2ccc3c1nnn3CCCCCCC(=O)N1CCc3ccc(cc3C1)C2CC(=O)O] |
| Compound 43 | 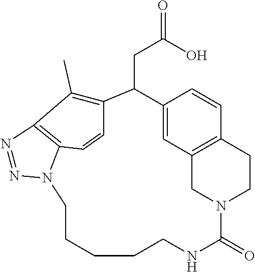 [SMILES: Cc1c2ccc3c1nnn3CCCCCNC(=O)N1CCc3ccc(cc3C1)C2CC(=O)O] |
| Compound 44 | 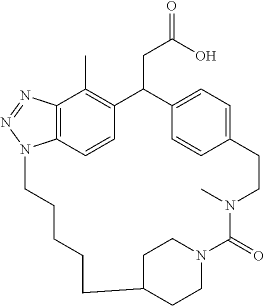 [SMILES: Cc1c2ccc3c1nnn3CCCCCC1CCN(CC1)C(=O)N(C)CCc1ccc(cc1)C2CC(=O)O] |
| Compound 46 | 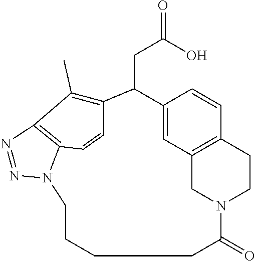 [SMILES: Cc1c2ccc3c1nnn3CCCCC(=O)N1CCc3ccc(cc3C1)C2CC(=O)O] |
| Compound 47 | 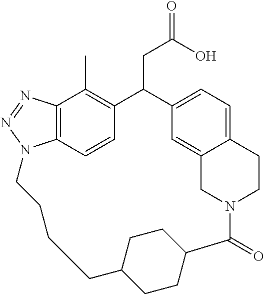 [SMILES: Cc1c2ccc3c1nnn3CCCCC1CCC(CC1)C(=O)N1CCc3ccc(cc3C1)C2CC(=O)O] |
| | Isomer 1 of Compound 34 |
| Compound 48 | 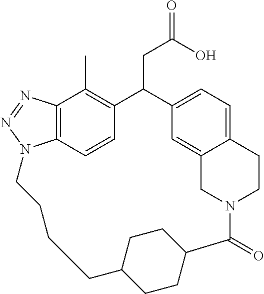 [SMILES: Cc1c2ccc3c1nnn3CCCCC1CCC(CC1)C(=O)N1CCc3ccc(cc3C1)C2CC(=O)O] |
| | Isomer 2 of Compound 34 |
| Compound 49 | 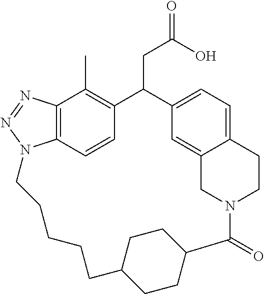 [SMILES: Cc1c2ccc3c1nnn3CCCCCC1CCC(CC1)C(=O)N1CCc3ccc(cc3C1)C2CC(=O)O] |
| | Isomer 1 of Compound 35 |
| Compound 50 | 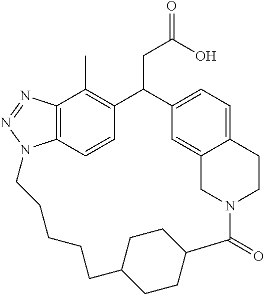 [SMILES: Cc1c2ccc3c1nnn3CCCCCC1CCC(CC1)C(=O)N1CCc3ccc(cc3C1)C2CC(=O)O] |
| | Isomer 2 of Compound 35 |
| Compound 51 | 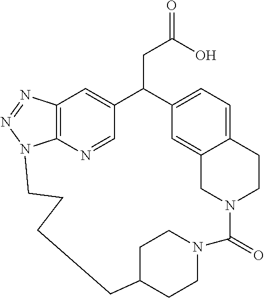 [SMILES: O=C(O)CC1c2ccc3c(c2)CN(CC3)C(=O)N2CCC(CCCCn3nnc4cc1cnc43)CC2] |
| | Isomer 1 |
| Compound 52 | 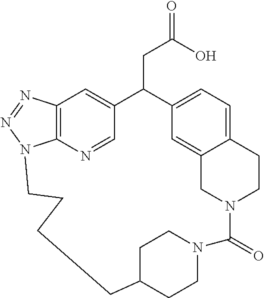 [SMILES: O=C(O)CC1c2ccc3c(c2)CN(CC3)C(=O)N2CCC(CCCCn3nnc4cc1cnc43)CC2] |
| | Isomer 2 |
| Compound 53 | 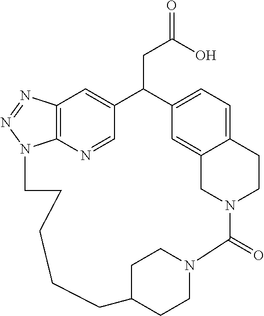 [SMILES: O=C(O)CC1c2ccc3c(c2)CN(CC3)C(=O)N2CCC(CCCCCn3nnc4cc1cnc43)CC2] |
| | Isomer 1 |
| Compound 54 | 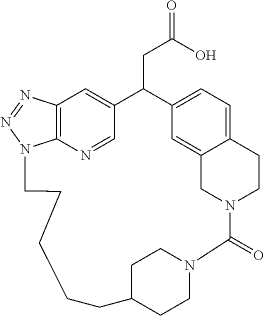 [SMILES: O=C(O)CC1c2ccc3c(c2)CN(CC3)C(=O)N2CCC(CCCCCn3nnc4cc1cnc43)CC2] |
| | Isomer 2 |
| Compound 55 | 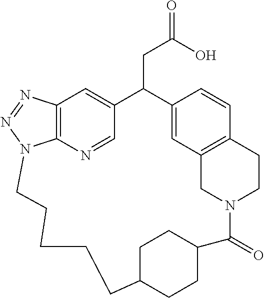 [SMILES: O=C(O)CC1c2ccc3c(c2)CN(CC3)C(=O)C2CCC(CCCCCn3nnc4cc1cnc43)CC2] |
| Compound 56 | 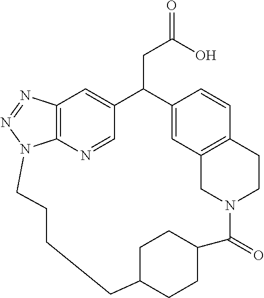 [SMILES: O=C(O)CC1c2ccc3c(c2)CN(CC3)C(=O)C2CCC(CCCCn3nnc4cc1cnc43)CC2] |

TABLE 1.2
Exemplary Compounds

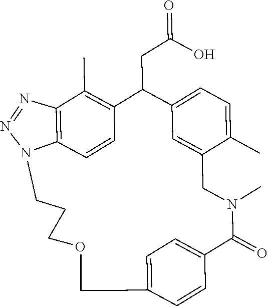 [SMILES: Cc1ccc2cc1CN(C)C(=O)c1ccc(cc1)CCOCCCn1nnc3c(C)c(ccc31)C2CC(=O)O]

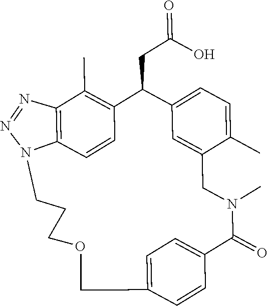 [SMILES: Cc1ccc2cc1CN(C)C(=O)c1ccc(cc1)CCOCCCn1nnc3c(C)c(ccc31)[C@H]2CC(=O)O]

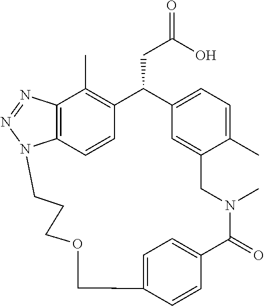 [SMILES: Cc1ccc2cc1CN(C)C(=O)c1ccc(cc1)COCCCn1nnc3c(C)c(ccc31)[C@@H]2CC(=O)O]

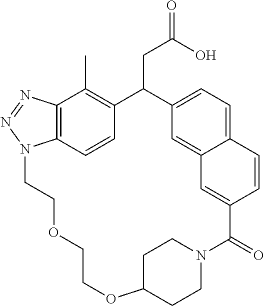 [SMILES: Cc1c2ccc3c1nnn3CCOCCOC1CCN(CC1)C(=O)c1ccc3ccc(cc3c1)C2CC(=O)O]

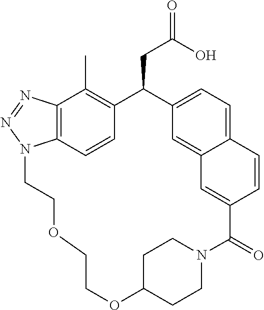 [SMILES: Cc1c2ccc3c1nnn3CCOCCOC1CCN(CC1)C(=O)c1ccc3ccc(cc3c1)[C@@H]2CC(=O)O]

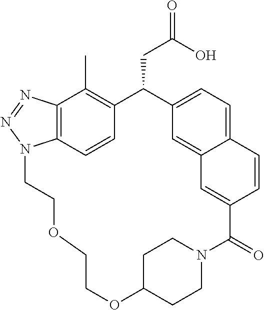 [SMILES: Cc1c2ccc3c1nnn3CCOCCOC1CCN(CC1)C(=O)c1ccc3ccc(cc3c1)[C@H]2CC(=O)O]

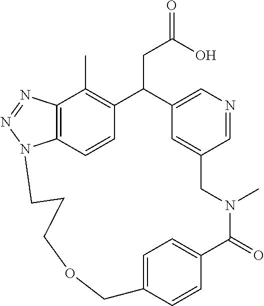 [SMILES: Cc1c2ccc3c1nnn3CCCOCc1ccc(cc1)C(=O)N(C)Cc1cncc(c1)C2CC(=O)O]

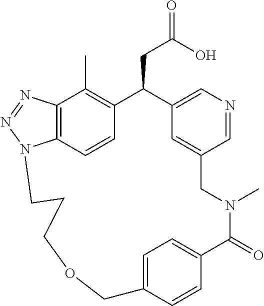 [SMILES: Cc1c2ccc3c1nnn3CCCOCc1ccc(cc1)C(=O)N(C)Cc1cncc(c1)[C@@H]2CC(=O)O]

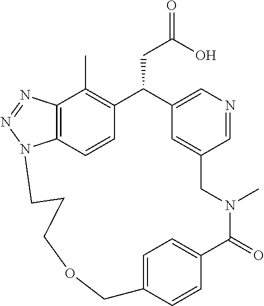 [SMILES: Cc1c2ccc3c1nnn3CCCOCc1ccc(cc1)C(=O)N(C)Cc1cncc(c1)[C@H]2CC(=O)O]

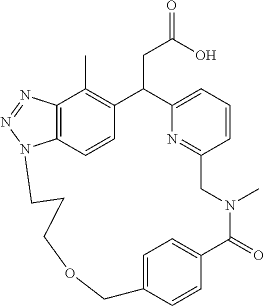 [SMILES: Cc1c2ccc3c1nnn3CCCOCc1ccc(cc1)C(=O)N(C)Cc1cccc(n1)C2CC(=O)O]

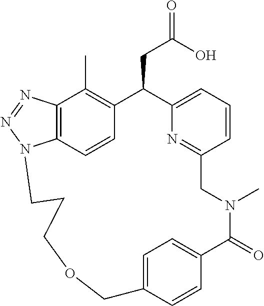 [SMILES: Cc1c2ccc3c1nnn3CCCOCc1ccc(cc1)C(=O)N(C)Cc1cccc(n1)[C@@H]2CC(=O)O]

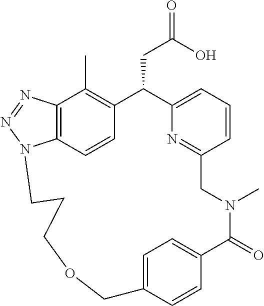 [SMILES: Cc1c2ccc3c1nnn3CCCOCc1ccc(cc1)C(=O)N(C)Cc1cccc(n1)[C@H]2CC(=O)O]

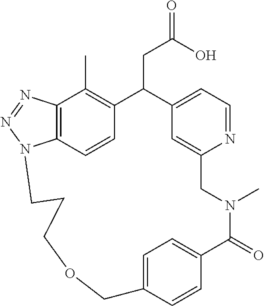 [SMILES: Cc1c2ccc3c1nnn3CCCOCc1ccc(cc1)C(=O)N(C)Cc1cc(ccn1)C2CC(=O)O]

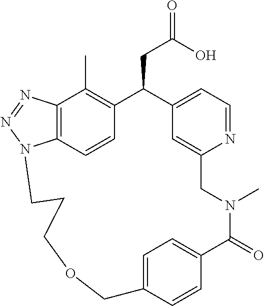 [SMILES: Cc1c2ccc3c1nnn3CCCOCc1ccc(cc1)C(=O)N(C)Cc1cc(ccn1)[C@@H]2CC(=O)O]

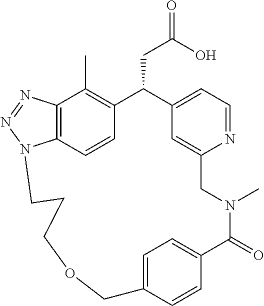 [SMILES: Cc1c2ccc3c1nnn3CCCOCc1ccc(cc1)C(=O)N(C)Cc1cc(ccn1)[C@H]2CC(=O)O]

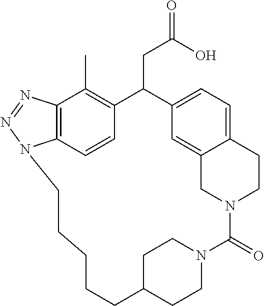 [SMILES: Cc1c2ccc3c1nnn3CCCCCC1CCN(CC1)C(=O)N1CCc3ccc(cc3C1)C2CC(=O)O]

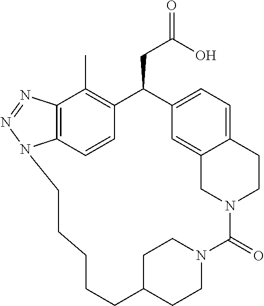 [SMILES: Cc1c2ccc3c1nnn3CCCCCC1CCN(CC1)C(=O)N1CCc3ccc(cc3C1)[C@@H]2CC(=O)O]

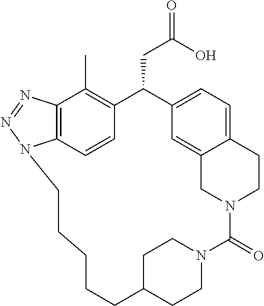 [SMILES: Cc1c2ccc3c1nnn3CCCCCC1CCN(CC1)C(=O)N1CCc3ccc(cc3C1)[C@H]2CC(=O)O]

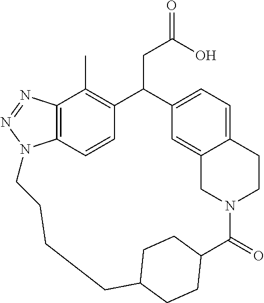 [SMILES: Cc1c2ccc3c1nnn3CCCCC1CCC(CC1)C(=O)N1CCc3ccc(cc3C1)C2CC(=O)O]

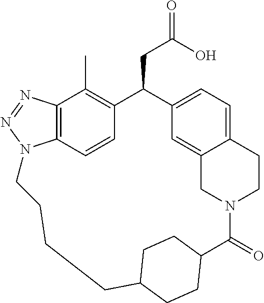 [SMILES: Cc1c2ccc3c1nnn3CCCCC1CCC(CC1)C(=O)N1CCc3ccc(cc3C1)[C@@H]2CC(=O)O]

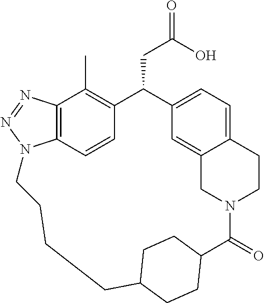 [SMILES: Cc1c2ccc3c1nnn3CCCCC1CCC(CC1)C(=O)N1CCc3ccc(cc3C1)[C@H]2CC(=O)O]

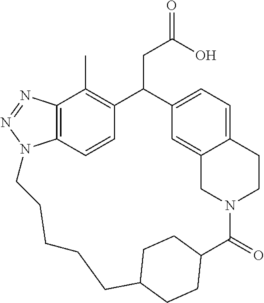 [SMILES: Cc1c2ccc3c1nnn3CCCCCC1CCC(CC1)C(=O)N1CCc3ccc(cc3C1)C2CC(=O)O]

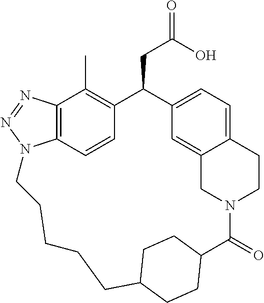 [SMILES: Cc1c2ccc3c1nnn3CCCCCC1CCC(CC1)C(=O)N1CCc3ccc(cc3C1)[C@@H]2CC(=O)O]

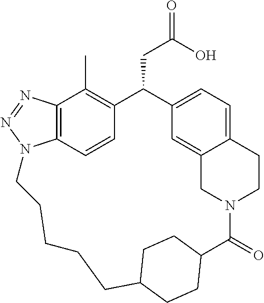 [SMILES: Cc1c2ccc3c1nnn3CCCCCC1CCC(CC1)C(=O)N1CCc3ccc(cc3C1)[C@H]2CC(=O)O]

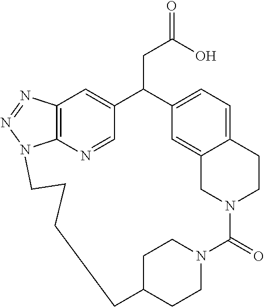 [SMILES: O=C(O)CC1c2ccc3c(c2)CN(CC3)C(=O)N2CCC(CCCCn3nnc4cc1cnc43)CC2]

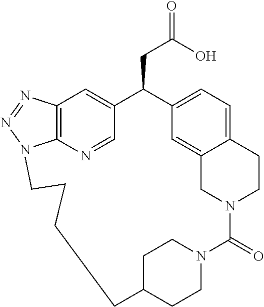 [SMILES: O=C(O)C[C@H]1c2ccc3c(c2)CN(CC3)C(=O)N2CCC(CCCCn3nnc4cc1cnc43)CC2]

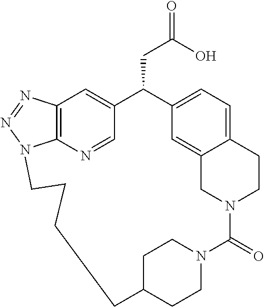 [SMILES: O=C(O)C[C@@H]1c2ccc3c(c2)CN(CC3)C(=O)N2CCC(CCCCn3nnc4cc1cnc43)CC2]

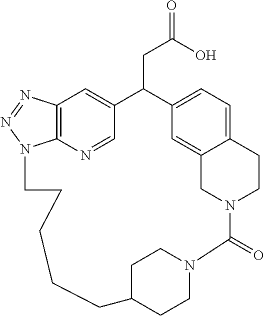 [SMILES: O=C(O)CC1c2ccc3c(c2)CN(CC3)C(=O)N2CCC(CCCCCn3nnc4cc1cnc43)CC2]

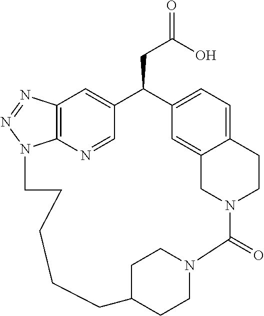 [SMILES: O=C(O)C[C@H]1c2ccc3c(c2)CN(CC3)C(=O)N2CCC(CCCCCn3nnc4cc1cnc43)CC2]

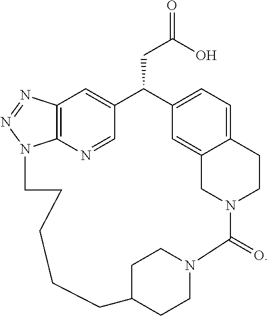 [SMILES: O=C(O)C[C@@H]1c2ccc3c(c2)CN(CC3)C(=O)N2CCC(CCCCCn3nnc4cc1cnc43)CC2]

Compounds provided herein are described with reference to both generic formulae and specific compounds. In addition, the compounds of the present disclosure may exist in a number of different forms or derivatives, including but not limited to, stereoisomers, racemic mixtures, regioisomers, tautomers, salts, prodrugs, soft drugs, active metabolic derivatives (active metabolites), solvated forms, different crystal forms or polymorphs, all within the scope of the present disclosure.

The compounds of present disclosure can comprise one or more asymmetric centers, and thus can exist in various stereoisomeric forms, e.g., enantiomers and/or diastereomers. Thus, the compounds of present disclosure and compositions thereof may be in the form of an individual enantiomer, diastereomer or geometric isomer, or may be in the form of a mixture of stereoisomers. In certain embodiments, the compounds of the present disclosure are enantiopure compounds. In certain embodiments, mixtures of enantiomers or diastereomers are provided.

The term “enantiomer” refers to two stereoisomers of a compound which are non-superimposable mirror images of one another. The term “diastereomer” refers to a pair of optical isomers which are not mirror images of one another. Diastereomers have different physical properties, e.g., melting points, boiling points, spectral properties, and reactivities.

Furthermore, certain compounds, as described herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of enantiomers. In addition to the above-mentioned compounds per se, this disclosure also encompasses compositions comprising one or more compounds.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a stereoisomer may, in some embodiments, be provided substantially free of one or more corresponding stereoisomers, and may also be referred to as “stereochemically enriched”.

Where a particular enantiomer is preferred, it may, in some embodiments be provided substantially free of the opposite enantiomer, and may also be referred to as “optically enriched”. “Optically enriched”, as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high performance liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E.L. Stereochemistry of Carbon Compounds (McGraw-Hill, NY, 1962); Wilen, S.H. Tables of Resolving Agents and Optical Resolutions p. 268 (E.L. Eliel, Ed., Univ. of Notre Dame Press, Notre Dame, IN 1972).

The compounds of the present disclosure may also exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

As used herein, the term “prodrug” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolism, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs are discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entireties.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques known in the art. See, e.g., Bertolini et al., 1997, J Med Chem 40:2011-2016; Shan et al., J Pharm Sci 86:756-757; Bagshawe, 1995, Drug Dev Res 34:220-230.

As used herein, the term “active intermediate” refers to an intermediate compound in the synthetic process, which exhibits the same or essentially the same biological activity as the final synthesized compound.

Compounds of the present disclosure can be formulated as or be in the form of pharmaceutically acceptable salts. Unless specified to the contrary, a compound provided herein includes pharmaceutically acceptable salts of such compound.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, then the solvate formed is a hydrate; and if the solvent is alcohol, then the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of compounds can be prepared by crystallization under different conditions.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

### Synthesis of Compounds

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

When hydrolysis reaction is carried out in each step, an acid or a base may be used as a reagent. In addition, when acid hydrolysis reaction for a tert-butyl ester is carried out, formic acid, triethylsilane or the like may be added in order to reductively trap a secondarily produced tert-butyl cation.

When esterification reaction, amidation reaction, or ureation reaction is carried out in each step, examples of the reagent to be used include but not limited to acyl halide forms such as acid chlorides and acid bromides; and activated carboxylic acids in the form of acid anhydride, active ester, sulfuric ester or the like. Examples of the activator for a carboxylic acid include but not limited to carbodiimide-based condensing agents such as 1-ethyl-3-(3-dimethylaminopropyl)carbodiimide hydrochloride (WSCD); triazine-based condensing agents such as 4-(4,6-dimethoxy-1,3,5-triazin-2-yl)-4-methylmorpholinium chloride (DMT-MM); carbonate ester-based condensing agents such as 1,1-carbonyldiimidazole (CDI); diphenylphosphoryl azide (DPPA); benzotriazol-1-yloxy-trisdimethylaminophosphonium salt (BOP reagent); 2-chloro-1-methyl-pyridinium iodide (Mukaiyama reagent); thionyl chloride; lower alkyl haloformates such as ethyl chloroformate; O-(7-azabenzotriazol-1-yl)-N,N,N′,N′-tetramethyluronium hexafluorophosphate (HATU); 1-[bis(dimethylamino)methylene]-1H-benzotriazolium 3-oxide tetrafluoroborate (TBTU); sulfuric acid; and a combination thereof. When a carbodiimide-based condensing agent is used, additives such as 1-hydroxybenzotriazole (HOBt), N-hydroxysuccinimide (HOSu) and dimethylaminopyridine (DMAP) may be further added to the reaction.

When alkylation reaction is carried out in each step, used can be an electrophile such as a halogenated alkyl or an optionally substituted sulfonyloxy group (for example, methanesulfonyloxy, ethanesulfonyloxy, trifluoromethanesulfonyloxy, benzenesulfonyloxy, p-toluenesulfonyloxy and the like), and a nucleophile (for example, amine, alcohol, active methylene compound adjacent to an electroattracting group and the like) and a base (for example, organic base, metal alkoxide, inorganic base and the like) as reagents. In addition, the alkylation can also be carried out, after transforming an alcohol into an active ester, in the presence of a silyl enol ether and an acid such as 1,1,1-trifluoro-N-[(trifluoromethyl)sulfonyl]methanesulfonamide. Moreover, the alkylation can also be carried out in the presence of an alcohol, a silyl enol ether and a Lewis acid.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), which is incorporated herein by reference in its entirety.

When coupling reaction is carried out in each step, examples of the metal catalyst to be used include but not limited to palladium compounds such as palladium(II) acetate, tetrakis(triphenylphosphine)palladium(0), dichlorobis(triphenylphosphine)palladium(II), dichlorobis(triethylphosphine)palladium(II), tris(dibenzylideneacetone)dipalladium(0) and 1,1′-bis(diphenylphosphino)ferrocene palladium(II) chloride; nickel compounds such as tetrakis(triphenylphosphine)nickel(0); rhodium compounds such as chloro(1,5-cyclooctadiene)rhodium(I) (dimer) and tris(triphenylphosphine)rhodium(III) chloride; cobalt compounds; copper compounds such as copper oxide and copper(I) iodide; and platinum compounds. In addition, a phosphine ligand may be added to the reaction, and examples of such a phosphine include triphenylphosphine, 1,1′-bis(diphenylphosphino)ferrocene, and tri-o-tolylphosphine. Further, a base may be added to the reaction, and examples of such a base include organic bases, inorganic bases and the like.

When boration reaction is carried out in each step, examples of the metal catalyst to be used include but not limited to palladium compounds such as tetrakis(triphenylphosphine)palladium(0), tris(dibenzylideneacetone)dipalladium(0) and 1,1′-bis(diphenylphosphino)ferrocene palladium(II) chloride. Further, a base may be added to the reaction, and examples of such a base include organic bases, inorganic bases and the like. In addition, examples of the boron source include pinacol diborane. Moreover, a borate ester group can be transformed into a boric acid group using ammonium acetate and sodium periodate as reagents.

When cyanation reaction is carried out in each step, examples of the metal catalyst to be used include but not limited to palladium compounds such as palladium acetate, tetrakis(triphenylphosphine)palladium(0), tris(dibenzylideneacetone)dipalladium(0) and 1,1′-bis(diphenylphosphino)ferrocene palladium(II) chloride; and cyanides such as sodium cyanide, zinc cyanide and copper cyanide. In addition, a phosphine ligand such as 1,1′-bis(diphenylphosphino)ferrocene or zinc powder may be added to the reaction.

When Mitsunobu reaction is carried out in each step, azodicarboxylate esters (for example, diethyl azodicarboxylate (DEAD), diisopropyl azodicarboxylate (DIAD) and the like) and triphenylphosphine may be used as reagents.

When ring-closing metathesis (RCM) reaction is carried out in each step, for the metal catalyst to be used, a ruthenium compound such as Grubbs 1st-generation catalyst (Grubbs I, benzylidene-bis-(tricyclohexylphosphine)dichlororuthenium), Grubbs 2nd-generation catalyst (Grubbs II, (1,3-bis(2,4,6-trimethylphenyl)-2-imidazolidinylidene)dichloro(phenylmethylene)(tricyclohexylphosphine)ruthenium) and Hoveyda-Grubbs 2nd-generation catalyst (Hoveyda-Grubbs II, [1,3-bis(2,4,6-trimethylphenyl)imidazolidin-2-ylidene]-dichloro-[(2-propan-2-yloxyphenyl)methylidene]ruthenium) may be used.

Compounds and/or intermediates may be in the salt forms, and although they are not particularly limited as long as the reaction is achieved.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The structures of the compounds in the examples are characterized by nuclear magnetic resonance (NMR) or/and liquid chromatography-mass spectrometry (LC-MS). NMR chemical shift (δ) is given in the unit of 10⁻⁶ (ppm). ¹H-NMR spectra are recorded in CDCl₃, CD₃OD or DMSO-d₆ solutions (reported in ppm) on a Bruker instrument (400 MHz or 500 MHz), using tetramethylsilane (TMS) as the reference standard (0.0 ppm).

Unless otherwise specified, the reactions of the present disclosure were typically done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

### Pharmaceutical Compositions

The present disclosure provides pharmaceutical compositions comprising one or more compounds of the present disclosure, or a pharmaceutically acceptable salt thereof. In some embodiments, the pharmaceutical composition comprises one or more compounds of the present disclosure, or a pharmaceutically acceptable salt thereof, and at least one pharmaceutical acceptable excipient.

A “pharmaceutical composition”, as used herein, is a formulation containing the compounds of the present disclosure in a form suitable for administration to a subject. In some embodiments, the pharmaceutical composition is in bulk or in unit dosage form. The unit dosage form is any of a variety of forms, including, for example, tablets, capsules, pills, powders, granules, sachets, cachets, lozenges, suspensions, emulsions, solutions, syrups, aerosols (as a solid or in a liquid medium), spray, ointment, paste, cream, lotion, gel, patch, inhalant, or suppository. The quantity of active ingredient (e.g., a formulation of the disclosed compound or salt, hydrate, solvate or isomer thereof) in a unit dose of composition is a therapeutically effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In some embodiments, the compound of the present disclosure is mixed under sterile conditions with a pharmaceutically acceptable excipient, and with any preservatives, buffers or propellants that are required.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount” refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

In some embodiments, the pharmaceutical compositions can be formulated so that a dosage of between 0.01-500 mg/kg body weight/day, for example, 0.05-500 mg/kg body weight/day, 0.1-500 mg/kg body weight/day, 0.1-400 mg/kg body weight/day, 0.1-300 mg/kg body weight/day, 0.1-200 mg/kg body weight/day, 0.1-100 mg/kg body weight/day, 0.1-80 mg/kg body weight/day, 1-100 mg/kg body weight/day or 1-80 mg/kg body weight/day of the compounds of the present disclosure, or a pharmaceutically acceptable salt thereof, can be administered.

In some embodiments, the pharmaceutical compositions comprise one or more compounds of the present disclosure, or a pharmaceutically acceptable salt thereof, as a first active ingredient, and further comprise a second active ingredient. The second active ingredient can be any agent known in the art, for example, chemotherapeutic agents or immunotherapeutic agents. In some embodiments, the second active ingredient is selected from the group consisting of a chemotherapeutic agent, an anti-tumor agent, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesic, an antioxidant, a metal chelator, and cytokines. In some embodiments, the second active ingredient is a Keap1 inhibitor.

In some embodiments, the present disclosure provides a pharmaceutical composition comprising a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof, or a pharmaceutically acceptable salt thereof, and at least one additional therapeutic agent. In some embodiments, there is one additional therapeutic agent. In some embodiments, there are two additional therapeutic agents. In some embodiments, there are three or more additional therapeutic agents.

In some embodiments, the amount of additional therapeutic agent present in the pharmaceutical composition of the present disclosure can be no more than the amount that would normally be administered in a pharmaceutical composition comprising that therapeutic agent as the only active agent. In certain embodiments, the amount of the additional therapeutic agent in the pharmaceutical composition of the present disclosure will range from about 50% to 100% of the amount normally present in a pharmaceutical composition comprising that therapeutic agent as the only therapeutically active agent.

Therefore, in another aspect, provided herein is a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof in combination with one or more therapeutic agents listed above.

As used herein, the term “combination” refers to simultaneous, separate or sequential administration. In some embodiments, “combination” refers to simultaneous administration. In some embodiments, “combination” refers to separate administration. In some embodiments, “combination” refers to sequential administration. Where the administration is sequential or separate, the delay in administering the second component should not be such as to lose the beneficial effect of the combination.

In a further aspect, provided herein is a pharmaceutical composition comprising a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof in combination with one or more therapeutic agents listed above, in association with a pharmaceutically acceptable excipient.

In a further aspect, provided herein is a kit comprising a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof in combination with one or more therapeutic agents listed above.

In a further aspect, provided herein is a kit comprising:
a) a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof in a first unit dosage form;
b) a therapeutic agent selected from those listed above in a second unit dosage form; and
c) container for containing the first and second unit dosage forms.

### Uses of Compounds

In one aspect, the present disclosure provides compounds of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or pharmaceutically acceptable salts thereof, which show Nrf2 modulating (e.g., activating) activity.

As used herein, the term “Nrf2 activating activity” refers to an increase in the level or activity of Nrf2 as a direct or indirect response to the presence of a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof, relative to the level or activity of Nrf2 in the absence of a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof. Such an increase in the level or activity may be due to the direct interaction of the compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof with Nrf2, or due to the interaction of the compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof with one or more other factors that in turn affect Nrf2 level or activity. For example, the compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof may increase Nrf2 by directly binding to the Nrf2 protein, by causing (directly or indirectly) another factor to increase Nrf2 activity, or by (directly or indirectly) increasing the amount of Nrf2 protein present in the cell or organism.

In some embodiments, the compounds of the present disclosure were converted to active metabolites (parent drug) in vivo. In some embodiments, the compounds of the present disclosure were partially converted to active metabolites in vivo. In some embodiments, the compounds of the present disclosure were completely converted to active metabolites in vivo.

Without being bound by a particular theory, the compounds of the present disclosure can perform Nrf2 activating activity by directly inhibiting the Keap1-Nrf2 protein-protein interaction (PPI), which has emerged as a promising strategy for activating Nrf2. The compounds of the present disclosure can bind tightly to the Nrf2 binding pocket on Keap1, and/or have the benefits of activating Nrf2 with higher target selectivity than covalent Keap1 inhibitors (or Nrf2 activators) and thus reduce potential safety risks from off-target activity.

In some embodiments, the compounds of the present disclosure show Nrf2 activating activity with an IC₅₀ when tested in an assay according to Examples described below of less than 10 μM. In some embodiments, the IC₅₀ is less than 2000 nM, less than 1000 nM, less than 500 nM, less than 200 nM, less than 100 nM, or less than 50 nM.

As a result of their Nrf2 activating activity, the compounds of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof are useful in a method of activating Nrf2 in a cell, comprising contacting a cell with an effective amount of a compound or pharmaceutical composition described herein to activate Nrf2 in the cell. In certain embodiments, the method comprises administering an effective amount of a compound or pharmaceutical composition described herein to a subject in need thereof.

In some embodiments, the compounds of the present disclosure show Nrf2 activating activity with an EC₅₀ when tested in an assay according to Examples described below of less than 100 μM. In some embodiments, the EC₅₀ is less than 50 μM, less than 40 μM, less than 30 μM, less than 20 μM, less than 10 μM, or less than 5 μM.

In some embodiments, the compounds of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof are useful in therapy, for example in the treatment of diseases or disorders associated with oxidative stress. Diseases or disorders associated with oxidative stress include but not limited to hepatic disease (for example, hepatitis (for example, non-alcoholic steatohepatitis, fatty liver, alcoholic hepatitis, hepatitis B, hepatitis C, hepatic veno-occlusive disease), hepatic cirrhosis, bile duct disease (for example, primary sclerosing cholangitis (PSC)), cardiovascular disease (for example, heart failure, pulmonary arterial hypertension, myocardial infarction, arteriosclerosis, angina pectoris, brain infarction, cerebral hemorrhage, aortic aneurysm, aortic dissection, nephrosclerosis (for example, hypertensive nephrosclerosis), peripheral arterial disease (PAD), arteriosclerosis obliterans, dysrhythmia), lung disease (for example, chronic obstructive pulmonary disease (COPD), idiopathic pulmonary fibrosis (IPF), cystic fibrosis, asthma, pneumonia, aspiration pneumonia, interstitial pneumonia, respiratory infection, acute lung injury, acute respiratory distress syndrome (ARDS), a 1-antitrypsin deficiency), kidney disease (for example, chronic kidney disease (CKD), diabetic kidney disease (DKD), acute kidney injury (AKI), glomerular nephritis, pyelonephritis, interstitial nephritis, glomerulosclerosis, nephrotic syndrome, lupus nephritis, Alport syndrome, IgA nephropathy, polycystic kidney), central nervous system disease (for example, Parkinson's disease, Alzheimer's disease, dementia, cerebral stroke, amyotrophic lateral sclerosis (ALS), spinocerebellar degeneration (SCD), polyglutamine disease, prion disease, Huntington's disease, traumatic brain injury, epilepsy, autism, depression, adrenoleukodystrophy), mitochondrial disease (for example, Friedreich motor ataxia, mitochondrial myopathy), inflammatory disease (for example, multiple sclerosis, chronic rheumatism, systemic lupus erythematosus, Sjogren's syndrome, scleroderma, autoimmune hepatitis, type 1 diabetes mellitus, ulcerous colitis, Crohn disease, inflammatory bowel disease (IBD), spondylarthritis, pollinosis, collagen disease), life style related disease (for example, diabetes mellitus, hyperlipidemia, obesity, high blood pressure, hypercholesterolemia) and complication thereof (for example, diabetic retinopathy, DKD, diabetic neuropathy), sickle cell disease, thalassemia, anemia (for example, aplastic anemia, hemolytic anemia), cancer (for example, liver cancer, lung cancer, renal cancer, colon cancer, melanoma, medulloblastoma, neuroblastoma, leukemia), cachexia, gastrointestinal disease (for example, functional gastrointestinal disorder, gastric ulcer, reflux esophagitis, pancreatitis), endocrine disease (for example, Cushing syndrome, Hashimoto disease), eye disease (for example, age-related macular degeneration, corneal endothelial disorder, Fuchs endothelial corneal dystrophy (FECD), eye inflammation, ophthalmalgia, retinopathy of prematurity, cataract, dry eye), skin disease (for example, psoriasis, dermatitis, radiation dermatitis, epidermolysis bullosa, atopic dermatitis, stomatitis), wound healing failure, bone disease (for example, osteoporosis, systemic bone disease, bone fracture), viral infection (for example, HIV virus, cytomegalovirus, respiratory syncytial virus, influenza vims), heavy metal poisoning (for example, lead poisoning, mercury poisoning), pesticide poisoning (for example, paraquat poisoning, organophosphorus poisoning), drug-induced disorder (for example, drug-induced renal disorder, drug-induced hepatic disorder (for example, hepatic disorder due to acetaminophen), drug-induced lung disorder, orthopedic disease (for example, low back pain, sciatic neuralgia, intervertebral disk displacement, neck ache, stiff shoulder), pain (for example, fibromyalgia, neuropathic pain), ischemia-reperfusion injury and shock upon organ transplantation and surgery, aging, progeria, hyperanakinesia (for example, sarcopenia), urologic disease (for example, urination disorder), dental disease (for example, periodontal disease), otolaryngologic disease (for example, hearing difficulty), altitude sickness, chronic fatigue syndrome, and thinning hair. In addition, the compounds of the present disclosure can exhibit enhancement of the effect of cancer treatment and the effect of improving survival rate by a combined use with an immunity anticancer agent (for example, immune checkpoint inhibiting antibody). Further, it can exhibit a regeneration promoting activity (for example, hepatic regeneration promoting agent after hepatectomy).

In some embodiments, diseases or disorders associated with oxidative stress include hepatic disease (for example, non-alcoholic steatohepatitis (NASH)), bile duct disease (primary sclerosing cholangitis (PSC) or the like), cardiovascular disease (for example, heart failure or pulmonary arterial hypertension), lung disease (for example, chronic obstructive pulmonary disease (COPD)), kidney disease (for example, chronic kidney disease (CKD) or acute kidney injury (AKI)), central nervous system disease (for example, Parkinson's disease, Alzheimer's disease, cerebral stroke), mitochondrial disease (for example, Friedreich motor ataxia, mitochondrial myopathy), inflammatory disease (for example, multiple sclerosis (MS), inflammatory bowel disease (IBD)), sickle cell disease, cancer, and the like.

As used herein, the term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

As used herein, the term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment”, “treat” or “treating” is used synonymously with “therapy”. Similarly, the term “treat” can be regarded as “applying therapy” where “therapy” is as defined herein.

Therefore, in one aspect, provided herein is a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof, for use in therapy.

In some embodiments, provided herein is a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof, for use in the treatment of Nrf2-associated diseases, disorders or conditions.

In some embodiments, the Nrf2-associated disease, disorder or condition is related to a decreased level or activity of Nrf2 protein. In some embodiments, the Nrf2-associated disease, disorder or condition is related to increased oxidative stress, inflammation, impaired redox potential, impaired detoxification or deregulated metabolism. In some embodiments, the disease, disorder or condition is selected from the group consisting of an ocular disease, a hepatic and bile duct disease, a cardiovascular disease, a lung disease, a kidney disease, a neurodegenerative disease, a neuropsychiatric disorder, a cancer, a sickle cell disease, a mitochondrial disease, an inflammatory disease, a respiratory disease, aging, an autoimmune disease, a brain disease, diabetes (e.g., Type I diabetes, Type II diabetes, maternal diabetes), metabolic syndrome and diabetic complications.

In some embodiments, provided herein is a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof, for use as a medicament.

In some embodiments, provided herein is use of a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a pharmaceutically acceptable salt thereof, in the manufacture of a medicament for preventing, treating or alleviating a Nrf2-associated disease, disorder or condition. In some embodiments, the disease, disorder or condition is related to a decreased level or activity of Nrf2 protein. In some embodiments, the disease, disorder or condition is related to increased oxidative stress, inflammation, impaired redox potential, impaired detoxification or deregulated metabolism. In some embodiments, the disease, disorder or condition is selected from the group consisting of an ocular disease, a hepatic and bile duct disease, a cardiovascular disease, a lung disease, a kidney disease, a neurodegenerative disease, a neuropsychiatric disorder, a cancer, a sickle cell disease, a mitochondrial disease, an inflammatory disease, a respiratory disease, aging, an autoimmune disease, a brain disease, diabetes (e.g., Type I diabetes, Type II diabetes, maternal diabetes), metabolic syndrome and diabetic complications.

### Methods of Treatment

In a further aspect, the present disclosure provides a method of preventing, treating or alleviating a Nrf2-associated disease, disorder or condition in a subject in need thereof, which comprises administering to the subject a therapeutically effective amount of a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof, or a pharmaceutical composition of the present disclosure, owning to the Nrf-2 activating activity of the compounds of the present disclosure.

In some embodiments, the Nrf2-associated disease, disorder or condition is related to increased oxidative stress, inflammation, impaired redox potential, impaired detoxification or deregulated metabolism. In some embodiments, the Nrf2-associated disease, disorder or condition is selected from the group consisting of an ocular disease, a hepatic and bile duct disease, a cardiovascular disease, a lung disease, a kidney disease, a neurodegenerative disease, a neuropsychiatric disorder, a cancer, a sickle cell disease, a mitochondrial disease, an inflammatory disease, a respiratory disease, aging, an autoimmune disease, a brain disease, diabetes (e.g., Type I diabetes, Type II diabetes, maternal diabetes), metabolic syndrome and diabetic complications.

In some embodiments, the ocular disease is age related macular degeneration (AMD), retinitis pigmentosa (RP), geographic atrophy (GA), macular edema, macular edema following retinal vein occlusion (RVO), diabetic macular edema (DME), diabetic retinopathy (DR), retinal central vein occlusion, corneal neovascularization (CNV), ocular angiogenesis (ocular neovascularization affecting choroidal, corneal or retinal tissue), retinopathy of prematurity (ROP), pathological myopia, glaucoma (e.g., vascular glaucoma), retinoblastoma, retinal vein occlusion, uveitis, eye injury, Fuchs' endothelial corneal dystrophy (FECD), cataracts, ocular neurodegenerative diseases, optic neuropathy and neuromyelitis optica.

In some embodiments, the kidney disease is autosomal dominant polycystic kidney disease (ADPKD), acute kidney injury (AKI), diabetic nephrophaty, IgA nephropathy (IgAN), chronic kidney disease (CKD), Alstrom and Alport syndromes, renal fibrosis, focal segmental glomerulosclerosis, contrast-induced nephropathy, sepsis-induced acute kidney injury, and kidney disease or malfunction seen during kidney transplantation.

In some embodiments, the neurodegenerative disease is selected from the group consisting of Alzheimer's disease, Parkinson's disease, Huntington's disease, cognitive decline, amyloidosis, amyotrophic lateral sclerosis and multiple sclerosis.

In some embodiments, the neuropsychiatric disorder is selected from the group consisting of schizophrenia, bipolar disorder, depression, anxiety, Friedreich's ataxia, autism and attention deficit hyperactivity disorder.

In some embodiments, the diabetic complication is selected from the group consisting of diabetic cardiomyopathy, diabetic retinopathy, diabetic neuropathy, diabetic nephropathy and diabetic wound healing.

In some embodiments, the hepatic and bile duct disease is selected from the group consisting of nonalcoholic steatohepatitis (NASH), non-alcoholic fatty liver disease, toxin-induced liver disease (e.g., acetaminophen-induced hepatic disease), alcoholic liver disease (ALD), cholestasis, primary sclerosing cholangitis (PSC), viral hepatitis, cirrhosis, primary biliary cirrhosis (PBC), end stage liver disease, and liver fibrosis.

In some embodiments, the lung disease is selected from the group consisting of pulmonary fibrosis, idiopathic pulmonary fibrosis (IPF), cystic fibrosis, acute lung injury, lung infection, chronic obstructive pulmonary disease (COPD), emphysema, pulmonary arterial hypertension, lung disease secondary to environmental exposures, chronic and acute asthma, and acute respiratory distress syndrome.

In some embodiments, the cardiovascular disease is selected from the group consisting of atherosclerosis, hypertension, heart failure, stroke, cardiomyopathy, coronary heart disease, vascular endothelial dysfunction, blood-brain barrier dysfunction, reperfusion injury (brain, heart, kidney, liver, retina), and myocardial ischemia.

In some embodiments, the inflammatory disease is selected from the group consisting of inflammatory bowel disease, ulcerative colitis, Crohn's disease, pancreatitis, arthritis, osteoarthritis, dermatitis (e.g., radiation-induced dermatitis, allergic contact dermatitis), reflux-induced esophagitis and lupus nephritis.

In some embodiments, the autoimmune disease is selected from the group consisting of psoriasis, Sjogren syndrome, lupus, pemphigus, vitiligo and alopecia areata.

In some embodiments, the brain disease is selected from the group consisting of traumatic brain injury, brain edema, brain ischemia, encephalopathy (e.g., hepatic encephalopathy), and cerebral infarction.

In some embodiments, the Nrf2-associated disease, disorder or condition is selected from the group consisting of neuronal damage, epilepsy, spinal cord injury, immunosuppression due to radiation exposure, preeclampsia, high altitude sickness, wound healing, mitochondrial myopathies, malaria, ferroptosis/iron overload, alcohol dependence, anemia, asperger syndrome, eczema, chronic fatigue syndrome, Duchenne muscular dystrophy, edema, encephalitis, male/female fertility, fracture healing, gastroesophageal reflux disease, hearing loss, influenza infections, intestinal barrier dysfunction, osteoporosis, radiation-induced injury, seizures, skin ulcer and Down syndrome.

In some embodiments, the cancer is selected from the group consisting of colon cancer, lung cancer, esophageal cancer, breast cancer, bladder cancer, liver cancer, prostate cancer and colorectal cancer.

As used herein, the term “subject in need thereof” is a subject having a Nrf2-associated disease, disorder or condition, or a subject having an increased risk of developing Nrf2-associated disease, disorder or condition relative to the population at large. In the case of cancer, a subject in need thereof can have a precancerous condition. A “subject” includes a warm-blooded animal. In some embodiments, the warm-blooded animal is a mammal, e.g., human.

In this context, the term “therapeutically effective amount” refers to an amount of a compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof which is effective to provide “therapy” in a subject, or to “treat” a Nrf2-associated disease, disorder or condition in a subject. In the case of cancer, the therapeutically effective amount may cause any of the changes observable or measurable in a subject as described in the definition of “therapy”, “treatment” and “prophylaxis” above. For example, the effective amount can reduce the number of cancer or tumor cells; reduce the overall tumor size; inhibit or stop tumor cell infiltration into peripheral organs including, for example, the soft tissue and bone; inhibit and stop tumor metastasis; inhibit and stop tumor growth; relieve to some extent one or more of the symptoms associated with the cancer; reduce morbidity and mortality; improve quality of life; or a combination of such effects. An effective amount may be an amount sufficient to decrease the symptoms of a disease responsive to activation of Nrf2. As recognized by those skilled in the art, effective amounts may vary depending on route of administration, excipient usage, and co-usage with other agents. For example, where a combination therapy is used, the amount of the compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof described in this specification and the amount of the other pharmaceutically active agent(s) are, when combined, jointly effective to treat a targeted disorder in the animal patient. In this context, the combined amounts are in a “therapeutically effective amount” if they are, when combined, sufficient to decrease the symptoms of a disease responsive to activation of Nrf2 as described above.

In generally, “therapeutically effective amount” may be determined by one skilled in the art by, for example, starting with the dosage range described in this specification for the compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof and an approved or otherwise published dosage range(s) of the other pharmaceutically active compound(s).

Nrf2 activator and/or uses thereof are also described in the prior arts, for example, Hayes, J. D. et al., Trends Biochem. Sci. 2014, 39, 199-218.; Cuadrado, A. et al., Pharmacol. Rev. 2018, 70, 348-383; Cuadrado, A. et al., Nat. Rev. Drug Discovery 2019, 18, 295-317; Lu, M. C. et al., Med. Res. Rev. 2016, 36, 924-963; Zhuang, C. et al., MedChemComm 2017, 8, 286-294; Montes Diaz, G. et al., Autoimmun. Rev. 2018, 17, 1240-1250; Doss, J. F. et al., PLoS One 2016, 11, No. e0152895; Wang, Y. Y. et al., Drug Des., Dev. Ther. 2014, 8, 2075-2088; Zhuang, C. et al., MedChemComm 2017, 8, 286-294; Pallesen, J. S. et al., J. Med. Chem. 2018, 61, 8088-8103; Jiang, Z. Y. et al., J. Med. Chem. 2016, 59, 10837-10858; Satoh, T. et al., F1000Research 2017, 6, 2138; Liu, P. et al., Cell. Chem. Biol. 2019, 26, 1427-1435; Cuadrado, A. et al., Nat. Rev. Drug Discovery 2019, 18, 295-317; Calabrese, V. et al., Nat. Rev. Neurosci. 2007, 8, 766-775; Trovato Salinaro, A. et al., Immun. Ageing 2018, 15, 8; Calabrese, V. et al., Antioxid. Redox Signaling 2010, 13, 1763-1811, the entire contents of each of which are incorporated herein by reference.

The method of preventing, treating or alleviating Nrf2-associated associated diseases, disorders or conditions described in this specification may be used as monotherapy. As used herein, the term “monotherapy” refers to the administration of a single active or therapeutic compound to a subject in need thereof. In some embodiments, monotherapy will involve administration of a therapeutically effective amount of one of the compounds of the present disclosure, or a tautomer, a stereoisomer, or a pharmaceutically acceptable salt thereof, to a subject in need of such treatment.

Depending upon the particular diseases or conditions to be treated, the method of treating Nrf2-associated diseases, disorders or conditions described in this specification may involve, in addition to administration of the compound of the present disclosure, one or more additional therapies, for example, conventional surgery, radiotherapy, chemotherapy, immunotherapy, or a combination of such additional therapies. As used herein, the term “combination therapy” refers to the administration of a combination of multiple active compounds.

Additional therapies, such as additional anti-tumor agents, may be administered separately from the compounds of the present disclosure, as part of a multiple dosage regimen. Alternatively, these additional therapies may be part of a single dosage form, mixed with the compounds of the present disclosure in a single composition.

In some embodiments, the compounds of the present disclosure may be administered simultaneously, sequentially or separately to treatment with conventional surgery, radiotherapy, chemotherapy or immunotherapy.

Therefore, in one aspect, the present disclosure provides a method of preventing, treating or alleviating Nrf2-associated diseases, disorders or conditions in a subject in need thereof, wherein the compound of Formula (I), Formula (Ia), Formula (Ib), Formula (II), Formula (IIa), Formula (IIa1), Formula (IIa2), Formula (IIb), Formula (III), Formula (IIIa), Formula (IIIb), Formula (IV), Formula (IVa) or Formula (IVb), or a tautomer, a stereoisomer, or a pharmaceutically acceptable salt thereof is administered simultaneously, separately or sequentially with a second therapy.

In some embodiments, the second therapy is chemotherapy or immunotherapy. In some embodiments, the second therapy is selected from the group consisting of a chemotherapeutic agent, an anti-tumor agent, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesic, an antioxidant, a metal chelator, and cytokines. In some embodiments, the second therapy is a Keap1 inhibitor.

### EXAMPLES

For the purpose of illustration, the following examples are included. However, it is to be understood that these examples do not limit the invention and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the present disclosure.

The following abbreviations have the definitions set forth below:

| | |
|---|---|
| ACN | acetonitrile |
| Cbz | benzyloxycarbonyl |
| CPME | cyclopentyl methyl ether |
| DCM | dichloromethane |
| DEAD | diethyl azodicarboxylate |
| DIEA | N,N-diisopropylethylamine |
| DIPEA | N,N-diisopropylethylamine |
| DMA | dimethylacetamide |
| DMAP | 4-dimethylaminopyridine |
| DMEM | Dulbecco's modified eagle medium |
| DMF | N,N-dimethylformamide |
| DMSO | dimethyl sulfoxide |
| EA | ethyl acetate |
| EDCI | 1-ethyl-3-(3-dimethylaminopropyl)carbodiimide |
| ESI | electrospray ionization |
| FA | formic acid |
| FBS | fetal bovine serum |
| HATU | O-(7-azabenzotriazol-1-yl)-N,N,N′,N′-tetramethyluronium |
| | hexafluorophosphate |
| HOBt | hydroxybenzotriazole |
| HPLC | high-performance liquid chromatography |
| LCMS | liquid chromatography-mass spectrometry |
| MHz | megahertz |
| MS | mass spectrometry |
| NBS | N-bromosuccinimide |
| NMR | nuclear magnetic resonance (spectroscopy) |
| Prep-HPLC | preparative-HPLC |
| SFC | supercritical fluid chromatography |
| TBAI | tetrabutylammonium iodide |
| TEA | triethylamine |
| TFA | trifluoroacetic acid |
| THF | tetrahydrofuran |
| TLC | thin layer chromatography |

### Example 1 Preparation of Compounds

### Example 1.1 Synthesis of Compound 1

### Procedure for tert-butyl 2-(1⁴-methyl-4,4-dioxido-3¹,3²,3⁴-tetrahydro-1¹H-6,9-dioxa-4-thia-3(7,2)-isoquinolina-1(5,1)-benzo[d][1,2,3]triazola-5(1,4)-benzenacycloundecaphane-2-yl)acetic acid (Compound 1)

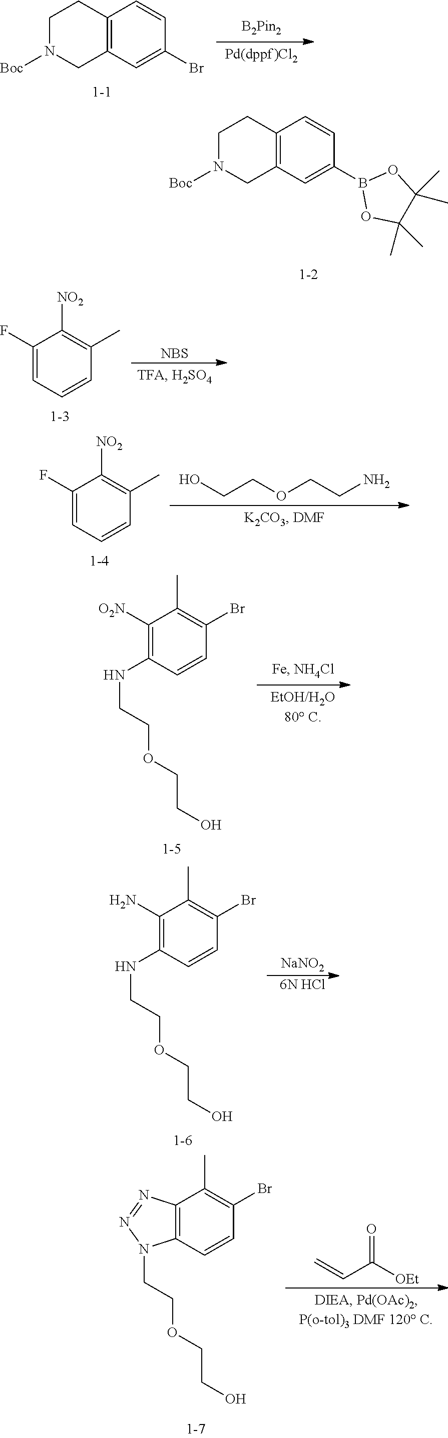

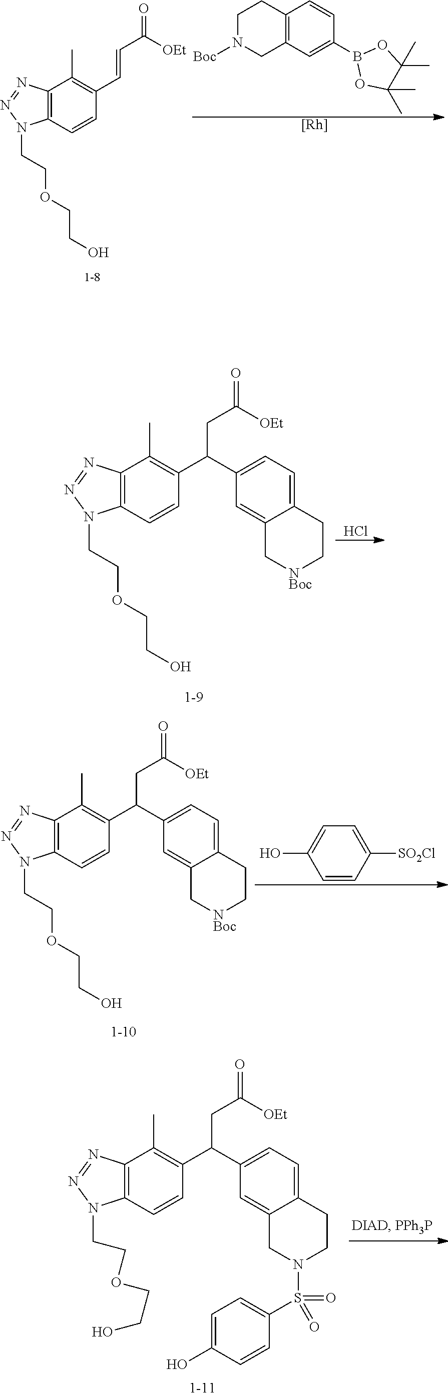

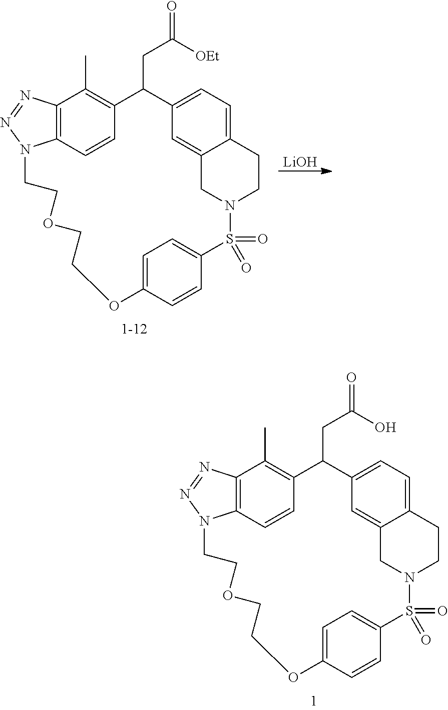

### tert-butyl 7-(4,4,5,5-tetramethyl-1,3,2-dioxaborolan-2-yl)-3,4-dihydroisoquinoline-2(1H)-carboxylate (1-2)

To the solution of tert-butyl 7-bromo-3,4-dihydroisoquinoline-2(1H)-carboxylate (10.0 g, 32.0 mmol), B₂Pin₂ (9.0 g, 35.3 mmol) and KOAc (9.4 g, 96.1 mmol) in dioxane (150 mL) was added Pd(dppf)Cl₂ (2.6 g, 3.2 mmol) at room temperature. The resulting mixture was stirred at 100° C. for 19 hours under nitrogen atmosphere. After cooling to room temperature, the mixture was then poured into ice water (500 mL) and then extracted with ethyl acetate (80 mL*3). The combined organic layers were dried over sodium sulfate, filtered and concentrated to dryness. The residue was purified by column chromatography (elute with ethyl acetate:petroleum ether=1:20 to 1:10) to have desired product 1-2 (9.2 g, 80.0%).

### 1-bromo-4-fluoro-2-methyl-3-nitrobenzene (1-4)

To the solution of 1-fluoro-3-methyl-2-nitrobenzene (100.0 g, 644.6 mmol) in TFA (500 mL) and H₂SO₄ (conc. 250 mL) was added NBS (120.5 g, 676.9 mmol) at 0° C. The resulting mixture was stirred at 30° C. for 19 hours under nitrogen atmosphere. The mixture was then poured into ice water (3 L) slowly. The precipitate was filtered and washed with cold water (1 L*3). The filter cake was then dried over vacuum to have the desired product 1-4 (145.6 g, 96.5%).

¹H NMR (400 MHz, DMSO-d₆) δ 8.00-7.96 (m, 1H), 7.49 (t, J=9.3 Hz, 1H), 2.37 (s, 3H).

### 2-(2-((4-bromo-3-methyl-2-nitrophenyl)amino)ethoxy)ethan-1-ol (1-5)

To the solution of 1-4 (10.0 g, 42.7 mmol) and 2-(2-aminoethoxy)ethan-1-ol (5.8 g, 55.5 mmol) in DMF (100 mL) was added K₂CO₃ (11.8 g, 85.4 mmol) at 20° C. The resulting mixture was stirred at 80° C. for 19 hours under nitrogen atmosphere. After cooling to room temperature, the mixture was then poured into ice water (1 L) and extracted with ethyl acetate (500 mL*3). The combined organic layers were dried over sodium sulfate, filtered and concentrated to dryness to have the crude product 1-5 (6.9 g, 50.8%).

MS(ESI) calculated for C₁₁H₁₅BrN₂O₄, 319.2; found 320.2.

### 2-(2-((2-amino-4-bromo-3-methylphenyl)amino)ethoxy)ethan-1-ol (1-6)

To the solution of 1-5 (6.9 g, 21.7 mmol) and NH₄Cl (10.9 g, 203.6 mmol) in EtOH (50 mL) and H₂O (16 mL) was added Fe powder (11.4 g, 203.6 mmol) at 20° C. The resulting mixture was stirred at 80° C. for 4 hours under nitrogen atmosphere. After cooling to 40° C., the mixture was filtered through a pad of celite. The filtrate was concentrated to dryness to have the crude product 1-6 (5.9 g, 94.4%).

MS(ESI) calculated for C₁₁H₁₇BrN₂O₂, 289.2; found 290.2.

### 2-(2-(5-bromo-4-methyl-1H-benzo[d][1,2,3]triazol-1-yl)ethoxy)ethan-1-ol (1-7)

To the solution of 1-6 (5.4 g, 18.7 mmol) in HCl (6M in H₂O, 60 mL) was added NaNO₂ (2.6 g, 37.4 mmol) in H₂O (60 mL) at 0° C. The resulting mixture was stirred at 25° C. for 2.5 hours. The reaction mixture was then neutralized with 4N NaOH at 0° C. and extracted with ethyl acetate (150 mL*3). The combined organic layers were dried over sodium sulfate, filtered and concentrated to dryness. The residue was purified by column chromatography (elute with ethyl acetate:petroleum ether=1:3 to 1:1) to have desired product 1-7 (5.5 g, 97.9%).

MS(ESI) calculated for C₁₁H₁₄BrN₃O₂, 299.0; found 300.1.

### ethyl (E)-3-(1-(2-(2-hydroxyethoxy)ethyl)-4-methyl-1H-benzo[d][1,2,3]triazol-5-yl)acrylate (1-8)

To the solution of 1-7 (3.9 g, 13.0 mmol) ethyl acrylate (7.8 g, 78.0 mmol) and DIEA (3.4 g, 26.0 mmol) in DMF (40 mL) was added Pd(OAc)₂ (438.0 mg, 2.0 mmol) and P(p-tol)₃ (1.2 g, 3.9 mmol) at 20° C. The resulting mixture was stirred at 120° C. for 19 hours under nitrogen atmosphere. After cooling to room temperature, the mixture was poured into ice water (200 mL) and then extracted with ethyl acetate (100 mL*3). The combined organic layers were dried over sodium sulfate, filtered and concentrated to dryness. The residue was purified by column chromatography (elute with ethyl acetate:petroleum ether=1:2 to 1:1) to have desired product 1-8 (3.5 g, 84.3%).

### tert-butyl 7-(3-ethoxy-1-(1-(2-(2-hydroxyethoxy)ethyl)-4-methyl-1H-benzo[d][1,2,3]triazol-5-yl)-3-oxopropyl)-3,4-dihydroisoquinoline-2(1H)-carboxylate (1-9)

To a mixture of 1-8 (1.5 g, 4.7 mmol), sodium dodecyl sulfate (6.8 g, 23.5 mmol), TEA (1.4 g, 14.1 mmol), compound 1-2 (3.4 g, 9.4 mmol) in CPME (40 mL) and water (20 mL) was added [Rh(1,5-cod)Cl]₂ (231.7 mg, 0.5 mmol) at room temperature. The resulting mixture was stirred at 110° C. for 4 hours under nitrogen atmosphere. After cooling to room temperature, the mixture was poured into ice water (100 mL) and then extracted with ethyl acetate (100 mL*3). The combined organic layers were dried over sodium sulfate, filtered and concentrated to dryness. The residue was purified by column chromatography (elute with ethyl acetate:petroleum ether=1:10 to 1:1) to have the desired product 1-9 (1.0 g, 38.5%).

### ethyl 3-(1-(2-(2-hydroxyethoxy)ethyl)-4-methyl-1H-benzo[d][1,2,3]triazol-5-yl)-3-(1,2,3,4-tetrahydroisoquinolin-7-yl)propanoate (1-10)

To the solution of 1-9 (1.0 mg, 1.8 mmol) in MeOH (10 mL) was added HCl (4M in dioxane, 5 mL) at 0° C. The resulting mixture was stirred at 20° C. for 2 hours and then concentrated to dryness to have desired product 1-10 (820.0 mg, 98.5%).

### ethyl 3-(1-(2-(2-hydroxyethoxy)ethyl)-4-methyl-1H-benzo[d][1,2,3]triazol-5-yl)-3-(2-((4-hydroxyphenyl)sulfonyl)-1,2,3,4-tetrahydroisoquinolin-7-yl)propanoate (1-11)

To the solution of 1-10 (720.0 mg, 1.6 mmol) and 4-hydroxybenzenesulfonyl chloride (367.5 mg, 1.9 mmol) in MeOH (7 mL) was added pyridine (1.3 g, 15.9 mmol) at 0° C. The resulting mixture was stirred at 25° C. for 6 hours. After cooling to room temperature, the mixture was poured into ice water (20 mL) and extracted with ethyl acetate (20 mL*3). The combined organic layers were dried over sodium sulfate, filtered and concentrated to dryness. The residue was purified by column chromatography (elute with ethyl acetate:petroleum ether=1:1 to 3:1) to have the desired product 1-11 (90.0 mg, 9.3%).

### ethyl 2-(1⁴-methyl-4,4-dioxido-3¹,3²,3³,3⁴-tetrahydro-1¹H-6,9-dioxa-4-thia-3(7,2)-isoquinolina-1(5,1)-benzo[d][1,2,3]triazola-5(1,4)-benzenacycloundecaphane-2-yl)acetate (1-12)

To the solution of 1-11 (70.0 mg, 0.1 mmol) in DCM (2 mL) and THE (2 mL) was added DEAD (100.1 mg, 0.6 mmol) and PPh₃ (150.8 mg, 0.6 mmol) at 0° C. The resulting mixture was stirred at 20° C. for 4 hours under nitrogen atmosphere. The mixture was then poured into ice water (10 mL) and extracted with ethyl acetate (10 mL*3). The combined organic layers were dried over sodium sulfate, filtered and concentrated to dryness. The residue was purified by column chromatography (elute with ethyl acetate:petroleum ether=1:1 to 3:1) to have the desired product 1-12 (50.0 mg, 73.6%).

MS(ESI) calculated for C₃₁H₃₄N₄O₆S, 590.2; found 591.2.

### tert-butyl 2-(1⁴-methyl-4,4-dioxido-3¹,3²,3³,3⁴-tetrahydro-1¹H-6,9-dioxa-4-thia-3(7,2)-isoquinolina-1(5,1)-benzo[d][1,2,3]triazola-5(1,4)-benzenacycloundecaphane-2-yl)acetic acid (Compound 1)

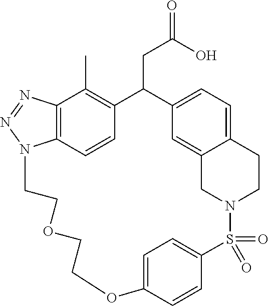 [SMILES: Cc1c2ccc3c1nnn3CCOCCOc1ccc(cc1)S(=O)(=O)N1CCc3ccc(cc3C1)C2CC(=O)O]

To the solution of 1-12 (50.0 mg, 85.0 mol) in THF (2 mL) MeOH (2 mL) and H₂O (1 mL) was added NaOH (10 mg, 0.2 mmol). The resulting mixture was stirred at room temperature for 2 hours under nitrogen atmosphere. The reaction mixture was neutralized with 1N HCl at 0° C. and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have desired product Compound 1 (6.0 mg, 12.5%).

MS(ESI) calculated for C₂₉H₃₀N₄O₆S, 562.2; found 563.2.

¹H NMR (400 MHz, DMSO-d₆) δ 7.72-7.66 (m, 2H), 7.65-7.60 (m, 1H), 7.33-7.28 (m, 1H), 7.03-6.97 (m, 1H), 6.86-6.81 (m, 1H), 6.76-6.70 (m, 3H), 4.88-4.76 (m, 3H), 4.70-4.61 (m, 1H), 4.50-4.42 (m, 1H), 3.98-3.81 (m, 4H), 3.64-3.51 (m, 4H), 3.08-2.92 (m, 2H), 2.92-2.87 (m, 3H), 2.70-2.59 (m, 2H).

### Example 1.2 Synthesis of Compound 2

### Procedure for 2-(carboxymethyl)-1⁴-methyl-4-oxo-3¹,3²,3³,3⁴-tetrahydro-1¹H-3(7,2)-isoquinolina-1(5,1)-benzo[d][1,2,3]triazola-5(4,1)-piperidin-1-iumacyclodecaphan-5¹-ium chloride (Compound 2)

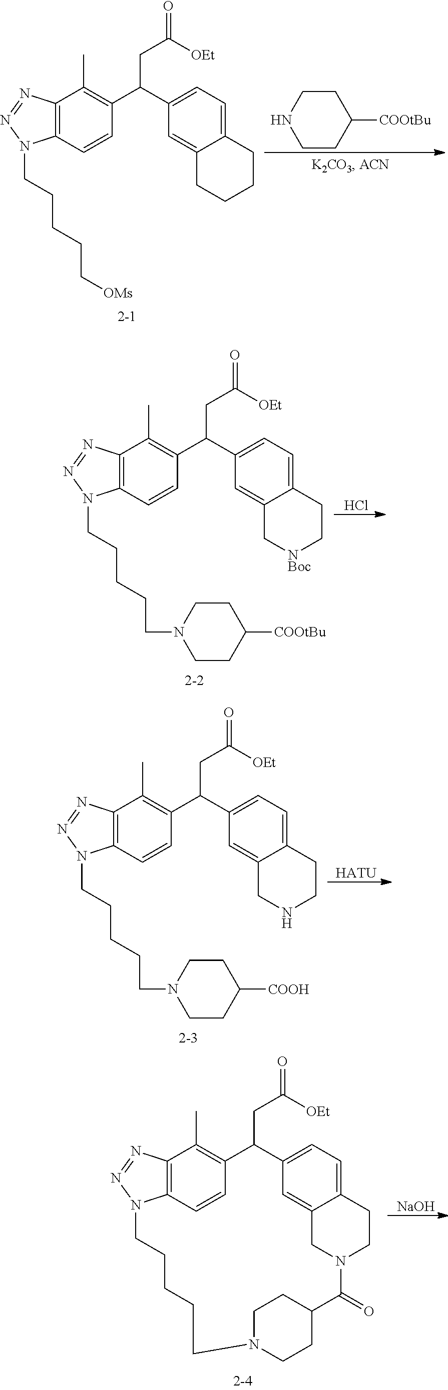

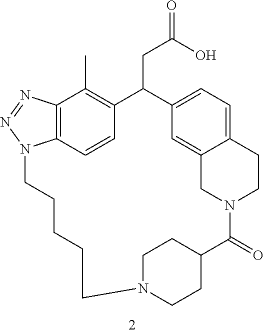 [SMILES: Cc1c2ccc3c1nnn3CCCCCN1CCC(CC1)C(=O)N1CCc3ccc(cc3C1)C2CC(=O)O]

### tert-butyl 7-(1-(1-(5-(4-(tert-butoxycarbonyl)piperidin-1-yl)pentyl)-4-methyl-1H-benzo[d][1,2,3]triazol-5-yl)-3-ethoxy-3-oxopropyl)-3,4-dihydroisoquinoline-2(1H)-carboxylate (2-2)

The mixture of tert-butyl 7-(3-ethoxy-1-(4-methyl-1-(5-((methylsulfonyl)oxy)pentyl)-1H-benzo[d][1,2,3]triazol-5-yl)-3-oxopropyl)-3,4-dihydroisoquinoline-2(1H)-carboxylate (380.0 mg, 0.6 mmol) in ACN (10 mL) was added K₂CO₃ (163.0 mg, 1.2 mmol) and tert-butyl piperidine-4-carboxylate (138.8 mg, 0.8 mmol) at 0° C. The resulting mixture was stirred at 60° C. for 16 hours under nitrogen atmosphere. After cooling to room temperature, the mixture was poured into water (50 mL) and then extracted with ethyl acetate (15 mL×2). The combined organic layers were dried over sodium sulfate, filtered and concentrated to dryness. The residue was purified by column chromatography (eluted with DCM:MeOH=10:1) to have the desired product 2-2 (266.0 mg, 61.3% yield).

MS(ESI) calculated for C₄₁H₅₉N₅O₆, 717.5; found 718.5.

### 1-(5-(5-(3-ethoxy-3-oxo-1-(1,2,3,4-tetrahydroisoquinolin-7-yl)propyl)-4-methyl-1H-benzo[d][1,2,3]triazol-1-yl)pentyl)piperidine-4-carboxylic acid (2-3)

To the solution of compound 2-2 (260.0 mg, 2.1 mmol) in dioxane (10 mL) was added HCl (4N in dioxane, 3 mL) at 0° C. The resulting mixture was stirred at 20° C. for 19 hours under nitrogen atmosphere and then concentrated to have the crude desired product 2-3 (270.4 mg, crude).

MS(ESI) calculated for C₃₂H₄₃N₅O₄, 561.3; found 561.4.

### ethyl 2-(1⁴-methyl-4-oxo-3¹,3²,3³,3⁴-tetrahydro-1¹H-3(7,2)-isoquinolina-1(5,1)-benzo[d][1,2,3]triazola-5(4,1)-piperidinacyclodecaphane-2-yl)acetate (2-4)

The mixture of compound 2-3 (270.0 mg, 0.5 mmol) and DIPEA (620.2 mg, 4.8 mmol) in DMF (5 mL) was added HATU (274.0 mg, 0.7 mmol) at 0° C. The mixture was then poured into ice water (50 mL) and extracted with ethyl acetate (20 mL*3). The combined organic layers were dried over sodium sulfate, filtered and concentrated to dryness. The residue was purified by column chromatography (elute with ethyl acetate:petroleum ether=1:1 to 99:1) to have desired product 2-4 (85.5 mg, 79.5%).

### 2-(carboxymethyl)-1⁴-methyl-4-oxo-3¹,3²,3³,3⁴-tetrahydro-1¹H-3(7,2)-isoquinolina-1(5,1)-benzo[d][1,2,3]triazola-5(4,1)-piperidin-1-iumacyclodecaphan-5¹-ium chloride (Compound 2)

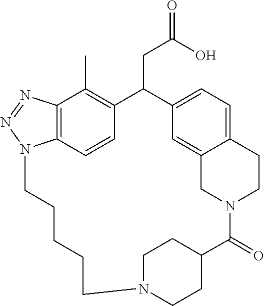 [SMILES: Cc1c2ccc3c1nnn3CCCCCN1CCC(CC1)C(=O)N1CCc3ccc(cc3C1)C2CC(=O)O]

To the solution of 2-4 (85.0 mg, 156.5 mol) in THE (2 mL) MeOH (3 mL) and water (2 mL) was added NaOH (150.0 mg, 3.8 mmol) at room temperature. The resulting mixture was stirred at room temperature for 19 hours under nitrogen atmosphere. After which period, the reaction mixture was neutralized with 1N HCl at 0° C., and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 2 (21.0 mg, 24.7%).

MS(ESI) calculated for C₃₀H₃₇N₅O₃, 515.3; found 516.5.

¹H NMR (400 MHz, DMSO-d₆) δ 9.74 (brs, 1H), 7.64-7.60 (m, 1H), 7.53-7.50 (m, 1H), 7.32-6.91 (m, 3H), 4.83-4.79 (m, 1H), 4.67-4.50 (m, 4H), 3.68-3.60 (m, 4H), 3.06-2.72 (m, 12H), 1.96-1.64 (m, 8H), 1.28-1.19 (m, 2H).

### Example 1.3 Synthesis of Compound 3

### Procedure for 2-(carboxymethyl)-1⁴-methyl-4-oxo-3¹,3²,3³,3⁴-tetrahydro-1¹H-8-oxa-3(7,2)-isoquinolina-1(5,1)-benzo[d][1,2,3]triazola-5(4,1)-piperidin-1-iumacyclodecaphan-5¹-ium chloride (Compound 3)

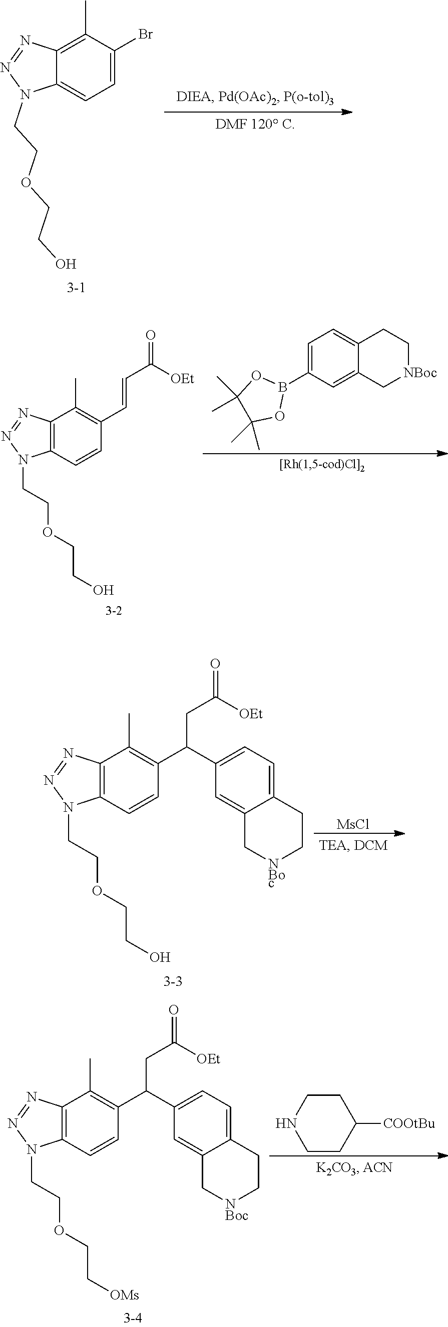

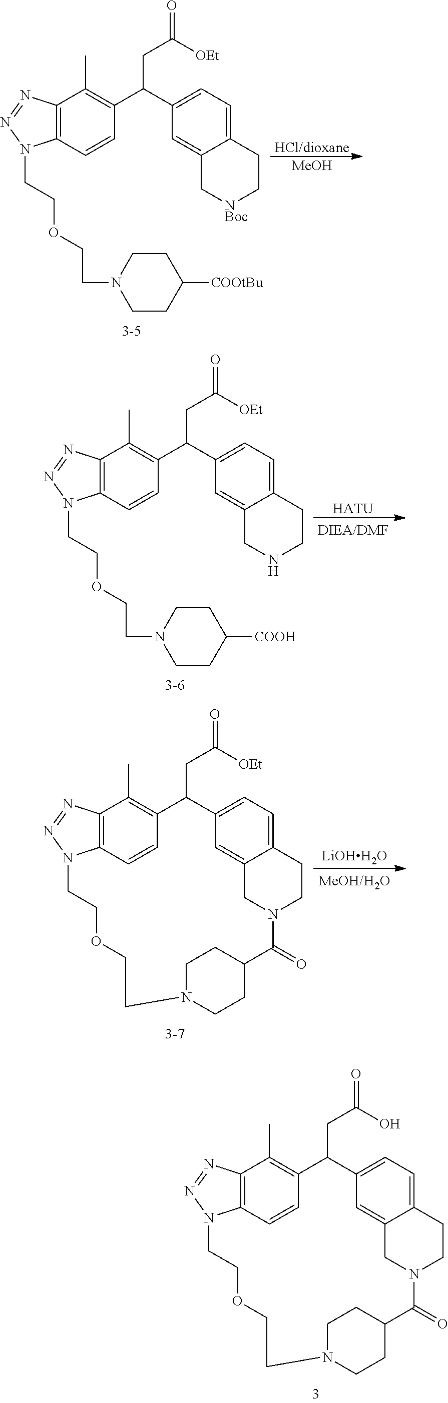

### ethyl (E)-3-(1-(2-(2-hydroxyethoxy)ethyl)-4-methyl-1H-benzo[d][1,2,3]triazol-5-yl)acrylate (3-2)

To the solution of 2-(2-(5-bromo-4-methyl-1H-benzo[d][1,2,3]triazol-1-yl)ethoxy)ethan-1-ol (5.0 g, 16.8 mmol) ethyl acrylate (10.1 g, 100.6 mmol) and DIEA (6.5 g, 50.3 mmol) in DMF (50 mL) was added Pd(OAc)₂ (376.5 mg, 1.7 mmol) and (p-tol)₃P (1.0 g, 3.4 mmol) at 20° C. The resulting mixture was stirred at 120° C. for 19 hours under nitrogen atmosphere. After cooling to room temperature, the mixture was then poured into ice water (500 mL) and extracted with ethyl acetate (100 mL*3). The combined organic layers were dried over sodium sulfate, filtered and concentrated to dryness. The residue was purified by column chromatography (elute with ethyl acetate:petroleum ether=1:10 to 1:2) to have the desired product 3-2 (4.5 g, 80.8%).

MS(ESI) calculated for C₁₆H₂₁N₃O₄,319.2; found 320.2.

### tert-butyl 7-(3-ethoxy-1-(1-(2-(2-hydroxyethoxy)ethyl)-4-methyl-1H-benzo[d][1,2,3]triazol-5-yl)-3-oxopropyl)-3,4-dihydroisoquinoline-2(1H)-carboxylate (3-3)

To a mixture of 3-2 (1.0 g, 3.1 mmol), sodium dodecyl sulfate (350.0 mg, 1.5 mmol), Et₃N (1.1 g, 9.0 mmol), tert-butyl 7-(4,4,5,5-tetramethyl-1,3,2-dioxaborolan-2-yl)-3,4-dihydroisoquinoline-2(1H)-carboxylate (3.3 g, 9.3 mmol) in CPME (20 mL) and water (4 mL) was added [Rh(1,5-cod)Cl]₂ (150.3 mg, 0.3 mmol) at room temperature. The resulting mixture was stirred at 110° C. for 3 hours under nitrogen atmosphere. After cooling to room temperature, the mixture was poured into ice water (100 mL) and then extracted with ethyl acetate (50 mL*3). The combined organic layers were dried over sodium sulfate, filtered and concentrated to dryness. The residue was purified by column chromatography (elute with ethyl acetate:petroleum ether=1:10 to 1:2) to have the desired product 3-3 (800.1 mg, 58.2%).

### tert-butyl 7-(3-ethoxy-1-(4-methyl-1-(2-(2-((methylsulfonyl)oxy)ethoxy)ethyl)-1H-benzo[d][1,2,3]triazol-5-yl)-3-oxopropyl)-3,4-dihydroisoquinoline-2(1H)-carboxylate (3-4)

The mixture of 3-3 (0.8 g, 1.3 mmol) and DIEA (0.5 mL, 0.75 mmol) in DCM (5 mL) was added MsCl (230.0 mg, 2.0 mmol) dropwise. The mixture was stirred at 25° C. overnight and then concentrated under reduced pressure. The residue was purified with column chromatography (elute with ethyl acetate:petroleum ether=1:10 to 1:2) to have desired product 3-4 (560.1 mg, 61.2%).

### tert-butyl 7-(1-(1-(2-(2-(4-(tert-butoxycarbonyl)piperidin-1-yl)ethoxy)ethyl)-4-methyl-1H-benzo[d][1,2,3]triazol-5-yl)-3-ethoxy-3- oxopropyl)-3,4-dihydroisoquinoline-2(1H)-carboxylate (3-5)

To the solution of 3-4 (560.1 mg, 0.8 mmol) in ACN (30 mL) was added K₂CO₃ (216.0 mg, 1.6 mmol) and tert-butyl piperidine-4-carboxylate (120.0 mg, 1.1 mmol). The mixture was stirred at 80° C. overnight. TLC showed the reaction was completed. After cooling down to room temperature, the mixture was poured into ice water (100 mL) and then extracted with ethyl acetate (25 mL*3). The combined organic layers were dried over sodium sulfate, filtered and concentrated to dryness. The residue was purified by column chromatography (elute with ethyl acetate:petroleum ether=1:10 to 1:2) to have the desired product 3-5 (400.0 mg, 48.5%).

### 1-(2-(2-(5-(3-ethoxy-3-oxo-1-(1,2,3,4-tetrahydroisoquinolin-7-yl)propyl)-4-methyl-1H-benzo[d][1,2,3]triazol-1-yl)ethoxy)ethyl)piperidine-4-carboxylic acid (3-6)

To the solution of 3-5 (0.4 g, 0.7 mmol) in MeOH (5 mL) was added HCl (4M in dioxane, 5 mL) at 0° C. The resulting mixture was stirred at 20° C. for 2 hours and then concentrated to dryness to have the desired product 3-6 (400.0 mg, crude).

### ethyl 2-(1⁴-methyl-4-oxo-3¹,3²,3³,3⁴-tetrahydro-1¹H-8-oxa-3(7,2)-isoquinolina-1(5,1)-benzo[d][1,2,3]triazola-5(4,1)-piperidinacyclodecaphane-2-yl)acetate (3-7)

The mixture of 3-6 (400.0 mg, crude) and DIEA (0.5 mL, 6.9 mmol) in DMF (5 mL) was added HATU (420.0 mg, 1.1 mmol). The resulting mixture was stirred at 25° C. overnight. The mixture was then poured into ice water (50 mL) and then extracted with ethyl acetate (25 mL*3). The combined organic layers were dried over sodium sulfate, filtered and concentrated to dryness. The residue was purified by column chromatography (elute with ethyl acetate:petroleum ether=1:5 to 50:1) to have the desired product 3-7 (200.0 mg, 38.2%).

### 2-(carboxymethyl)-1⁴-methyl-4-oxo-3¹,3²,3³,3⁴-tetrahydro-1¹H-8-oxa-3(7,2)-isoquinolina-1(5,1)-benzo[d][1,2,3]triazola-5(4,1)-piperidin-1-iumacyclodecaphan-5¹-ium

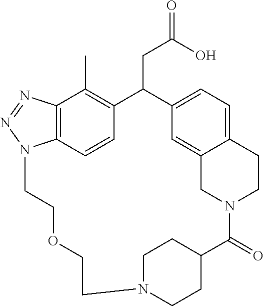 [SMILES: Cc1c2ccc3c1nnn3CCOCCN1CCC(CC1)C(=O)N1CCc3ccc(cc3C1)C2CC(=O)O]

The mixture of 3-7 (200.0 mg, 0.4 mmol), LiOH·H₂O (80 mg, 2.0 mmol) in MeOH (5.0 mL) and water (2 mL) was stirred at 25° C. for 2 hours. After which period, the reaction mixture was neutralized with 1N HCl at 0° C. and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 3 (70.1 mg, 35.8%).

MS(ESI) calculated for C₂₉H₃₅N₅O₄, 517.2; found 518.2.

¹H NMR (400 MHz, DMSO-d₆) δ 7.62-7.57 (m, 1H), 7.51-7.47 (m, 1H), 7.17-6.99 (m, 3H), 4.80-4.70 (m, 3H), 4.59-4.50 (m, 2H), 3.90-3.75 (m, 3H), 3.58-3.40 (m, 8H), 3.12-2.99 (m, 2H), 2.89-2.62 (m, 6H), 2.32-2.27 (m, 2H), 1.91-1.88 (m, 1H), 1.51-1.35 (m, 3H).

### Example 1.4 Synthesis of Compound 4

Procedure for 2-(1⁴,3⁵-dimethyl-4-oxo-3¹,3²,3³,3⁴-tetrahydro-1¹H-7-oxa-3(7,2)-isoquinolina-1(5,1)-benzo[d][1,2,3]triazola-5(1,4)-benzenacyclodecaphane-2-yl)acetic acid (Compound 4)

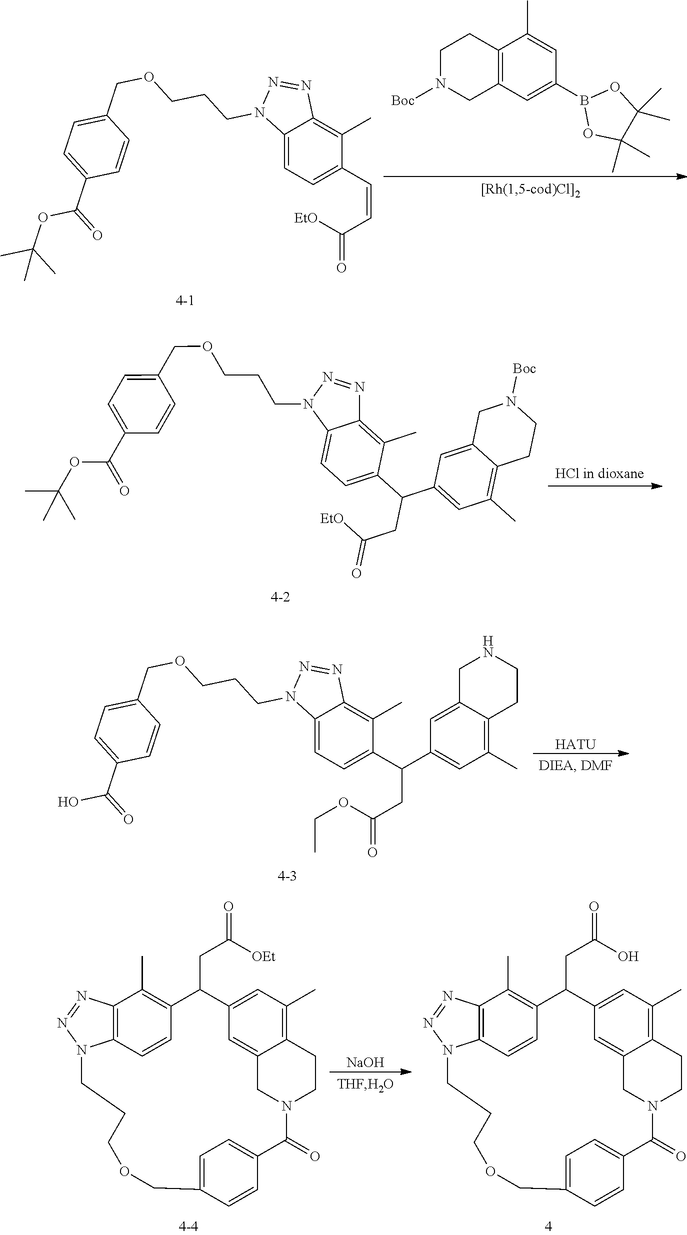

### tert-butyl 7-(1-(1-(3-((4-(tert-butoxycarbonyl)benzyl)oxy)propyl)-4-methyl-1H-benzo[d][1,2,3]triazol-5-yl)-3-ethoxy-3-oxopropyl)-5-methyl-3,4-dihydroisoquinoline-2(1H)-carboxylate (4-2)

To a mixture of 4-1 (0.5 g, 1.0 mmol), sodium dodecyl sulfate (150.3 mg, 0.5 mmol), Et₃N (316.1 mg, 3.0 mmol) and tert-butyl 5-methyl-7-(4,4,5,5-tetramethyl-1,3,2-dioxaborolan-2-yl)-3,4-dihydroisoquinoline-2(1H)-carboxylate (779.0 mg, 2.0 mmol) in CPME (4 mL) and water (2 mL) was added [Rh(1,5-cod)Cl]₂ (51.5 mg, 0.1 mmol) at room temperature. The resulting mixture was stirred at 110° C. for 3 hours under nitrogen atmosphere. After cooling to room temperature, the mixture was poured into ice water (50 mL) and then extracted with ethyl acetate (25 mL*3). The combined organic layers were dried over sodium sulfate, filtered and concentrated to dryness. The residue was purified by column chromatography (elute with ethyl acetate:petroleum ether=1:10 to 1:2) to have desired product 4-2 (355.0 mg, 46.8%).

MS(ESI) calculated for C₄₂H₅₄N₄O₇, 726.4; found 727.1.

### 4-((3-(5-(3-ethoxy-1-(5-methyl-1,2,3,4-tetrahydroisoquinolin-7-yl)-3-oxopropyl)-4-methyl-1H-benzo[d][1,2,3]triazol-1-yl)propoxy)methyl)benzoic acid (4-3)

To the solution of 4-2 (355.0 mg, 0.5 mmol) in dioxane (2.5 mL) was added HCl (4.0 M in dioxane, 2.5 mL) at 0° C. The resulting mixture was stirred at 20° C. for 2 hours. The mixture was then concentrated to dryness to have the desired product 4-3 (420.0 mg, crude).

MS(ESI) calculated for C₃₃H₃₈N₄O₅, 570.3; found 571.1.

### Ethyl 2-(1⁴,3⁵-dimethyl-4-oxo-3¹,3²,3³,3⁴-tetrahydro-1¹H-7-oxa-3(7,2)-isoquinolina-1(5,1)-benzo[d][1,2,3]triazola-5(1,4)-benzenacyclodecaphane-2-yl)acetate (4-4)

To the solution of 4-3 (150.0 mg, 263.1 mol) in DMF (2 mL) was added DIPEA (102.0 mg, 0.8 mmol) and HATU (150.2 mg, 0.4 mmol) at 20° C. The resulting mixture was stirred at room temperature for 2 hours under N₂ atmosphere. The mixture was then poured into ice water (20 mL) and extracted with ethyl acetate (10 mL*3). The combined organic layers were dried over sodium sulfate, filtered and concentrated to dryness. The residue was purified by column chromatography (elute with MeOH:DCM=1:50 to 1:10) to have the desired product 4-4 (113.0 mg, 77.9%).

MS(ESI) calculated for C₃₃H₃₆N₄O₄, 552.3; found 553.4.

### 2-(1⁴,3⁵-dimethyl-4-oxo-3¹,3²,3³,3⁴-tetrahydro-1¹H-7-oxa-3(7,2)-isoquinolina-1(5,1)-benzo[d][1,2,3]triazola-5(1,4)-benzenacyclodecaphane-2-yl)acetic acid (Compound 4)

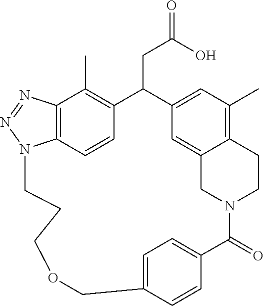 [SMILES: Cc1cc2cc3c1CCN(C3)C(=O)c1ccc(cc1)COCCCn1nnc3c(C)c(ccc31)C2CC(=O)O]

To the solution of 4-4 (113.0 mg, 204.7 mol) in THF (5 mL) MeOH (2 mL) and H₂O (1 mL) was added NaOH (85.0 mg, 2.0 mmol). The resulting mixture was stirred at room temperature for 2 hours under nitrogen atmosphere. The reaction mixture was then neutralized with 1N HCl at 0° C. The aqueous layer was extracted with ethyl acetate (5 mL*3). The combined organic layers were dried over sodium sulfate, filtered and concentrated to dryness. The residue was purified with Prep-HPLC (CH₃CN/H₂O, 0.1% HCl) to have the desired product Compound 4 (8.0 mg, 7.5%).

MS(ESI) calculated for C₃₁H₃₂N₄O₄, 524.2; found 525.3.

¹H NMR (400 MHz, CDCl₃) δ 7.38 (d, J=8.7 Hz, 1H), 7.26-7.22 (m, 2H), 6.90-6.88 (m, 2H), 6.80-6.78 (m, 2H), 5.90 (s, 1H), 4.90 (t, J=7.8 Hz, 1H), 4.85-4.78 (m, 1H), 4.75-4.69 (m, 1H), 4.54-4.51 (m, 1H), 4.24-4.20 (m, 1H), 4.17-4.11 (m, 1H), 4.03 (s, 2H), 3.89-3.82 (m, 1H), 3.46-3.43 (m, 1H), 3.41-3.34 (m, 1H), 3.30-3.26 (m, 1H), 3.11-3.03 (m, 1H), 2.88-2.84 (m, 2H), 2.73 (s, 3H), 2.57-2.50 (m, 1H), 2.44 (s, 1H), 2.31 (s, 3H).

### Example 1.5 Synthesis of Compound 5

### Procedure for 2-(carboxymethyl)-1⁴-methyl-4-oxo-3¹,3²,3³,3⁴-tetrahydro-1¹H-3(7,2)-isoquinolina-1(5,1)-benzo[d][1,2,3]triazola-5(4,1)-piperazin-1-iumacyclodecaphan-5¹-ium chloride (Compound 5)

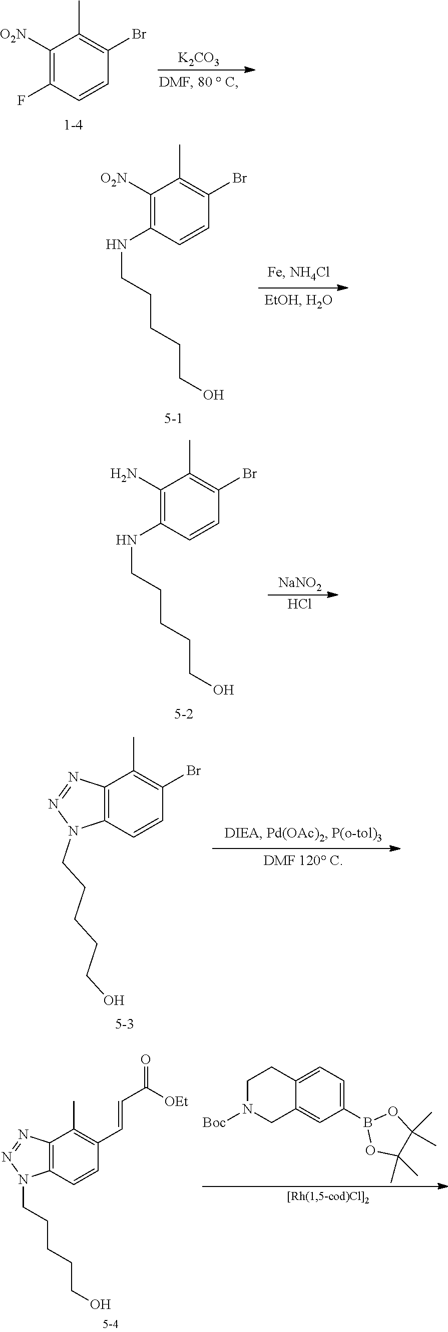

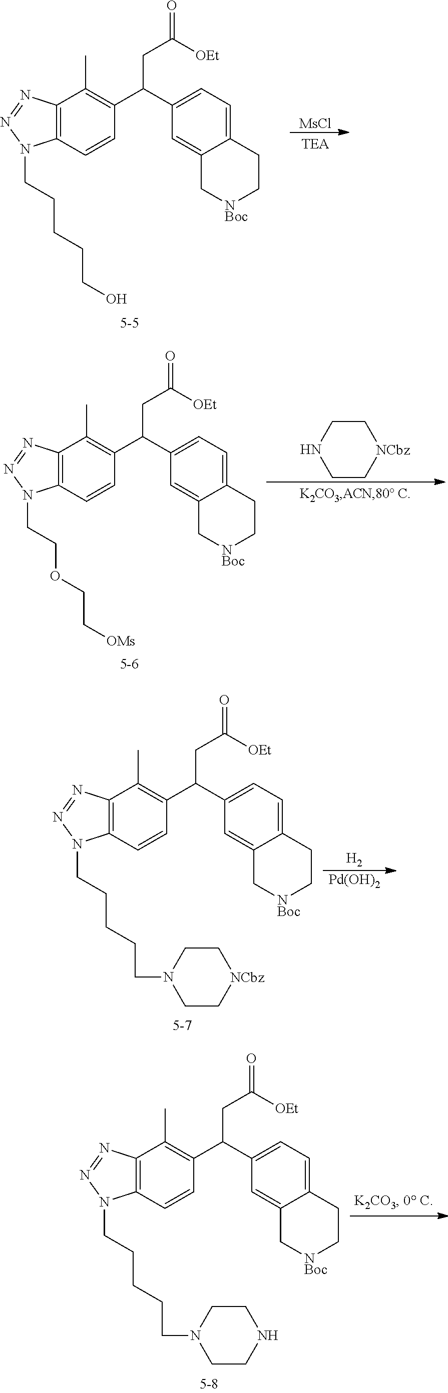

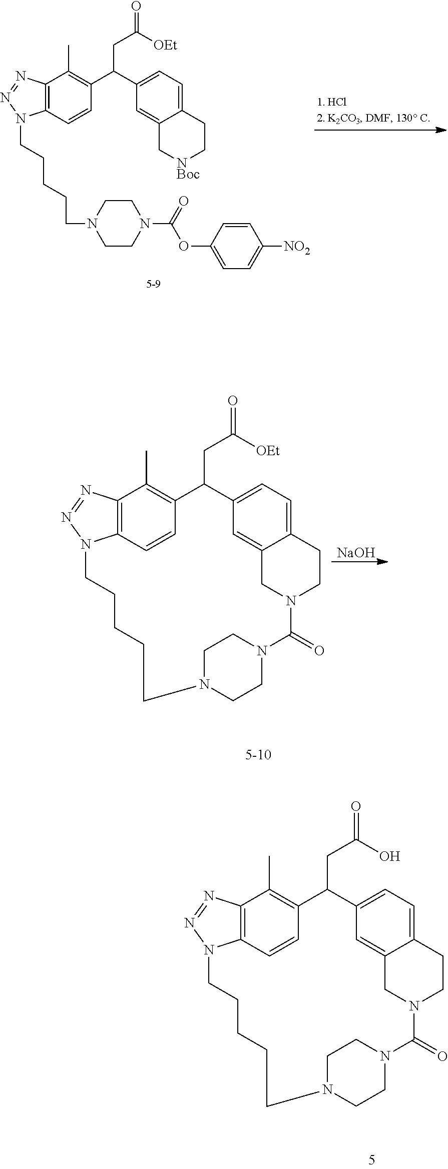

### 5-((4-bromo-3-methyl-2-nitrophenyl)amino)pentan-1-ol (5-1)

To the solution of 1-4 (75.0 g, 320.5 mmol) and 5-aminopentan-1-ol (39.7 g, 384.6 mmol) in DMF (500 mL) was added K₂CO₃ (66.4 g, 480.7 mmol) at 20° C. The resulting mixture was stirred at 80° C. for 19 hours under nitrogen atmosphere. After cooling to room temperature, the mixture was poured into ice water (2 L) and then extracted with ethyl acetate (300 mL*3). The combined organic layers were dried over sodium sulfate, filtered and concentrated to dryness to have crude product 5-1 (90.7 g, 89.2%).

MS(ESI) calculated for C₁₂H₁₇BrN₂O₃, 316.0; found 317.1, 319.1.

### 5-((2-amino-4-bromo-3-methylphenyl)amino)pentan-1-ol (5-2)

To the solution of 5-1 (90.0 g, 283.8 mmol) and NH₄Cl (45.5 g, 851.2 mmol) in EtOH (5 L) and H₂O (0.6 L) was added Fe powder (110.9 g, 2.0 mol) at 20° C. The resulting mixture was stirred at 100° C. for 4.5 hours under nitrogen atmosphere. After cooling to 40° C., the mixture was filtered through a pad of celite. The filtrate was concentrated to dryness to have crude product 5-2 (70.9 g, 87.0%), which was used directly to next run.

MS(ESI) calculated for C₁₂H₁₉BrN₂O, 286.1; found 287.1, 289.1.

### 5-(5-bromo-4-methyl-1H-benzo[d][1,2,3]triazol-1-yl)pentan-1-ol (5-3)

To the solution of 5-2 (70.0 g, 243.7 mmol) in HCl (6M in H₂O, 1.5 L) was added NaNO₂ (33.6 g, 487.5 mmol) in H₂O (200 mL) at 0° C. The reaction mixture was stirred at 25° C. for 2.5 hours and then neutralized with 4N NaOH at 0° C. The resulting mixture was extracted with ethyl acetate (150 mL*3). The combined organic layers were dried over sodium sulfate, filtered and concentrated to dryness. The residue was purified by column chromatography (elute with ethyl acetate:petroleum ether=1:10 to 1:1) to have desired product 5-3 (50.2 g, 69.1%).

MS(ESI) calculated for C₁₂H₁₆BrN₃O, 297.1; found 298.1, 300.1.

### 5-(5-bromo-4-methyl-1H-benzo[d][1,2,3]triazol-1-yl)pentan-1-ol (5-4)

To the solution of 5-3 (5.0 g, 16.8 mmol) ethyl acrylate (10.1 g, 100.6 mmol) and DIEA (6.5 g, 50.3 mmol) in DMF (50 mL) was added Pd(OAc)₂ (376.5 mg, 1.7 mmol) and P(O-tol)₃ (1.0 g, 3.4 mmol) at 20° C. The resulting mixture was stirred at 120° C. for 19 hours under nitrogen atmosphere. After cooling to room temperature, the mixture was then poured into ice water (500 mL) and then extracted with ethyl acetate (100 mL*3). The combined organic layers were dried over sodium sulfate, filtered and concentrated to dryness. The residue was purified by column chromatography (elute with ethyl acetate:petroleum ether=1:10 to 1:2) to have the desired product 5-4 (5.1 g, 95.8%).

### tert-butyl 7-(3-ethoxy-1-(1-(5-hydroxypentyl)-4-methyl-1H-benzo[d][1,2,3]triazol-5-yl)-3-oxopropyl)-3,4-dihydroisoquinoline-2(1H)-carboxylate (5-5)

To a mixture of 5-4 (318.0 mg, 1.0 mmol), sodium dodecyl sulfate (145.0 mg, 0.5 mmol), Et₃N (303.1 mg, 3.0 mmol), 1-2 (1.1 g, 3.0 mmol) in CPME (20 mL) and water (4 mL) was added [Rh(1,5-cod)Cl]₂ (49.3 mg, 0.1 mmol) at room temperature. The resulting mixture was stirred at 110° C. for 3 hours under nitrogen atmosphere. After cooling to room temperature, the mixture was poured into ice water (100 mL) and then extracted with ethyl acetate (25 mL*3). The combined organic layers were dried over sodium sulfate, filtered and concentrated to dryness. The residue was purified by column chromatography (elute with ethyl acetate:petroleum ether=1:10 to 1:2) to have the desired product 5-5 (320.1 mg, 58.2%).

### tert-butyl 7-(1-(1-(5-(4-((benzyloxy)carbonyl)piperazin-1-yl)pentyl)-4-methyl-1H-benzo[d][1,2,3]triazol-5-yl)-3-ethoxy-3-oxopropyl)-3,4-dihydroisoquinoline-2(1H)-carboxylate (5-6)

To the solution of 5-5 (200.0 mg, 363.2 mol) and Et₃N (73.5 mg, 726.4 mol) in DCM (10 mL) was added MsCl (62.4 mg, 544.8 mol) at 0° C. The resulting mixture was stirred at 20° C. for 2.5 hours. After which period, the mixture was poured into ice water (50 mL) and then extracted with ethyl acetate (15 mL*3). The combined organic layers were dried over sodium sulfate, filtered and concentrated to dryness. The residue was purified by column chromatography (elute with ethyl acetate:petroleum ether=1:1 to 2:1) to have the desired product 5-6 (190.0 mg, 83.2%).

### tert-butyl 7-(1-(1-(5-(4-((benzyloxy)carbonyl)piperazin-1-yl)pentyl)-4-methyl-1H-benzo[d][1,2,3]triazol-5-yl)-3-ethoxy-3-oxopropyl)-3,4-dihydroisoquinoline-2(1H)-carboxylate (5-7)

To the solution of 5-6 (190.0 mg, 0.3 mmol) and benzyl piperazine-1-carboxylate (79.9 mg, 0.4 mmol) in acetonitrile (10 mL) was added K₂CO₃ (62.6 mg, 0.5 mmol) at 0° C. The resulting mixture was then stirred at 80° C. for 6.5 hours. After cooling to room temperature, the mixture was poured into ice water (50 mL) and then extracted with ethyl acetate (15 mL*3). The combined organic layers were dried over sodium sulfate, filtered and concentrated to dryness. The residue was purified by column chromatography (elute with ethyl acetate:petroleum ether=1:1 to 3:1) to have the desired product 5-7 (120.0 mg, 53.3%).

### tert-butyl 7-(3-ethoxy-1-(4-methyl-1-(5-(piperazin-1-yl)pentyl)-1H-benzo[d][1,2,3]triazol-5-yl)-3-oxopropyl)-3,4-dihydroisoquinoline-2(1H)-carboxylate (5-8)

To the solution of 5-7 (120.0 mg, 0.2 mmol) in MeOH (10 mL) was added Pd(OH)₂ (20.2 mg). The resulting mixture was stirred at 20° C. for 6.5 hours under H₂ atmosphere. The catalyst was then removed by filtration, and the filtrate was concentrated to have the desired product 5-8 (90.0 mg, 93.8%).

MS(ESI) calculated for C₃₅H₅₀N₆O₄, 618.4; found 619.5.

### tert-butyl 7-(3-ethoxy-1-(4-methyl-1-(5-(4-((4-nitrophenoxy)carbonyl)piperazin-1-yl)pentyl)-1H-benzo[d][1,2,3]triazol-5-yl)-3-oxopropyl)-3,4-dihydroisoquinoline-2(1H)-carboxylate (5-9)

To the solution of 5-8 (90.0 mg, 0.1 mmol) and K₂CO₃ (30.1 mg, 0.2 mmol) in acetonitrile (10 mL) was added 4-Nitrophenyl chloroformate (27.3 mg, 0.2 mmol) at 0° C. The resulting mixture was stirred at 20° C. for 19 hours. After which period, the mixture was poured into ice water (50 mL) and then extracted with ethyl acetate (30 mL*3). The combined organic layers were dried over sodium sulfate, filtered and concentrated to dryness. The residue was purified by column chromatography (elute with ethyl acetate:petroleum ether=1:1 to 3:1) to have the desired product 5-9 (50.0 mg, 44.0%).

### ethyl 2-(1⁴-methyl-4-oxo-3¹,3²,3³,3⁴-tetrahydro-1¹H-3(7,2)-isoquinolina-1(5,1)-benzo[d][1,2,3]triazola-5(1,4)-piperazinacyclodecaphane-2-yl)acetate (5-10)

To the solution of 5-9 (40.0 mg, 0.1 mmol) in MeOH (5 mL) was added HCl (4M in dioxane, 2.5 mL) at 0° C. The resulting mixture was stirred at 20° C. for 2 hours and then concentrated to dryness to have desired amine (30.0 mg) as colorless oil, which was re-dissolved in DMF (5 mL). K₂CO₃ (12.1 mg, 0.1 mmol) was added to the above solution. The resulting mixture was placed in a sealed tube and heated to 130° C. in a microwave reactor for 3 hours. After cooling to room temperature, the mixture was concentrated and purified with Prep-HPLC (CH₃CN/H₂O, 0.1% HCl) to have the desired product 5-10 (20.0 mg, 72% over 2 steps).

MS(ESI) calculated for C₃₁H₄₀N₆O₃, 544.3; found 545.3.

### 2-(carboxymethyl)-1⁴-methyl-4-oxo-3¹,3²,3³,3⁴-tetrahydro-1¹H-3(7,2)-isoquinolina-1(5,1)-benzo[d][1,2,3]triazola-5(4,1)-piperazin-1-iumacyclodecaphan-5¹-ium chloride (Compound 5)

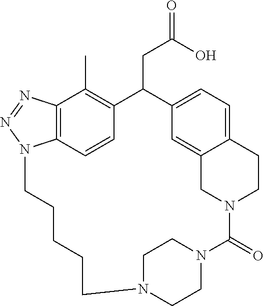 [SMILES: Cc1c2ccc3c1nnn3CCCCCN1CCN(CC1)C(=O)N1CCc3ccc(cc3C1)C2CC(=O)O]

To the solution of 5-10 (24.0 mg, 44.1 mol) in THF (5 mL), MeOH (2 mL) and H₂O (1 mL) was added NaOH (8.8 mg, 0.2 mmol). The resulting mixture was stirred at room temperature for 2 hours under nitrogen atmosphere. After which period, the reaction mixture was neutralized with 1N HCl at 0° C. and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 5 (1.8 mg, 7.4%).

MS(ESI) calculated for C₂₉H₃₆N₆O₃, 516.2; found 517.3.

¹H NMR (400 MHz, MeOH-d₄) δ 7.69 (d, J=8.6 Hz, 1H), 7.56 (d, J=8.7 Hz, 1H), 7.42 (d, J=7.8 Hz, 1H), 7.19 (d, J=7.8 Hz, 1H), 6.75 (s, 1H), 4.95 (t, J=7.8 Hz, 1H), 4.78-4.67 (m, 2H), 4.25 (s, 2H), 3.76-3.71 (m, 1H), 3.66-3.52 (m, 1H), 3.20-2.80 (m, 12H), 2.72 (s, 3H), 2.68-2.64 (m, 2H), 2.35-2.20 (m, 1H), 2.19-2.10 (m, 1H), 1.42-1.19 (m, 4H).

### Example 1.6 Synthesis of Compound 6

### Procedure for 2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-piperidinacyclodecaphane-2-yl)acetic acid (Compound 6)

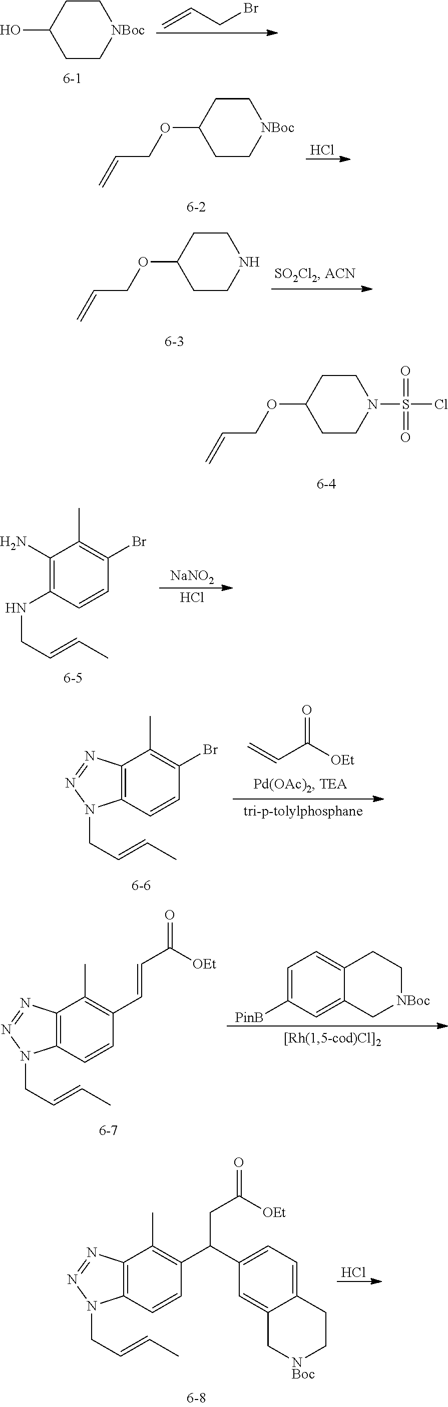

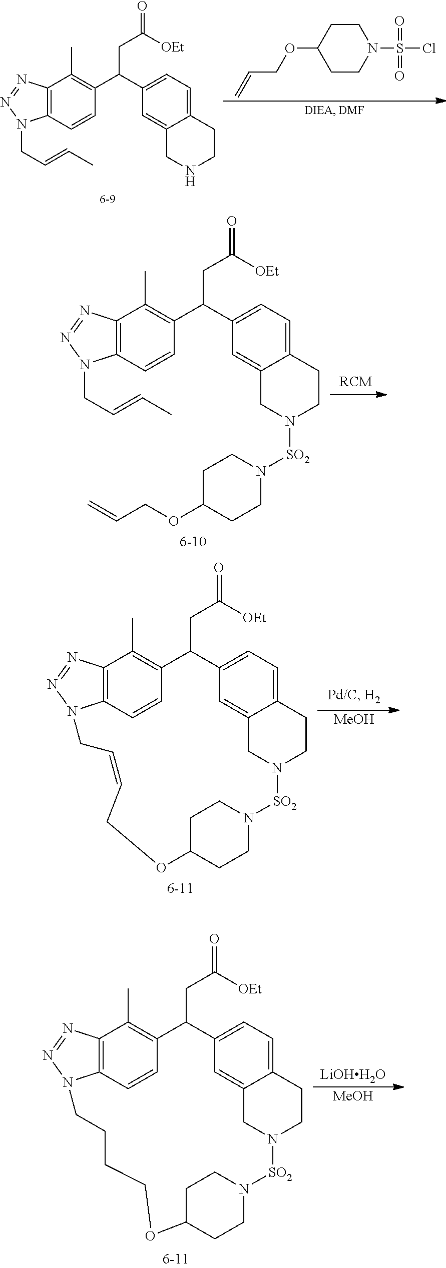

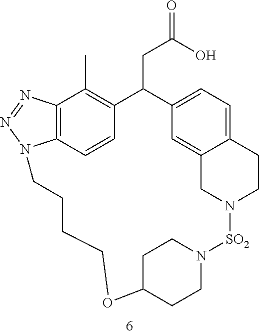 [SMILES: Cc1c2ccc3c1nnn3CCCCOC1CCN(CC1)S(=O)(=O)N1CCc3ccc(cc3C1)C2CC(=O)O]

### tert-butyl 4-(allyloxy)piperidine-1-carboxylate (6-2)

To the solution of tert-butyl 4-hydroxypiperidine-1-carboxylate (2.0 g, 8.3 mmol) in DMF (30 mL) was added NaH (60% in mineral oil, 1.0 g, 25.0 mmol) at 0° C. The resulting mixture was stirred for about 30 min. To the above mixture 3-bromoprop-1-ene was added; the resulting mixture was stirred for another 12 hours. The mixture was then poured into ice water (200 mL) and extracted with ethyl acetate (50 mL*3). The combined organic layers were dried over sodium sulfate, filtered and concentrated to dryness. The residue was purified by column chromatography (elute with ethyl acetate:petroleum ether=1:10 to 1:5) to have the desired product 6-2 (2.1 g, 80% yield).

¹H NMR (400 MHz, CDCl₃) δ 6.03-5.82 (m, 1H), 5.31-5.30 (m, 1H), 5.17 (m, 1H), 4.03-4.01 (m, 2H), 3.86-3.68 (m, 2H), 3.55-3.41 (m, 1H), 3.08 (m, 2H), 1.82 (m, 2H), 1.58-1.47 (m, 2H), 1.45 (s, 9H).

### 4-(allyloxy)piperidine (6-3)

To a stirred solution of 6-2 (1.0 g, 4.2 mmol) in DCM (20 mL) was added HCl in dioxane (4N, 10 mL) at room temperature. The resulting mixture was stirred for about 2 h and then concentrated under reduced pressure to have the desired product 6-3 (1.0 g crude).

### 4-(allyloxy)piperidine-1-sulfonyl chloride (6-4)

To the mixture of 6-3 (0.3 g, 2.1 mmol) and DIEA (0.5 mL, 0.75 mmol) in ACN (10 mL) was added SO₂Cl₂ (0.3 g, 2.5 mmol) dropwise. The resulting mixture was stirred at 25° C. for 4.5 hours under nitrogen atmosphere. The mixture was then concentrated under reduced pressure to get the desired product 6-4 (0.3 g, crude). It was used for the next step without further purification.

### (E)-5-bromo-1-(but-2-en-1-yl)-4-methyl-1H-benzo[d][1,2,3]triazole (6-6)

To the solution of (E)-4-bromo-N1-(but-2-en-1-yl)-3-methylbenzene-1,2-diamine (2.3 g, 9.1 mmol) in THE (30 mL) was added HCl (aq. 6N, 60 mL) and NaNO₂ (1.9 g, 27.3 mmol) at 0° C. in turn. The resulting mixture was stirred at 0° C. for 3 hours under nitrogen atmosphere. The mixture was then poured into ice water (100 mL) and extracted with ethyl acetate (50 mL*3). The combined organic layers were dried over sodium sulfate, filtered and concentrated to dryness. The residue was purified by column chromatography (elute with ethyl acetate:petroleum ether=1:10) to have the desired product 6-6 (2.1 g, 85.7%).

MS(ESI) calculated for C₁₁H₁₂BrN₃, 265.0; found 266.1, 268.1.

### ethyl (E)-3-(1-((E)-but-2-en-1-yl)-4-methyl-1H-benzo[d][1,2,3]triazol-5-yl)acrylate (6-7)

To the solution of 6-6 (2.1 g, 7.9 mmol) ethyl prop-2-enoate (4.7 g, 47.3 mmol) tri-p-tolylphosphane (0.2 g, 0.7 mmol) and Pd(OAc)₂ (0.2 g, 0.8 mmol) in DMF was added DIPEA (6.5 g, 50.3 mmol). The resulting mixture was stirred at 120° C. for 19 hours under nitrogen atmosphere. After colling to room temperature, the reaction mixture was poured into ice water (200 mL) and extracted with ethyl acetate (80 mL*3). The combined organic layers were dried over sodium sulfate, filtered and concentrated to dryness. The residue was purified by column chromatography (elute with ethyl acetate:petroleum ether=1:10 to 1:3) to have the desired product 6-7 (1.6 g, 71.1% yield).

MS(ESI) calculated for C₁₆H₁₉N₃O₂, 285.0; found 286.5.

### tert-butyl (E)-7-(1-(1-(but-2-en-1-yl)-4-methyl-1H-benzo[d][1,2,3]triazol-5-yl)-3-ethoxy-3-oxopropyl)-3,4-dihydroisoquinoline-2(1H)-carboxylate (6-8)

To a mixture of 6-7 (1.6 g, 3.5 mmol) sodium dodecyl sulfate (0.5 g, 1.8 mmol) Et₃N (303.1 mg, 3.0 mmol) and tert-butyl 7-(4,4,5,5-tetramethyl-1,3,2-dioxaborolan-2-yl)-3,4-dihydroisoquinoline-2(1H)-carboxylate (2.0 g, 6.0 mmol) in CPME (30 mL) and water (3 mL) was added [Rh(1,5-cod)Cl]₂ (120.0 mg, 0.3 mmol) at room temperature. The resulting mixture was stirred at 110° C. for 4.5 hours under nitrogen atmosphere. After cooling to room temperature, the mixture was then poured into ice water (100 mL) and then extracted with ethyl acetate (25 mL*3). The combined organic layers were dried over sodium sulfate, filtered and concentrated to dryness. The residue was purified by column chromatography (elute with ethyl acetate:petroleum ether=1:10 to 1:2) to have the desired product 6-8 (1.2 g, 59.6%).

### ethyl (E)-3-(1-(but-2-en-1-yl)-4-methyl-1H-benzo[d][1,2,3]triazol-5-yl)-3-(1,2,3,4-tetrahydroisoquinolin-7-yl)propanoate (6-9)

The mixture of 6-8 (0.2 g, 0.5 mmol) in Dioxane (5 mL) was added HCl (4M in dioxane, 5 mL) at 0° C. The resulting mixture was stirred at 20° C. for 2 hours under nitrogen atmosphere and then concentrated to dryness to have the desired product 6-9 (150.0 mg, crude). It was used for the next step without further purification.

### ethyl (E)-3-(2-((4-(allyloxy)piperidin-1-yl)sulfonyl)-1,2,3,4-tetrahydro isoquinolin-7-yl)-3-(1-(but-2-en-1-yl)-4-methyl-1H-benzo[d][1,2,3]triazol-5-yl)propanoate (6-10)

To the mixture of 6-9 (150.0 mg, crude) and DIEA (0.1 g, 1.1 mmol) in DMF (5 mL) was added 6-4 (171.8 mg, crude) slowly. The resulting mixture was stirred at 25° C. for 19 hours under nitrogen atmosphere. The mixture was then poured into water (30 mL) and extracted with ethyl acetate (10 mL*3). The combined organic layers were dried over sodium sulfate, filtered and concentrated to dryness. The residue was purified with Prep-HPLC (CH₃CN/H₂O, 0.1% HCl) to have the desired product 6-10 (120.0 mg, 55.0%).

### ethyl (E)-2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-piperidinacyclodecaphan-8-en-2-yl)acetate (6-11)

To the mixture of 6-10 (120.0 mg, 0.2 mmol) in DCM (30.0 mL) was added Grubbs catalyst (40.0 mg, 0.1 mmol) at room temperature. The resulting mixture was stirred at 40° C. for 19 hours under nitrogen atmosphere. The mixture was then concentrated to dryness. The residue was purified with Prep-HPLC (CH₃CN/H₂O, 0.1% HCl) to have the desired product 6-11 (100.0 mg, 89.4%).

### ethyl 2-(1⁴-methyl-4,4-dioxido-3¹,3²,3³,3⁴-tetrahydro-11H-6-oxa-4-thia-3(7,2)-isoquinolina-1(5,1)-benzo[d][1,2,3]triazola-5(1,4)-piperidinacyclodecaphane-2-yl)acetate (6-12)

The mixture of 6-11 (100.0 mg, 0.2 mmol) and Pd/C (20.0 mg) in MeOH (20 mL) was stirred at room temperature for 2 hours under H₂ atmosphere. Then the mixture was filtered through a pad celite. The filtrate was concentrated in vacuum to get the desired product 6-12 (100.0 mg, 99%).

### 2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-piperidinacyclodecaphane-2-yl)acetic acid (Compound 6)

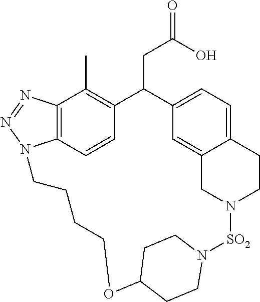 [SMILES: Cc1c2ccc3c1nnn3CCCCOC1CCN(CC1)S(=O)(=O)N1CCc3ccc(cc3C1)C2CC(=O)O]

The mixture of 6-12 (100.0 mg, crude) and LiOH·H₂O (50 mg) in MeOH (5.0 mL) and water (2.0 mL) was stirred at 20° C. for 2 hours. After which period, the reaction mixture was neutralized with 1N HCl at 0° C. and extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 6 (5.1 mg, 5.8%).

MS(ESI) calculated for C₂₉H₃₆N₆O₃, 553.7; found 554.2.

¹H NMR (400 MHz, DMSO-d₆) δ 7.61 (d, J=8.8 Hz, 1H), 7.51 (d, J=8.8 Hz, 1H), 7.34 (d, J=8.0 Hz, 1H), 7.15 (s, 1H), 7.10 (d, J=8.0 Hz, 1H), 4.86-4.82 (m, 1H), 4.77-4.66 (m, 2H), 4.18 (d, J=16.0 Hz, 1H), 4.10 (d, J=16.0 Hz, 1H), 3.20-3.11 (m, 4H), 2.90-2.80 (m, 1H), 2.80-2.75 (m, 4H), 2.75-2.70 (m, 3H), 2.70-2.62 (m, 1H), 2.37 (m, 2H), 2.02-1.90 (m, 1H), 1.82-1.70 (m, 1H), 1.31-1.25 (m, 2H), 1.21-1.12 (m, 2H), 0.98 (m, 2H), 0.85-0.72 (m, 1H).

### Example 1.7 Synthesis of Compound 7

### Synthesis of 2-(14,5-dimethyl-6-oxo-11H-9-oxa-5-aza-1(5,1)-benzo[d][1,2,3]triazola-3(1,3), 7(1,4)-dibenzenacyclododecaphane-2-yl)acetic acid (Compound 7)

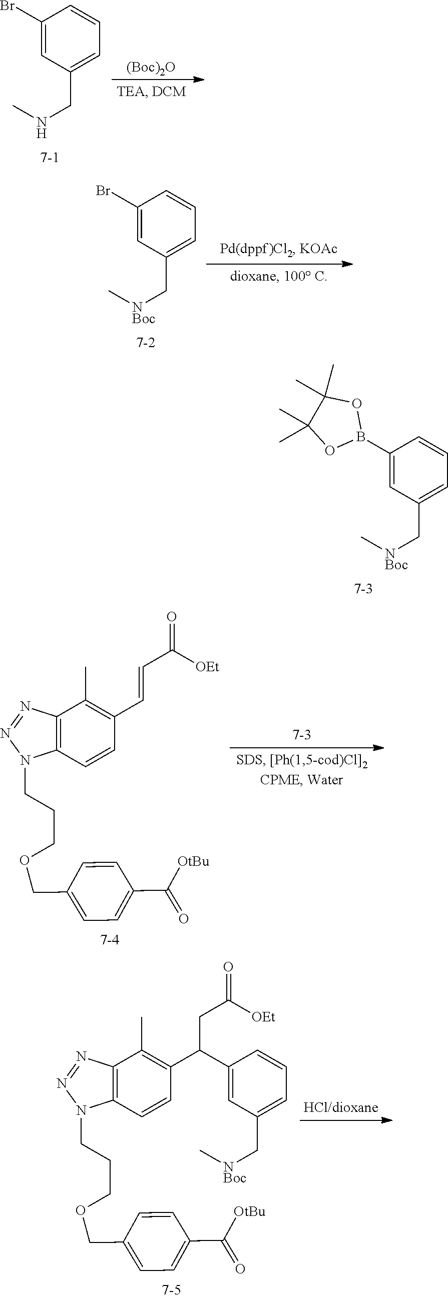

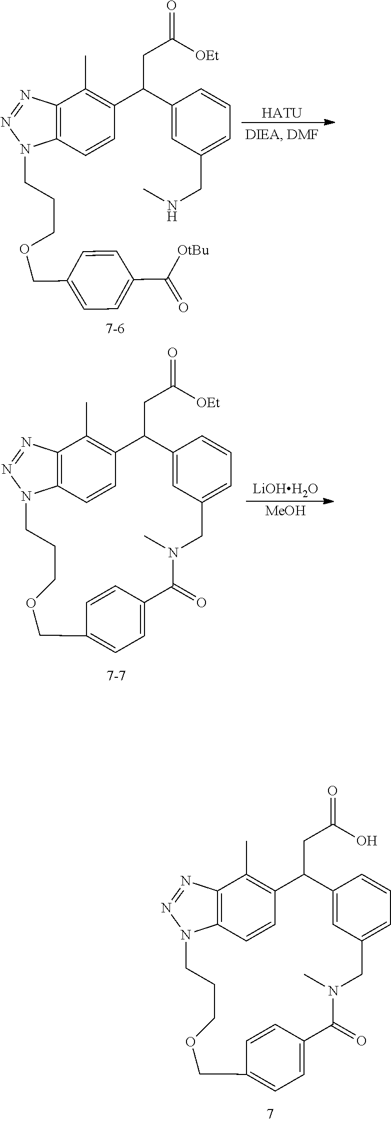

### tert-butyl (3-bromobenzyl)(methyl)carbamate (7-2)

To the solution of 1-(3-bromophenyl)-N-methylmethanamine (25.0 g, 125.0 mmol) and TEA (52.6 mL, 375.0 mmol) in DCM (300 mL) was added (Boc)₂O (39.1 g, 188.0 mmol) dropwise. The resulting mixture was stirred at 20° C. for 19 hours under nitrogen atmosphere. After which period the mixture was then poured into ice water (500 mL) and then extracted with ethyl acetate (100 mL*3). The combined organic layers were dried over sodium sulfate, filtered and concentrated to dryness. The residue was purified by column chromatography (elute with ethyl acetate:petroleum ether=1:10 to 1:5) to have desired product 7-2 (37.0 g, 80.0%).

### tert-butyl 4-(allyloxy) piperidine-1-carboxylate (7-3)

To the solution of 7-2 (37.0 g, 123.3 mmol), 4,4,4′,4′,5,5,5′,5′-octamethyl-2,2′-bi(1,3,2-dioxaborolane) (34.0 g, 135.6 mmol) and AcOK (4.5 g, 369.9 mmol) in DMF (300 mL) was added Pd(dppf)Cl₂ (0.5 g, 6.7 mmol) at 20° C. The resulting mixture was stirred at 100° C. for 19 hours under nitrogen atmosphere. After cooling to room temperature, the mixture was then poured into ice water (500 mL) and then extracted with ethyl acetate (100 mL*3). The combined organic layers were dried over sodium sulfate, filtered and concentrated to dryness. The residue was purified by column chromatography (elute with ethyl acetate:petroleum ether=1:10 to 1:2) to have the desired product 7-3 (20.1 g, 45.8%).

¹H NMR (400 MHz, CDCl₃) δ 7.78-7.60 (m, 2H), 7.45-7.29 (m, 2H), 4.42 (s, 2H), 2.81 (s, 3H), 1.49 (s, 9H), 1.34 (s, 12H).

### tert-butyl 4-((3-(5-(1-(3-(((tert-butoxycarbonyl)(methyl)amino)methyl)phenyl)-3-ethoxy-3-oxopropyl)-4-methyl-1H-benzo[d][1,2,3]triazol-1-yl)propoxy)methyl)benzoate (7-5)

To a mixture of 7-4 (0.3 g, 0.7 mmol), sodium dodecyl sulfate (250.0 mg, 0.4 mmol), Et₃N (0.2 g, 2.1 mmol) and 7-3 (0.5 g, 1.4 mmol) in CPME (10 mL) and water (3 mL) was added [Rh(1,5-cod)Cl]₂ (30.0 mg, 0.1 mmol) at room temperature. The resulting mixture was stirred at 110° C. for 3 hours under nitrogen atmosphere. After cooling to room temperature, the mixture was poured into ice water (200 mL) and extracted with ethyl acetate (50 mL*3). The combined organic layers were dried over sodium sulfate, filtered and concentrated to dryness. The residue was purified by column chromatography (elute with ethyl acetate:petroleum ether=1:5 to 1:2) to have the desired product 7-5 (230.0 mg, 68.2%).

### 4-((3-(5-(3-ethoxy-1-(3-((methylamino)methyl)phenyl)-3-oxopropyl)-4-methyl-1H-benzo[d][1,2,3]triazol-1-yl)propoxy)methyl)benzoic acid (7-6)

To a stirred mixture of 7-5 (230.0 mg, 0.3 mmol) in MeOH (10 mL) was added HCl in dioxane (4M, 10 mL). The resulting mixture was stirred at 20° C. for 2 h. TLC showed started material was consumed. The mixture was concentrated under reduced pressure to get the desired product 7-6 (0.2 g, crude).

### ethyl 2-(1⁴,5-dimethyl-6-oxo-1¹H-9-oxa-5-aza-1(5,1)-benzo[d][1,2,3]triazola-3(1,3), 7(1,4)-dibenzenacyclododecaphane-2-yl)acetate (7-7)

To the solution of 7-6 (200.0 mg, crude) and DIEA (0.5 mL, 6.9 mmol) in DMF (5 mL) was added HATU (200.0 mg, 0.5 mmol) at 0° C. The resulting mixture was stirred at 25° C. overnight. TLC showed the reaction was completed. The mixture was poured into ice water (50 mL) and then extracted with ethyl acetate (25 mL*3). The combined organic layers were dried over sodium sulfate, filtered and concentrated to dryness. The residue was purified by column chromatography (elute with ethyl acetate:petroleum ether=1:5 to 2:1) to have the desired product 7-7 (120.0 mg, 58.2%).

### 2-(1⁴,5-dimethyl-6-oxo-1¹H-9-oxa-5-aza-1(5,1)-benzo[d][1,2,3]triazola-3(1,3), 7(1,4)-dibenzenacyclododecaphane-2-yl)acetic acid (Compound 7)

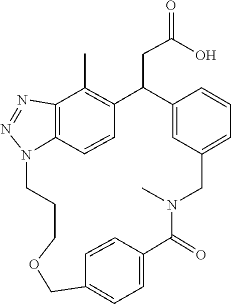 [SMILES: Cc1c2ccc3c1nnn3CCCOCc1ccc(cc1)C(=O)N(C)Cc1cccc(c1)C2CC(=O)O]

The mixture of 7-7 (120.0 mg, 0.2), LiOH·H₂O (45.0 mg, 1.0 mmol) in MeOH (5.0 mL) and water (2.0 mL) was stirred at 20° C. for 2 hours. After which period, the reaction mixture was neutralized with 1N HCl at 0° C. and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have desired product Compound 7 (16.1 mg, 15.0%).

MS(ESI) calculated for C₂₉H₃₀N₄O₄, 498.2; found 499.2.

¹H NMR (400 MHz, DMSO-d₆) δ 7.75-7.60 (m, 1H), 7.51-7.40 (m, 1H), 7.40-7.33 (m, 1H), 7.30-7.20 (m, 1H), 7.15-7.10 (m, 1H), 7.00-6.66 (m, 4H), 6.53 (s, 1H), 4.90-4.77 (m, 2H), 4.71-4.48 (m, 3H), 4.30-4.20 (m, 3H), 3.39-3.30 (m, 1H), 3.17-3.02 (m, 3H), 2.97-2.82 (m, 2H), 2.70-2.61 (m, 3H), 2.43-2.29 (m, 2H).

### Example 1.8 Synthesis of Compound 8

### Procedure for 2-(1⁴-methyl-4-oxo-1¹H-8-oxa-1(5,1)-benzo[d][1,2,3]triazola-5(1,4)-piperazina-3(2,7)-naphthalenacyclodecaphane-2-yl)acetic acid (Compound 8)

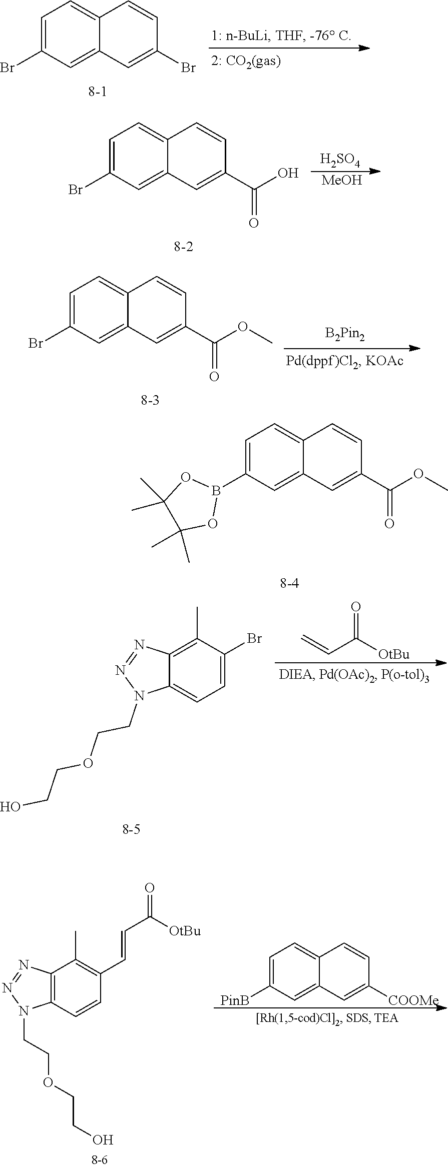

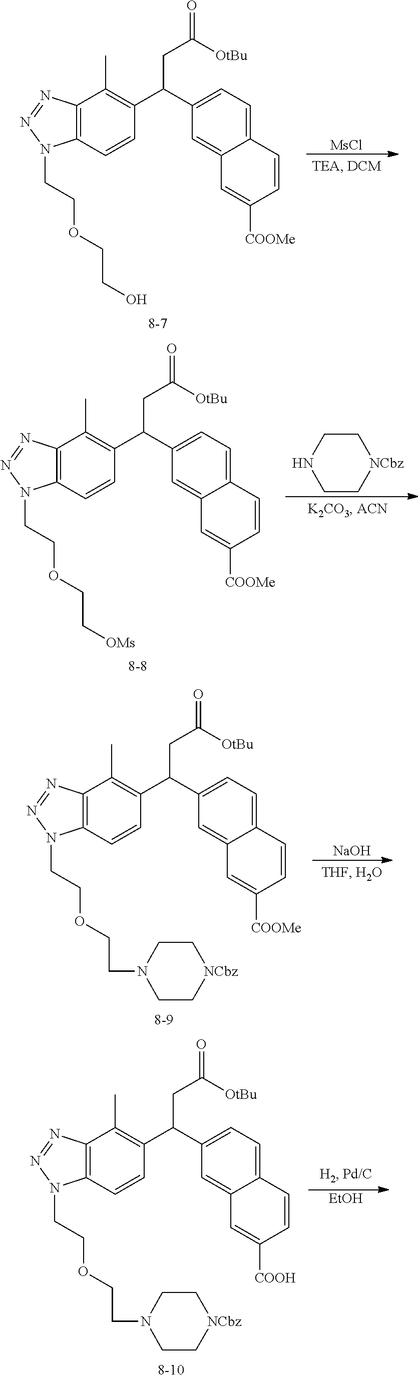

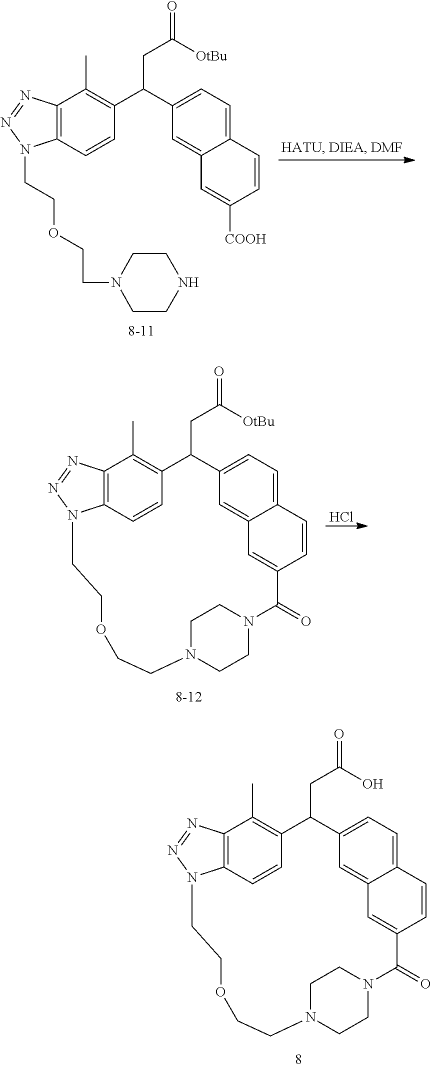 [SMILES: Cc1c(C(CC(=O)OC(C)(C)C)c2ccc3ccc(C(=O)O)cc3c2)ccc2c1nnn2CCOCCN1CCNCC1 ; Cc1c2ccc3c1nnn3CCOCCN1CCN(CC1)C(=O)c1ccc3ccc(cc3c1)C2CC(=O)OC(C)(C)C ; Cl ; Cc1c2ccc3c1nnn3CCOCCN1CCN(CC1)C(=O)c1ccc3ccc(cc3c1)C2CC(=O)O]

### 7-bromo-2-naphthoic acid (8-2)

To a mixture of 2,7-dibromonaphthalene (10.0 g, 35.0 mmol) in THE (140 mL) was added n-BuLi (1.6 M in hexanes, 23 mL, 36.8 mmol). The resulting mixture was stirred at −76° C. for 15 minutes, CO₂ (gas) was then bubbled through the reaction solution. The resulting mixture was stirred at −76° C. for 0.5 hours under CO₂ atmosphere and then allowed to warm to room temperature. The solution was continued to stir at 25° C. for 16 hours under CO₂ atmosphere and then acidified with 1N HCl at 0° C. The resulting mixture was then poured into ice water (500 mL) and n extracted with ethyl acetate (200 mL*3). The combined organic layers were dried over sodium sulfate, filtered and concentrated to dryness. The residue was purified by column chromatography (elute with ethyl acetate:petroleum ether=1:10 to 1:5) to have desired product 8-2 (8.2 g, 93.4%).

¹H NMR (400 MHz, DMSO-d₆) δ 13.20 (s, 1H), 8.59 (s, 1H), 8.45-8.40 (m, 1H), 7.93-8.11 (m, 3H), 7.80-7.75 (m, 1H).

### methyl 7-bromo-2-naphthoate (8-3)

To a mixture of 8-2 (8.2 g, 32.7 mmol) in MeOH (10 mL) was added sulfuric acid (1.2 mL, 22.9 mmol) at room temperature. The resulting mixture was stirred at 50° C. for 16 hours under nitrogen atmosphere. After cooling to room temperature, the mixture was poured into ice water (50 mL) and then extracted with ethyl acetate (30 mL*3). The combined organic layers were dried over sodium sulfate, filtered and concentrated to dryness to have crude desired product 8-3 (8.5 g, crude).

### methyl 7-(4,4,5,5-tetramethyl-1,3,2-dioxaborolan-2-yl)-2-naphthoate (8-4)

To the solution of 8-3 (8.5 g, 32.0 mmol) B₂Pin₂ (9.0 g, 35.3 mmol) and KOAc (9.4 g, 96.1 mmol) in dioxane (100 mL) was added Pd(dppf)Cl₂ (2.6 g, 3.2 mmol). The resulting mixture was stirred at 100° C. for 19 hours under nitrogen atmosphere. After cooling to room temperature, the mixture was poured into ice water (300 mL) and then extracted with ethyl acetate (80 mL*3). The combined organic layers were dried over sodium sulfate, filtered and concentrated to dryness. The residue was purified by column chromatography (elute with ethyl acetate:petroleum ether=1:20 to 1:10) to have the desired product 8-4 (8.9 g, 88.6%).

### tert-butyl(E)-3-(1-(2-(2-hydroxyethoxy)ethyl)-4-methyl-1H-benzo[d][1,2,3]triazol-5-yl)acrylate (8-6)

To the solution of 2-(2-(5-bromo-4-methyl-1H-benzo[d][1,2,3]triazol-1-yl)ethoxy)ethan-1-ol (1.0 g, 3.3 mmol) tert-butyl acrylate (2.6 g, 20.0 mmol) and DIEA (863.0 mg, 6.6 mmol) in DMF (10 mL) was added Pd(OAc)₂ (112.0 mg, 0.5 mmol) and P(O-tol)₃ (305 mg, 1.0 mmol) at 20° C. The resulting mixture was stirred at 120° C. for 4 hours under nitrogen atmosphere. After cooling to room temperature, the mixture was then poured into ice water (50 mL) and extracted with ethyl acetate (10 mL*3). The combined organic layers were dried over sodium sulfate, filtered and concentrated to dryness. The residue was purified by column chromatography (elute with ethyl acetate:petroleum ether=1:10 to 1:3) to have desired product 8-6 (850.3 mg, 81.8%).

MS(ESI) calculated for C₁₈H₂₅N₃O₄,347.2; found 348.5.

### Methyl 7-(3-(tert-butoxy)-1-(1-(2-(2-hydroxyethoxy)ethyl)-4-methyl-1H-benzo[d][1,2,3]triazol-5-yl)-3-oxopropyl)-2-naphthoate (8-7)

To a mixture of 8-6 (750.0 mg, 2.2 mmol), sodium dodecyl sulfate (311.2 mg, 1.1 mmol), Et₃N (654.9 mg, 6.6 mmol), 3 (1.3 g, 4.4 mmol) in CPME (5 mL) and water (2.5 mL) was added [Rh(1,5-cod)Cl]₂ (106.6 mg, 0.22 mmol) at room temperature. The resulting mixture was stirred at 110° C. for 3 hours under nitrogen atmosphere. After cooling to room temperature, the mixture was poured into ice water (50 mL) and then extracted with ethyl acetate (30 mL*3). The combined organic layers were dried over sodium sulfate, filtered and concentrated to dryness. The residue was purified by column chromatography (elute with ethyl acetate:petroleum ether=1:10 to 1:1) to have desired product 8-7 (560.0 mg, 48.5%).

MS(ESI) calculated for C₃₀H₃₅N₃O₆, 533.3; found 534.5.

### Methyl 7-(3-(tert-butoxy)-1-(4-methyl-1-(2-(2-((methylsulfonyl)oxy)ethoxy)ethyl)-1H-benzo[d][1,2,3]triazol-5-yl)-3-oxopropyl)-2-naphthoate(8-8)

To the solution of 8-7 (560.0 mg, 1.1 mmol) and Et₃N (318.3 mg, 3.3 mmol) in DCM (5 mL) was added MsCl (145.0 mg, 1.3 mmol) at 0° C. The resulting mixture was stirred at 25° C. for 2 hours under nitrogen atmosphere. After which period, the mixture was poured into water (15 mL) and then extracted with ethyl acetate (10 mL*3). The combined organic layers were dried over sodium sulfate, filtered and concentrated to dryness to have crude product 8-8 (580.2 mg, 90.3%).

### benzyl 4-(2-(2-(5-(3-(tert-butoxy)-1-(7-(methoxycarbonyl)naphthalen-2-yl)-3-oxopropyl)-4-methyl-1H-benzo[d][1,2,3]triazol-1-yl)ethoxy)ethyl)piperazine-1-carboxylate(8-9)

To the solution of 8-8 (0.4 g, 0.7 mmol) and benzyl piperazine-1-carboxylate (173.0 mg, 0.8 mmol) in ACN (4 mL) was added K₂CO₃ (271.0 mg, 2.1 mmol) at 0° C. The resulting mixture was stirred at 80° C. for 6.5 hours. After cooling to room temperature, the mixture was then poured into ice water (15 mL) and then extracted with ethyl acetate (10 mL*3). The combined organic layers were dried over sodium sulfate, filtered and concentrated to dryness. The residue was purified by column chromatography (elute with ethyl acetate:petroleum ether=1:1 to 3:1) to have the desired product 8-9 (232.0 mg, 48.2%).

MS(ESI) calculated for C₄₂H₄₉N₅O₇, 735.4; found 736.5.

### 7-(1-(1-(2-(2-(4-((benzyloxy)carbonyl)piperazin-1-yl)ethoxy)ethyl)-4-methyl-1H-benzo[d][1,2,3]triazol-5-yl)-3-(tert-butoxy)-3-oxopropyl)-2-naphthoic acid (8-10)

To the solution of 8-9 (232.0 mg, 0.3 mmol) in THE (2 mL), MeOH (1 mL) and H₂O (0.5 mL) was added NaOH (60.0 mg, 1.5 mmol). The resulting mixture was stirred at room temperature for 2 hours under nitrogen atmosphere. The reaction mixture was then neutralized with 1N HCl at 0° C. The aqueous layer was extracted with ethyl acetate (5 mL*3). The combined organic layers were dried over sodium sulfate, filtered and concentrated to have crude desired product 8-10 (235.5 mg, crude).

### 7-(3-(tert-butoxy)-1-(4-methyl-1-(2-(2-(piperazin-1-yl)ethoxy)ethyl)-1H-benzo[d][1,2,3]triazol-5-yl)-3-oxopropyl)-2-naphthoic acid (8-11)

To the solution of 8-10 (235.0 mg, crude) in MeOH (2 mL) was added Pd(OH)₂ (24.0 mg). The resulting mixture was stirred at 20° C. for 6.5 hours under H₂ atmosphere. The catalyst was removed by filtration, and the filtrate was concentrated to have desired product 8-11 (180.5 mg, 94.1%).

MS(ESI) calculated for C₃₃H₄₁N₅O₅, 587.3; found 588.5.

### tert-butyl 2-(1⁴-methyl-4-oxo-1¹H-8-oxa-1(5,1)-benzo[d][1,2,3]triazola-5(1,4)-piperazina-3(2,7)-naphthalenacyclodecaphane-2-yl)acetate (8-12)

To the solution of 8-11 (85.0 mg, 0.1 mmol) and DIEA (51.6 mg, 0.4 mmol) in DMF (2 mL) was added HATU (76.0 mg, 0.2 mmol) at 25° C. The resulting mixture was stirred at room temperature for 19 hours under nitrogen atmosphere. After which period, the mixture was poured into water (8 mL) and then extracted with ethyl acetate (5 mL*3). The combined organic layers were dried over sodium sulfate, filtered and concentrated to dryness. The residue was purified by column chromatography (methyl alcohol:dichloromethane=1:30 to 1:15) to have desired product 8-12 (16.0 mg, 19.5%).

MS(ESI) calculated for C₃₃H₃₉N₅O₄, 569.3; found 570.5.

### 2-(1⁴-methyl-4-oxo-1¹H-8-oxa-1(5,1)-benzo[d][1,2,3]triazola-5(1,4)-piperazina-3(2,7)-naphthalenacyclodecaphane-2-yl)acetic acid (Compound 8)

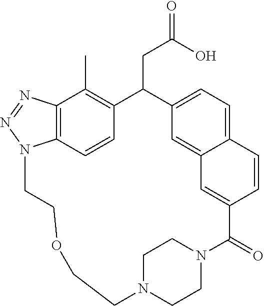 [SMILES: Cc1c2ccc3c1nnn3CCOCCN1CCN(CC1)C(=O)c1ccc3ccc(cc3c1)C2CC(=O)O]

To the solution of 8-12 (16.0 mg, 28.1 mol) in MeOH (0.5 mL) was added HCl (4.0 M in dioxane, 0.5 mL) at 0° C. The resulting mixture was stirred at 20° C. for 2 hours under nitrogen atmosphere and then concentrated to dryness. The residue was purified with Prep-HPLC (CH₃CN/H₂O, 0.1% HCl) to have the desired product Compound 8 (1.3 mg, 9.0%).

MS(ESI) calculated for C₂₉H₃₁N₅O₄, 513.2; found 514.5.

¹H NMR (400 MHz, DMSO-d₆) δ 10.82 (s, 1H), 8.03-7.76 (m, 4H), 7.69-7.40 (m, 4H), 5.04-5.00 (m, 1H), 4.90-4.85 (m, 2H), 3.93-3.89 (m, 4H), 3.80-3.73 (m, 4H), 3.20-3.15 (m, 6H), 2.79-2.75 (m, 3H), 2.71-2.66 (m, 2H).

### Example 1.9 Synthesis of Compound 9

### Procedure for 2-(carboxymethyl)-1⁴-methyl-4-oxo-3¹,3²,3³,3⁴-tetrahydro-1¹H-6,9-dioxa-3(2,7)-isoquinolin-2-iuma-1(5,1)-benzo[d][1,2,3]triazola-5(1,4)-piperidinacycloundecaphan-3²-ium chloride (Compound 9)

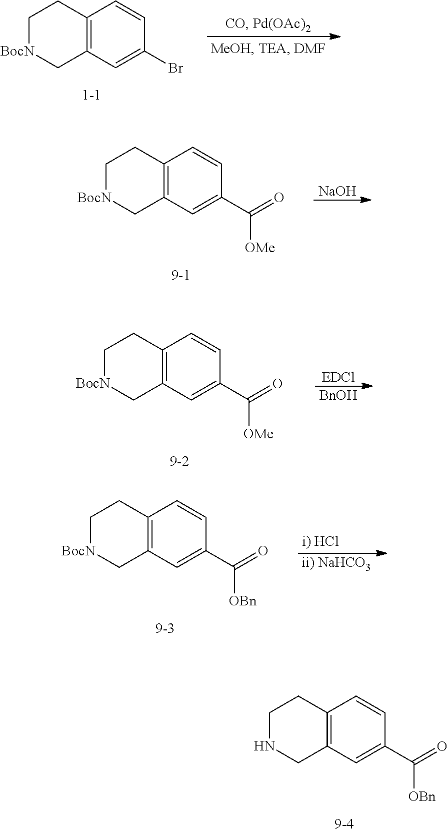

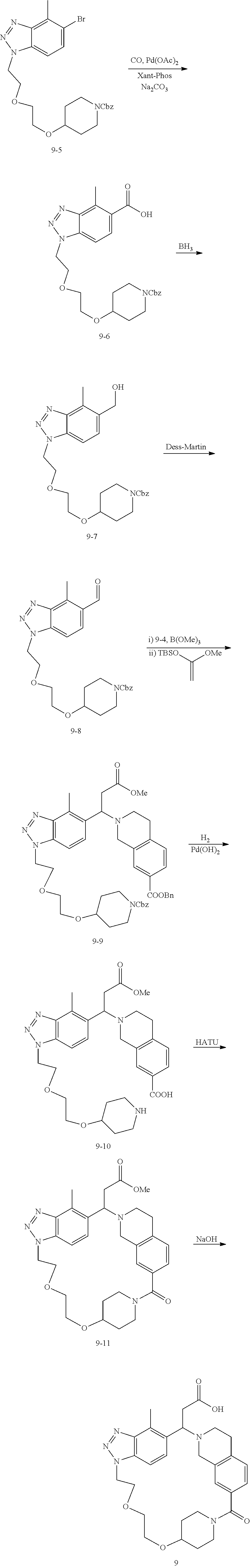

### 2-(tert-butyl) 7-methyl 3,4-dihydroisoquinoline-2,7(1H)-dicarboxylate (9-1)

To the solution of tert-butyl 7-bromo-3,4-dihydroisoquinoline-2(1H)-carboxylate(5.0 g, 16.0 mmol) TEA(4.4 g, 43.9 mmol) and MeOH(30 mL) in DMA (50 mL) was added Pd(OAc)₂ (0.3 g, 1.6 mmol) at 20° C. The resulting mixture was stirred at 120° C. for 19 hours under CO atmosphere. After cooling to room temperature, the mixture was then poured into ice water (200 mL) and extracted with ethyl acetate (30 mL*3). The combined organic layers were dried over sodium sulfate, filtered and concentrated to dryness. The residue was purified by column chromatography (elute with ethyl acetate:petroleum ether=1:3 to 1:1) to have the desired compound 9-1 (3.1 g, 66.5%).

MS(ESI) calculated for C₁₆H₂₁NO₄, 291.2; found 292.2.

### 2-(tert-butoxycarbonyl)-1,2,3,4-tetrahydroisoquinoline-7-carboxylic acid (9-2)

To the solution of 9-1 (3.1 g, 10.6 mmol) in THF:MeOH:H₂O (18 mL: 6 mL: 6 mL) was added NaOH (2.1 g, 53.2 mmol) at 0° C. The resulting mixture was stirred at 20° C. for 4 hours under nitrogen atmosphere and then concentrated to dryness to have the desired product 9-2 (3.0 g, crude), which was used directly to next run.

### 7-benzyl 2-(tert-butyl) 3,4-dihydroisoquinoline-2,7(1H)-dicarboxylate (9-3)

To the solution of 9-2 (2.5 g, 9.08 mmol) HOBt (2.5 g, 18.2 mmol), EDCI (3.5 g, 18.2 mmol) and TEA (2.8 g, 27.3 mmol) in DMF (10 mL) added BnOH (2.9 g, 27.3 mmol) at 0° C. The resulting mixture was stirred at 20° C. for 19 hours under nitrogen atmosphere. The mixture was then poured into ice water (200 mL) and then extracted with ethyl acetate (40 mL*3). The combined organic layers were dried over sodium sulfate, filtered and concentrated to dryness. The residue was purified by column chromatography (elute with ethyl acetate:petroleum ether=1:5 to 1:3) to have desired product 9-3 (3.0 g, 89.6%).

### benzyl 1,2,3,4-tetrahydroisoquinoline-7-carboxylate (9-4)

To the solution of 9-3 (3.6 g, 13.4 mmol) in MeOH (30 mL) was added HCl (4M in dioxane, 30 mL) at 0° C. The resulting mixture was stirred at 20° C. for 2 hours and then concentrated to dryness. The residues were then dissolved in NaHCO₃ (aq. 150 mL) and extracted with ethyl acetate (15 mL*3). The combined organic layers were dried over sodium sulfate, filtered and concentrated to have the desired product 9-4 (1.5 g, 41.9%), which was used directly to next run.

MS(ESI) calculated for C₁₇H₁₇NO₂, 267.1; found 268.2.

### 1-(2-(2-((1-((benzyloxy)carbonyl)piperidin-4-yl)oxy)ethoxy)ethyl)-4-methyl-1H-benzo[d][1,2,3]triazole-5-carboxylic acid (9-6)

To the solution of 9-5 (0.5 g, 1.0 mmol), Na₂CO₃ (0.2 g, 1.9 mmol), Xantphos (111.8 mg, 0.2 mmol) and Pd(OAc)₂ (21.7 mg, 0.1 mmol) in DMF (10 mL) was added Et₃SiH (1.1 g, 9.7 mmol) at 20° C. The resulting mixture was stirred at 120° C. for 19 hours under CO atmosphere. After cooling to room temperature, the mixture was then poured into ice water (70 mL) and extracted with ethyl acetate (20 mL*3). The combined organic layers were dried over sodium sulfate, filtered and concentrated to dryness. The residue was purified by column chromatography (elute with ethyl acetate:petroleum ether=1:5 to 2:1) to have the desired product 9-6 (350.0 mg, 73.0%).

MS(ESI) calculated for C₂₅H₃₀N₄O₆, 482.2; found 483.2.

### benzyl 4-(2-(2-(5-(hydroxymethyl)-4-methyl-1H-benzo[d][1,2,3]triazol-1-yl)ethoxy)ethoxy)piperidine-1-carboxylate (9-7)

To the solution of 9-6 (350.0 mg, 0.7 mmol) in THE (5 mL) was added borane tetrahydrofuran complex (1M, 5 mL) at 0° C. The resulting mixture was stirred at room temperature for 19 hours under nitrogen atmosphere. The reaction was then quenched with MeOH (10 mL), and the resulting mixture was concentrated to dryness to have the desired product 9-7 (0.3 g, 87.7%).

MS(ESI) calculated for C₂₅H₃₂N₄O₅, 468.2; found 469.2.

### benzyl 4-(2-(2-(5-formyl-4-methyl-1H-benzo[d][1,2,3]triazol-1-yl)ethoxy)ethoxy)piperidine-1-carboxylate (9-8)

To the solution of 9-7 (0.2 g, 0.4 mmol) in DCM (10 mL) was added Dess-Martin periodinane (212.8 mg, 0.5 mmol) at 0° C. The resulting mixture was stirred at room temperature for 2 hours under nitrogen atmosphere. The mixture was then poured into ice water (50 mL) and extracted with ethyl acetate (20 mL*3). The combined organic layers were dried over sodium sulfate, filtered and concentrated to dryness. The residue was purified by column chromatography (elute with ethyl acetate:petroleum ether=1:5 to 3:1) to have the desired product 9-8 (179.2 mg, 88.4%).

MS(ESI) calculated for C₂₅H₃₂N₄O₅, 468.2; found 469.2.

### benzyl 2-(1-(1-(2-(2-((1-((benzyloxy)carbonyl)piperidin-4-yl)oxy)ethoxy)ethyl)-4-methyl-1H-benzo[d][1,2,3]triazol-5-yl)-3-methoxy-3-oxopropyl)-1,2,3,4-tetrahydroisoquinoline-7-carboxylate (9-9)

To the solution of 9-4 (343.8 mg, 1.3 mmol) in DMSO (3 mL) was added B(OMe)₃ (133.6 mg, 1.3 mmol) at 20° C. The resulting mixture was stirred at room temperature for 1 hour under nitrogen atmosphere. And then the solution of 9-8 (0.2 g, 0.4 mmol) in DMSO (1 mL) was added to the above mixture. The resulting mixture was then stirred for another 1 hour at room temperature. After which period, tert-butyl((1-methoxyvinyl)oxy)dimethylsilane (242.2 mg, 1.3 mmol) was added. The reaction mixture was then stirred for another 19 hours at room temperature under nitrogen atmosphere. The mixture was then poured into ice water (50 mL) and extracted with ethyl acetate (20 mL*3). The combined organic layers were dried over sodium sulfate, filtered and concentrated to dryness. The residue was purified by column chromatography (elute with ethyl acetate:petroleum ether=1:2 to 3:1) to have desired product 9-9 (60.0 mg, 23.6%).

MS(ESI) calculated for C₄₅H₅₁N₅O₈, 789.4; found 790.4.

### 2-(3-methoxy-1-(4-methyl-1-(2-(2-(piperidin-4-yloxy)ethoxy)ethyl)-1H-benzo[d][1,2,3]triazol-5-yl)-3-oxopropyl)-1,2,3,4-tetrahydroisoquinoline-7-carboxylic acid (9-10)

To the solution of 9-9 (60.0 mg, 76.0 mol) in MeOH (10 mL) was added Pd(OH)₂ (15 mg) at 20° C. The resulting mixture was stirred at room temperature for 2 hours under H₂ atmosphere. The catalyst was removed by filtration, and the filtrate was concentrated to have the desired product 9-10 (40.0 mg, 93.1%).

MS(ESI) calculated for C₃₀H₃₉N₅O₆, 565.3; found 565.4.

### methyl 2-(1⁴-methyl-4-oxo-3¹,3²,3³,3⁴-tetrahydro-1¹H-6,9-dioxa-3(2,7)-isoquinolina-1(5,1)-benzo[d][1,2,3]triazola-5(1,4)-piperidinacycloundecaphane-2-yl)acetate (9-11)

To the solution of 9-10 (40.0 mg, 70.7 mol) in DMF (2 mL) was added DIPEA (18.3 mg, 140 mol) and HATU (53.8 mg, 140 μmol) at 20° C. The resulting mixture was stirred at room temperature for 2 hours under nitrogen atmosphere. After which period, the mixture was poured into ice water (20 mL) and extracted with ethyl acetate (10 mL*3). The combined organic layers were dried over sodium sulfate, filtered and concentrated to dryness. The residue was purified by column chromatography (elute with MeOH:DCM=1:50 to 1:10) to have the desired product 9-11 (35.0 mg, 90.4%).

MS(ESI) calculated for C₃₀H₃₇N₅O₅, 547.3; found 548.3.

### 2-(carboxymethyl)-1⁴-methyl-4-oxo-3¹,3²,3³,3⁴-tetrahydro-1¹H-6,9-dioxa-3(2,7)-isoquinolin-2-iuma-1(5,1)-benzo[d][1,2,3]triazola-5(1,4)-piperidinacycloundecaphan-3²-ium chloride (Compound 9)

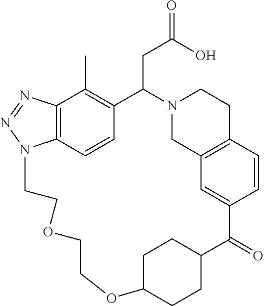 [SMILES: Cc1c2ccc3c1nnn3CCOCCOC1CCC(CC1)C(=O)c1ccc3c(c1)CN(CC3)C2CC(=O)O]

To the solution of 9-11 (35.0 mg, 63.9 mol) in THF (5 mL) MeOH (2 mL) and H₂O (1 mL) was added NaOH (12.8 mg, 0.3 mmol). The resulting mixture was stirred at room temperature for 2 hours under nitrogen atmosphere. The reaction mixture was neutralized with 1N HCl at 0° C. and extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 9 (3.0 mg, 8.8%).

MS(ESI) calculated for C₂₉H₃₅N₅O₅, 533.3; found 534.3.

¹H NMR (400 MHz, MeOH-d₄) δ 7.93-7.82 (m, 2H), 7.57-7.49 (m, 2H), 6.54 (s, 1H), 5.24-5.20 (m, 1H), 5.03-4.91 (m, 2H), 4.54-4.48 (m, 1H), 4.27-3.47 (m, 14H), 3.02-2.68 (m, 4H), 1.94 (s, 3H), 1.48-1.16 (m, 2H), 0.82-0.66 (m, 2H).

### Example 1.10 Synthesis of Compound 10

### Procedure for 2-(1⁴-methyl-4-oxo-3¹,3²,3³,3⁴-tetrahydro-1¹H-6-oxa-3(7,2)-isoquinolina-1(5,1)-benzo[d][1,2,3]triazola-5(1,4)-piperidinacyclodecaphane-2-yl)acetic acid (Compound 10)

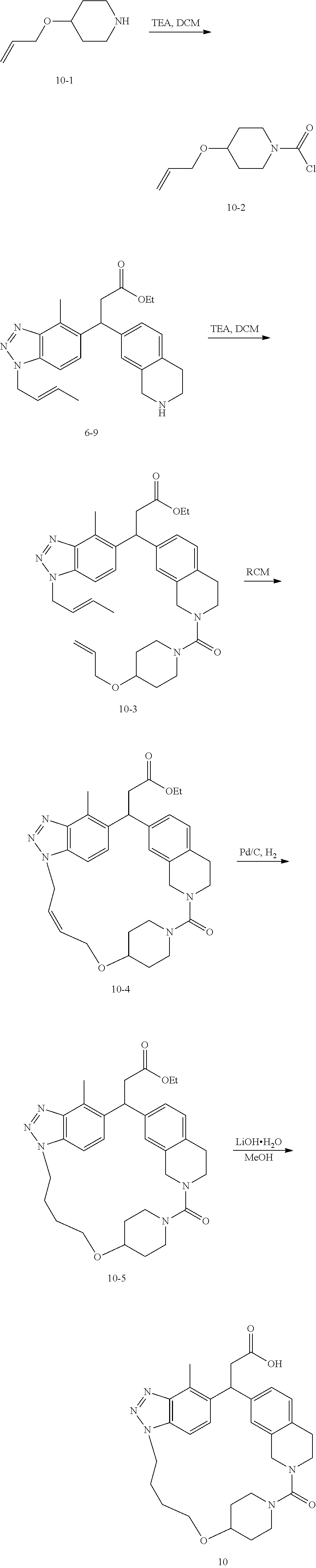

### 4-(allyloxy)piperidine-1-carbonyl chloride (10-2)

To the mixture of 4-(allyloxy)piperidine (200.0 mg, 1.3 mmol) and TEA (0.5 mL, 4.0 mmol) in DCM (5 mL) was added triphosgene (594.0 mg, 2.0 mmol) at 0° C. The resulting mixture was stirred at 25° C. overnight under nitrogen atmosphere and then concentrated under reduced pressure to get the desired product 10-2 (800.0 mg, crude).

### ethyl (E)-3-(2-(4-(allyloxy)piperidine-1-carbonyl)-1,2,3,4-tetrahydroisoquinolin-7-yl)-3-(1-(but-2-en-1-yl)-4-methyl-1H-benzo[d][1,2,3]triazol-5-yl)propanoate (10-3)

The mixture of 6-9 (100.0 mg, crude) and TEA (0.5 mL, 6.9 mmol) in DMF (5 mL) was added a solution of 10-2 (80.0 mg, crude) in DMF (5 mL) at 0° C. The resulting mixture was stirred at 25° C. overnight. TLC showed the reaction was completed. The mixture was then poured into ice water (50 mL) and extracted with ethyl acetate (25 mL*3). The combined organic layers were dried over sodium sulfate, filtered and concentrated to dryness. The residue was purified by column chromatography (elute with ethyl acetate:petroleum ether=1:5 to 1:2) to have the desired product 10-3 (120.0 mg, 52.2%).

### ethyl (Z)-2-(14-methyl-4-oxo-3¹,3²,3³,3⁴-tetrahydro-11H-6-oxa-3(7,2)-isoquinolina-1(5,1)-benzo[d][1,2,3]triazola-5(1,4)-piperidinacyclodecaphan-8-en-2-yl)acetate (10-4)

To the solution of 10-3 (120.0 mg, 0.2 mmol) in DCM (30.0 mL) was added Grubbs catalyst (40.0 mg, 0.1 mmol). The resulting mixture was stirred at 40° C. for 19 hours under nitrogen atmosphere. After which period, the mixture was poured into ice water (50 mL) and then extracted with ethyl acetate (20 mL*3). The combined organic layers were dried over sodium sulfate, filtered and concentrated to dryness. The residue was purified with Prep-HPLC (CH₃CN/H₂O, 0.1% HCl) to have the desired product 10-4 (180.0 mg, crude).

### ethyl 2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-piperidinacyclodecaphane-2-yl)acetate (10-5)

The mixture of 10-4 (100.0 mg, crude) and Pd/C (20.0 mg) in MeOH (20.0 mL) was stirred for 2 hours at 20° C. under H₂ atmosphere. The catalyst was then removed by filtration, and the filtrate was concentrated to have the desired product 10-5 (90.0 mg, crude).

### 2-(1⁴-methyl-4-oxo-3¹,3²,3³,3⁴-tetrahydro-1¹H-6-oxa-3(7,2)-isoquinolina-1(5,1)-benzo[d][1,2,3]triazola-5(1,4)-piperidinacyclodecaphane-2-vi)acetic acid (Compound 10)

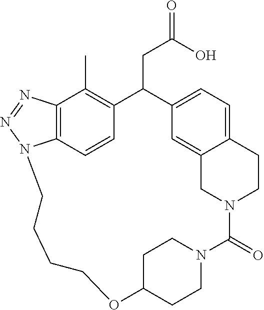 [SMILES: Cc1c2ccc3c1nnn3CCCCOC1CCN(CC1)C(=O)N1CCc3ccc(cc3C1)C2CC(=O)O]

The mixture of 10-5 (90.0 mg, 0.2 mmol) in MeOH (5 mL) and water (2 mL) was added LiOH·H₂O (36.1 mg, 859.5 mol) at room temperature. The reaction mixture was stirred at room temperature for 2 hours under nitrogen atmosphere and then acidified with HCl (1M). The resulting mixture was concentrated to dryness, and the residue purified with Prep-HPLC (CH₃CN/H₂O, 0.1% HCl) to have the desired product Compound 10 (2.1 mg, 2.6%).

MS(ESI) calculated for C₂₉H₃₅N₅O₄, 517.2; found 518.2.

¹H NMR (400 MHz, MeOH-d₄) δ 7.59 (d, J=8.6 Hz, 1H), 7.51 (d, J=8.6 Hz, 1H), 7.40 (d, J=8.0 Hz, 1H), 7.13 (d, J=7.6 Hz, 1H), 6.54 (s, 1H), 4.99-4.90 (m, 1H), 4.78-4.73 (m, 2H), 4.18-4.08 (m, 2H), 3.69-3.61 (m, 1H), 3.56-3.42 (m, 2H), 3.42-3.35 (m, 1H), 3.26-3.18 (m, 1H), 3.16-3.01 (m, 4H), 2.90-2.82 (m, 2H), 2.80-2.72 (m, 1H), 2.67 (s, 3H), 2.63-2.54 (m, 1H), 2.40-2.22 (m, 1H), 2.20-2.10 (m, 1H), 2.00-1.91 (m, 1H), 1.64-1.60 (m, 1H), 1.57-1.49 (m, 1H), 1.45-1.39 (m, 1H), 1.18-1.08 (m, 1H), 0.90-0.80 (m, 1H), 0.60-0.51 (m, 1H).

### Example 1.11 Synthesis of Compound 11

### Procedure for (Z)-2-(1⁴-methyl-4-oxo-3¹,3²,3³,3⁴-tetrahydro-1¹H-6-oxa-3(7,2)-isoquinolina-1(5,1)-benzo[d][1,2,3]triazola-5(1,4)-piperidinacyclodecaphan-8-en-2-yl)acetic acid (Compound 11)

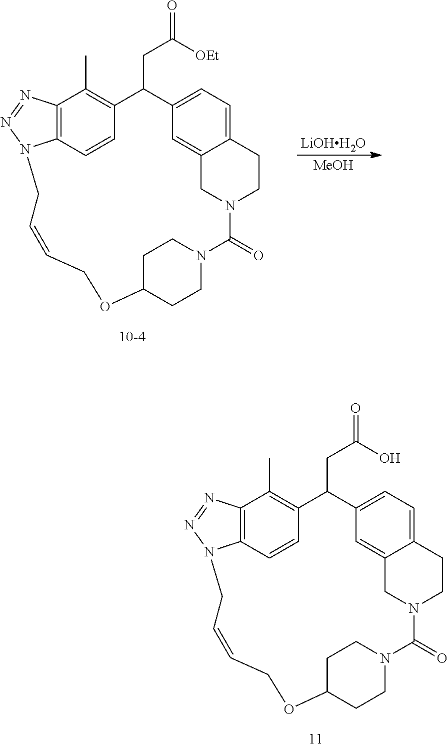

The mixture of 10-4 (80.0 mg, crude) and LiOH·H₂O (30 mg) in MeOH (5.0 mL) and water (2 mL) was stirred at 20° C. for 2 hours. After which period, the reaction mixture was acidified with 1M HCl at 0° C. and extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 11 (1.9 mg, 2.2%).

MS(ESI) calculated for C₂₉H₃₃N₅O₄, 515.2; found 516.3.

¹H NMR (400 MHz, MeOH-d₄) δ 7.49 (d, J=8.7 Hz, 1H), 7.38 (d, J=6.7 Hz, 1H), 7.31 (d, J=8.7 Hz, 1H), 7.14 (d, J=7.8 Hz, 1H), 6.58 (s, 1H), 6.02-5.84 (m, 1H), 5.54-5.36 (m, 2H), 5.23-5.17 (m, 1H), 4.99 (t, J=7.5 Hz, 1H), 4.18-3.94 (m, 4H), 3.69-3.62 (m, 1H), 3.56-3.40 (m, 1H), 3.25-3.11 (m, 2H), 3.11-3.01 (m, 1H), 3.01-2.88 (m, 2H), 2.88-2.76 (m, 5H), 2.60-2.41 (m, 2H), 1.67-1.46 (m, 2H), 1.46-1.30 (m, 2H), 1.21-0.91 (m, 3H).

### Example 1.12 Synthesis of Compound 12

### Procedure for 2-(1⁴-methyl-4-oxo-3¹,3²,3³,3⁴-tetrahydro-1¹H-7-oxa-3(7,2)-isoquinolina-1(5,1)-benzo[d][1,2,3]triazola-5(1,4)-piperidinacyclodecaphane-2-yl)acetic acid (Compound 12)

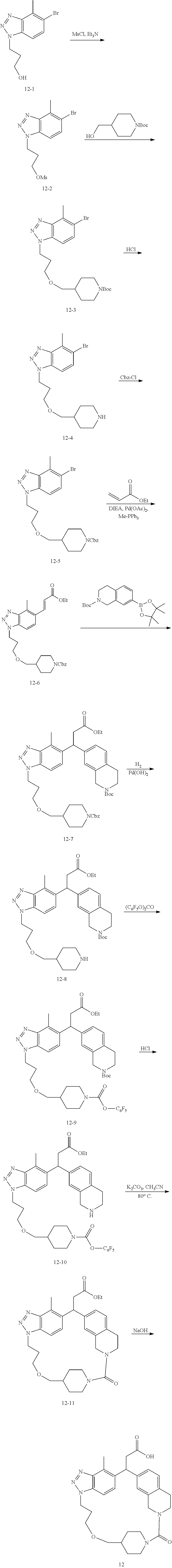

### 3-(5-bromo-4-methyl-1H-benzo[d][1, 2, 3]triazol-1-yl)propyl methanesulfonate (12-2)

To the solution of 3-(5-bromo-4-methyl-1H-benzo[d][1,2,3]triazol-1-yl)propan-1-ol (2 g, 7.4 mmol) and TEA (752.0 mg, 14.8 mmol) in DCM (10 mL) was added MsCl (1.3 g, 11.2 mmol) at 0° C. The resulting mixture was stirred at 20° C. for 2.5 hours. After which period, the mixture was poured into ice water (50 mL) and then extracted with ethyl acetate (15 mL*3). The combined organic layers were dried over sodium sulfate, filtered and concentrated to dryness. The residue was purified by column chromatography (elute with ethyl acetate:petroleum ether=1:1 to 2:1) to have the desired product 12-2 (2.2 g, 85.3%).

MS(ESI) calculated for C₁₁H₁₄BrN₃O₃S 348.2; found 349.3,351.4.

### tert-butyl 4-((3-(5-bromo-4-methyl-1H-benzo[d][1,2,3]triazol-1-yl)propoxy) methyl)piperidine-1-carboxylate (12-3)

The solution of tert-butyl 4-(hydroxymethyl)piperidine-1-carboxylate (1.6 g, 7.56 mmol) in DMF (10 mL) was added NaH (60% in mineral oil, 68.9 mg, 18.9 mmol) at 0° C. After 0.5 hour, 12-2 (2.2 g, 6.3 mmol) and TBAI (233.4 mg, 0.6 mmol) was added. The resulting mixture was stirred at 25° C. for 19 hours under nitrogen atmosphere. The mixture was then poured into ice water (80 mL) and extracted with ethyl acetate (15 mL*3). The combined organic layers were dried over sodium sulfate, filtered and concentrated to dryness. The residue was purified by column chromatography (elute with ethyl acetate:petroleum ether=1:1 to 3:1) to have the desired product 12-3 (1.1 g, 37.4%).

### 5-bromo-4-methyl-1-(3-(piperidin-4-ylmethoxy)propyl)-1H-benzo[d][1,2,3]triazole (12-4)

To the solution of 12-3 (1.7 g, 3.6 mmol) was added HCl (4M in dioxane, 20 mL) at 0° C. The resulting mixture was stirred at 20° C. for 2 hours and then concentrated to dryness to have the desired product 12-4 (1.5 g, wet).

### benzyl 4-((3-(5-bromo-4-methyl-1H-benzo[d][1,2,3]triazol-1-yl)propoxy)methyl)piperidine-1-carboxylate(12-5)

To the solution of 12-4 (1.5 g, 4.1 mmol) in ACN:H₂O (20 mL: 20 mL) was added NaHCO₃ (857.7 mg, 10.2 mmol) and Cbz-Cl (1.1 g, 6.1 mmol) at 0° C. The resulting mixture was stirred at 80° C. for 6.5 hours. After cooling to room temperature, the mixture was then poured into ice water (50 mL) and then extracted with ethyl acetate (15 mL*3). The combined organic layers were dried over sodium sulfate, filtered and concentrated to dryness. The residue was purified by column chromatography (elute with ethyl acetate:petroleum ether=1:1 to 3:1) to have the desired product 12-5 (0.9 g, 43.8%).

MS(ESI) calculated for C₂₄H₂₉BrN₄O₃ 501.4; found 502.5,504.4.

### benzyl (E)-4-((3-(5-(3-ethoxy-3-oxoprop-1-en-1-yl)-4-methyl-1H-benzo[d][1,2,3]triazol-1-yl)propoxy)methyl)piperidine-1-carboxylate (12-6)

To the solution of 12-5 (0.9 g, 1.9 mmol), ethyl acrylate (718.8 mg, 7.2 mmol) and DIEA (463.1 mg, 3.6 mmol) in DMF (20 mL) was added Pd(OAc)₂ (60.4 mg, 0.3 mmol) and (p-tol)₃P (163.7 mg, 0.6 mmol) at 20° C. The resulting mixture was stirred at 120° C. for 4 hours under nitrogen atmosphere. After cooling to room temperature, the mixture was poured into ice water (100 mL) and then extracted with ethyl acetate (50 mL*3). The combined organic layers were dried over sodium sulfate, filtered and concentrated to dryness. The residue was purified by column chromatography (elute with ethyl acetate:petroleum ether=1:10 to 1:2) to have desired product 12-6 (950.0 mg, 96.0%).

MS(ESI) calculated for C₂₉H₃₆N₄O₅ 520.6; found 521.7.

### tert-butyl (S)-7-(1-(1-(3-((1-((benzyloxy)carbonyl)piperidin-4-yl)methoxy)propyl)-4-methyl-1H-benzo[d][1,2,3]triazol-5-yl)-3-ethoxy-3-oxopropyl)-3,4-dihydroisoquinoline-2(1H)-carboxylate(12-7)

To a mixture of 12-6 (950.0 mg, 1.8 mmol), sodium dodecyl sulfate (262.8 mg, 0.9 mmol), TEA (552.9 mg, 5.5 mmol) and tert-butyl 7-(4,4,5,5-tetramethyl-1,3,2-dioxaborolan-2-yl)-3,4-dihydroisoquinoline-2(1H)-carboxylate (2.0 g, 5.4 mmol) in CPME (6 mL) and water (3 mL) was added [Rh(1,5-cod)Cl]₂ (89.9 mg, 0.2 mmol) at room temperature. The resulting mixture was stirred at 100° C. for 4.5 hours under nitrogen atmosphere. After cooling to room temperature, the mixture was poured into ice water (50 mL) and then extracted with ethyl acetate (25 mL*3). The combined organic layers were dried over sodium sulfate, filtered and concentrated to dryness. The residue was purified by column chromatography (elute with ethyl acetate:petroleum ether=1:10 to 1:2) to have the desired product 12-7 (550.0 mg, 40.5%).

MS(ESI) calculated for C₄₅H₅₅N₅O₇ 753.94; found 754.9.

### tert-butyl (S)-7-(3-ethoxy-1-(4-methyl-1-(3-(piperidin-4-ylmethoxy)propyl)-1H-benzo[d][1,2,3]triazol-5-yl)-3-oxopropyl)-3,4-dihydroisoquinoline-2(1H)-carboxylate(12-8)

To the solution of 12-7 (550.0 mg, 0.7 mmol) in MeOH (10 mL) was added Pd(OH)₂ (91.6 mg). The resulting mixture was stirred at 20° C. for 6.5 hours under H₂ atmosphere. The catalyst was removed by filtration, and the filtrate was concentrated to have the desired product 12-8 (330.0 mg, 76.1%).

MS(ESI) calculated for C₃₅H₄₉N₅O₅ 619.8; found 700.1.

### tert-butyl (S)-7-(3-ethoxy-1-(4-methyl-1-(3-((1-((perfluorophenoxy)carbonyl)piperidin-4-yl)methoxy)propyl)-1H-benzo[d][1,2,3]triazol-5-yl)-3-oxopropyl)-3,4-dihydroisoquinoline-2(1H)-carboxylate(12-9)

To the solution of 12-8 (0.1 g, 0.2 mmol) bis(perfluorophenyl) carbonate (70.0 mg, 220.0 μmol) in DMF (5 mL) was added DIEA (42.0 mg, 0.4 mmol) at 0° C. The resulting mixture was stirred at 20° C. for 4 hours under nitrogen atmosphere. The mixture was then poured into ice water (80 mL) and extracted with ethyl acetate (20 mL*3). The combined organic layers were dried over sodium sulfate, filtered and concentrated to dryness. The residue was purified by column chromatography (elute with ethyl acetate:petroleum ether=1:3 to 1:2) to have the desired product 12-9 (0.1 g, 60.3%).

### perfluorophenyl (S)-4-((3-(5-(3-ethoxy-3-oxo-1-(1,2,3,4-tetrahydroisoquinolin-7-yl)propyl)-4-methyl-1H-benzo[d][1,2,3]triazol-1-yl)propoxy)methyl)piperidine-1-carboxylate(12-10)

To the solution of 12-9 (0.1 g, 0.1 mmol) in MeOH (5 mL) was added HCl (4M in dioxane, 2.5 mL) at 0° C. The resulting mixture was stirred at 20° C. for 2 hours and then concentrated to dryness to have the desired product 12-10 (30.0 mg, 41.1%).

### ethyl 2-((2S)-1⁴-methyl-4-oxo-3¹,3²,3³,3⁴-tetrahydro-1¹H-7-oxa-3(7,2)-isoquinolina-1(5,1)-benzo[d][1,2,3]triazola-5(1,4)-piperidinacyclodecaphane-2-yl)acetate(12-11)

To the solution of 12-10 (90.0 mg, 123.3 mol) in DMF (5 mL) was added K₂CO₃ (12.1 mg, 276.6 μmol). The resulting mixture was sealed and heated to 120° C. in a microwave reactor for 3 hours. After cooling to room temperature, the mixture was concentrated to dryness. The residue was purified with Prep-HPLC (CH₃CN/H₂O, 0.1% HCl) to have the desired product 12-11 (14 mg, 20.9%).

### 2-(1⁴-methyl-4-oxo-3¹,3²,3³,3⁴-tetrahydro-1¹H-7-oxa-3(7,2)-isoquinolina-1(5,1)-benzo[d][1,2,3]triazola-5(1,4)-piperidinacyclodecaphane-2-yl)acetic acid (Compound 12)

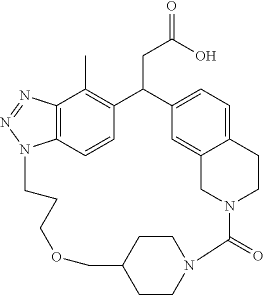 [SMILES: Cc1c2ccc3c1nnn3CCCOCC1CCN(CC1)C(=O)N1CCc3ccc(cc3C1)C2CC(=O)O]

To the solution of 12-11 (14.0 mg, 25.7 mol) in THF (9 mL) MeOH (3 mL) and H₂O (3 mL) was added NaOH (4.8 mg, 0.1 mmol). The resulting mixture was stirred at room temperature for 2 hours under nitrogen atmosphere. After which period, the reaction mixture was acidified with 1N HCl at 0° C. and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 12 (1.8 mg, 7.4%).

MS(ESI) calculated for C₂₉H₃₅N₅O₄ 517.6; found 518.8.

¹H NMR (400 MHz, MeOH-d₄) δ 7.55-7.45 (m, 1H), 7.44-7.34 (m, 1H), 7.33-7.26 (m, 1H), 7.24-7.13 (m, 1H), 6.55 (s, 1H), 4.97-4.92 (m, 1H), 4.81-4.71 (m, 1H), 4.64-4.56 (m, 1H), 4.08 (s, 2H), 3.71-3.66 (m, 1H), 3.58-3.46 (m, 2H), 3.24-2.85 (m, 8H), 2.68 (s, 3H), 2.30 (m, 3H), 1.46-1.23 (m, 4H), 0.78-0.45 (m, 3H).

### Example 1.13 Synthesis of Compound 13

### Procedure for 2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-benzenacyclodecaphane-2-yl)acetic acid (Compound 13)

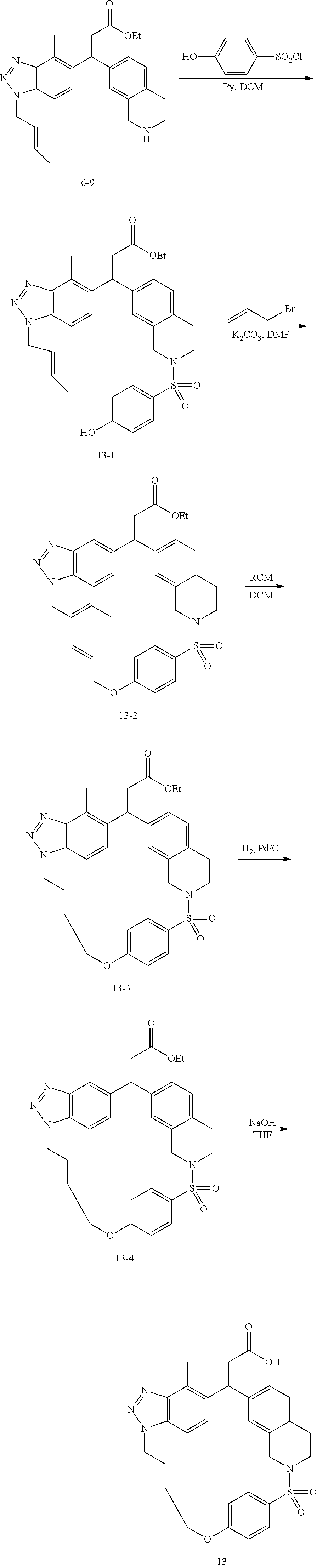

### ethyl (E)-3-(1-(but-2-en-1-yl)-4-methyl-1H-benzo[d][1,2,3]triazol-5-yl)-3-(2-((4-hydroxyphenyl)sulfonyl)-1,2,3,4-tetrahydroisoquinolin-7-yl)propanoate (13-1)

To the solution of 6-9 (1.6 g, 3.9 mmol) and 4-hydroxybenzenesulfonyl chloride (0.9 g, 4.7 mmol) in DCM (20 mL) was added TEA (1.6 g, 15.6 mmol) at 0° C. The resulting mixture was stirred at 25° C. for 6 hours under nitrogen atmosphere. After which period, the mixture was poured into water (20 mL) and then extracted with ethyl acetate (20 mL*3). The combined organic layers were dried over sodium sulfate, filtered and concentrated to dryness. The residue was purified by column chromatography (elute with ethyl acetate:petroleum ether=1:3 to 1:1) to have the desired product 13-1 (1.6 g, 71.6%).

MS(ESI) calculated for C₃₁H₃₄N₄O₅S, 574.2; found 575.2.

### ethyl (E)-3-(2-((4-(allyloxy)phenyl)sulfonyl)-1,2,3,4-tetrahydroisoquinolin-7-yl)-3-(1-(but-2-en-1-yl)-4-methyl-1H-benzo[d][1,2,3]triazol-5-yl)propanoate (13-2)

To the solution of 13-1 (1.5 g, 2.6 mmol) and 3-bromoprop-1-ene (632.3 mg, 5.2 mmol) in DMF (15 mL) was added K₂CO₃ (0.7 g, 5.2 mmol). The resulting mixture was stirred at 50° C. for 19 hours under nitrogen atmosphere. After cooling to room temperature, the mixture was then poured into ice water (30 mL) and extracted with ethyl acetate (30 mL*3). The combined organic layers were dried over sodium sulfate, filtered and concentrated to dryness. The residue was purified by column chromatography (elute with ethyl acetate:petroleum ether=1:3 to 1:1) to have the desired product 13-2 (530 mg, 33.0%).

MS(ESI) calculated for C₃₄H₃₈N₄O₅S, 614.3; found 615.4.

### ethyl (E)-2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-benzenacyclodecaphan-8-en-2-yl)acetate (13-3)

To the solution of 13-2 (0.5 g, 0.8 mmol) in DCM (20 mL) was added Grubbs II catalyst (210.2 mg, 0.2 mmol). The resulting mixture was stirred at 25° C. for 19 hours under nitrogen atmosphere. The mixture was then poured into ice water (10 mL) and then extracted with ethyl acetate (10 mL*3). The combined organic layers were dried over sodium sulfate, filtered and concentrated to dryness. The residue was purified by column chromatography (elute with ethyl acetate:petroleum ether=1:2 to 1:1) to have desired product 13-3 (60.1 mg, 12.9%).

MS(ESI) calculated for C₃₁H₃₂N₄O₅S, 572.2; found 573.2.

### ethyl 2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-benzenacyclodecaphane-2-yl)acetate (13-4)

To the solution of 13-3 (60.1 mg, 105.0 mol) in THF (6 mL) was added Pd/C (30 mg). The resulting mixture was stirred at 25° C. for 19 hours under hydrogen atmosphere. The catalyst was removed by filtration, and the filtrate was concentrated to have the desired product 13-4 (60.0 mg, 99.4%).

MS(ESI) calculated for C₃₁H₃₄N₄O₅S, 574.2; found 575.2.

### 2-(1⁴-methyl-4,4-dioxido-3¹,3²,3³,3⁴-tetrahydro-1¹H-6-oxa-4-thia-3(7,2)-isoquinolina-1(5,1)-benzo[d][1,2,3]triazola-5(1,4)-benzenacyclodecaphane-2-yl)acetic acid (Compound 13)

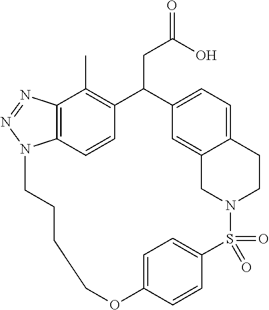 [SMILES: Cc1c2ccc3c1nnn3CCCCOc1ccc(cc1)S(=O)(=O)N1CCc3ccc(cc3C1)C2CC(=O)O]

To the solution of 13-4 (60.0 mg, 104.0 mol) in THF (2 mL) MeOH (1 mL) and H₂O (1 mL) was added NaOH (8.8 mg, 0.2 mmol). The resulting mixture was stirred at room temperature for 2 hours under nitrogen atmosphere. The reaction mixture was acidified with 1N HCl at 0° C. and extracted with ethyl acetate (10 mL*3). The combined organic layers were dried over sodium sulfate, filtered and concentrated to dryness. The residue was purified with Prep-HPLC (CH₃CN/H₂O, 0.1% HCl) to have the desired product Compound 13 (4.0 mg, 7.0%).

MS(ESI) calculated for C₂₉H₃₀N₄O₅S, 546.2; found 547.2.

¹H NMR (400 MHz, DMSO-d₆) δ 7.63 (d, J=8.8 Hz, 1H), 7.47 (d, J=8.8 Hz, 1H), 7.43 (d, J=8.9 Hz, 2H), 7.21 (d, J=7.9 Hz, 1H), 7.17 (s, 1H), 6.86 (d, J=7.9 Hz, 1H), 6.22 (d, J=8.9 Hz, 2H), 4.86-4.67 (m, 3H), 4.52-4.34 (m, 2H), 3.45-3.27 (m, 3H), 3.10 (d, J=8.0 Hz, 2H), 2.94 (s, 3H), 2.79-2.74 (m, 1H), 2.63 (t, J=6.3 Hz, 2H), 2.13-1.95 (m, 2H), 1.80-1.58 (m, 1H), 1.55-1.34 (m, 1H).

### Example 1.14 Synthesis of Compound 14

### Procedure for 2-(1⁴-methyl-4-oxo-3¹,3²,3³,3⁴-tetrahydro-1¹H-3(7,2)-isoquinolina-1(5,1)-benzo[d][1,2,3]triazola-5(1,4)-piperidinacyclodecaphane-2-yl)acetic acid (Compound 14)

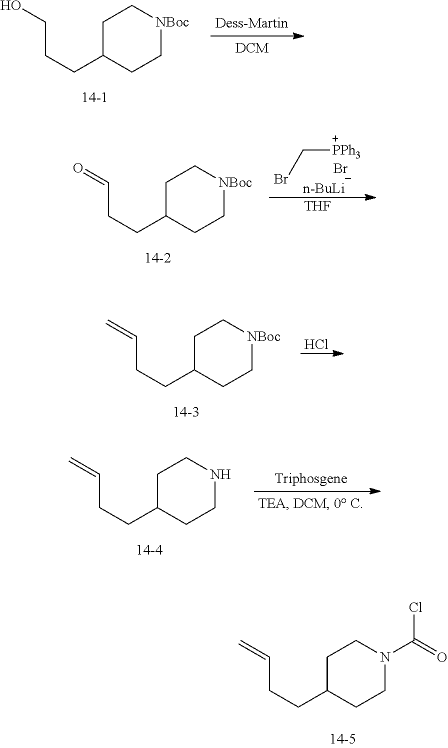

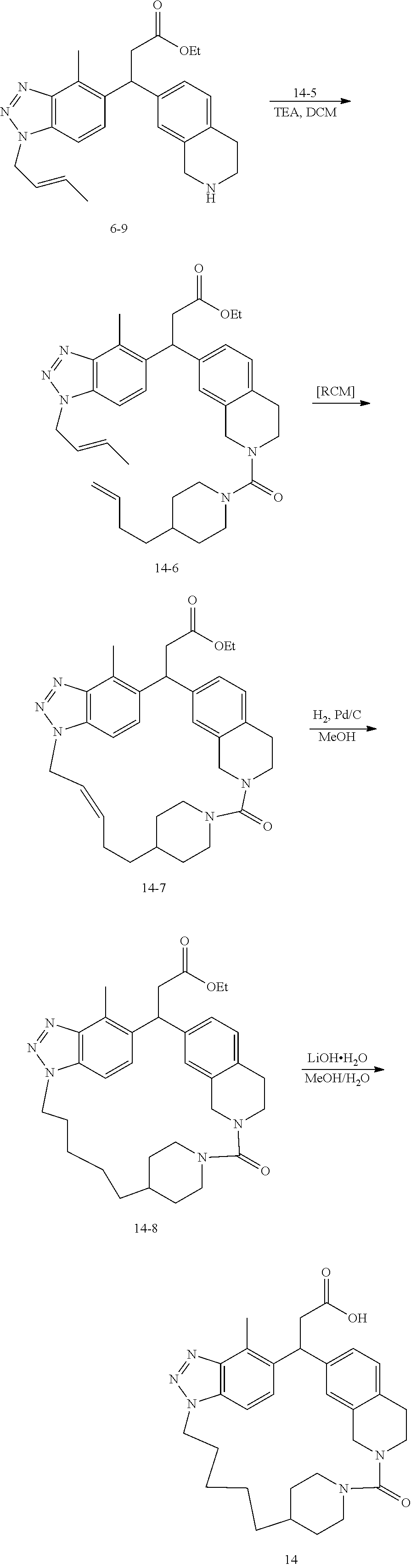

### tert-butyl 4-(3-oxopropyl)piperidine-1-carboxylate (14-2)

To a stirred mixture of tert-butyl 4-(3-hydroxypropyl)piperidine-1-carboxylate (5.0 g, 21.8 mmol) in DCM (50 mL) was added Dess-Martin reagent (9.0 g, 24.0 mmol) at 0° C. The resulting mixture was stirred at room temperature for 2 hours. After which period, the mixture was poured into saturated NaHCO₃ (100 mL) and then extracted with ethyl acetate (80 mL*3). The combined organic layers were dried over sodium sulfate, filtered and concentrated to dryness. The residue was purified by column chromatography (elute with ethyl acetate:petroleum ether=1:20 to 1:10) to have desired product 14-2 (4.8 g, 96% yield).

¹H NMR (400 MHz, CDCl₃) δ 9.78 (s, 1H), 4.22-3.94 (m, 2H), 2.69-2.49 (m, 2H), 2.52-2.41 (m, 2H), 1.72-1.55 (m, 4H), 1.47-1.38 (m, 10H), 1.10-1.01 (m, 2H).

### tert-butyl 4-(but-3-en-1-yl)piperidine-1-carboxylate (14-3)

To the solution of (bromomethyl)triphenylphosphonium bromide (11.2 g, 4.0 mmol) in THE (150 mL) was added n-BuLi (2.5 mL, 6.0 mmol) at −78° C. The resulting mixture was stirred at −78° C. for about 2 hours under nitrogen atmosphere. The solution of 14-2 (4.8 g, 2.0 mmol) in THE (25 mL) was added dropwise above solution. The resulting mixture was stirred at room temperature for another 19 hours under nitrogen atmosphere. TLC showed starting material was consumed. The mixture was then concentrated under reduced pressure. The residue was purified by column chromatography (elute with ethyl acetate:petroleum ether=1:20 to 1:10) to have desired product 14-3 (650.0 mg, 13.0% yield).

¹H NMR (400 MHz, CDCl₃) δ 5.80 (m, 1H), 5.06-4.90 (m, 2H), 4.15-3.98 (m, 2H), 2.67 (t, J=12.3 Hz, 2H), 2.14-2.05 (m, 2H), 1.69-1.55 (m, 2H), 1.45 (s, 9H), 1.37-1.31 (m, 3H), 1.15-1.01 (m, 2H).

### 4-(but-3-en-1-yl)piperidine (14-4)

To the solution of 14-3 (650.0 mg, 2.7 mmol) in MeOH (5 mL) was added HCl (4M in dioxane, 2.5 mL) at 0° C. The resulting mixture was stirred at 20° C. for 2 hours. The mixture was then concentrated to dryness to have desired product 14-4 (720.0 mg, crude), which was used directly to next run.

### 4-(but-3-en-1-yl)piperidine-1-carbonyl chloride (14-5)

To the solution of 14-4 (720.0 mg, crude) in DCM (30 mL) was added TEA (6 mL, 8.1 mmol) and triphosgene (1.5 g, 5.4 mmol) at 0° C. The resulting mixture was stirred at 20° C. for 4 hours. The mixture was then concentrated to dryness to have desired product 14-5 (1.0 g, crude), which was used directly to next run.

### ethyl (E)-3-(1-(but-2-en-1-yl)-4-methyl-1H-benzo[d][1,2,3]triazol-5-yl)-3-(2-(4-(but-3-en-1-yl)piperidine-1-carbonyl)-1,2,3,4-tetrahydroisoquinolin-7-yl)propanoate (14-6)

A solution of 6-9 (0.7 g, 1.6 mmol) and TEA (810.0 mg, 8.0 mmol) in DCM (30 mL) was added 14-5 (1.0 g, crude) at 0° C. The reaction was stirred at 25° C. overnight. After which period, the mixture was then concentrated to dryness. The residue was purified with Prep-HPLC (CH₃CN/H₂O, 0.1% HCl) to have the desired product 14-6 (0.5 g, 45.2% yield).

MS(ESI) calculated for C₃₅H₄₅N₅O₃, 583.4; found 584.8.

### ethyl (Z)-2-(1⁴-methyl-4-oxo-3¹,3² 3³,3⁴-tetrahydro-1¹H-3(7,2)-isoquinolina-1(5,1)-benzo[d][1,2,3]triazola-5(1,4)-piperidinacyclodecaphan-8-en-2-yl)acetate (14-7)

To the solution of 14-6 (0.5 g, 0.9 mmol) in DCM (30 mL) was added Grubbs II catalyst (15.0 mg, 0.2 mmol) at 25° C. The resulting mixture was stirred at 40° C. for 19 hours under nitrogen atmosphere. The mixture was then concentrated in vacuum to have desired product 14-7 (560.0 mg, crude), which was used directly to next run.

### ethyl 2-(1⁴-methyl-4-oxo-3¹,3²,3³,3⁴-tetrahydro-1¹H-3(7,2)-isoquinolina-1(5,1)-benzo[d][1,2,3]triazola-5(1,4)-piperidinacyclodecaphane-2-yl)acetate (14-8)

To the solution of 14-7 (560.0 mg, crude) in MeOH (10 mL) was added Pd/C (60.0 mg). The resulting mixture was stirred at room temperature for 19 hours under H₂ atmosphere. The catalyst was then removed by filtration, and the filtrate was concentrated to have desired product 14-8 (550.0 mg, crude).

### 2-(1⁴-methyl-4-oxo-3¹,3²,3³,3⁴-tetrahydro-1¹H-3(7,2)-isoquinolina-1(5,1)-benzo[d][1,2,3]triazola-5(1,4)-piperidinacyclodecaphane-2-yl)acetic acid (Compound 14)

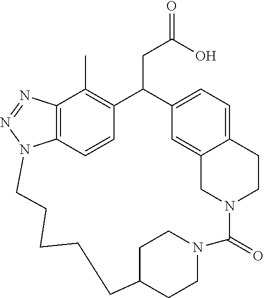 [SMILES: Cc1c2ccc3c1nnn3CCCCCC1CCN(CC1)C(=O)N1CCc3ccc(cc3C1)C2CC(=O)O]

To the solution of 14-8 (550.0 mg, crude) in MeOH (10.0 mL) and water (2 mL) was added LiOH·H₂O (0.2 g) at 0° C. The resulting mixture was stirred at room temperature for 2 hours. After which period, the reaction mixture was acidified with 1N HCl at 0° C. and extracted with ethyl acetate (15 mL*3). The combined organic layers were dried over sodium sulphate, filtered and concentrated to dryness. The residue was purified with Prep-HPLC (CH₃CN/H₂O, 0.1% HCl) to have the desired product Compound 14 (15.0 mg, 9.6%).

¹H NMR (400 MHz, DMSO-d₆) δ 7.68 (d, J=8.7 Hz, 1H), 7.44 (d, J=8.7 Hz, 1H), 7.34 (d, J=7.0 Hz, 1H), 7.12 (d, J=7.8 Hz, 1H), 6.55 (s, 1H), 4.77 (t, J=7.8 Hz, 1H), 4.73-4.65 (m, 2H), 4.06-3.95 (m, 2H), 3.42-3.36 (m, 2H), 3.14-2.60 (m, 4H), 2.80-2.70 (m, 2H), 2.60 (s, 3H), 2.49-2.10 (m, 1H), 2.15-2.10 (m, 2H), 2.00-1.95 (m, 1H), 1.29-1.15 (m, 3H), 1.06-1.04 (m, 2H), 1.00-0.98 (m, 2H), 0.81-0.60 (m, 3H), 0.50-0.36 (m, 1H).

MS(ESI) calculated for C₃₀H₃₇N₅O₃, 515.3; found 516.3.

### Example 1.15 Synthesis of Compound 15

### Procedure for 2-(1⁴-methyl-4,4-dioxido-3¹,3²,3³,3⁴-tetrahydro-1¹H-6,9-dioxa-4-thia-3(7,2)-isoquinolina-1(5,1)-benzo[d][1,2,3]triazola-5(1,4)-piperidinacycloundecaphane-2-yl)acetic acid (Compound 15)

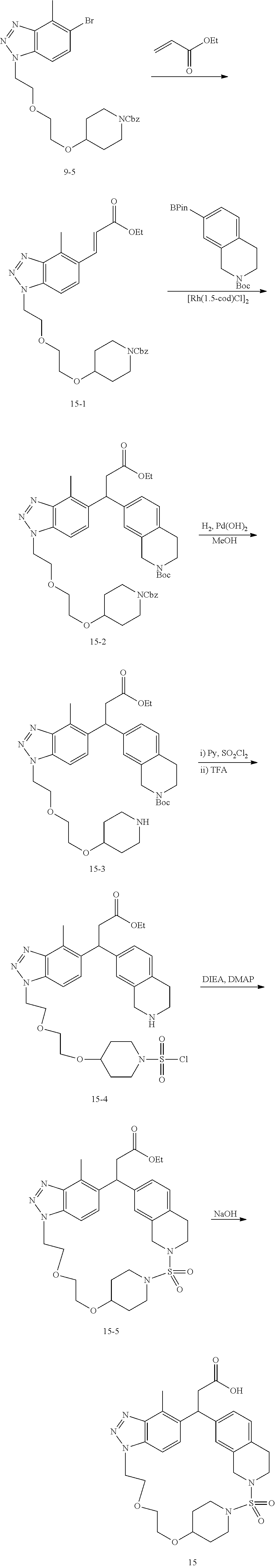

### benzyl-4-(2-(2-(5-(3-ethoxy-3-oxoprop-1-en-1-yl)-4-methyl-1H-benzo[d][1,2,3]triazol-1-yl)ethoxy)ethoxy)piperidine-1-carboxylate (15-1)

To the solution of benzyl 4-(2-(2-(5-bromo-4-methyl-1H-benzo[d][1,2,3]triazol-1-yl)ethoxy)ethoxy)piperidine-1-carboxylate (1.5 g, 2.9 mmol), ethyl acrylate (1.74 g, 17.4 mmol) and DIEA (3.3 g, 25.2 mmol) in DMF (30 mL) was added Pd(OAc)₂ (37.7 mg, 0.2 mmol) and (p-tol)₃P (0.1 g, 0.3 mmol) at 20° C. The resulting mixture was stirred at 120° C. for 19 hours under nitrogen atmosphere. After cooling to room temperature, the mixture was poured into ice water (150 mL) and then extracted with ethyl acetate (30 mL*3). The combined organic layers were dried over sodium sulfate, filtered and concentrated to dryness. The residue was purified by column chromatography (elute with ethyl acetate:petroleum ether=1:10 to 1:2) to have the desired product 15-1 (1.2 g, 77.1%).

MS(ESI) calculated for C₂₉H₃₆N₄O₆, 536.3; found 537.3.

### tert-butyl 7-(1-(1-(2-(2-((1-((benzyloxy)carbonyl)piperidin-4-yl)oxy)ethoxy)ethyl)-4-methyl-1H-benzo[d][1,2,3]triazol-5-yl)-3-ethoxy-3-oxopropyl)-3,4-dihydroisoquinoline-2(1H)-carboxylate (15-2)

To a mixture of 15-1 (1.2 g, 2.2 mmol), sodium dodecyl sulfate (0.3 g, 1.1 mmol), TEA (0.7 g, 0.7 mmol) and tert-butyl 7-(4,4,5,5-tetramethyl-1,3,2-dioxaborolan-2-yl)-3,4-dihydroisoquinoline-2(1H)-carboxylate (2.4 g, 6.7 mmol) in CPME (100 mL) and water (20 mL) was added [Rh(1,5-cod)Cl]₂ (113.1 mg, 0.2 mmol) at room temperature. The resulting mixture was stirred at 110° C. for 3 hours under nitrogen atmosphere. After cooling to room temperature, the mixture was poured into ice water (100 mL) and extracted with ethyl acetate (25 mL*3). The combined organic layers were dried over sodium sulfate, filtered and concentrated to dryness. The residue was purified by column chromatography (elute with ethyl acetate:petroleum ether=1:10 to 1:2) to have the desired product 15-2 (0.9 g, 52.3%).

### tert-butyl 7-(3-ethoxy-1-(4-methyl-1-(2-(2-(piperidin-4-yloxy)ethoxy)ethyl)-1H-benzo[d][1,2,3]triazol-5-yl)-3-oxopropyl)-3,4-dihydroisoquinoline-2(1H)-carboxylate (15-3)

To the solution of 15-2 (0.9 g, 0.2 mmol) in MeOH (30 mL) was added Pd(OH)₂ (20.2 mg) at room temperature. The resulting mixture was stirred at 20° C. for 6.5 hours under H₂ atmosphere. The catalyst was removed by filtration, and the filtrate was concentrated to have the desired product 15-3 (450.0 mg, 60.6%), which was used directly to next run.

MS(ESI) calculated for C₃₅H₄₉N₅O₆, 635.4; found 636.5.

### ethyl 3-(1-(2-(2-((1-(chlorosulfonyl)piperidin-4-yl)oxy)ethoxy)ethyl)-4-methyl-1H-benzo[d][1,2,3]triazol-5-yl)-3-(1,2,3,4-tetrahydroisoquinolin-7-yl)propanoate (15-4)

To the solution of 15-3 (250.0 mg, 0.4 mmol) and pyridine (155.5 mg, 2.0 mmol) in DCM (20 mL) was added SO₂Cl₂ (106.1 mg, 0.8 mmol) in DCM (20 mL) at 0° C. The resulting mixture was stirred at room temperature for 4 hours under nitrogen atmosphere. To the above mixture TFA (357.2 mg, 3.2 mmol) was added at 0° C. The resulting mixture was stirred at room temperature for another 2 hours under nitrogen atmosphere. The mixture was then concentrated to dryness to have crude desired product 15-4 (460.2 mg, impure), which was used directly to next run.

MS(ESI) calculated for C₃₀H₄₀ClN₅O₆S, 633.2; found 634.3.

### ethyl 2-(1⁴-methyl-4,4-dioxido-3¹,3²,3³,3⁴-tetrahydro-1¹H-6,9-dioxa-4-thia-3(7,2)-isoquinolina-1(5,1)-benzo[d][1,2,3]triazola-5(1,4)-piperidinacycloundecaphane-2-yl)acetate (15-5)

To the solution of 15-4 (crude 460.0 mg, at most 0.4 mmol) in DMF (10 mL) was added TEA (0.5 g, 3.9 mmol) and DMAP (4.8 mg, 39.4 mol) at 0° C. The resulting mixture was stirred at room temperature for 19 hours under nitrogen atmosphere. After which period, the mixture was poured into ice water (60 mL) and then extracted with ethyl acetate (20 mL*3). The combined organic layers were dried over sodium sulfate, filtered and concentrated to dryness. The residue was purified with Prep-HPLC (CH₃CN/H₂O, 0.1% HCl) to have the desired product 15-5 (100.4 mg, 48.2%).

MS(ESI) calculated for C₃₀H₃₉N₅O₆S, 597.3; found 598.3.

### 2-(1⁴-methyl-4,4-dioxido-3¹,3²,3³,3⁴-tetrahydro-1¹H-6,9-dioxa-4-thia-3(7,2)-isoquinolina-1(5,1)-benzo[d][1,2,3]triazola-5(1,4)-piperidinacycloundecaphane-2-yl)acetic acid (Compound 15)

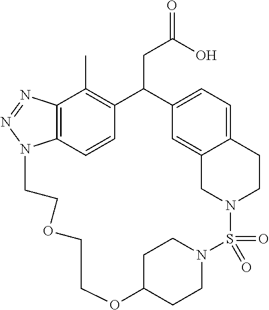 [SMILES: Cc1c2ccc3c1nnn3CCOCCOC1CCN(CC1)S(=O)(=O)N1CCc3ccc(cc3C1)C2CC(=O)O]

To the solution of 15-5 (90.0 mg, 0.2 mmol) in THE (5 mL) MeOH (2 mL) and H₂O (2 mL) was added NaOH (3.3 mg, 0.1 mmol) at 0° C. The resulting mixture was stirred at room temperature for 2.5 hours under nitrogen atmosphere. After which period, the reaction mixture was acidified with 1N HCl at 0° C. and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 15 (25.0 mg, 29.1%).

MS(ESI) calculated for C₂₈H₃₅N₅O₆S, 569.2; found 570.2.

¹H NMR (400 MHz, DMSO-d₆) δ 7.51 (d, J=8.7 Hz, 1H), 7.37 (s, 1H), 7.33 (d, J=8.8 Hz, 1H), 7.25-7.21 (m, 1H), 7.08 (d, J=7.9 Hz, 1H), 4.91-4.66 (m, 3H), 4.36-4.20 (m, 2H), 3.90-3.82 (m, 1H), 3.75-3.69 (m 1H), 3.63-3.57 (m, 1H), 3.50-3.45 (m, 2H), 3.36-3.30 (m, 1H), 3.25-3.18 (m, 1H), 3.16-2.99 (m, 5H), 2.90 (s, 3H), 2.82-2.77 (m, 2H), 2.67-2.56 (m, 1H), 2.42-2.33 (m, 2H), 1.25-1.15 (m, 2H), 0.92-0.67 (m, 2H).

### Example 1.16 Synthesis of Compound 16 and Compound 17

### Procedure for 2-(1⁴,3⁴, 5-trimethyl-6-oxo-1¹H-9-oxa-5-aza-1(5,1)-benzo[d][1,2,3]triazola-3(1,3), 7(1,4)-dibenzenacyclododecaphane-2-yl)acetic acid (Compound 16 and Compound 17)

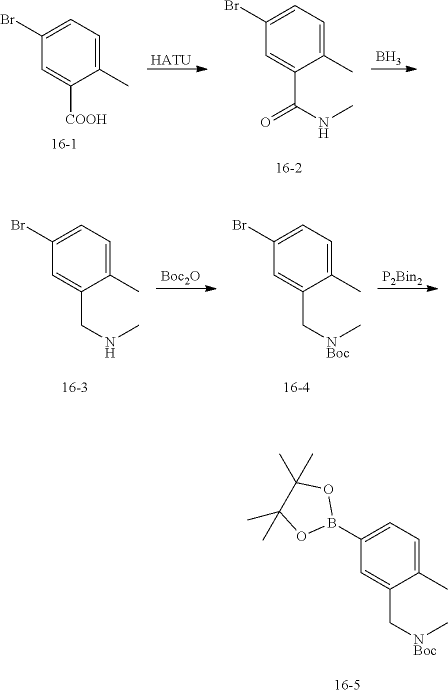

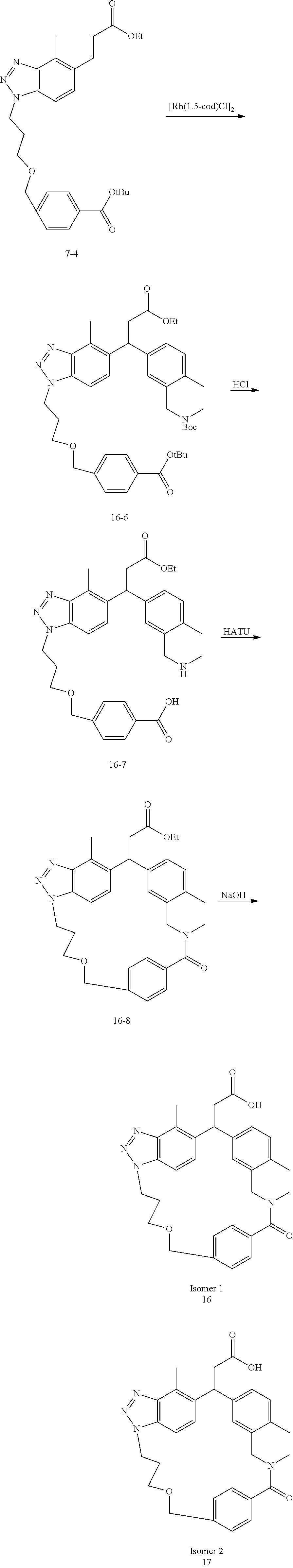

### 5-bromo-N,2-dimethylbenzamide (16-2)

To the solution of 5-bromo-2-methylbenzoic acid (10.0 g, 46.5 mmol) in DMF (100 mL) was added MeNH₂·HCl (6.3 g, 93.0 mmol), DIPEA (24.0 g, 186.0 mmol) and HATU (26.5 g, 69.8 mmol) at 0° C. The resulting mixture was stirred at 25° C. for 19 hours under nitrogen atmosphere. After which period, the mixture was poured into ice water (500 mL) and then extracted with ethyl acetate (100 mL*3). The combined organic layers were dried over sodium sulfate, filtered and concentrated to dryness to have the desired product 16-2 (12.8 g, crude).

MS(ESI) calculated for C₉H₁₀BrNO, 226.9; found 228.1,230.5.

### 1-(5-bromo-2-methylphenyl)-N-methylmethanamine (16-3)

To the solution of 16-2 (12.8 g, 56.1 mmol) in THF (50 mL) was added borane tetrahydrofuran complex (1M, 150 mL) at 0° C. The resulting mixture was stirred at room temperature for 19 hours under nitrogen atmosphere. After which period, the reaction was quenched with the addition of MeOH (300 mL), and the resulting mixture was concentrated to dryness to have the desired product 16-3 (9.5 g, 79.1%).

MS(ESI) calculated for C₉H₁₂BrN, 213.0; found 214.3,216.2.

### tert-butyl (5-bromo-2-methylbenzyl)(methyl)carbamate (16-4)

To the solution of 16-3 (9.5 g, 44.4 mmol) in THF (100 mL) and water (10 mL) was added (Boc)₂O (11.6 g, 53.4 mmol) at 25° C. The resulting mixture was stirred at room temperature for 19 hours. After which period, the mixture was then poured into ice water (500 mL) and extracted with ethyl acetate (100 mL*3). The combined organic layers were dried over sodium sulfate, filtered and concentrated to dryness. The residue was purified by column chromatography (elute with ethyl acetate:petroleum ether=1:10 to 1:1) to have the desired product 16-4 (9.0 g, 94.7%).

MS(ESI) calculated for C₁₄H₂₀BrNO₂,313.1; found 314.3,316.2.

### tert-butyl methyl(2-methyl-5-(4,4,5,5-tetramethyl-1,3,2-dioxaborolan-2-yl)benzyl)carbamate (16-5)

To the solution of 16-4 (5.0 g, 16.0 mmol), B₂Pin₂ (4.5 g, 17.6 mmol) and AcOK (3.1 g, 31.6 mmol) in dioxane (60 mL) was added Pd(dppf)Cl₂ (1.2 g, 1.6 mmol). The resulting mixture was stirred at 100° C. for 19 hours under nitrogen atmosphere. After cooling to room temperature, the mixture was poured into ice water (100 mL) and then extracted with ethyl acetate (80 mL*3). The combined organic layers were dried over sodium sulfate, filtered and concentrated to dryness. The residue was purified by column chromatography (elute with ethyl acetate:petroleum ether=1:10 to 1:1) to have the desired product 16-5 (2.7 g, 14.3%).

MS(ESI) calculated for C₂₀H₃₂BNO₄,361.2; found 362.3.

### tert-butyl 4-((3-(5-(1-(3-(((tert-butoxycarbonyl)(methyl)amino)methyl)-4-methylphenyl)-3-ethoxy-3-oxopropyl)-4-methyl-1H-benzo[d][1,2,3]triazol-1-yl)propoxy)methyl)benzoate (16-6)

To the solution of 16-5 (1.3 g, 4.5 mmol), 7-4 (0.7 g, 1.5 mmol) and Sodium dodecyl sulphate (210.0 mg, 0.8 mmol) in CPME (20 mL) and water (5 mL) was added TEA (375.2 mg, 0.5 mmol) and [Rh(1,5-cod)Cl]₂ (73.0 mg, 0.2 mmol). The resulting mixture was stirred at 100° C. for 19 hours under nitrogen atmosphere. After cooling to room temperature, the mixture was poured into ice water (80 mL) and then extracted with ethyl acetate (20 mL*3). The combined organic layers were dried over sodium sulfate, filtered and concentrated to dryness. The residue was purified by column chromatography (elute with ethyl acetate:petroleum ether=1:2 to 1:1) to have the desired compound 16-6 (280.0 mg, 26.1%).

MS(ESI) calculated for C₄₁H₅₄N₄O₇ 714.4; found 715.2.

### 4-((3-(5-(3-ethoxy-1-(4-methyl-3-((methylamino)methyl)phenyl)-3-oxopropyl)-4-methyl-1H-benzo[d][1,2,3]triazol-1-yl)propoxy)methyl)benzoic acid (16-7)

To the solution of 16-6 (280.0 mg, 0.4 mmol) in MeOH (5 mL) was added HCl (4M in dioxane, 5 mL) at 0° C. The resulting mixture was stirred at 20° C. for 2 hours and then concentrated to dryness to have the desired product 16-7 (260.0 mg, crude).

MS(ESI) calculated for C₃₂H₃₈N₄O₅ 558.2; found 559.7.

### ethyl 2-(1⁴,3⁴, 5-trimethyl-6-oxo-1¹H-9-oxa-5-aza-1(5,1)-benzo[d][1,2,3]triazola-3(1,3), 7(1,4)-dibenzenacyclododecaphane-2-yl)acetate (16-8)

To the solution of 16-7 (260.0 mg, crude) and DIEA (0.3 g, 2.3 mmol) in DMF (10 mL) was added HATU (0.3 g, 0.7 mmol) at 0° C. The resulting mixture was stirred at 20° C. for 2 hours. The mixture was then poured into ice water (100 mL) and extracted with ethyl acetate (20 mL*3). The combined organic layers were dried over sodium sulfate, filtered and concentrated to dryness. The residue was purified by column chromatography (elute with ethyl acetate:petroleum ether=1:2 to 2:1) to have the desired product 16-8 (240.0 mg, 95.2%).

MS(ESI) calculated for C₃₂H₃₆N₄O₄ 540.3; found 541.2.

### 2-(1⁴,3⁴,5-trimethyl-6-oxo-1¹H-9-oxa-5-aza-1(5,1)-benzo[d][1,2,3]triazola-3(1,3), 7(1,4)-dibenzenacyclododecaphane-2-yl)acetic acid (Compound 16 and Compound 17)

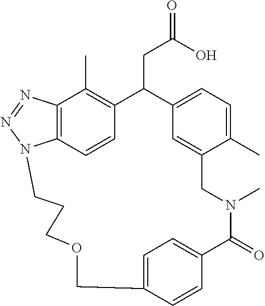 [SMILES: Cc1ccc2cc1CN(C)C(=O)c1ccc(cc1)COCCCn1nnc3c(C)c(ccc31)C2CC(=O)O]

To the solution of 16-8 (120 mg, 221.8 μmol) in MeOH (2 mL), water (2 mL) and THE (4 mL) was added NaOH (50 mg) at 0° C. The resulting mixture was stirred at 20° C. for 19 hours under nitrogen atmosphere. After which period, the reaction mixture was acidified with 1N HCl at 0° C. and then extracted with ethyl acetate (15 mL*3). The combined organic layers were dried over sodium sulfate, filtered and concentrated to dryness. The residue was purified with Prep-HPLC (CH₃CN/H₂O, 0.1% HCl) to have the desired product Compound 16 and Compound 17 (3.9 mg, 3.4%).

MS(ESI) calculated for C₃₀H₃₂N₄O₄, 512.2; found 513.4.

¹H NMR (400 MHz, DMSO-d₆): δ 13.04-11.15 (m, 1H), 8.29-6.03 (m, 9H), 5.10-3.86 (m, 7H), 3.15-2.66 (m, 7H), 2.45-1.90 (m, 6H), 1.27-1.20 (m, 2H).

Compound 16 and Compound 17 was separated by prep-HPLC: column: BP-C18, 10 μm, 21.2*250 mm (Pntulips). Isomer 1 (i.e., Compound 16) (retention time=5.728 min) and Isomer 2 (i.e., Compound 17) (retention time=6.655 min) were obtained.

### Example 1.17 Synthesis of Compounds 28 and 29

### Procedure for Preparation of Compound 28 and Compound 29

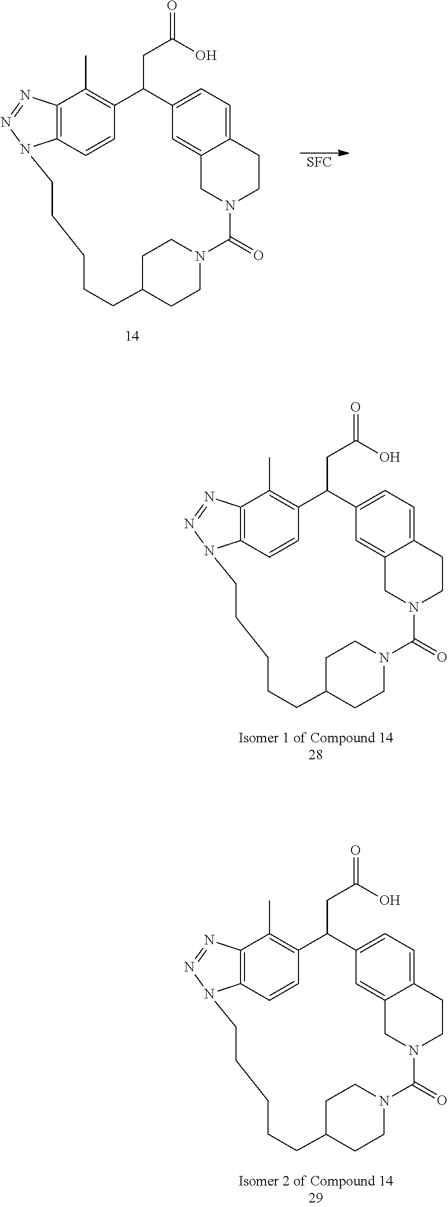

360 mg of 14 was separated by SFC: column: DAICEL CHIRALPAK AD (250 mm*30 mm,10 um); mobile phase: [0.1% NH₃·H₂O MEOH]; B %: 45%-45%, 45 min. Isomer 1 of Compound 14 (i.e., Compound 28) (138 mg, retention time=3.037 min) and Isomer 2 of Compound 14 (i.e., Compound 29) (141 mg, retention time=3.249 min) were obtained.

¹HNMR of Isomer 1 of Compound 14 (i.e., Compound 28): (400 MHz, DMSO-d₆) δ ppm 7.58 (d, J=8.7 Hz, 1H), 7.43-7.33 (m, 2H), 7.05 (d, J=7.9 Hz, 1H), 6.43 (s, 1H), 4.81 (br t, J=7.4 Hz, 1H), 4.71-4.59 (m, 2H), 4.04-3.90 (m, 2H), 3.56-3.44 (m, 2H), 3.11 (br d, J=12.6 Hz, 1H), 2.80 (br d, J=13.3 Hz, 1H), 2.74 (br t, J=6.5 Hz, 2H), 2.69-2.59 (m, 2H), 2.58-2.54 (m, 3H), 2.34-2.21 (m, 1H), 2.18-2.03 (m, 2H), 2.02-1.89 (m, 1H), 1.30-1.13 (m, 2H), 1.09-0.92 (m, 5H), 0.80-0.58 (m, 3H), 0.50-0.36 (m, 1H) LCMS: [M+H]⁺=516.3, retention time=0.834 min HPLC: retention time=3.853 min, 94.150% purity.

¹HNMR of Isomer 2 of Compound 14 (i.e., Compound 29): (400 MHz, DMSO-d₆) δ ppm 7.65 (d, J=8.7 Hz, 1H), 7.42 (d, J=8.7 Hz, 1H), 7.33 (br d, J=7.7 Hz, 1H), 7.09 (d, J=7.8 Hz, 1H), 6.52 (s, 1H), 4.81-4.73 (m, 1H), 4.70-4.63 (m, 2H), 4.05-3.93 (m, 2H), 3.40 (br s, 2H), 3.05 (br s, 3H), 2.80-2.73 (m, 3H), 2.59 (s, 3H), 2.33-2.25 (m, 1H), 2.15-2.06 (m, 2H), 1.97 (br dd, J=5.4, 11.8 Hz, 1H), 1.27-1.15 (m, 2H), 1.09-0.93 (m, 5H), 0.78-0.59 (m, 3H), 0.50-0.36 (m, 1H) LCMS: [M+H]⁺=516.2, retention time=0.834 min HPLC: retention time=3.848 min, 94.231% purity.

### Example 1.18 Synthesis of Compound 30

### Procedure for 2-(1⁴-chloro-4-oxo-3¹,3²,3³,3⁴-tetrahydro-1¹H-3(7,2)-isoquinolina-1(5,1)-benzo[d][1,2,3]triazola-5(1,4)-piperidinacyclodecaphane-2-yl)acetic acid (Compound 30)

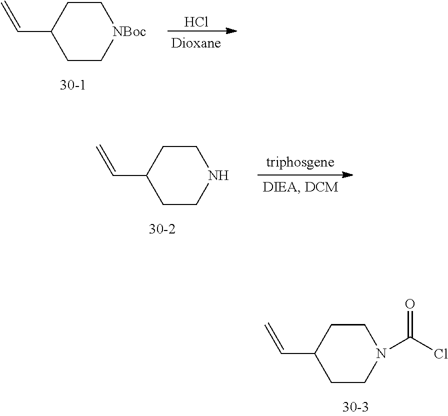

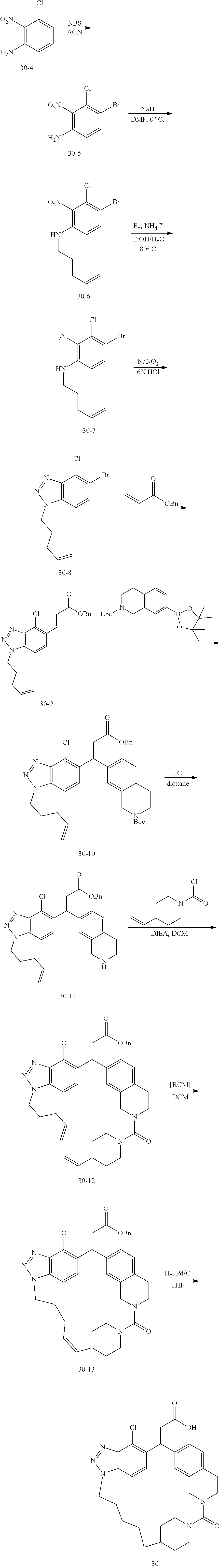

### 4-vinylpiperidine (30-2)

To the solution of tert-butyl 4-vinylpiperidine-1-carboxylate (1.0 g, 4.7 mmol) in dioxane (10 mL) was added HCl (4M in dioxane, 10 mL) at 0° C. The resulting mixture was stirred at 20° C. for 2 hours and then concentrated to dryness to have the desired product 30-2 (660.0 mg, wet).

### 4-vinylpiperidine-1-carbonyl chloride (30-3)

To the solution of 30-2 (660 mg, 5.9 mmol) and DIEA (2.3 g, 17.8 mmol) in DCM (20 mL) was added bis(trichloromethyl) carbonate (881.2 mg, 3.0 mmol) at 0° C. The resulting mixture was stirred at room temperature for 19 hours under nitrogen atmosphere. After which period, the mixture was poured into ice water (100 mL) and then extracted with DCM (100 mL*3). The combined organic layers were dried over sodium sulfate, filtered and concentrated to dryness to have crude product 30-3 (1.0 g, 97.0%).

### 4-bromo-3-chloro-2-nitroaniline (30-5)

To the solution of 3-chloro-2-nitroaniline (25 g, 144.8 mmol) in AcOH (250 mL) was added NBS (28.4 g, 159.3 mol) at 20° C. The resulting mixture was stirred at 110° C. for 4 hours under nitrogen atmosphere. After cooling to room temperature, the reaction mixture was poured into ice water. The resultant precipitate was filtered off and dried under high vacuum to have crude product 30-5 (35.0 g, 96%).

### 4-bromo-3-chloro-2-nitro-N-(pent-4-en-1-yl)aniline (30-6)

To the solution of 30-5 (20.0 g, 79.5 mmol) in DMF (200 mL) was added NaH (3.8 g, 95.4 mmol) at 0° C. The resulting mixture was stirred at room temperature for 0.5 hour under nitrogen atmosphere. To above mixture was added the solution of 5-bromopent-1-ene (17.8 g, 119.3 mmol) in THE (10 mL). The resulting mixture was stirred at room temperature for 19 hours under nitrogen atmosphere. The mixture was then poured into ice water (1 L) and extracted with ethyl acetate (500 mL*3). The combined organic layers were dried over sodium sulfate, filtered and concentrated to dryness. The residue was purified by column chromatography (elute with ethyl acetate:petroleum ether=1:10 to 1:5) to have the desired product 30-6 (11.3 g, 44.5%).

### 4-bromo-3-chloro-N¹-(pent-4-en-1-yl)benzene-1,2-diamine (30-7)

To the solution of 30-6 (11.3 g, 35.4 mmol) and NH₄Cl (18.9 g, 353.6 mmol) in EtOH (120 mL) and H₂O (40 mL) was added Fe powder (19.8 g, 353.6 mol) at 20° C. The resulting mixture was stirred at 80° C. for 4 hours under nitrogen atmosphere. After cooling to 40° C., the mixture was then filtered through a pad of celite. The filtrate was concentrated to dryness to have the crude product 30-7 (6.3 g, 61.5%).

### 5-bromo-4-chloro-1-(pent-4-en-1-yl)-1H-benzo[d][1,2,3]triazole (30-8)

To the solution of 30-7 (6.3 g, 21.8 mmol) in HCl (6M in H₂O, 70 mL) was added NaNO₂ (3.0 g, 43.5 mmol) in H₂O (20 mL) at 0° C. The resulting mixture was stirred at 25° C. for 4 hours. The reaction mixture was then neutralized with 4N NaOH at 0° C. The aqueous layer was extracted with ethyl acetate (100 mL*3). The combined organic layers were dried over sodium sulfate, filtered and concentrated to dryness. The residue was purified by column chromatography (elute with ethyl acetate:petroleum ether=1:5 to 1:3) to have desired product 30-8 (2.8 g, 42.4%).

### benzyl (E)-3-(4-chloro-1-(pent-4-en-1-yl)-1H-benzo[d][1,2,3]triazol-5-yl)acrylate (30-9)

To the solution of 30-8 (2.8 g, 9.2 mmol) benzyl acrylate (9.0 g, 55.3 mmol) and DIEA (2.4 g, 26.0 mmol) in DMF (30 mL) was added Pd(OAc)₂ (310.0 mg, 1.4 mmol) and P(O-tol)₃ (840 mg, 2.8 mmol) at 20° C. The resulting mixture was stirred at 120° C. for 19 hours under nitrogen atmosphere. After cooling to room temperature, the mixture was poured into ice water (150 mL) and then extracted with ethyl acetate (100 mL*3). The combined organic layers were dried over sodium sulfate, filtered and concentrated to dryness. The residue was purified by column chromatography (elute with ethyl acetate:petroleum ether=1:3 to 1:1) to have the desired product 30-9 (2.3 g, 65.4%).

MS(ESI) calculated for C₂₁H₂₀ClN₃O₂, 381.1; found 382.1.

### tert-butyl 7-(3-(benzyloxy)-1-(4-chloro-1-(pent-4-en-1-yl)-1H-benzo[d][1,2,3]triazol-5-yl)-3-oxopropyl)-3,4-dihydroisoquinoline-2(1H)-carboxylate (30-10)

To a mixture of 30-9 (1.4 g, 3.7 mmol), sodium dodecyl sulfate (534 mg, 1.9 mmol), Et₃N (1.1 g, 11.1 mmol), tert-butyl 7-(4,4,5,5-tetramethyl-1,3,2-dioxaborolan-2-yl)-3,4-dihydroisoquinoline-2(1H)-carboxylate (4.0 g, 11.1 mmol) in CPME (20 mL) and water (10 mL) was added [Rh(1,5-cod)Cl]₂ (183.0 mg, 0.4 mmol) at room temperature. The resulting mixture was stirred at 110° C. for 4 hours under nitrogen atmosphere. After cooling to room temperature, the mixture was poured into ice water (50 mL) and then extracted with ethyl acetate (50 mL*3). The combined organic layers were dried over sodium sulfate, filtered and concentrated to dryness. The residue was purified by column chromatography (elute with ethyl acetate:petroleum ether=1:3 to 1:1) to have the desired product 30-10 (1.2 g, 53.8%).

MS(ESI) calculated for C₃₅H₃₉ClN₄O₄, 614.3; found 559.3, 515.3.

### benzyl 3-(4-chloro-1-(pent-4-en-1-yl)-1H-benzo[d][1,2,3]triazol-5-yl)-3-(1,2,3,4-tetrahydroisoquinolin-7-yl)propanoate (30-11)

To the solution of 30-10 (1.2 g, 2.0 mmol) in dioxane (20 mL) was added HCl (4M in dioxane, 20 mL) at 0° C. The resulting mixture was stirred at 20° C. for 2 hours. The mixture was then concentrated to dryness to have desired product 30-11 (1.1 g, wet).

MS(ESI) calculated for C₃₀H₃₁ClN₄O₂, 514.2; found 515.2.

### benzyl 3-(4-chloro-1-(pent-4-en-1-yl)-1H-benzo[d][1,2,3]triazol-5-yl)-3-(2-(4-vinylpiperidine-1-carbonyl)-1,2,3,4-tetrahydroisoquinolin-7-yl)propanoate (30-12)

To the solution of 30-11 (500.0 mg, 1.0 mmol) and 30-3 (252.6 mg, 1.5 mmol) in DCM (10 mL) was added DIEA (375.3 mg, 2.9 mmol) at 0° C. The resulting mixture was stirred at room temperature for 19 hours under nitrogen atmosphere. After which period, the mixture was poured into ice water (20 mL) and then extracted with ethyl acetate (20 mL*3). The combined organic layers were dried over sodium sulfate, filtered and concentrated to dryness. The residue was purified by column chromatography (elute with ethyl acetate:petroleum ether=1:2 to 1:1) to have desired product 30-12 (550.0 mg, 86.7%).

MS(ESI) calculated for C₃₈H₄₂ClN₅O₃, 651.3; found 652.2.

### benzyl (E)-2-(1⁴-chloro-4-oxo-3¹,3²,3³,3⁴-tetrahydro-1¹H-3(7,2)-isoquinolina-1(5,1)-benzo[d][1,2,3]triazola-5(1,4)-piperidinacyclodecaphan-6-en-2-yl)acetate (30-13)

To the solution of 30-12 (550 mg, 0.8 mmol) in DCM (15 mL) was added Grubbs II catalyst (215 mg, 0.3 mmol). The resulting mixture was stirred at room temperature for 19 hours under nitrogen atmosphere. The mixture was then poured into ice water (20 mL) and extracted with DCM (20 mL*3). The combined organic layers were dried over sodium sulfate, filtered and concentrated to dryness. The residue was purified by column chromatography (elute with ethyl acetate:petroleum ether=1:2 to 1:1) to have the desired product 30-13 (70 mg, 13.3%).

MS(ESI) calculated for C₃₆H₃₈ClN₅O₃, 623.3; found 624.3.

### 2-(1⁴-chloro-4-oxo-3¹,3²,3³,3⁴-tetrahydro-1¹H-3(7,2)-isoquinolina-1(5,1)-benzo[d][1,2,3]triazola-5(1,4)-piperidinacyclodecaphane-2-yl)acetic acid (Compound 30)

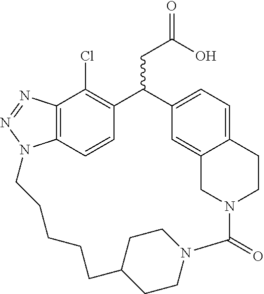 [SMILES: O=C(O)CC1c2ccc3c(c2)CN(CC3)C(=O)N2CCC(CCCCCn3nnc4c(Cl)c1ccc43)CC2]

To the mixture of 30-13 (70.0 mg, 0.1 mmol) and 10% Pd/C (20.0 mg) in THE (10 mL) was stirred at room temperature for 19 hours under hydrogen atmosphere. The mixture was filtered through a pad of celite, and the filtrate was concentrated to dryness. The residue was purified with Prep-HPLC (CH₃CN/H₂O, 0.1% HCl) to have the desired product Compound 30 (1.7 mg, 2.8%).

MS(ESI) calculated for C₂₉H₃₄ClN₅O₃, 535.2; found 536.2.

¹H NMR (400 MHz, DMSO-d₆) δ 12.31 (brs, 1H), 7.92 (d, J=8.7 Hz, 1H), 7.65 (d, J=8.7 Hz, 1H), 7.34 (dd, J=7.8, 1.4 Hz, 1H), 7.12 (d, J=7.8 Hz, 1H), 6.61 (s, 1H), 5.00 (t, J=7.8 Hz, 1H), 4.82-4.68 (m, 2H), 4.05-4.02 (m, 2H), 3.62-3.55 (m, 2H), 3.16-3.04 (m, 4H), 2.80-2.73 (m, 3H), 2.33-2.30 (m, 1H), 2.16-2.06 (m, 2H), 1.99 (s, 1H), 1.28-0.95 (m, 8H), 0.85-0.65 (m, 2H).

### Example 1.19 Synthesis of Compounds 34, 35, 47, 48, 49 and 50

### Procedure for Preparation of Compounds 34, 35, 47, 48, 49 and 50

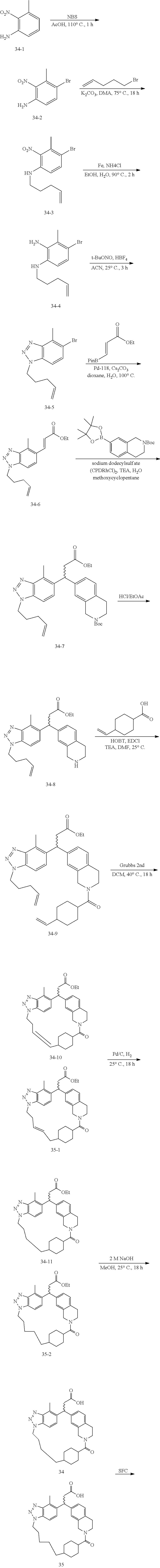

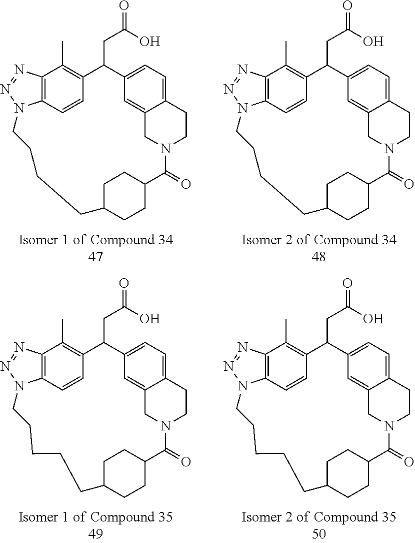 [SMILES: Cc1c2ccc3c1nnn3CCCCC1CCC(CC1)C(=O)N1CCc3ccc(cc3C1)C2CC(=O)O ; Cc1c2ccc3c1nnn3CCCCC1CCC(CC1)C(=O)N1CCc3ccc(cc3C1)C2CC(=O)O ; Cc1c2ccc3c1nnn3CCCCCC1CCC(CC1)C(=O)N1CCc3ccc(cc3C1)C2CC(=O)O ; Cc1c2ccc3c1nnn3CCCCCC1CCC(CC1)C(=O)N1CCc3ccc(cc3C1)C2CC(=O)O]

### Procedure for Preparation of Compound 34-2

To a solution of 34-1 (50.0 g, 328 mmol, 1.00 eq) in AcOH (500 mL) was added NBS (58.4 g, 328 mmol, 1.00 eq). The mixture was stirred at 110° C. for 1 h. The reaction mixture was quenched by addition ice water 20.0 μL at 25° C., filtered and concentrated under reduced pressure to give a residue. The residue was not purified to next step. 34-2 (71.0 g, 291 mmol, 88.8% yield, 95.0% purity) was obtained. LCMS: [M+H]⁺=230.9, retention time=0.723 min

### Procedure for Preparation of Compound 34-3

To a solution of 34-2 (50.0 g, 216 mmol, 1.00 eq) in DMA (200 mL) was added 5-bromopent-1-ene (38.7 g, 259 mmol, 1.20 eq) and K₂CO₃ (179 g, 1.30 mol, 6.00 eq). The mixture was stirred at 100° C. for 72 h. The reaction mixture was partitioned between EA (200 mL) and H₂O (200 mL). The organic phase was separated, washed with H₂O (300 mL), washed with brine (300 mL), dried over Na₂SO₄, filtered and concentrated under reduced pressure to give a residue. The residue was purified by flash silica gel chromatography (ISCO®; 220 g SepaFlash® Silica Flash Column, Eluent of 0~10% Ethyl acetate/petroleum ether gradient at 100 mL/min). 34-3 (29.7 g, 99.2 mmol, 45.8% yield) was obtained.

¹H NMR: (400 MHz, DMSO-d₆) δ ppm 7.52 (d, J=9.05 Hz, 1H) 6.71 (d, J=9.17 Hz, 1H) 5.80 (m, 1H) 5.02 (m, 1H) 4.95 (m, 1H) 3.12 (m, 2H) 2.24 (s, 3H) 2.05 (m, 2H) 1.60 (m, 2H) LCMS: [M+H]⁺=299.0, retention time=0.867 min

### Procedure for Preparation of Compound 34-4

To a solution of 34-3 (29.7 g, 99.2 mmol, 1.00 eq) in EtOH (300 mL) and H₂O (100 mL) was added Fe (38.8 g, 694 mmol, 7.00 eq) and NH₄Cl (15.9 g, 297 mmol, 3.00 eq). The mixture was stirred at 90° C. for 2 h. The reaction mixture was partitioned between EA (200 mL) and H₂O (200 mL). The organic phase was separated, washed with brine (300 mL), dried over Na₂SO₄, filtered and concentrated under reduced pressure to give a residue. Compound 34-4 (25.0 g, 90.0 mmol, 90.7% yield, 97.0% purity) was obtained.

### Procedure for Preparation of Compound 34-5

Solution 1: HBF₄ (40.7 g, 185 mmol, 28.9 mL, 40.0% purity, 2.00 eq) and tert-butyl nitrite (14.3 g, 139 mmol, 16.5 mL, 1.50 eq) were dissolved in 300 mL of acetonitrile and cooled to 0° C. Solution 2: A solution of 34-4 (25.0 g, 92.8 mmol, 1.00 eq) dissolved in a solution of MeCN (250 mL). Solution 1 was added slowly, dropwise to solution 2 while keeping the internal temperature below 5° C. The reaction mixture was stirred at 25° C. for 2 h. An aqueous solution of H₂O (500 mL) was added dropwise into the reaction mixture and the combined organic layers were washed with NaCl aq (500 mL), dried over MgSO₄, filtered and concentrated. The residue was purified by flash silica gel chromatography (ISCO®; 220 g SepaFlash® Silica Flash Column, Eluent of 0~30% Ethyl acetate/petroleum ether gradient at 100 mL/min). 34-5 (19.0 g, 67.8 mmol, 73.0% yield) was obtained.

¹H NMR: (400 MHz, DMSO-d₆) δ ppm 7.67 (m, 1H) 7.67 (d, J=1.10 Hz, 1H) 5.79 (ddt, J=16.97, 10.43, 6.22, 6.22 Hz, 1H) 4.97 (m, 2H) 4.68 (m, 2H) 2.69 (s, 3H) 1.99 (m, 4H) LCMS: [M+H]⁺=281.9, retention time=0.782 min

### Procedure for Preparation of Compound 34-6

To a solution of 34-5 (19.0 g, 67.8 mmol, 1.00 eq) and ethyl (E)-3-(4,4,5,5-tetramethyl-1,3,2-dioxaborolan-2-yl)prop-2-enoate (15.3 g, 67.8 mmol, 1.00 eq) in dioxane (200 mL) and H₂O (50 mL) was added Cs₂CO₃ (44.1 g, 135 mmol, 2.00 eq) and ditert-butyl(cyclopentyl)phosphane;dichloropalladium; iron (2.21 g, 3.39 mmol, 0.05 eq). The mixture was stirred at 100° C. for 2 h under N₂. An aqueous solution of H₂O (500 mL) was added dropwise into the reaction mixture and the combined organic layers were washed with NaCl aq (500 mL), dried over MgSO₄, filtered and concentrated. The residue was purified by flash silica gel chromatography (ISCO®; 220 g SepaFlash® Silica Flash Column, Eluent of 0~30% Ethyl acetate/petroleum ether gradient at 100 mL/min). 34-6 (13.3 g, 44.4 mmol, 65.5% yield) was obtained.

¹H NMR: (400 MHz, DMSO-d₆) δ ppm 7.98 (m, 2H) 7.71 (m, 1H) 6.64 (d, J=15.76 Hz, 1H) 5.37 (m, 2H) 4.71 (m, 2H) 4.21 (q, J=7.09 Hz, 2H) 2.80 (s, 3H) 2.59 (m, 2H) 1.49 (d, J=4.75 Hz, 2H) 1.28 (t, J=7.13 Hz, 3H)

### Procedure for Preparation of Compound 34-7

A mixture of 34-6 (16.0 g, 53.4 mmol, 1.00 eq), tert-butyl 7-(4,4,5,5-tetramethyl-1,3,2-dioxaborolan-2-yl)-3,4-dihydro-1H-isoquinoline-2-carboxylate (30.7 g, 85.5 mmol, 1.60 eq), sodiumdodecyl sulfate (7.71 g, 26.7 mmol, 7.63 mL, 0.50 eq), chlororhodium(1Z,5Z)-cycloocta-1,5-diene (2.64 g, 5.34 mmol, 0.10 eq) in TEA (10 mL), H₂O (50 mL) and methoxy-cyclopentane (100 mL) was degassed and purged with N₂ for 3 times, and then the mixture was stirred at 90° C. for 4 h under N₂ atmosphere. An aqueous solution of H₂O (500 mL) was added dropwise into the reaction mixture and the combined organic layers were washed with NaCl aq (500 mL), dried over MgSO₄, filtered and concentrated. The residue was purified by flash silica gel chromatography (ISCO®; 220 g SepaFlash® Silica Flash Column, Eluent of 0~40% Ethyl acetate/Petroleum ether gradient at 100 mL/min). 34-7 (26.0 g, 48.8 mmol, 91.3% yield) was obtained.

¹H NMR: (400 MHz, DMSO-d₆) δ ppm 7.60 (d, J=8.58 Hz, 1H) 7.50 (m, 1H) 7.12 (br d, J=8.36 Hz, 2H) 7.04 (m, 1H) 5.36 (m, 1H) 4.81 (br t, J=7.81 Hz, 1H) 4.64 (br t, J=6.82 Hz, 2H) 4.42 (br s, 2H) 3.92 (q, J=6.97 Hz, 2H) 3.49 (br t, J=5.61 Hz, 2H) 3.15 (br d, J=7.92 Hz, 2H) 2.76 (m, 3H) 2.68 (br t, J=5.61 Hz, 2H) 2.55 (br d, J=6.16 Hz, 2H) 1.49 (m, 2H) 1.40 (s, 9H) 0.99 (t, J=7.15 Hz, 3H) 0.84 (m, 2H)

### Procedure for Preparation of Compound 34-8

To a solution of 34-7 in dioxane (100 mL) was added HCl/dioxane (4.00 M, 100 mL, 10.6 eq) at 0° C. The mixture was stirred at 25° C. for 2 h. The reaction mixture was concentrated to give 34-8 (19.4 g, 35.1 mmol, 93.6% yield, 85.0% purity, HCl salt), which was directly used in the next step.

¹H NMR: (400 MHz, DMSO-d₆) δ ppm 7.62 (m, 1H) 7.61 (m, 1H) 7.48 (m, 1H) 7.24 (brd, J=8.13 Hz, 1H) 7.19 (s, 1H) 7.19 (m, 1H) 7.12 (d, J=8.00 Hz, 1H) 7.12 (m, 1H) 5.37 (m, 1H) 4.83 (t, J=7.88 Hz, 1H) 4.65 (m, 2H) 4.31 (br s, 1H) 4.17 (br s, 2H) 3.93 (q, J=7.05 Hz, 2H) 3.56 (s, 1H) 3.31 (brd, J=4.63 Hz, 2H) 3.16 (brd, J=8.00 Hz, 2H) 2.92 (brt, J=6.07 Hz, 2H) 2.77 (m, 3H) 1.50 (d, J=3.88 Hz, 2H) 1.07 (s, 3H) 1.00 (t, J=7.07 Hz, 3H)

### Procedure for Preparation of COMPOUND 34-9

To a solution of 34-8 (10.0 g, 21.3 mmol, 1.00 eq, HCl), 4-vinylcyclohexanecarboxylic acid (3.95 g, 25.5 mmol, 1.20 eq), anhydrous 1-hydroxybenzotriazole (5.76 g, 42.6 mmol, 2.00 eq) and 3-(ethyliminomethyleneamino)-N,N-dimethyl-propan-1-amine;hydrochloride (8.17 g, 42.6 mmol, 2.00 eq) in DMF (100 mL), TEA (8.63 g, 85.2 mmol, 11.8 mL, 4.00 eq) was added, and the reaction mixture was stirred at 25° C. under N₂ for 15 h. The mixture was diluted with H₂O (500 mL) and extracted with EA (500 mL). The combined organic layer was dried over Na₂SO₄ and filtered. The filtrate was concentrated to give the residue. The residue was purified by flash silica gel chromatography (ISCO®; 220 g SepaFlash® Silica Flash Column, Eluent of 0~30% Ethyl acetate/Petroleum ether gradient at 100 mL/min). 34-9 (10.0 g, 17.5 mmol, 82.4% yield) was obtained.

¹H NMR: (400 MHz, DMSO-d₆) δ ppm 7.59 (m, 1H) 7.49 (m, 1H) 7.13 (m, 2H) 7.05 (m, 1H) 5.36 (m, 1H) 4.99 (m, 1H) 4.91 (m, 1H) 4.82 (br d, J=6.00 Hz, 1H) 4.63 (br d, J=6.38 Hz, 2H) 4.52 (br s, 1H) 3.92 (q, J=7.09 Hz, 2H) 3.83 (br d, J=6.13 Hz, 1H) 3.64 (m, 2H) 3.30 (s, 1H) 3.15 (br d, J=8.13 Hz, 2H) 2.76 (br d, J=5.00 Hz, 4H) 2.65 (m, 1H) 2.55 (br d, J=6.00 Hz, 1H) 1.94 (m, 3H) 1.69 (m, 4H) 1.59 (br d, J=7.38 Hz, 1H) 1.49 (br d, J=4.00 Hz, 2H) 1.36 (m, 3H) 1.16 (m, 2H) 1.06 (s, 1H) 0.98 (t, J=7.07 Hz, 3H) LCMS: [M+H]⁺=569.2, retention time=0.844 min

### Procedure for Preparation of Compound 34-10 and Compound 35-1

To a solution of 34-9 (10.0 g, 17.6 mmol, 1.00 eq) in DCE (200 mL) was added [1,3-bis(2,4,6-trimethylphenyl)imidazolidin-2-ylidene]-dichloro-[(2-isopropoxyphenyl)methylene]ruthenium (440 mg, 703 mol, 0.20 eq) under N₂. The mixture was stirred at 50° C. for 18 h. The mixture was concentrated to give the residue. The residue was purified by flash silica gel chromatography (ISCO®; 120 g SepaFlash® Silica Flash Column, Eluent of 0~50% Ethyl acetate/Petroleum ether gradient at 60 mL/min). A mixture of 34-10 and 35-1 (1.40 g, 2.38 mmol, 14.0% yield, 92.0% purity) was obtained.

### Procedure for Preparation of Compound 34-11 and 35-2

To a mixture solution of 34-10 and 35-1 (1.34 g, 2.48 mmol, 1.00 eq) in MeOH (15.0 mL) was added wet Pd/C (1.00 g, 10.0% purity) under H₂ (15.0 psi). The mixture was stirred at 25° C. for 3 h. The reaction mixture was filtered and concentrated. The residue was not purified to next step. A mixture of 34-11 and 35-2 (1.30 g, 2.16 mmol, 86.9% yield, 90.0% purity) was obtained. LCMS: [M+H]⁺=529, retention time=0.760 min, [M+H]⁺=543, retention time=0.780 min

### Procedure for Preparation of Compounds 34 and 35

To the solution of 34-11 and 35-2 (mixture, 1.30 g, 2.40 mmol, 1.00 eq) in MeOH (50.0 mL) and H₂O (2.00 mL) was added NaOH (2.00 M, 5.39 mL, 4.50 eq) at 0° C. The resulting mixture was stirred at 25° C. for 18 h. After which period, the reaction mixture was acidified with 1.00 M HCl eq at 0° C. and extracted with ethyl acetate (50 mL). The combined organic layers were dried over sodium sulfate, filtered and concentrated to dryness. The residue was purified by prep-HPLC (column: Phenomenex C18 75*30 mm*3 um; mobile phase: [water(FA)-ACN];B %: 23%-63%,9 min), to afford Compound 34 and Compound 35.

### Procedure for Preparation of Compounds 47, 48, 49 and 50

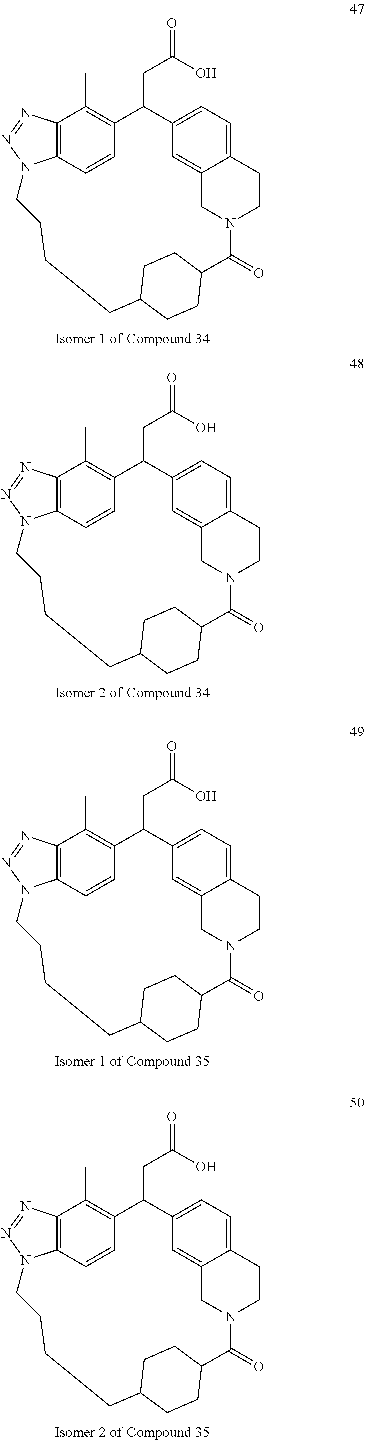 [SMILES: Cc1c2ccc3c1nnn3CCCCC1CCC(CC1)C(=O)N1CCc3ccc(cc3C1)C2CC(=O)O ; Cc1c2ccc3c1nnn3CCCCC1CCC(CC1)C(=O)N1CCc3ccc(cc3C1)C2CC(=O)O ; Cc1c2ccc3c1nnn3CCCCC1CCC(CC1)C(=O)N1CCc3ccc(cc3C1)C2CC(=O)O ; Cc1c2ccc3c1nnn3CCCCC1CCC(CC1)C(=O)N1CCc3ccc(cc3C1)C2CC(=O)O]

Compound 34 and Compound 35 were further separated by SFC (column: Boston Prime C18 150*30 mm*5 um;mobile phase: [water (ammonia hydroxide v/v)-ACN];B %: 33%-53%,9 min) and SFC (column: DAICEL CHIRALPAK IC(250 mm*30 mm,10 um);mobile phase: [0.1% NH3H2O ETOH];B %: 60%-60%, min). Isomer 1 of Compound 34 (i.e., Compound 47) (246.5 mg, 491 μmol, 20.5% yield, retention time=4.060 min), Isomer 2 of Compound 34 (i.e., Compound 48) (261.6 mg, 521 μmol, 21.7% yield, retention time=6.821 min), Isomer 1 of Compound 35 (i.e., Compound 49) (18.5 mg, 35.9 μmol, 1.50% yield, retention time=4.229 min), Isomer 2 of Compound 35 (i.e., Compound 50) (23.5 mg, 45.6 μmol, 1.91% yield, retention time=6.076 min) were obtained.

¹H NMR of Isomer 1 of Compound 34 (i.e., Compound 47): (400 MHz, DMSO-d₆) δ ppm 7.61 (d, J=8.56 Hz, 1H) 7.50 (br d, J=8.68 Hz, 1H) 7.43 (br d, J=7.70 Hz, 1H) 7.18 (d, J=7.70 Hz, 1H) 6.80 (s, 1H) 4.99 (br t, J=7.95 Hz, 1H) 4.75 (m, 3H) 4.38 (m, 2H) 3.73 (m, 2H) 3.19 (m, 2H) 2.91 (br t, J=6.85 Hz, 2H) 2.78 (s, 3H) 2.27 (br t, J=11.74 Hz, 1H) 1.96 (m, 2H) 1.31 (br d, J=13.69 Hz, 2H) 1.22 (m, 2H) 1.03 (br d, J=7.46 Hz, 2H) 0.94 (br s, 2H) 0.72 (br s, 2H) 0.56 (br s, 1H) 0.45 (br d, J=11.86 Hz, 1H) 0.34 (br d, J=11.00 Hz, 1H) LCMS: [M+H]⁺=501.2, retention time=0.878 min

¹H NMR of Isomer 2 of Compound 34 (i.e., Compound 48): (400 MHz, DMSO-d₆) δ ppm 7.57 (br d, J=8.68 Hz, 1H) 7.48 (br s, 1H) 7.39 (br d, J=6.97 Hz, 1H) 7.14 (d, J=7.70 Hz, 1H) 6.75 (s, 1H) 4.95 (m, 1H) 4.71 (m, 3H) 4.34 (m, 2H) 3.67 (m, 2H) 3.13 (m, 2H) 2.87 (br t, J=6.79 Hz, 2H) 2.74 (s, 3H) 2.23 (br t, J=11.55 Hz, 1H) 1.92 (m, 2H) 1.27 (br d, J=13.20 Hz, 3H) 1.15 (m, 2H) 0.98 (br d, J=7.83 Hz, 2H) 0.90 (br s, 2H) 0.67 (br s, 2H) 0.51 (br s, 1H) 0.43 (br s, 1H) 0.30 (br d, J=11.25 Hz, 1H) LCMS: [M+H]⁺=501.2, retention time=0.870 min

¹H NMR of Isomer 1 of Compound 35 (i.e., Compound 49): (400 MHz, DMSO-d₆) δ ppm 7.57 (m, 1H) 7.50 (m, 1H) 7.41 (br d, J=7.95 Hz, 1H) 7.16 (d, J=7.82 Hz, 1H) 6.85 (s, 1H) 4.92 (m, 3H) 4.68 (m, 2H) 4.41 (m, 2H) 3.70 (t, J=6.72 Hz, 2H) 3.12 (m, 2H) 2.87 (br t, J=6.60 Hz, 2H) 2.73 (s, 3H) 2.38 (br t, J=11.49 Hz, 1H) 2.10 (br dd, J=12.65, 7.40 Hz, 2H) 1.28 (br d, J=7.09 Hz, 4H) 1.06 (m, 5H) 0.89 (m, 2H) 0.73 (m, 1H) 0.52 (m, 2H) LCMS: [M+H]⁺=515.2, retention time=0.902 min

¹H NMR of Isomer 2 of Compound 35 (i.e., Compound 50): (400 MHz, DMSO-d₆) δ ppm 7.57 (m, 1H) 7.50 (m, 1H) 7.41 (br d, J=7.70 Hz, 1H) 7.16 (d, J=7.70 Hz, 1H) 6.85 (s, 1H) 4.68 (br t, J=3.79 Hz, 2H) 4.55 (br s, 3H) 4.41 (m, 2H) 3.70 (t, J=6.72 Hz, 2H) 3.12 (m, 2H) 2.87 (br t, J=6.54 Hz, 2H) 2.73 (s, 3H) 2.38 (br t, J=11.55 Hz, 1H) 2.11 (m, 2H) 1.28 (br d, J=6.36 Hz, 4H) 1.04 (m, 5H) 0.87 (br d, J=12.96 Hz, 2H) 0.74 (m, 1H) 0.52 (m, 2H) LCMS: [M+H]⁺=515.2, retention time=0.900 min

### Example 1.20 Synthesis of Compound 40

### Procedure for Preparation of Compound 40

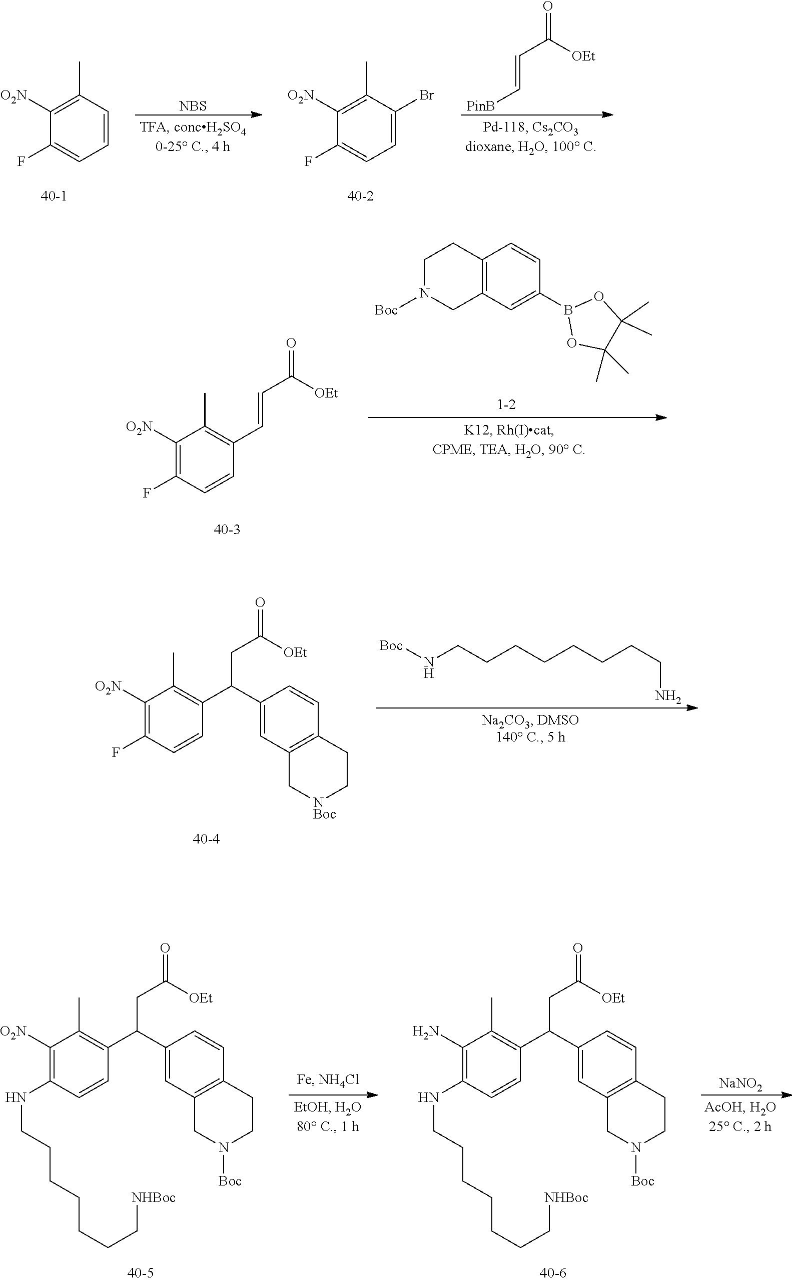

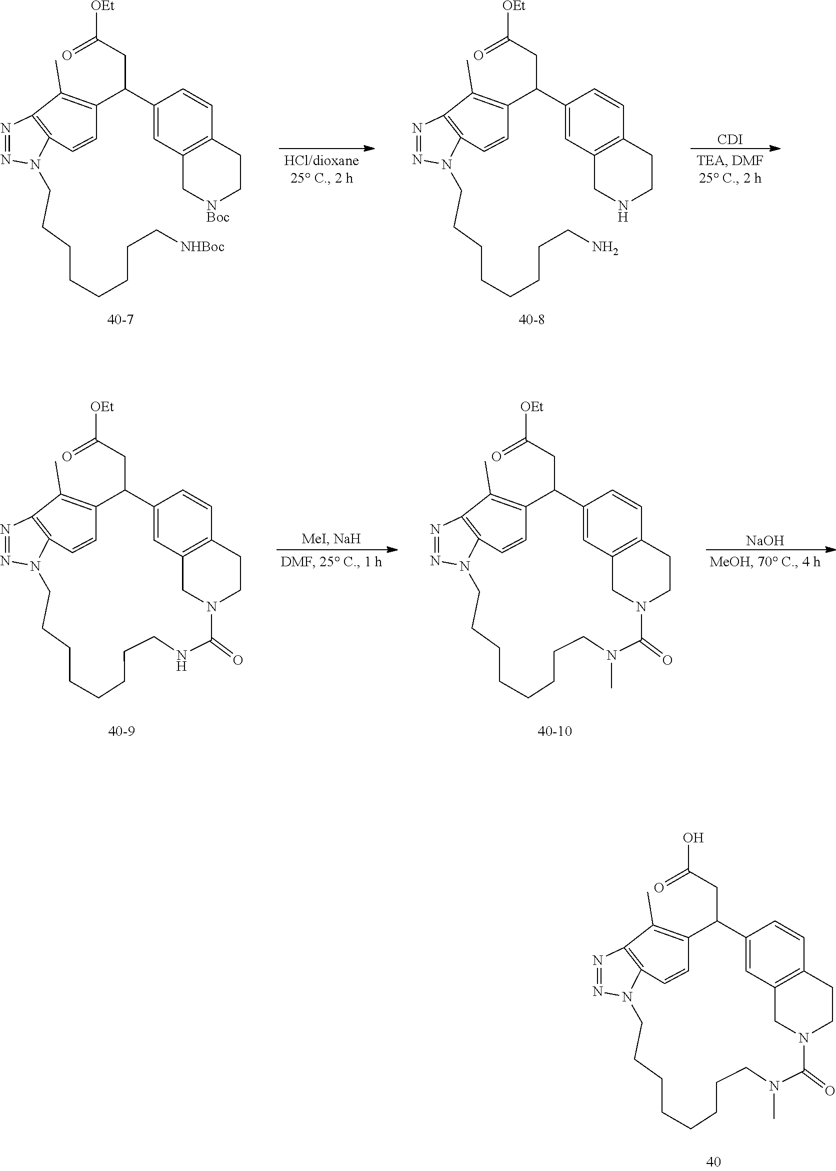

### Procedure for Preparation of Compound 40-2

To a solution of 40-1 (23 g, 148 mmol, 1 eq) in TFA (100 mL) was added H₂SO₄ (40 mL) at 0° C. slowly, followed by addition of NBS (29.0 g, 163 mmol, 1.1 eq) in portions. The mixture was stirred at 0-25° C. for 16 h. The resulting mixture was poured onto ice and the precipitate solid was collected by filtration. Washed with water and dried under vacuum to give compound 40-2 (29 g, 124 mmol, 83.6% yield).

¹H NMR: (400 MHz, DMSO-d₆) δ ppm 7.96 (dd, J=9.0, 5.0 Hz, 1H) 7.47 (t, J=9.3 Hz, 1H) 2.35 (s, 3H)

### Procedure for Preparation of Compound 40-3

A mixture of 40-2 (15 g, 64.10 mmol, 1 eq), ethyl (E)-3-(4,4,5,5-tetramethyl-1,3,2-dioxaborolan-2-yl)prop-2-enoate (14.5 g, 64.1 mmol, 1 eq), di-tert-butyl(cyclopentyl)phosphane dichloropalladium iron (2.09 g, 3.20 mmol, 0.05 eq) and cesium carbonate (41.8 g, 128 mmol, 2 eq) in dioxane (160 mL) and H₂O (40 mL) degassed and purged with N₂ for 3 times, and then the mixture was stirred at 100° C. for 18 h under N₂ atmosphere. The reaction mixture was concentrated under reduced pressure to remove solvent. The residue was diluted with H₂O (100 mL) and extracted with EtOAc (100 mL*3). The combined organic layers were washed with brine (300 mL*2), dried over MgSO₄, filtered and concentrated under reduced pressure to give a residue. The residue was purified by flash silica gel chromatography (ISCO®; 330 g SepaFlash® Silica Flash Column, Eluent of 0~15% Ethyl acetate/petroleum ether gradient at 100 mL/min) to give Compound 40-3 (13.2 g, 52.1 mmol, 81.3% yield).

¹H NMR: (400 MHz, DMSO-d₆) δ ppm 8.05 (dd, J=8.9, 5.6 Hz, 1H) 7.79 (d, J=15.8 Hz, 1H) 7.52 (t, J=9.3 Hz, 1H) 6.63 (d, J=15.8 Hz, 1H) 4.21 (q, J=7.0 Hz, 2H) 2.35 (s, 3H) 1.27 (t, J=7.0 Hz, 3H)

### Procedure for Preparation of Compound 40-4

A mixture of 40-3 (2.5 g, 9.87 mmol, 1 eq), tert-butyl 7-(4,4,5,5-tetramethyl-1,3,2-dioxaborolan-2-yl)-3,4-dihydro-1H-isoquinoline-2-carboxylate (5.32 g, 14.8 mmol, 1.5 eq), sodium dodecyl sulfate (1.42 g, 4.94 mmol, 1.41 mL, 0.5 eq), chlororhodium;(1Z,5Z)-cycloocta-1,5-diene (487 mg, 987 mol, 0.1 eq) in TEA (6 mL), methoxycyclopentane (60 mL) and H₂O (30 mL) was degassed and purged with N₂ for 3 times, and then the mixture was stirred at 90° C. for 4 h under N₂ atmosphere. The reaction mixture was partitioned between H₂O (30 mL) and EtOAc (100 mL). The organic phase was separated, washed with brine (100 mL), dried over Na₂SO₄, filtered and concentrated under reduced pressure to give a residue. The residue was purified by flash silica gel chromatography (ISCO®; 120 g SepaFlash® Silica Flash Column, Eluent of 0~18% Ethyl acetate/petroleum ether gradient at 100 mL/min) to give compound 40-4 (3.9 g, 8.02 mmol, 81.2% yield).

¹H NMR: (400 MHz, DMSO-d₆) δ ppm 7.70 (dd, J=8.9, 5.6 Hz, 1H) 7.43 (t, J=9.2 Hz, 1H) 7.01-7.17 (m, 3H) 4.65 (t, J=7.9 Hz, 1H) 4.43 (br s, 2H) 3.97 (q, J=7.2 Hz, 2H) 3.51 (br t, J=5.5 Hz, 2H) 3.08-3.23 (m, 2H) 2.70 (br t, J=5.9 Hz, 2H) 2.29 (s, 3H) 1.41 (s, 9H) 1.05 (t, J=7.0 Hz, 3H)

### Procedure for Preparation of Compound 40-5

A mixture of 40-4 (2 g, 4.11 mmol, 1 eq), tert-butyl N-(8-aminooctyl)carbamate (2.01 g, 8.22 mmol, 2 eq), Na₂CO₃ (1.31 g, 12.33 mmol, 3 eq) in DMSO (20 mL) was degassed and purged with N₂ for 3 times, and then the mixture was stirred at 140° C. for 5 h under N₂ atmosphere. The residue was diluted with H2O (15 mL) and extracted with EtOAc (20 mL*3). The combined organic layers were washed with brine (30 mL*2), dried over Na₂SO₄, filtered and concentrated under reduced pressure to give a residue. The crude product was purified by chromatography on a silica gel eluted with petroleum ether:ethyl acetate (from 0/1 to 4/1) to give compound 40-5 (2.5 g, 3.52 mmol, 85.5% yield).

¹H NMR: (400 MHz, DMSO-d₆) δ ppm 7.33 (d, J=8.8 Hz, 1H) 6.99-7.10 (m, 3H) 6.65-6.79 (m, 2H) 5.67 (t, J=5.5 Hz, 1H) 4.47-4.56 (m, 1H) 4.43 (br s, 2H) 3.88-4.10 (m, 1H) 3.88-4.10 (m, 2H) 3.50 (brs, 2H) 2.97 (brd, J=7.8 Hz, 1H) 2.94-3.15 (m, 3H) 2.87 (q, J=6.5 Hz, 2H) 2.69 (brt, J=5.9 Hz, 2H) 2.14 (s, 3H) 1.99 (s, 1H) 1.41 (s, 8H) 1.38-1.45 (m, 1H) 1.36 (s, 9H) 1.13-1.29 (m, 10H) 1.05 (t, J=7.2 Hz, 3H)

### Procedure for Preparation of Compound 40-6

To a solution of 40-5 (1 g, 1.41 mmol, 1 eq) in EtOH (9 mL) and H₂O (3 mL) was added Fe (550 mg, 9.85 mmol, 7 eq) and NH₄Cl (150 mg, 2.81 mmol, 2 eq). The mixture was stirred at 90° C. for 2 h. The reaction mixture was concentrated under reduced pressure to remove EtOH. The residue was diluted with H₂O (10 mL) and extracted with EtOAc (10 mL*3). The combined organic layers were washed with brine (10 mL*2), dried over Na₂SO₄, filtered and concentrated under reduced pressure to give a residue. The residue was purified by flash silica gel chromatography (ISCO®; 12 g SepaFlash® Silica Flash Column, Eluent of 0~25% Ethyl acetate/petroleum ether gradient at 80 mL/min) to give compound 40-6 (0.86 g, 1.26 mmol, 89.8% yield). LCMS: [M+H]⁺=667, retention time=1.002 min

### Procedure for Preparation of Compound 40-7

To a solution of 40-6 (860 mg, 1.26 mmol, 1 eq) in AcOH (15 mL) was added dropwise NaNO₂ (104 mg, 1.52 mmol, 1.2 eq) in H₂O (3 mL) at 25° C. The mixture was stirred at 25° C. for 3 h. The reaction mixture was concentrated under reduced pressure to remove AcOH. The residue was diluted with H₂O (30 mL) and extracted with EtOAc (50 mL*2). The combined organic layers were washed with bine (50 mL*2), dried over Na₂SO₄, filtered and concentrated under reduced pressure to give compound 40-7 (820 mg, 1.19 mmol, 93.8% yield).

¹H NMR: (400 MHz, DMSO-d₆) δ ppm 7.56-7.62 (m, 1H) 7.49 (d, J=8.8 Hz, 1H) 7.08-7.16 (m, 2H) 7.02-7.06 (m, 1H) 6.71 (br s, 1H) 4.81 (t, J=7.9 Hz, 1H) 4.61 (brt, J=6.9 Hz, 2H) 4.42 (br s, 2H) 3.92 (q, J=7.1 Hz, 2H) 3.49 (br t, J=5.8 Hz, 2H) 3.13-3.18 (m, 2H) 2.84 (q, J=6.6 Hz, 2H) 2.76 (s, 3H) 2.68 (br t, J=5.6 Hz, 2H) 1.79-1.89 (m, 2H) 1.40 (s, 9H) 1.34 (s, 9H) 1.12-1.27 (m, 10H) 0.98 (t, J=7.2 Hz, 3H)

### Procedure for Preparation of Compound 40-8

A mixture of 40-7 (0.2 g, 289 mol, 1 eq) in HCl/dioxane (4 M, 10 mL) was degassed and purged with N₂ for 3 times, and then the mixture was stirred at 25° C. for 1.5 h under N₂ atmosphere. The reaction mixture was filtered and concentrated under reduced pressure to give residue. The residue was used into the next step without further purification. Compound 40-8 (140 mg, 284 mol, 98.5% yield) was obtained.

### Procedure for Preparation of Compound 40-9

To a solution of 40-8 (0.26 g, 528 mol, 1 eq) in DMF (26 mL) was added CDI (85.7 mg, 528 μmol, 1 eq) and TEA (321 mg, 3.17 mmol, 441 μL, 6 eq) at 0° C. The mixture was stirred at 25° C. for 2 h. The reaction mixture was diluted with H₂O (50 mL) and extracted with EtOAc (50 mL*3). The combined organic layers were washed with brine (100 mL*3), dried over Na₂SO₄, filtered and concentrated under reduced pressure to give a residue. The residue was purified by flash silica gel chromatography (ISCO®; 8 g SepaFlash® Silica Flash Column, Eluent of 0~70% Ethyl acetate/petroleum ether gradient at 50 mL/min) to give compound 40-9 (30 mg, 57.9 mol, 10.9% yield).

¹H NMR: (400 MHz, DMSO-d₆) δ ppm 7.59 (d, J=8.8 Hz, 1H) 7.25-7.38 (m, 2H) 7.08 (d, J=7.8 Hz, 1H) 6.52 (s, 1H) 6.17 (brt, J=4.9 Hz, 1H) 4.84 (brt, J=7.9 Hz, 1H) 4.57-4.77 (m, 2H) 4.08-4.27 (m, 2H) 3.96 (q, J=7.0 Hz, 2H) 2.97-3.19 (m, 2H) 2.77 (s, 3H) 2.66 (br s, 2H) 1.91 (br d, J=6.3 Hz, 2H) 1.18-1.27 (m, 3H) 0.72-1.17 (m, 14H)

### Procedure for Preparation of Compound 40-10

To a solution of 40-9 (40 mg, 77.2 μmol, 1 eq) in DMF (10 mL) was added NaH (30.91 mg, 772 mol, 60% purity, 10 eq) and Mel (10.9 mg, 77.3 μmol, 4.81 μL, 1 eq) at 0° C. The mixture was stirred at 25° C. for 1 h. The reaction mixture was quenched by addition sat. NH₄Cl (20 mL), extracted with EtOAc (20 mL*3). The combined organic layers were washed with brine (30 mL*2), dried over Na₂SO₄, filtered and concentrated under reduced pressure to give a residue. The crude product 40-10 (32 mg, 60.2 μmol, 77.9% yield) was used in the next step without further purification. LCMS: [M+H]⁺=518.3, retention time=1.789 min

### Procedure for Preparation of Compound 40

To a solution of 40-10 (32 mg, 60.2 μmol, 1 eq) in MeOH (2 mL) was added NaOH (1 M, 300 μL, 5 eq). The mixture was stirred at 25° C. for 4 h. And then the reaction mixture was stirred at 70° C. for 2 h. The reaction mixture was concentrated under reduced pressure to remove MeOH. The residue was purified by prep-HPLC (FA condition) to give compound 40 (11 mg, 21.2 μmol, 35.3% yield, 97.272% purity).

¹H NMR: (400 MHz, DMSO-d₆) δ ppm 7.66 (d, J=8.8 Hz, 1H) 7.49 (d, J=8.5 Hz, 1H) 7.32 (br d, J=7.5 Hz, 1H) 7.08 (d, J=7.8 Hz, 1H) 6.54 (s, 1H) 4.75-4.82 (m, 1H) 4.61-4.75 (m, 2H) 4.00-4.17 (m, 2H) 3.22-3.44 (m, 2H) 3.08 (tt, J=16.0, 8.1 Hz, 2H) 2.76-2.89 (m, 2H) 2.71 (br t, J=5.8 Hz, 2H) 2.66 (s, 3H) 2.58 (s, 3H) 1.86-2.06 (m, 2H) 1.09-1.35 (m, 4H) 0.63-0.95 (m, 6H) LCMS: [M+H]⁺=504.3, retention time=0.675 min HPLC: retention time=3.813, 97.272% purity

### Example 1.21 Synthesis of Compound 41

### Procedure for Preparation of Compound 41

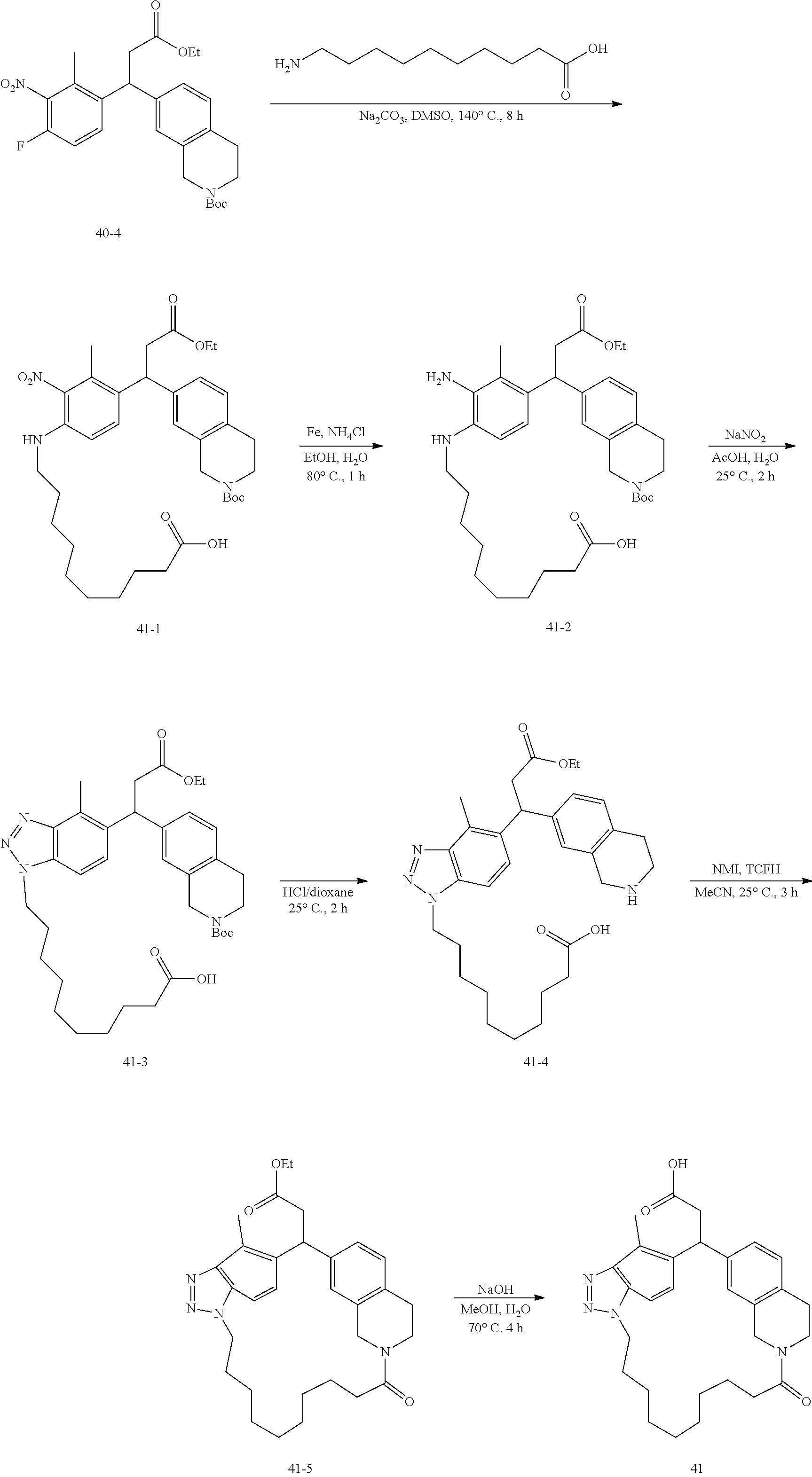

### Procedure for Preparation of Compound 41-1

A mixture of 40-4 (1 g, 2.06 mmol, 1 eq), 10-aminodecanoic acid (1.25 g, 6.67 mmol, 3.25 eq) and Na₂CO₃ (653.54 mg, 6.17 mmol, 3 eq) in DMSO (3 mL) was degassed and purged with N₂ for 3 times, and then the mixture was stirred at 140° C. for 5 h under N₂ atmosphere. The residue was diluted with H₂O 15 mL and extracted with EtOAc 60 mL (20 mL*3). The combined organic layers were washed with brine (30 mL*2), dried over Na₂SO₄, filtered and concentrated under reduced pressure to give a residue. The crude product was purified by chromatography on a silica gel eluted with petroleum ether:ethyl acetate (from 0/1 to 4/1) to give compound 41-1 (0.994 g, 1.52 mmol, 73.9% yield).

¹H NMR: (400 MHz, DMSO-d₆) δ ppm 11.95 (br s, 1H) 7.33 (d, J=8.8 Hz, 1H) 6.96-7.12 (m, 3H) 6.70 (d, J=9.0 Hz, 1H) 5.66 (t, J=5.4 Hz, 1H) 4.51 (t, J=7.9 Hz, 1H) 4.43 (br s, 2H) 4.09 (q, J=5.0 Hz, 2H) 3.96 (q, J=7.2 Hz, 2H) 3.50 (br s, 2H) 3.17 (d, J=4.8 Hz, 4H) 2.98-3.10 (m, 4H) 2.69 (br t, J=5.8 Hz, 2H) 2.17 (t, J=7.3 Hz, 2H) 2.14 (s, 3H) 1.41 (s, 9H) 1.24 (br s, 16H) 1.05 (t, J=7.2 Hz, 3H)

### Procedure for Preparation of Compound 41-2

To a solution of 41-1 (0.9 g, 1.38 mmol, 1 eq) in EtOH (9 mL) and H₂O (3 mL) was added Fe (538 mg, 9.64 mmol, 7 eq) and NH₄Cl (147 mg, 2.75 mmol, 2 eq). The mixture was stirred at 90° C. for 2 h. The reaction mixture was diluted with H₂O 50 mL and extracted with EtOAc (50 mL*3). The combined organic layers were washed with brine (100 mL*3), dried over Na₂SO₄, filtered and concentrated under reduced pressure to give a residue. The residue was purified by flash silica gel chromatography (ISCO®; 12 g SepaFlash® Silica Flash Column, Eluent of 0~60% Ethyl acetate/petroleum ether gradient at 60 mL/min) to give compound 41-2 (460 mg, 737 μmol, 53.6% yield).

¹H NMR: (400 MHz, DMSO-d₆) δ ppm 6.89-7.03 (m, 3H) 6.49 (d, J=8.3 Hz, 1H) 6.30 (d, J=8.3 Hz, 1H) 4.50 (br t, J=7.8 Hz, 1H) 4.40 (br s, 2H) 3.94 (q, J=7.0 Hz, 2H) 3.44-3.57 (m, 2H) 2.84-3.00 (m, 4H) 2.68 (br t, J=5.5 Hz, 2H) 2.13-2.22 (m, 2H) 1.96 (s, 3H) 1.53-1.60 (m, 2H) 1.41 (s, 12H) 1.20-1.31 (m, 12H) 1.06 (t, J=7.2 Hz, 3H) 0.81-0.92 (m, 1H)

### Procedure for Preparation of Compound 41-3

To a solution of 41-2 (450 mg, 721 μmol, 1 eq) in AcOH (20 mL) was added dropwise NaNO₂ (59.7 mg, 865 μmol, 1.2 eq) in H₂O (4 mL) at 25° C. The mixture was stirred at 25° C. for 3 h. The reaction mixture was diluted with H₂O (10 mL) and extracted with EtOAc (10 mL*3). The combined organic layers were washed with brine (15 mL*2), dried over Na₂SO₄, filtered and concentrated under reduced pressure to give a residue. The residue was purified by flash silica gel chromatography (ISCO®; 4 g SepaFlash® Silica Flash Column, Eluent of 0~40% EtOAc/petroleum ether gradient at 50 mL/min) to give compound 41-3 (170 mg, 268 μmol, 37.1% yield).

¹H NMR: (400 MHz, DMSO-d₆) δ ppm 11.94 (br s, 1H) 7.57-7.63 (m, 1H) 7.50 (d, J=8.8 Hz, 1H) 7.09-7.17 (m, 2H) 7.01-7.08 (m, 1H) 4.82 (t, J=7.9 Hz, 1H) 4.61 (brt, J=6.9 Hz, 2H) 4.42 (br s, 2H) 3.92 (q, J=7.0 Hz, 2H) 3.49 (br t, J=5.6 Hz, 2H) 3.12-3.19 (m, 2H) 2.77 (s, 3H) 2.68 (br t, J=5.6 Hz, 2H) 2.15 (t, J=7.4 Hz, 2H) 1.79-1.90 (m, 2H) 1.40 (s, 9H) 1.13-1.28 (m, 12H) 0.99 (t, J=7.2 Hz, 3H)

### Procedure for Preparation of Compound 41-4

A mixture of 41-3 (150 mg, 236 μmol, 1 eq) in HCl/dioxane (4 M, 10 mL) was degassed and purged with N₂ for 3 times, and then the mixture was stirred at 25° C. for 2 h under N₂ atmosphere. The reaction mixture was concentrated under reduced pressure to give 41-4 (101 mg, 176 μmol, 74.8% yield, HCl salt). LCMS: [M+H]⁺=549.4, retention time=0.687 min

### Procedure for Preparation of Compound 41-5

To a solution of 41-4 (101 mg, 189 μmol, 1 eq) in MeCN (3 mL) was added dropwise 1-methylimidazole (155 mg, 1.89 mmol, 150 μL, 10 eq) and [chloro(dimethylamino)methylene]-dimethyl-ammonium;hexafluorophosphate (58.3 mg, 207 μmol, 1.1 eq) in ACN (3 mL). The mixture was stirred at 25° C. for 3 h. The reaction mixture was concentrated under reduced pressure to remove ACN. The residue was diluted with H₂O (20 mL) and extracted with EtOAc (20 mL*3). The combined organic layers were washed with brine (50 mL*2), dried over Na₂SO₄, filtered and concentrated under reduced pressure to give crude compound 41-5 (163 mg, 110 μmol, 58.4% yield, 35% purity). LCMS: [M+H]⁺=517.3, retention time=1.856 min

### Procedure for Preparation of Compound 41

To a solution of 41-5 (190 mg, 128 μmol, 35% purity, 1 eq) in MeOH (5 mL) was added NaOH (1 M, 643 μL, 5 eq). The mixture was stirred at 70° C. for 2 h. The reaction mixture was filtered and concentrated under reduced pressure to give residue. The residue was purified by prep-HPLC (FA condition) to give compound 41 (16 mg, 31.5 μmol, 24.5% yield, 96.2% purity).

¹H NMR: (400 MHz, DMSO-d₆) δ ppm 7.57 (br s, 1H) 7.41-7.51 (m, 1H) 7.00-7.24 (m, 3H) 4.79 (br s, 1H) 4.43-4.64 (m, 4H) 3.04 (br s, 2H) 2.77 (br s, 3H) 2.61-2.73 (m, 2H) 2.21-2.36 (m, 2H) 1.80 (br s, 2H) 1.39 (br s, 2H) 1.01-1.30 (m, 12H) LCMS: [M+H]⁺=489.6, retention time=0.769 min

### Example 1.22 Synthesis of Compound 44

### Synthesis of 2-(14,6-dimethyl-7-oxo-11H-6-aza-1(5,1)-benzo[d][1,2,3]triazola-8(1,4)-piperidina-3(1,4) benzenacyclotridecaphane-2-yl)acetic acid (Compound 44)

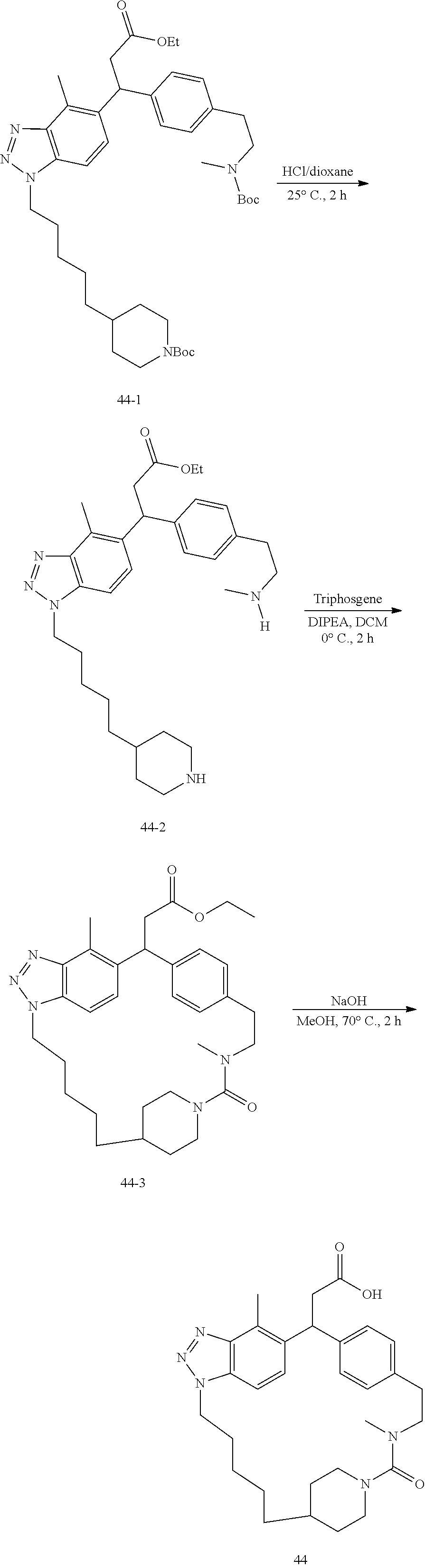 [SMILES: CCOC(=O)CC(c1ccc(CCN(C)C(=O)OC(C)(C)C)cc1)c1ccc2c(nnn2CCCCCC2CCN(C(=O)OC(C)(C)C)CC2)c1C ; CCOC(=O)CC(c1ccc(CCNC)cc1)c1ccc2c(nnn2CCCCCC2CCNCC2)c1C ; CCOC(=O)CC1c2ccc(cc2)CCN(C)C(=O)N2CCC(CCCCCn3nnc4c(C)c1ccc43)CC2 ; "NaOH" ; Cc1c2ccc3c1nnn3CCCCCC1CCN(CC1)C(=O)N(C)CCc1ccc(cc1)C2CC(=O)O]

### Procedure for Preparation of COMPOUND 44-1

A mixture of 44-1 (420 mg, 583 μmol, 1 eq), HCl/dioxane (10 mL) was degassed and purged with N₂ for 3 times, and then the mixture was stirred at 25° C. for 2 h under N₂ atmosphere. The reaction mixture was concentrated under reduced pressure to give a residue. The crude product 44-2 (325 mg, 548 μmol, 94.00% yield, 2HCl) was used into the next step without further purification. Compound 44-2 (325 mg, 548 μmol, 94.00% yield, 2HCl) was obtained. LCMS: [M+H]⁺=520.3, retention time=1.380 min

### Procedure for Preparation of Compound 44-3

To a solution of 44-2 (400 mg, 769 μmol, 1 eq) in DCM (5 mL) was added bis(trichloromethyl) carbonate (91.3 mg, 307 μmol, 0.4 eq) and DIPEA (994 mg, 7.70 mmol, 1.34 mL, 10 eq). The mixture was stirred at 0° C. for 2 h. The reaction mixture was concentrated under reduced pressure to give a residue. The crude product 44-3 (100 mg, 183 μmol, 23.8% yield) was used into the next step without further purification. Compound 44-3 (100 mg, 183 gmol, 23.8% yield) was obtained. LCMS: [M+H]⁺=546.3, retention time=0.981 min

### Procedure for preparation of 2-(14,6-dimethyl-7-oxo-11H-6-aza-1(5,1)-benzo[d][1,2,3]triazola-8(1,4)-piperidina-3(1,4)-benzenacyclotridecaphane-2-yl)acetic acid (Compound 44)

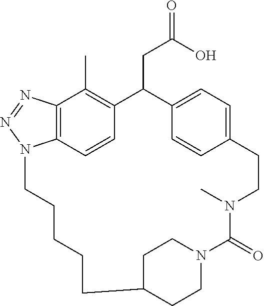 [SMILES: Cc1c2ccc3c1nnn3CCCCCC1CCN(CC1)C(=O)N(C)CCc1ccc(cc1)C2CC(=O)O]

To a solution of 44-3 (100 mg, 183 μmol, 1 eq) in MeOH (5 mL) was added NaOH (1 M, 916 μL, 5 eq). The mixture was stirred at 70° C. for 2 h. The reaction mixture was concentrated under reduced pressure to give a residue. The residue was purified by prep-HPLC (TFA condition). Compound 44 (5 mg, 9.44 μmol, 5.15% yield, 97.6% purity) was obtained.

¹H NMR: (400 MHz, DMSO-d₆) δ ppm 7.59-7.66 (m, 1H) 7.52 (d, J=8.6 Hz, 1H) 7.24 (d, J=8.1 Hz, 2H) 7.03 (d, J=8.0 Hz, 2H) 4.77 (t, J=7.9 Hz, 1H) 4.66 (br t, J=5.6 Hz, 2H) 3.03 (br d, J=7.9 Hz, 2H) 2.86 (br d, J=13.3 Hz, 1H) 2.64-2.78 (m, 6H) 2.60 (s, 3H) 2.25-2.33 (m, 1H) 1.83-1.97 (m, 3H) 1.44 (br t, J=11.6 Hz, 1H) 1.05-1.14 (m, 1H) 0.99 (br d, J=11.3 Hz, 1H) 0.80-0.93 (m, 2H) 0.73 (br dd, J=12.9, 6.2 Hz, 1H) 0.53-0.66 (m, 1H) 0.33-0.52 (m, 4H) −0.02-0.08 (m, 1H) −0.06-0.10 (m, 1H) LCMS: [M+H]⁺=518.3, retention time=0.872 min

### Example 1.23 Synthesis of Compounds 55 and 56

### Procedure for preparation of 2-(4-oxo-31,32,33,34-tetrahydro-13H-3(7,2)-isoquinolina-1(6,3)-[1,2,3]triazolo[4,5-b]pyridina-5(1,4)-cyclohexanacyclodecaphane-2-yl)acetic acid (Compound 55) and 2-(4-oxo-31,32,33,34-tetrahydro-13H-3(7,2)-isoquinolina-1(6,3)-[1,2,3]triazolo[4,5-b]pyridina-5(1,4)-cyclohexanacyclononaphane-2-yl)acetic acid (Compound 56)

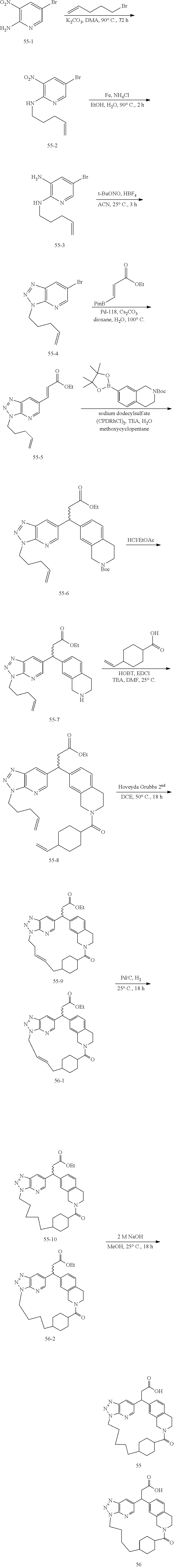

### Procedure for Preparation of Compound 55-2

To a solution of 55-1 (20 g, 91.7 mmol, 1 eq) in DMA (300 mL) was added K₂CO₃ (76.1 g, 550 mmol, 6 eq) and 5-bromopent-1-ene (27.3 g, 183 mmol, 21.7 mL, 2 eq). The mixture was stirred at 90° C. for 18 h. The reaction mixture was diluted with H₂O (500) mL and extracted with EtOAc (200 mL*3). The combined organic layers were washed with aqueous brine (200 mL*2), dried over Na₂SO₄, filtered and concentrated under reduced pressure to give a residue. The residue was purified by flash silica gel chromatography (ISCO®; 330 g SepaFlash® Silica Flash Column, Eluent of 0~10% ethyl acetate/petroleum ether gradient at 100 mL/min) to give compound 55-2 (24 g, 83.9 mmol, 91.4% yield).

¹H NMR: (400 MHz, DMSO-d₆) δ ppm 8.49-8.56 (m, 2H) 8.47 (d, J=2.2 Hz, 1H) 5.64-5.96 (m, 1H) 4.82-5.12 (m, 2H) 3.46-3.59 (m, 2H) 2.06 (q, J=7.0 Hz, 2H) 1.67 (quin, J=7.3 Hz, 2H)

### Procedure for Preparation of Compound 55-3

A mixture of 55-2 (22 g, 76.9 mmol, 1 eq), NH₄Cl (8.23 g, 154 mmol, 2 eq), Fe (30.1 g, 538 mmol, 7 eq) in EtOH (180 mL) and H₂O (60 mL) was stirred at 90° C. for 2 h under N₂ atmosphere. The reaction mixture was filtered and concentrated under reduced pressure to give a residue. The residue was diluted with H₂O (100 mL) and extracted with EtOAc (100 mL*3). The combined organic layers were washed with brine (100 mL*2), dried over Na₂SO₄, filtered and concentrated under reduced pressure to give compound 55-3 (19.5 g, 76.1 mmol, 99.0% yield).

¹H NMR: (400 MHz, DMSO-d₆) δ ppm 7.36 (d, J=2.0 Hz, 1H) 6.78 (d, J=2.0 Hz, 1H) 5.75-5.81 (m, 1H) 4.85-5.14 (m, 5H) 3.21-3.32 (m, 2H) 2.04-2.11 (m, 2H) 1.60-1.68 (m, 2H)

### Procedure for Preparation of Compound 55-4

To a solution of 55-3 (20 g, 78.1 mmol, 1 eq) in ACN (200 mL) was added trifluoroborane hydrofluoride (13.7 g, 156 mmol, 9.73 mL, 2 eq) and tert-butyl nitrite (12.1 g, 117 mmol, 13.9 mL, 1.5 eq) at 0° C. The mixture was stirred at 0-25° C. for 3 h. The reaction mixture was concentrated under reduced pressure to remove ACN. The residue was diluted with H₂O (50 mL) and extracted with EtOAc (50 mL*3). The combined organic layers were washed with brine (100 mL*2), dried over Na₂SO₄, filtered and concentrated under reduced pressure to give a residue. The residue was purified by flash silica gel chromatography (ISCO®; 330 g SepaFlash® Silica Flash Column, Eluent of 0~20% ethyl acetate/petroleum ether gradient at 100 mL/min) to give compound 55-4 (11.8 g, 44.2 mmol, 56.6% yield).

¹H NMR: (400 MHz, DMSO-d₆) δ ppm 8.93 (d, J=2.0 Hz, 1H) 8.84 (d, J=2.0 Hz, 1H) 5.78 (ddt, J=17.0, 10.4, 6.3, 6.3 Hz, 1H) 4.90-5.06 (m, 2H) 4.64-4.77 (m, 2H) 2.05 (br d, J=3.3 Hz, 4H)

### Procedure for Preparation of Compound 55-5

A mixture 55-4 (11.9 g, 44.7 mmol, 1 eq), ethyl 3-(4,4,5,5-tetramethyl-1,3,2-dioxaborolan-2-yl)propanoate (12.2 g, 53.6 mmol, 1.2 eq), ditert-butyl(cyclopentyl)phosphane;dichloropalladium;iron (1.17 g, 1.79 mmol, 0.04 eq), Cs₂CO₃ (29.1 g, 89.4 mmol, 2 eq) in dioxane (200 mL) and H₂O (50 mL) was degassed and purged with N₂ for 3 times, and then the mixture was stirred at 100° C. for 18 h under N₂ atmosphere. The reaction mixture was concentrated under reduced pressure to remove dioxane (200 mL). The residue was diluted with H₂O (50 mL) and extracted with EtOAc (100 mL*3). The combined organic layers were washed with brine (50 mL*2), dried over Na₂SO₄, filtered and concentrated under reduced pressure to give a residue. The residue was purified by flash silica gel chromatography (ISCO®; 120 g SepaFlash® Silica Flash Column, Eluent of 0~20% ethyl acetate/petroleum ether gradient at 100 mL/min) to give compound 55-5 (11.5 g, 40.1 mmol, 89.6% yield).

¹H NMR: (400 MHz, DMSO-d₆) δ ppm 9.14 (d, J=1.8 Hz, 1H) 8.98 (d, J=1.8 Hz, 1H) 7.89 (d, J=16.3 Hz, 1H) 6.95 (d, J=16.1 Hz, 1H) 5.66-5.97 (m, 1H) 4.92-5.06 (m, 2H) 4.72 (t, J=6.6 Hz, 2H) 4.22 (q, J=7.0 Hz, 2H) 1.99-2.12 (m, 4H) 1.28 (t, J=7.2 Hz, 3H)

### Procedure for Preparation of Compound 55-6

A mixture of 55-5 (11.4 g, 39.7 mmol, 1 eq), tert-butyl 7-(4,4,5,5-tetramethyl-1,3,2-dioxaborolan-2-yl)-3,4-dihydro-1H-isoquinoline-2-carboxylate (21.4 g, 59.6 mmol, 1.5 eq), chlororhodium(1Z,5Z)-cycloocta-1,5-diene (1.96 g, 3.97 mmol, 0.1 eq), sodiumdodecyl sulfate (5.73 g, 19.9 mmol, 5.67 mL, 0.5 eq) in TEA (20 mL), methoxyl-cyclopentane (200 mL) and H₂O (100 mL) was degassed and purged with N₂ for 3 times, and then the mixture was stirred at 90° C. for 4 h under N₂ atmosphere. The reaction mixture was diluted with H₂O (100 mL) and extracted with EtOAc (100 mL*3). The combined organic layers were washed with brine (50 mL*2), dried over Na₂SO₄, filtered and concentrated under reduced pressure to give a residue. The residue was purified by flash silica gel chromatography (ISCO®; 220 g SepaFlash® Silica Flash Column, Eluent of 0~20% Ethyl acetate/Petroleum ether gradient at 100 mL/min) to give compound 55-6 (16.6 g, 31.9 mmol, 80.1% yield).

¹H NMR: (400 MHz, DMSO-d₆) δ ppm 8.79 (d, J=1.8 Hz, 1H) 8.58 (d, J=1.8 Hz, 1H) 7.17-7.33 (m, 2H) 7.06 (d, J=7.8 Hz, 1H) 5.67-5.90 (m, 1H) 4.89-5.05 (m, 2H) 4.62-4.73 (m, 3H) 4.44 (br s, 2H) 3.93-3.96 (m, 2H) 3.49 (brt, J=5.5 Hz, 2H) 2.68 (brt, J=5.8 Hz, 2H) 2.36-2.56 (m, 2H) 1.99-2.06 (m, 4H) 1.40 (s, 9H) 1.02 (t, J=7.0 Hz, 3H)

### Procedure for Preparation of Compound 55-7

A mixture 55-6 (4 g, 7.70 mmol, 1 eq) in HCl/EtOAc (15 mL) was degassed and purged with N₂ for 3 times, and then the mixture was stirred at 25° C. for 2 h under N₂ atmosphere. The reaction mixture was concentrated under reduced pressure to give compound 55-7 (3.5 g, 7.68 mmol, 99.7% yield, HCl salt). LCMS: [M+H]⁺=420.5, retention time=0.817 min

### Procedure for Preparation of Compound 55-8

A mixture of 1-hydroxybenzotriazole (1.48 g, 10.97 mmol, 2 eq), TEA (2.22 g, 21.9 mmol, 3.05 mL, 4 eq) and 3-(ethyliminomethyleneamino)-N,N-dimethyl-propan-1-amine;hydrochloride (2.10 g, 10.97 mmol, 2 eq) in DMF (15 mL) was degassed and purged with N₂ for 3 times, 4-vinylcyclohexanecarboxylic acid (1.01 g, 6.58 mmol, 1.2 eq) was added at 25° C., after 0.5 h stirred, 55-7 (2.5 g, 5.48 mmol, 1 eq, HCl salt) in DMF (25 mL) was added dropwise, and then the mixture was stirred at 25° C. for 18 h under N₂ atmosphere. The reaction mixture was diluted with H₂O (50 mL) and extracted with EtOAc (100 mL*3). The combined organic layers were washed with brine (100 mL*2), dried over Na₂SO₄, filtered and concentrated under reduced pressure to give a residue. The residue was purified by flash silica gel chromatography (ISCO®; 120 g SepaFlash® Silica Flash Column, Eluent of 0~60% Ethyl acetate/Petroleum ether gradient at 100 mL/min). Compound 55-8 (2 g, 3.60 mmol, 65.6% yield) was obtained.

¹H NMR: (400 MHz, DMSO-d₆) δ ppm 8.78 (br s, 1H) 8.52-8.62 (m, 1H) 7.95 (s, 1H) 7.20-7.35 (m, 2H) 7.07 (d, J=8.0 Hz, 1H) 5.69-5.83 (m, 2H) 4.93-5.04 (m, 3H) 4.89 (br d, J=10.5 Hz, 1H) 4.61-4.72 (m, 3H) 4.54 (s, 1H) 3.89-4.00 (m, 2H) 3.55-3.73 (m, 2H) 2.89 (s, 4H) 2.73 (s, 4H) 1.95-2.08 (m, 4H) 1.70 (br t, J=12.5 Hz, 4H) 1.31-1.48 (m, 2H) 1.02 (t, J=7.2 Hz, 3H)

### Procedure for Preparation of Compound 55-9 and Compound 56-1

To a solution of 55-8 (1 g, 1.80 mmol, 1 eq) in DCE (300 mL) was added [1,3-bis(2,4,6-trimethylphenyl)imidazolidin-2-ylidene]-dichloro-[(2-isopropoxyphenyl)methylene]ruthenium (226 mg, 360 mol, 0.2 eq). The mixture was stirred at 50° C. for 18 h. The reaction mixture was concentrated under reduced pressure to give a residue. The residue was purified by flash silica gel chromatography (ISCO®; 40 g SepaFlash® Silica Flash Column, Eluent of 0~40% ethyl acetate/petroleum ether gradient at 80 mL/min) to give a mixture of 55-9 and 56-1 (180 mg, 341 mol, 18.9% yield). LCMS: [M+H]⁺=513.8, [M+H]⁺=528.0, retention time=3.451 min

### Procedure for Preparation of Compound 55-10 and 56-2

To a solution of 55-9 and 56-1 (216 mg, mixture, 409 μmol, 1 eq) in MeOH (30 mL) was added Pd/C (100 mg, 10% purity) under N₂ atmosphere. The suspension was degassed and purged with H₂ 3 times. The mixture was stirred under H₂ (15 Psi) at 25° C. for 18 h. The reaction mixture was filtered and concentrated to give a mixture of 55-10 and 56-2 (160 mg, 302 mol, 73.8% yield). LCMS: [M+H]⁺=516.3, retention time=0.908 min, [M+H]⁺=530.3, retention time=0.944 min

### Procedure for preparation of 2-(4-oxo-31,32,33,34-tetrahydro-13H-3(7,2)-isoquinolina-1(6,3)-[1,2,3]triazolo[4,5-b]pyridina-5(1,4)-cyclohexanacyclodecaphane-2-yl)acetic acid (Compound 55) and 2-(4-oxo-31,32,33,34-tetrahydro-13H-3(7,2)-isoquinolina-1(6,3)-[1,2,3]triazolo[4,5-b]pyridina-5(1,4)-cyclohexanacyclononaphane-2-yl)acetic acid (Compound 56)

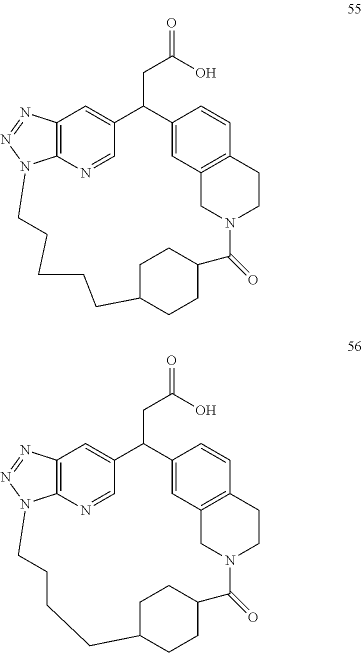

To a solution of 55-10 and 56-2 (160 mg, 302 μmol, 1 eq) in MeOH (5 mL) was added NaOH (1 M, 1.81 mL, 6 eq). The mixture was stirred at 25° C. for 18 h. The reaction mixture was concentrated under reduced pressure to give a residue. The residue was purified by prep-HPLC (TFA condition) to give the desired compound. Compound 55 (5.2 mg, 10.2 mol, 3.39% yield, 98.8% purity) was obtained. Compound 56 (5.9 mg, 12.1 μmol, 4.01% yield, 100% purity) was obtained.

¹H NMR of Compound 55: (400 MHz, DMSO-d₆) δ ppm 8.67 (d, J=1.8 Hz, 1H) 8.24 (d, J=1.7 Hz, 1H) 7.46 (br d, J=6.4 Hz, 1H) 7.21 (d, J=7.8 Hz, 1H) 6.91 (s, 1H) 4.67-4.73 (m, 2H) 4.37-4.51 (m, 2H) 3.65-3.88 (m, 2H) 3.21 (br d, J=7.9 Hz, 2H) 2.88 (t, J=6.8 Hz, 2H) 2.40 (br t, J=11.6 Hz, 1H) 2.07-2.26 (m, 2H) 1.21 (br d, J=11.2 Hz, 4H) 0.99-1.11 (m, 4H) 0.64-0.96 (m, 4H) 0.35-0.58 (n, 2H) LCMS: [M+H]⁺=502.3, retention time=0.841 m

¹H NMR of Compound 56: (400 MHz, DMSO-d₆) δ ppm 8.66 (s, 1H) 8.37 (s, 1H) 7.42 (br d, J=6.2 Hz, 1H) 7.15 (d, J=7.7 Hz, 1H) 6.87 (s, 1H) 4.75-4.80 (m, 4H) 4.31-4.50 (m, 2H) 3.89 (br s, 1H) 3.51-3.75 (m, 1H) 3.17-3.25 (m, 2H) 2.86 (t, J=6.8 Hz, 2H) 2.23 (br t, J=11.5 Hz, 1H) 2.04 (br d, J=3.8 Hz, 2H) 0.67-1.17 (m, 11H) 0.35 (br s, 1H) 0.02-0.20 (m, 1H) 0.00-0.00 (in, 1H) LCMS: [M+H]⁺=488.2, retention time=0.806 min

The following compounds were synthesized by similar methods described above with modified conditions and different starting materials.

| Compound | | LCMS | ¹H NMR |
|---|---|---|---|
| No. | Strcture | [M + H]⁺ | (400 MHz, DMSO-d₆) |
| Compound 18 | 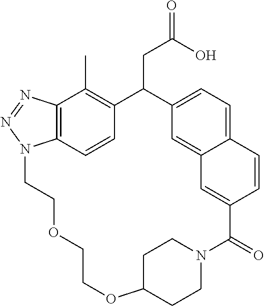 [SMILES: Cc1c2ccc3c1nnn3CCOCCOC1CCN(CC1)C(=O)c1ccc3ccc(cc3c1)C2CC(=O)O] | 529.6 | |
| | Isomer 1 | | |
| Compound 19 | 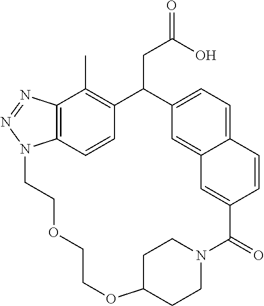 [SMILES: Cc1c2ccc3c1nnn3CCOCCOC1CCN(CC1)C(=O)c1ccc3ccc(cc3c1)C2CC(=O)O] | 529.6 | |
| | Isomer 2 | | |
| Compound 20 | 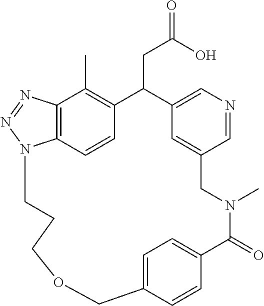 [SMILES: Cc1c2ccc3c1nnn3CCCOCc1ccc(cc1)C(=O)N(C)Cc1cncc(c1)C2CC(=O)O] | 500.3 | δ 8.72-8.70 (m, 1H), 8.36-8.34 (m, 1H), 7.79-7.62 (m, 1H), 7.52-7.31 (m, 1H), 7.26-6.72 (m, 5H), 4.90-4.86 (m, 1H), 4.79-4.61 (m, 2H), 4.60-4.44 (m, 1H), 4.24-4.22 (m, 1H), 4.23-4.11 (m, 2H), 3.39-3.35 (m, 2H), 3.18-2.99 (m, 2H), 2.86 (brs, 3H), 2.58 (brs, 3H), 2.40-2.51 (m, 2H). |
| | Isomer 1 | | |
| Compound 21 | 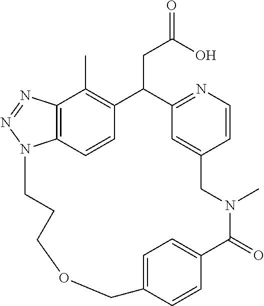 [SMILES: Cc1c2ccc3c1nnn3CCCOCc1ccc(cc1)C(=O)N(C)Cc1ccnc(c1)C2CC(=O)O] | 500.2 | δ 8.51-8.50 (m, 1H), 8.00-7.25 (m, 2H), 7.15-7.05 (m, 1H), 7.05-6.85 (m, 1H), 6.85-6.70 (m, 2H), 6.65-6.45 (m, 2H), 5.03-4.18 (m, 8H), 3.18-2.61 (m, 9H), 2.44-2.24 (m, 2H). |
| Compound 22 | 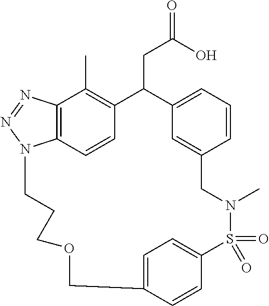 [SMILES: Cc1c2ccc3c1nnn3CCCOCc1ccc(cc1)S(=O)(=O)N(C)Cc1cccc(c1)C2CC(=O)O] | 535.6 | δ 7.64 (d, J = 8.7 Hz, 1H), 7.58 (d, J = 7.8 Hz, 1H), 7.53 (d, J = 8.8 Hz, 1H), 7.27 (d, J = 7.6 Hz, 1H), 7.24 (d, J = 5.5 Hz, 1H), 7.09 (d, J = 7.6 Hz, 1H), 7.05 (d, J = 8.4 Hz, 2H), 6.35 (d, J = 8.3 Hz, 2H), 4.89 (t, J = 7.9 Hz, 1H), 4.82 -4.71 (m, 2H), 4.47 (d, J = 14.8 Hz, 1H), 4.22 (d, J = 13.4 Hz, 1H), 4.10 (d, J = 14.8 Hz, 1H), 4.01 (d, J = 13.4 Hz, 1H), 3.47 (dt, J = 9.5, 4.6 Hz, 1H), 3.30-3.18 (m, 1H), 3.15-3.05 (m, 2H), 2.83 (s, 3H), 2.62 (s, 3H), 2.34-2.21 (m, 2H). |
| Compound 23 | 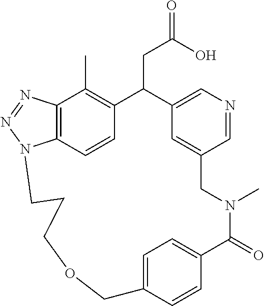 [SMILES: Cc1c2ccc3c1nnn3CCCOCc1ccc(cc1)C(=O)N(C)Cc1cncc(c1)C2CC(=O)O] | 500.3 | δ 8.72-8.70 (m, 1H), 8.36-8.34 (m, 1H), 7.79-7.62 (m, 1H), 7.52-7.31 (m, 1H), 7.26-6.72 (m, 5H), 4.90-4.86 (m, 1H), 4.79-4.61 (m, 2H), 4.60-4.44 (m, 1H), 4.24-4.22 (m, 1H), 4.23-4.11 (m, 2H), 3.39-3.35 (m, 2H), 3.18-2.99 (m, 2H), 2.86 (brs, 3H), 2.58 (brs, 3H), 2.40-2.51 (m, 2H). |
| | Isomer 2 | | |
| Compound 24 | 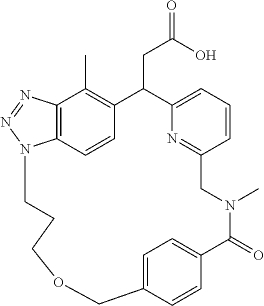 [SMILES: Cc1c2ccc3c1nnn3CCCOCc1ccc(cc1)C(=O)N(C)Cc1cccc(n1)C2CC(=O)O] | 500.1 | δ 7.82 (t, J = 7.7 Hz, 1H), 7.56- 7.53 (m, 2H), 7.13 (d, J = 7.5 Hz, 2H), 6.86 (d, J = 7.6 Hz, 2H), 6.47 (s, 2H), 5.02 (t, J = 7.6 Hz, 1H), 4.79-4.72 (m, 2H), 4.61 (s, 1H), 4.51-4.48 (m, 1H), 4.35 (s, 2H), 4.14-4.10 (m, 1H), 3.34-3.26 (m, 2H), 3.08-3.02 (m, 1H), 2.90 (s, 3H), 2.77 (s, 3H), 2.40-2.34 (m, 1H), 2.29- 2.25 (m, 1H). |
| | Isomer 1 | | |
| Compound 25 | 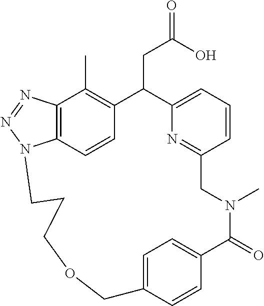 [SMILES: Cc1c2ccc3c1nnn3CCCOCc1ccc(cc1)C(=O)N(C)Cc1cccc(n1)C2CC(=O)O] | 500.1 | δ 7.82 (t, J = 7.7 Hz, 1H), 7.56- 7.53 (m, 2H), 7.13 (d, J = 7.5 Hz, 2H), 6.86 (d, J = 7.6 Hz, 2H), 6.47 (s, 2H), 5.02 (t, J = 7.6 Hz, 1H), 4.79-4.72 (m, 2H), 4.61 (s, 1H), 4.51-4.48 (m, 1H), 4.35 (s, 2H), 4.14-4.10 (m, 1H), 3.34-3.26 (m, 2H), 3.08-3.02 (m, 1H), 2.90 (s, 3H), 2.77 (s, 3H), 2.40-2.34 (m, 1H), 2.29- 2.25 (m, 1H). |
| | Isomer 2 | | |
| Compound 26 | 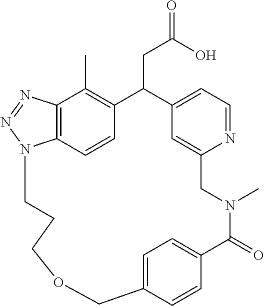 [SMILES: Cc1c2ccc3c1nnn3CCCOCc1ccc(cc1)C(=O)N(C)Cc1cc(ccn1)C2CC(=O)O] | 500.2 | δ 8.74 (s, 1H), 8.00-7.73 (m, 3H), 7.42-7.12 (m, 2H), 6.98- 6.96 (m, 2H), 5.00-4.96 (m, 2H), 4.83-4.81 (m, 1H), 4.92-4.23 (m, 6H), 3.39-3.37 (m, 3H), 3.01 (s, 3H), 2.72-2.51 (m, 2H), 2.37- 2.28 (m, 2H). |
| | Isomer 1 | | |
| Compound 27 | 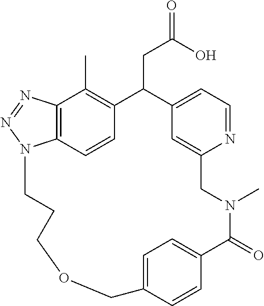 [SMILES: Cc1c2ccc3c1nnn3CCCOCc1ccc(cc1)C(=O)N(C)Cc1cc(ccn1)C2CC(=O)O] | 500.2 | δ 8.74 (s, 1H), 8.00-7.73 (m, 3H), 7.42-7.12 (m, 2H), 6.98- 6.96 (m, 2H), 5.00-4.96 (m, 2H), 4.83-4.81 (m, 1H), 4.92- 4.23 (m, 6H), 3.39-3.37 (m, 3H), 3.01 (s, 3H), 2.72-2.51 (m, 2H), 2.37-2.28 (m, 2H). |
| | Isomer 2 | | |
| Compound 31 | 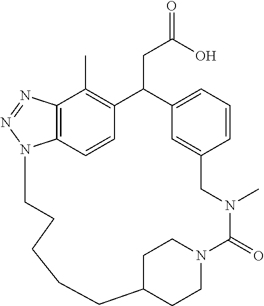 [SMILES: Cc1c2ccc3c1nnn3CCCCCC1CCN(CC1)C(=O)N(C)Cc1cccc(c1)C2CC(=O)O] | 504.2 | ¹H NMR (400 MHz, MeOH-d₄) δ 7.70 (d, J = 8.7 Hz, 1H), 7.50 (d, J = 8.5 Hz, 2H), 7.39 (t, J = 7.6 Hz, 1H), 7.13 (d, J = 7.6 Hz, 1H), 6.95 (s, 1H), 5.03 (t, J = 7.8 Hz, 1H), 4.79 (dd, J = 10.0, 4.5 Hz, 2H), 4.29 (d, J = 16.2 Hz, 1H), 4.16 (d, J = 16.2 Hz, 1H), 3.43 (d, J = 12.9 Hz, 1H), 3.28- 3.17 (m, 2H), 3.07 (dd, J = 15.8, 8.2 Hz, 1H), 2.80 (d, J = 7.5 Hz, 6H), 2.46 (t, J = 11.8 Hz, 1H), 2.34 (t, J = 11.8 Hz, 1H), 2.13 (dd, J = 11.7, 5.9 Hz, 2H), 1.49- 1.11 (m, 4H), 0.89 (d, J = 16.2 Hz, 3H), 0.80-0.65 (m, 3H), 0.24 (dt, J = 11.8, 8.7 Hz, 1H). |
| Compound 32 | 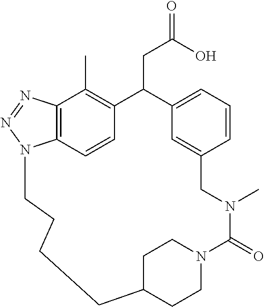 [SMILES: Cc1c2ccc3c1nnn3CCCCC1CCN(CC1)C(=O)N(C)Cc1cccc(c1)C2CC(=O)O] | 490.2 | ¹H NMR (400 MHz, MeOH-d₄) δ 7.76 (d, J = 8.6 Hz, 1H), 7.69 (d, J = 8.6 Hz, 1H), 7.48 (d, J = 7.5 Hz, 1H), 7.36 (t, J = 7.6 Hz, 1H), 7.12 (d, J = 7.5 Hz, 1H), 6.94 (s, 1H), 4.99 (t, J = 7.8 Hz, 1H), 4.84-4.73 (m, 2H), 4.38 (d, J = 15.9 Hz, 1H), 4.14 (d, J = 15.9 Hz, 1H), 3.43 (d, J = 12.7 Hz, 1H), 3.16 (d, J = 17.1, 8.0 Hz, 2H), 3.07 (d, J = 13.0 Hz, 1H), 2.77 (m, J = 5.0 Hz, 6H), 2.50 (d, J = 14.2 Hz, 1H), 2.42 (d, J = 14.2 Hz, 1H), 2.31 (d, J = 5.8 Hz, 1H), 2.03 (s, 1H), 1.20-1.09 (m, 4H), 0.80- 1.62 (m, 4H), 0.19-0.16 (m, 1H). |
| Compound 36 | 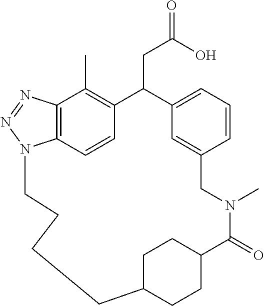 [SMILES: Cc1c2ccc3c1nnn3CCCCC1CCC(CC1)C(=O)N(C)Cc1cccc(c1)C2CC(=O)O] | 489.4 | ¹H NMR (400 MHz, MeOH-d₄) δ 7.66-7.54 (m, 2H), 7.54-7.46 (m, 1H), 7.43-7.33( m, 1H), 7.18 (s, 1H), 7.12-7.06 (m, 1H), 5.16-5.01 (m, 1H), 4.83-4.76 (m, 1H), 4.74-4.65 (m, 1H), 4.51 (s, 2H), 3.27-3.14 (m, 5H), 3.01-2.94 (m, 3H), 2.09- 1.81 (m, 3H), 1.22-0.67 (m, 10H), −0.15-−0.56 (m, 3H). |
| Compound 37 | 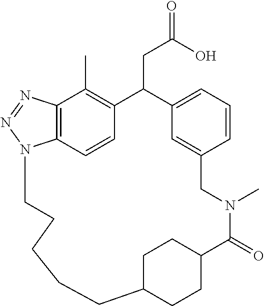 [SMILES: Cc1c2ccc3c1nnn3CCCCCC1CCC(CC1)C(=O)N(C)Cc1cccc(c1)C2CC(=O)O] | 503.4 | ¹H NMR (400 MHz, MeOH-d₄) δ 7.56-7.51 (m, 2H), 7.48-7.42 (m, 1H), 7.32-7.22 (m, 1H), 7.18-7.11 (m, 1H), 6.82 (s, 1H), 5.12-5.03 (m, 1H), 4.79-4.62 (m, 3H), 4.42-4.36 (m, 1H), 3.23-3.19 (m, 1H), 3.06-2.95 (m, 4H), 2.93-2.86 (m, 3H), 2.20-1.94 (m, 3H), 1.42-0.97 (m, 9H), 0.85-0.76 (m, 3H), 0.73-0.66 (m, 1H), 0.42 (t, J = 10.1 Hz, 2H), −0.25-−0.45 (m, 1H). |
| Compound 38 | 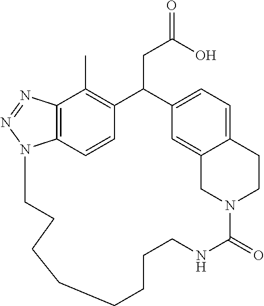 [SMILES: Cc1c2ccc3c1nnn3CCCCCCCCNC(=O)N1CCc3ccc(cc3C1)C2CC(=O)O] | 490.3 | δ ppm 7.58 (d, J = 8.8 Hz, 1 H) 7.30 (d, J = 8.8 Hz, 2 H) 7.04- 7.15 (m, 1 H) 6.51 (s, 1 H) 6.18 (br s, 1 H) 4.81 (br t, J = 7.7 Hz, 1 H) 4.55-4.76 (m, 2 H) 4.08- 4.23 (m, 2 H) 3.19 (br dd, J = 15.7, 7.4 Hz, 2 H) 2.97-3.09 (m, 2 H) 2.62-2.80 (m, 6 H) 1.84- 2.00 (m, 2 H) 0.74-1.26 (m, 12 H) |
| Compound 42 | 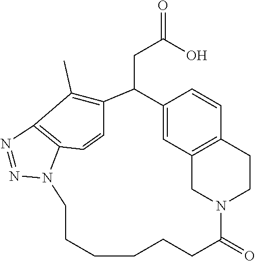 [SMILES: Cc1c2ccc3c1nnn3CCCCCCC(=O)N1CCc3ccc(cc3C1)C2CC(=O)O] | 447.2 | δ ppm 12.10 (br s, 1 H) 7.52- 7.63 (m, 1 H) 7.37-7.51 (m, 1 H) 7.17-7.29 (m, 1 H) 6.95- 7.12 (m, 2 H) 4.79 (br d, J = 5.5 Hz, 1 H) 4.54-4.67 (m, 2 H) 4.35-4.54 (m, 2 H) 3.57 (br d, J = 12.5 Hz, 2 H) 2.94-3.12 (m, 2 H) 2.73-2.83 (m, 3 H) 2.53- 2.71 (m, 2 H) 2.11-2.35 (m, 2 H) 1.73-1.90 (m, 2 H) 1.31- 1.49 (m, 2 H) 1.23 (br s, 4 H) |
| Compound 43 | 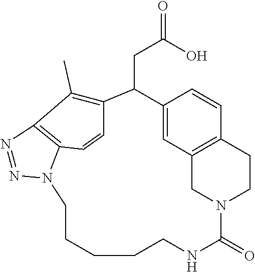 [SMILES: Cc1c2ccc3c1nnn3CCCCCNC(=O)N1CCc3ccc(cc3C1)C2CC(=O)O] | 448.4 | δ ppm 7.56 (br d, J = 8.6 Hz, 1 H) 7.40 (dd, J = 8.6, 6.1 Hz, 1 H) 7.20 (br s, 1 H) 7.04 (dd, J = 7.9, 4.8 Hz, 1 H) 6.90 (br d, J = 7.9 Hz, 1 H) 6.39 (br t, J = 5.3 Hz, 1 H) 4.80 (br t, J = 7.8 Hz, 1 H) 4.58 (br d, J = 4.8 Hz, 2 H) 4.22- 4.37 (m, 2 H) 2.85-3.10 (m, 6 H) 2.75 (s, 2 H) 2.59 (br d, J = 5.4 Hz, 3 H) 1.82 (br s, 2 H) 1.32-1.46 (m, 2 H) 1.18-1.24 (m, 2 H) |
| Compound 46 | 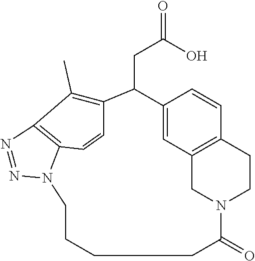 [SMILES: Cc1c2ccc3c1nnn3CCCCC(=O)N1CCc3ccc(cc3C1)C2CC(=O)O] | 419.2 | δ ppm 7.37-7.62 (m, 2 H) 7.15- 7.30 (m, 1 H) 6.88-7.12 (m, 2 H) 4.77 (br d, J = 8.1 Hz, 1 H) 4.64 (br s, 2 H) 4.28-4.53 (m, 2 H) 3.02 (br d, J = 6.9 Hz, 3 H) 2.72-2.82 (m, 3 H) 2.65 (br d, J = 13.0 Hz, 2 H) 2.21-2.43 (m, 3 H) 1.69-2.00 (m, 2 H) 1.38- 1.57 (m, 1 H) 1.15-1.36 (m, 1 H) |
| Compound 51 | 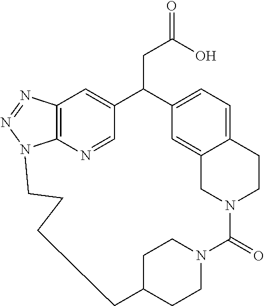 [SMILES: O=C(O)CC1c2ccc3c(c2)CN(CC3)C(=O)N2CCC(CCCCn3nnc4cc1cnc43)CC2] | [M + H]⁺ = 489.1 | δ ppm 8.42 (m, 2 H) 7.38 (br d, J = 7.63 Hz, 1 H) 7.12 (d, J = 8.00 Hz, 1 H) 6.11 (s, 1H) 4.84 (m, 4 H) 4.17 (m, 1 H) 4.00 (m,1 H) 3.74 (br d, J = 13.26 Hz, 1 H) 3.49(br t, J = 10.69 Hz, 2 H) 3.14 (m, 1 H)3.03 (m, 2 H) 2.75 (br d, J = 16.63 Hz, 1H) 2.61 (br t, J = 12.13 Hz, 1 H) 2.50 (brt, J = 12.13 Hz, 1 H) 1.99 (m, 2 H) 1.49(m, 1 H) 1.29 (m, 4 H) 1.14 (m, 1 H)0.92 (m, 1 H) 0.74 (m, 3 H) |
| | Isomer 1 | | |
| Compound 52 | 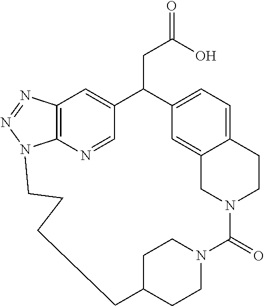 [SMILES: O=C(O)CC1c2ccc3c(c2)CN(CC3)C(=O)N2CCC(CCCCn3nnc4cc1cnc43)CC2] | [M + H]⁺ = 489.1 | δ ppm 8.42 (br d, J = 15.76 Hz, 2 H) 7.38(br d, J = 7.25 Hz, 1 H) 7.12 (br d, J = 7.63Hz, 1 H) 6.11 (br s, 1 H) 4.82 (m, 4 H)4.18 (br d, J = 16.01 Hz, 1 H) 3.99 (br d,J = 16.26 Hz, 1 H) 3.72 (br s, 1 H) 3.49 (brs, 2 H) 3.18 (br s, 1 H) 3.03 (m, 2 H) 2.75(br d, J = 16.51 Hz, 1 H) 2.61 (br t, J = 12.94 Hz, 1 H) 2.50 (br t, J = 12.44 Hz, 1 H) 1.99 (br s, 2 H) 1.50 (br s, 1 H) 1.30(m, 4 H) 1.14 (br d, J = 10.63 Hz, 1 H)0.94 (br s, 1 H) 0.75 (m, 3 H) |
| | Isomer 2 | | |
| Compound 53 | 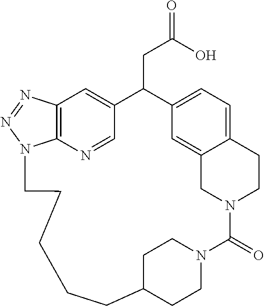 [SMILES: O=C(O)CC1c2ccc3c(c2)CN(CC3)C(=O)N2CCC(CCCCCn3nnc4cc1cnc43)CC2] | 503.3 | (400 MHz, METHANOL-d₄) δ = 8.58 (d, J = 1.6 Hz, 1H), 8.30 (d, J = 1.4 Hz, 1H), 7.43 (d, J = 7.9 Hz, 1H), 7.21 (d, J = 7.9 Hz, 1H), 6.68 (s, 1H), 4.81-4.70 (m, 3H), 4.16 (s, 2H), 3.74-3.52 (m, 2H), 3.20 (dd, J = 3.0, 7.8 Hz, 2H), 3.10 (br d, J = 12.6 Hz, 2H), 2.88 (t, J = 6.6 Hz, 2H), 2.44-2.14 (m, 4H), 1.21-0.97 (m, 9H), 0.74- 0.57 (m, 2H) |
| Compound 54 | 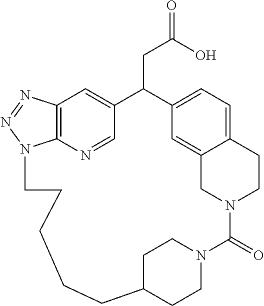 [SMILES: O=C(O)CC1c2ccc3c(c2)CN(CC3)C(=O)N2CCC(CCCCCn3nnc4cc1cnc43)CC2] | 503.3 | (400 MHz, METHANOL-d₄) δ = 8.59 (d, J = 1.9 Hz, 1H), 8.30 (d, J = 1.6 Hz, 1H), 7.43 (dd, J = 1.3, 7.8 Hz, 1H), 7.21 (d, J = 7.9 Hz, 1H), 6.68 (s, 1H), 4.82-4.71 (m, 3H), 4.17 (s, 2H), 3.75-3.52 (m, 2H), 3.21 (dd, J = 3.3, 7.9 Hz, 2H), 3.10 (br d, J = 12.8 Hz, 2H), 2.88 (t, J = 6.5 Hz, 2H), 2.43- 2.16 (m, 4H), 1.21-0.94 (m, 9H), 0.66 (br t, J = 10.9 Hz, 2H) |

### Example 2 Biological Study of Compounds

### Example 2.1 Keap1-Nrf2 Binding Inhibitory Activity Assay

### Material and Instruments:

PerkinElmer EnVision 2105 Multilabel Reader
PerkinElmer FITC FP dual emission Label 2100-8060
LABCYTE Echo 650
Corning 384-well microplate #4514
Keap1_FL, Sino Biological, catalog 11981-H20B
FITC-Nrf2 peptide, GL Biochem, Lot No: p210427-XQ892761

### Method:

a) Reference titration
1. Keap1-Nrf2 Assay Buffer was prepared: 10 mM Tris-HCl pH 7.4, 150 mM NaCl, 50 mM EDTA, 0.05% Tween20, 0.1 mg/ml BSA (freshly add before use).
2. 2.04× Tracer working solution was prepared: 2.04 nM FITC-Nrf2 peptide in Assay Buffer from 1 μM DMSO stock.
3. 2× Protein working solution was prepared: 25 nM Keap1-FL in Assay Buffer.
4. 100× compound working solutions with DMSO were prepared. Starting from 100 μM, serial 3-fold dilutions (10 points) were made by the Echo on the 384-well LDV microplate, then 200 nL of compounds, in duplicated wells, were transferred to a Corning 4514 plate.
5. 9.8 μL per well of the Tracer working solution was added into the Corning 4514 plate.
6. 10 μL per well of the Protein working solution was added into the Corning 4514 plate, except for the first two columns where Dilution Buffer were added to serve as “positive control”.
7. The plate was incubated at room temperature for 60 min.
b) Read data on an EnVision with the following setting: Excitation Light (%): 84; Measurement Height: 9.4; G-Factor: 1; Detector Gain 1:220; Detector Gain 2: 220; Flash Number: 50.

The results of Keap1-Nrf2 Binding Inhibitory Activity Assay (IC₅₀) for Test Compounds are shown in Table 2 below. All the Test Compounds were considered as having Keap1-Nrf2 binding inhibitory activity. For each Test Compound, “+” stands for an IC₅₀ larger than zero and equal to or less than 0.1 μM; “++” stands for an IC₅₀ larger than 0.1 μM and equal to or less than 1 μM; “+++” stands for an IC₅₀ larger than 1 μM and equal to or less than 10 μM.

TABLE 2
Keap1-Nrf2 Binding Inhibitory Activity for Test Compounds

| Compound No. | IC₅₀ (nM) |
|---|---|
| 1 | ++ |
| 2 | +++ |
| 3 | +++ |
| 4 | + |
| 5 | ++ |
| 6 | + |
| 7 | + |
| 8 | +++ |
| 9 | +++ |
| 10 | + |
| 11 | + |
| 12 | + |
| 13 | + |
| 14 | + |
| 15 | + |
| 16 | +++ |
| 17 | +++ |
| 18 | +++ |
| 19 | +++ |
| 20 | +++ |
| 21 | ++ |
| 22 | +++ |
| 23 | +++ |
| 24 | ++ |
| 25 | +++ |
| 26 | ++ |
| 27 | +++ |
| 28 | + |
| 29 | + |
| 30 | + |
| 31 | ++ |
| 32 | +++ |
| 34 | + |
| 35 | + |
| 36 | ++ |
| 37 | ++ |
| 38 | + |
| 40 | + |
| 41 | + |
| 42 | + |
| 43 | +++ |
| 44 | +++ |
| 46 | ++ |
| 47 | + |
| 48 | + |
| 49 | + |
| 50 | + |
| 51 | ++ |
| 52 | + |
| 53 | + |
| 54 | + |
| 55 | + |
| 56 | + |

As shown in Table 2 above, the compounds of present disclosure are effective in inhibiting Keap1-Nrf2 protein-protein interaction (PPI), which is a strategy for activating Nrf2. This approach of using non-reactive molecules that bind tightly to the Nrf2 binding pocket on Keap1 is thought to have the benefits of activating Nrf2 with higher target selectivity than covalent Keap1 inhibitors (or Nrf2 activators) and thus reduce potential safety risks from off-target activity.

### Example 2.2 Nrf2 Cellular Assay

### Procedure for Nrf2 Cellular Assay

### Day 1: Plasmids Preparation

1. pGL4.37[luc2P/ARE/Hygro] vector was diluted to 25 ng/μL in Opti-MEM.
2. Lipofectamine 3000 was added to a 2:1 lipid:DNA. The mixture was mixed by pipetting and incubate at room temperature for 15 minutes.
3. HepG2 cells were cultured in complete medium (DMEM+10% FBS).
4. Trypsin was added to the cells, which were filtered via a cell strainer to remove cell cluster, the cells were then quantified and diluted to 2×10⁵ cells/ml in complete medium.
5. The cells and plasmid were mixed, seeded to 384-well plate 50 μL per well (50 ng DNA/well) and incubated for 24 hours in a 37° C., 5% CO₂ incubator.

### Day 2: Transfection Medium Replacement

1. Medium was removed from cells and replaced with 36 μL of DMEM+10% FBS per well.
2. Incubated overnight in a 37° C., 5% CO₂ incubator.

### Day 3: Compound Treatment

1. Test compound was serially diluted tin DMSO, and further diluted with DMEM (contains 10% FBS) to 10× final concentration.
2. 4 μL of the 10× compound was added to each well, the plate was shaken for 10 sec on a plate shaker and incubated for 6 hours in a 37° C., 5% CO₂ incubator.

### Day 3: Luciferase Measurement

1. Plates were removed from the 37° C., 5% CO₂ incubator and allowed to cool to room temperature for approximately 15 minutes.
2. 40 μL of the Glo Luciferase Assay System detection reagents were added, and the plate was shaken for 3 min and luminescence was measured.

The results of Nrf2 cellular assay (EC₅₀) for Test Compounds are shown in Table 3 below. For each Test Compound, “*” stands for an EC₅₀ larger than zero and equal to or less than 10 μM; “**” stands for an EC₅₀ larger than 10 μM and equal to or less than 30 μM; “***” stands for an EC₅₀ larger than 30 μM.

TABLE 3
Nrf2 Cellular Activity for Test Compounds

| Compound No. | EC₅₀ (μM) |
|---|---|
| 1 | *** |
| 2 | *** |
| 3 | *** |
| 4 | * |
| 5 | *** |
| 7 | ** |
| 8 | *** |
| 10 | * |
| 11 | * |
| 12 | * |
| 14 | * |
| 17 | *** |
| 30 | * |
| 34 | * |
| 35 | * |
| 37 | * |
| 40 | ** |
| 53 | ** |
| 54 | * |
| 55 | * |
| 56 | ** |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

What is claimed is:

1. A compound represented by Formula (I) below: 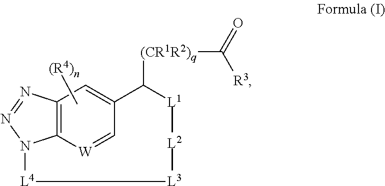
wherein
W is N or C;
L¹ is selected from the group consisting of 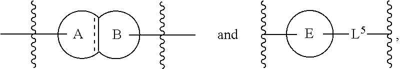
wherein
Ring A is selected from the group consisting of cycloalkyl, heterocyclyl, aryl and heteroaryl, each of which is independently optionally substituted with one or more R^{a1};
Ring B is selected from the group consisting of cycloalkyl, heterocyclyl, aryl and heteroaryl, each of which is independently optionally substituted with one or more R^{a2};
--- is a bond via which Ring A is fused to Ring B;
Ring E is selected from the group consisting of cycloalkyl, heterocyclyl, aryl and heteroaryl, each of which is independently optionally substituted with one or more R^{a3};
L² is selected from the group consisting of —C(O)—, —CR⁷R⁸—, —S(O)— and —S(O)₂—;
L³ is a bond or selected from the group consisting of cycloalkyl, heterocyclyl, aryl and heteroaryl, each of which is independently optionally substituted with one or more R^{a4}; provided that when W is C, L¹ is 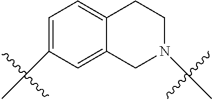 [SMILES: *c1ccc2c(c1)CN(*)CC2]
and L² is —C(O)—, L³ is not an aryl or heteroaryl;
L⁴ is selected from the group consisting of alkyl, alkylalkoxyl, alkenyl, alkynyl, heteroalkyl, heteroalkenyl and heteroalkynyl, wherein the alkyl, alkenyl and alkynyl, either alone or as part of another group, are independently optionally substituted with one or more R^{a5};
L⁵ is a bond or selected from the group consisting of alkyl, alkenyl, alkynyl, -alkyl-N(R^{b})—, heteroalkyl, heteroalkenyl and heteroalkynyl, wherein the alkyl, alkenyl, alkynyl and -alkyl-N(R^{b})—, either alone or as part of another group, are independently optionally substituted with one or more R^{a6};
each of R¹, R², R⁴, R⁷ and R⁸ is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl, wherein the alkyl, alkenyl and alkynyl, either alone or as part of another group, are independently optionally substituted with one or more R^{a7};
R³ is selected from the group consisting of hydrogen, halogen, hydroxyl, sulfhydryl, cyano, alkyl, alkenyl, alkynyl, heteroalkyl, heteroalkenyl, heteroalkynyl, haloalkyl, alkylalkoxyl, —OR^{c}, —NHR^{d} and —N(R^{d})₂, wherein the alkyl, alkenyl and alkynyl, either alone or as part of another group, are independently optionally substituted with one or more R^{a5};
each of R^{a1}, R^{a2}, R^{a3}, R^{a4}, R^{a5}, R^{a6}, R^{a7} and R^{a8} is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl;
each of R^{b}, R^{c} and R^{d} is independently selected from the group consisting of hydrogen, alkyl, alkenyl, alkynyl, heteroalkyl, heteroalkenyl and heteroalkynyl, wherein the alkyl, alkenyl and alkynyl, either alone or as part of another group, are independently optionally substituted by one or more groups independently selected from the group consisting of hydrogen, halogen, hydroxyl sulfhydryl, cyano, —NH₂ and —NO₂; or two R^{b} together with the nitrogen atom to which they are attached form a heterocyclyl; or two R^{d} together with the nitrogen atom to which they are attached form a heterocyclyl;
n is 0, 1, 2 or 3; and
q is 0, 1, 2 or 3,
or a tautomer, a stereoisomer, or a pharmaceutically acceptable salt thereof.

2. The compound, tautomer, stereoisomer, or pharmaceutically acceptable salt thereof of claim 1, wherein the compound is represented by Formula (Ia) or Formula (Ib) below: 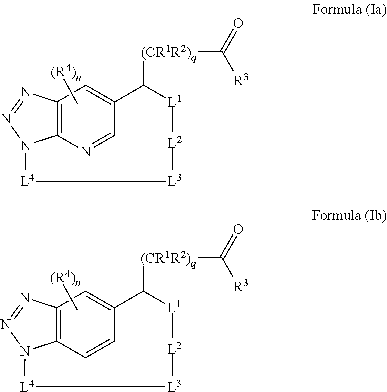
wherein each of R¹, R², R³, R⁴, L¹, L², L³ and L⁴, n and q are as defined in claim 1.

3. The compound, tautomer, stereoisomer, or a pharmaceutically acceptable salt thereof of claim 1 or 2, wherein L¹ is 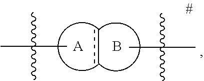
and # is linked to L².

4. The compound, tautomer, stereoisomer, or a pharmaceutically acceptable salt thereof of any one of claims 1 to 3, wherein Ring A is aryl optionally substituted with one or more R^{a1}.

5. The compound, tautomer, stereoisomer, or a pharmaceutically acceptable salt thereof of claim 4, wherein Ring A is a 3- to 12-membered aryl optionally substituted with one or more R^{a1}.

6. The compound, tautomer, stereoisomer, or a pharmaceutically acceptable salt thereof of claim 4 or 5, wherein Ring A is a monocyclic aryl optionally substituted with one or more R^{a1}.

7. The compound, tautomer, stereoisomer, or a pharmaceutically acceptable salt thereof of claim 6, wherein Ring A is a phenyl optionally substituted with one or more R^{a1}.

8. The compound, tautomer, stereoisomer, or a pharmaceutically acceptable salt thereof of claim 7, wherein Ring A is an unsubstituted phenyl.

9. The compound, tautomer, stereoisomer, or a pharmaceutically acceptable salt thereof of claim 7, wherein Ring A is a phenyl substituted with a group selected from the group consisting of halogen, hydroxyl, sulfhydryl, cyano, —NH₂, —NO₂, C₁₋₆ alkoxyl, C₁₋₆ alkyl, C₁₋₆ alkenyl, C₁₋₆ alkynyl, C₁₋₆ heteroalkyl, C₁₋₆ heteroalkenyl, C₁₋₆ heteroalkynyl and halo-C₁₋₆ alkyl.

10. The compound, tautomer, stereoisomer, or a pharmaceutically acceptable salt thereof of claim 9, wherein Ring A is a phenyl substituted with a C₁₋₆ alkyl.

11. The compound, tautomer, stereoisomer, or a pharmaceutically acceptable salt thereof of claim 10, wherein Ring A is a phenyl substituted with a methyl.

12. The compound, tautomer, stereoisomer, or a pharmaceutically acceptable salt thereof of claim 11, wherein Ring A is 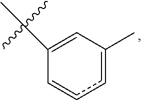
wherein --- is a bond via which Ring A is fused to Ring B.

13. The compound, tautomer, stereoisomer, or a pharmaceutically acceptable salt thereof of any one of claims 1 to 3, wherein Ring A is a heterocyclyl optionally substituted with one or more R^{a1}.

14. The compound, tautomer, stereoisomer, or a pharmaceutically acceptable salt thereof of claim 13, wherein Ring A is a 3- to 12-membered heterocyclyl optionally substituted with one or more R^{a1}.

15. The compound, tautomer, stereoisomer, or a pharmaceutically acceptable salt thereof of claim 13 or 14, wherein Ring A is a monocyclic heterocyclyl optionally substituted with one or more R^{a1}.

16. The compound, tautomer, stereoisomer, or a pharmaceutically acceptable salt thereof of claim 15, wherein Ring A is a 3- to 12-membered monocyclic heterocyclyl optionally substituted with one or more R^{a1}.

17. The compound, tautomer, stereoisomer, or a pharmaceutically acceptable salt thereof of claim 16, wherein Ring A is a 3- to 12-membered monocyclic heterocyclyl containing one or two nitrogen atoms, which is optionally substituted with one or more R^{a1}.

18. The compound, tautomer, stereoisomer, or a pharmaceutically acceptable salt thereof of claim 17, wherein Ring A is piperidinyl optionally substituted with one or more R^{a1}.

19. The compound, tautomer, stereoisomer, or a pharmaceutically acceptable salt thereof of claim 18, wherein Ring A is 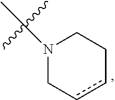
wherein --- is a bond via which Ring A is fused to Ring B.

20. The compound, tautomer, stereoisomer, or a pharmaceutically acceptable salt thereof of any one of the preceding claims, wherein Ring B is heterocyclyl or aryl, each of which is independently optionally substituted with one or more R^{a2}.

21. The compound, tautomer, stereoisomer, or a pharmaceutically acceptable salt thereof of claim 20, wherein Ring A and Ring B are not heterocyclyl or aryl at the same time.

22. The compound, tautomer, stereoisomer, or a pharmaceutically acceptable salt thereof of claim 20, wherein Ring B is a heterocyclyl optionally substituted with one or more R^{a2}.

23. The compound, tautomer, stereoisomer, or a pharmaceutically acceptable salt thereof of claim 22, wherein Ring B is a 3- to 12-membered heterocyclyl optionally substituted with one or more R^{a2}.

24. The compound, tautomer, stereoisomer, or a pharmaceutically acceptable salt thereof of claim 22 or 23, wherein Ring B is a monocyclic heterocyclyl optionally substituted with one or more R^{a2}.

25. The compound, tautomer, stereoisomer, or a pharmaceutically acceptable salt thereof of claim 24, wherein Ring B is a 3- to 12-membered monocyclic heterocyclyl optionally substituted with one or more R^{a2}.

26. The compound, tautomer, stereoisomer, or a pharmaceutically acceptable salt thereof of claim 25, wherein Ring B is a 3- to 12-membered monocyclic heterocyclyl containing one or two nitrogen atoms, which is optionally substituted with one or more R².

27. The compound, tautomer, stereoisomer, or a pharmaceutically acceptable salt thereof of claim 26, wherein Ring B is piperidinyl optionally substituted with one or more R^{a2}.

28. The compound, tautomer, stereoisomer, or a pharmaceutically acceptable salt thereof of claim 27, wherein Ring B is 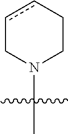
wherein --- is a bond via which Ring B is fused to Ring A.

29. The compound, tautomer, stereoisomer, or a pharmaceutically acceptable salt thereof of any of claims 1 to 21, wherein Ring B is aryl optionally substituted with one or more R^{a2}.

30. The compound, tautomer, stereoisomer, or a pharmaceutically acceptable salt thereof of claim 29, wherein Ring B is a 3- to 12-membered aryl optionally substituted with one or more R^{a2}.

31. The compound, tautomer, stereoisomer, or a pharmaceutically acceptable salt thereof of claim 29 or 30, wherein Ring B is a monocyclic aryl optionally substituted with one or more R^{a2}.

32. The compound, tautomer, stereoisomer, or a pharmaceutically acceptable salt thereof of claim 31, wherein Ring B is a 3- to 12-membered monocyclic aryl optionally substituted with one or more R^{a2}.

33. The compound, tautomer, stereoisomer, or a pharmaceutically acceptable salt thereof of claim 32, wherein Ring B is a phenyl optionally substituted with one or more R^{a2}.

34. The compound, tautomer, stereoisomer, or a pharmaceutically acceptable salt thereof of claim 33, wherein Ring B is an unsubstituted phenyl.

35. The compound, tautomer, stereoisomer, or a pharmaceutically acceptable salt thereof of any one of the preceding claims, wherein L¹ is

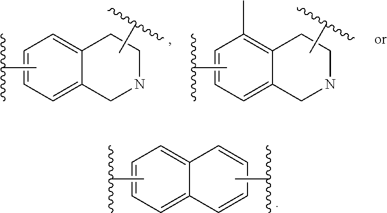

36. The compound, tautomer, stereoisomer, or a pharmaceutically acceptable salt thereof of claim 35, wherein L¹ is 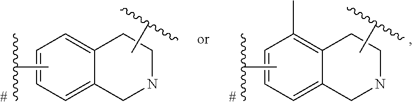
and # is linked to L².

37. The compound, tautomer, stereoisomer, or a pharmaceutically acceptable salt thereof of claim 35, wherein L¹ is

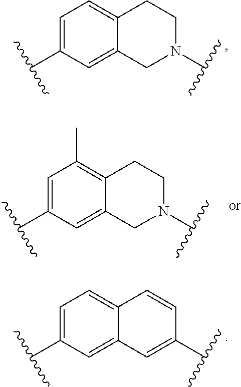 [SMILES: *c1ccc2c(c1)CN(*)CC2 ; *c1cc(C)c2c(c1)CN(*)CC2 ; *c1ccc2ccc(*)cc2c1]

38. The compound, tautomer, stereoisomer, or a pharmaceutically acceptable salt thereof of claim 37, wherein L¹ is 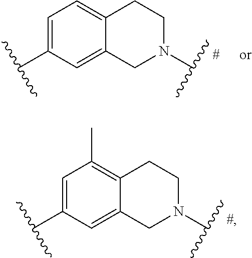 [SMILES: *c1ccc2c(c1)CN(*)CC2 ; *c1cc(C)c2c(c1)CN(*)CC2]
and # is linked to L².

39. The compound, tautomer, stereoisomer, or a pharmaceutically acceptable salt thereof of claim 37, wherein L¹ is 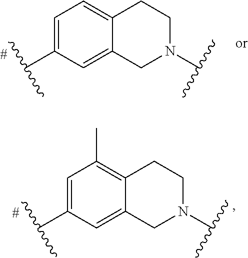 [SMILES: *c1ccc2c(c1)CN(*)CC2 ; *c1cc(C)c2c(c1)CN(*)CC2]
and # is linked to L².

40. The compound, tautomer, stereoisomer, or a pharmaceutically acceptable salt thereof of claim 1 or 2, wherein L¹ is

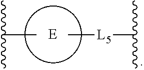

41. The compound, tautomer, stereoisomer, or a pharmaceutically acceptable salt thereof of claim 40, wherein Ring E is aryl optionally substituted with one or more R^{a3}.

42. The compound, tautomer, stereoisomer, or a pharmaceutically acceptable salt thereof of claim 41, wherein Ring E is a 3- to 12-membered aryl optionally substituted with one or more R^{a3}.

43. The compound, tautomer, stereoisomer, or a pharmaceutically acceptable salt thereof of claim 41 or 42, wherein Ring E is a monocyclic aryl optionally substituted with one or more R^{a3}.

44. The compound, tautomer, stereoisomer, or a pharmaceutically acceptable salt thereof of claim 43, wherein Ring E is a 3- to 12-membered monocyclic aryl optionally substituted with one or more R^{a3}.

45. The compound, tautomer, stereoisomer, or a pharmaceutically acceptable salt thereof of claim 44, wherein Ring E is a phenyl optionally substituted with one or more R^{a3}.

46. The compound, tautomer, stereoisomer, or a pharmaceutically acceptable salt thereof of claim 40, wherein Ring E is heteroaryl optionally substituted with one or more R^{a3}.

47. The compound, tautomer, stereoisomer, or a pharmaceutically acceptable salt thereof of claim 46, wherein Ring E is a 3- to 12-membered heteroaryl optionally substituted with one or more R^{a3}.

48. The compound, tautomer, stereoisomer, or a pharmaceutically acceptable salt thereof of claim 46 or 47, wherein Ring E is a monocyclic heteroaryl optionally substituted with one or more R^{a3}.

49. The compound, tautomer, stereoisomer, or a pharmaceutically acceptable salt thereof of claim 48, wherein Ring E is a 3- to 12-membered monocyclic heteroaryl optionally substituted with one or more R^{a3}.

50. The compound, tautomer, stereoisomer, or a pharmaceutically acceptable salt thereof of claim 49, wherein Ring E is a monocyclic heteroaryl containing one or two nitrogen atoms, which is optionally substituted with one or more R^{a3}.

51. The compound, tautomer, stereoisomer, or a pharmaceutically acceptable salt thereof of claim 50, wherein Ring E is pyridinyl optionally substituted with one or more R^{a3}.

52. The compound, tautomer, stereoisomer, or a pharmaceutically acceptable salt thereof of claim 40, wherein L¹ is 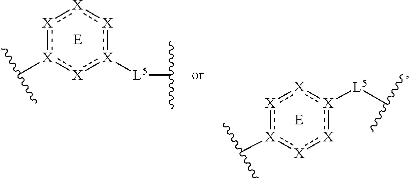
each of X is independently selected from the group consisting of C, CR^{a3}, CH, N, NR^{a3}, and NH.

53. The compound, tautomer, stereoisomer, or a pharmaceutically acceptable salt thereof of claim 52, wherein L¹ is 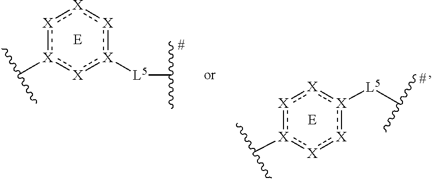
each of X is independently selected from the group consisting of C, CR^{a}, CH, N, NR^{a3}, and NH, and # is linked to L².

54. The compound, tautomer, stereoisomer, or a pharmaceutically acceptable salt thereof of any one of claims 40 to 53, wherein L⁵ is a heteroalkyl optionally substituted with one or more R^{a6}.

55. The compound, tautomer, stereoisomer, or a pharmaceutically acceptable salt thereof of claim 54, wherein L⁵ is a heteroalkyl containing at least one nitrogen atom and optionally substituted with one or more R^{a6}.

56. The compound, tautomer, stereoisomer, or a pharmaceutically acceptable salt thereof of claim 55, wherein L⁵ is a heteroalkyl containing one to six carbon atoms and one nitrogen atom and optionally substituted with one or more R^{a6}.

57. The compound, tautomer, stereoisomer, or a pharmaceutically acceptable salt thereof of claim 55, wherein L⁵ is selected from the group consisting of —CH₂—N(R^{a6})—, —(CH₂)₂—N(R^{a6})—, —(CH₂)₃—N(R^{a6})—, —(CH₂)₄—N(R^{a6})—, —(CH₂)₅—N(R^{a6})— and —(CH₂)₆—N(R^{a6})—.

58. The compound, tautomer, stereoisomer, or a pharmaceutically acceptable salt thereof of any one of claims 54 to 57, wherein R^{a6} is C₁₋₆ alkyl.

59. The compound, tautomer, stereoisomer, or a pharmaceutically acceptable salt thereof of claim 58, wherein R^{a6} is methyl.

60. The compound, tautomer, stereoisomer, or a pharmaceutically acceptable salt thereof of any one of claims 40 to 59, wherein L¹ is

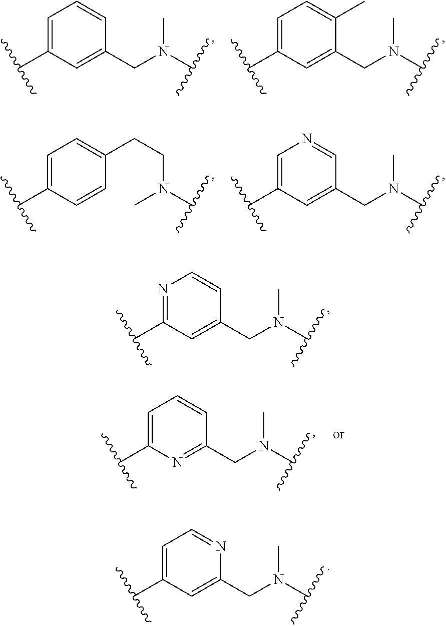

61. The compound, tautomer, stereoisomer, or a pharmaceutically acceptable salt thereof of any one of the preceding claims, wherein L² is —C(O)— or —S(O)₂—.

62. The compound, tautomer, stereoisomer, or a pharmaceutically acceptable salt thereof of any one of the preceding claims, wherein L³ is a bond.

63. The compound, tautomer, stereoisomer, or a pharmaceutically acceptable salt thereof of any one of claims 1 to 61, wherein L³ is a cycloalkyl.

64. The compound, tautomer, stereoisomer, or a pharmaceutically acceptable salt thereof of claim 63, wherein L³ is a C₃₋₁₂ cycloalkyl.

65. The compound, tautomer, stereoisomer, or a pharmaceutically acceptable salt thereof of claim 64, wherein L³ is a cyclohexyl.

66. The compound, tautomer, stereoisomer, or a pharmaceutically acceptable salt thereof of claim 65, wherein L³ is

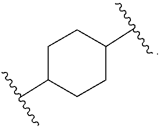 [SMILES: *C1CCC(*)CC1]

67. The compound, tautomer, stereoisomer, or a pharmaceutically acceptable salt thereof of any one of claims 1 to 61, wherein L³ is a heterocyclyl.

68. The compound, tautomer, stereoisomer, or a pharmaceutically acceptable salt thereof of claim 67, wherein L³ is a 3- to 12-membered heterocyclyl.

69. The compound, tautomer, stereoisomer, or a pharmaceutically acceptable salt thereof of claim 68, wherein L³ is a 3- to 12-membered heterocyclyl containing one or two nitrogen atoms.

70. The compound, tautomer, stereoisomer, or a pharmaceutically acceptable salt thereof of claim 69, wherein L³ is piperidinyl or piperazinyl.

71. The compound, tautomer, stereoisomer, or a pharmaceutically acceptable salt thereof of claim 70, wherein L³ is

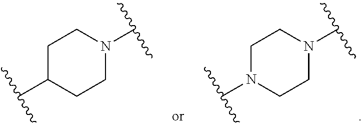 [SMILES: *C1CCN(*)CC1 ; *N1CCN(*)CC1]

72. The compound, tautomer, stereoisomer, or a pharmaceutically acceptable salt thereof of claim 71, wherein L³ is 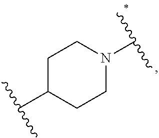 [SMILES: *C1CCN(*)CC1]
and * is linked to L².

73. The compound, tautomer, stereoisomer, or a pharmaceutically acceptable salt thereof of any one of claims 1 to 61, wherein L³ is aryl.

74. The compound, tautomer, stereoisomer, or a pharmaceutically acceptable salt thereof of claim 73, wherein L³ is a 3- to 12-membered aryl.

75. The compound, tautomer, stereoisomer, or a pharmaceutically acceptable salt thereof of claim 73 or 74, wherein L³ is a monocyclic aryl.

76. The compound, tautomer, stereoisomer, or a pharmaceutically acceptable salt thereof of claim 75, wherein L³ is a 3- to 12-membered monocyclic aryl.

77. The compound, tautomer, stereoisomer, or a pharmaceutically acceptable salt thereof of claim 76, wherein L³ is phenyl.

78. The compound, tautomer, stereoisomer, or a pharmaceutically acceptable salt thereof of claim 77, wherein L³ is

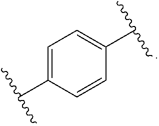 [SMILES: *c1ccc(*)cc1]

79. The compound, tautomer, stereoisomer, or a pharmaceutically acceptable salt thereof of any one of the preceding claims, wherein L⁴ is selected from the group consisting of alkyl, alkenyl, heteroalkyl or heteroalkenyl, wherein the alkyl, alkenyl, heteroalkyl and heteroalkenyl are independently optionally substituted with one or more R^{a5}.

80. The compound, tautomer, stereoisomer, or a pharmaceutically acceptable salt thereof of claim 79, wherein L⁴ is C₁₋₁₂ alkyl or C₂₋₁₂ alkenyl, which is optionally substituted with one or more R^{a5}.

81. The compound, tautomer, stereoisomer, or a pharmaceutically acceptable salt thereof of claim 80, wherein L⁴ is C₃₋₁₀ alkyl.

82. The compound, tautomer, stereoisomer, or a pharmaceutically acceptable salt thereof of claim 81, wherein L⁴ is

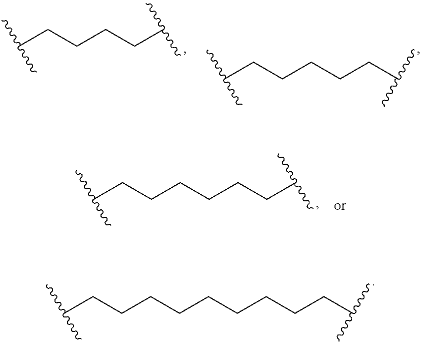 [SMILES: *CCCC* ; *CCCCC* ; *CCCCCC* ; *CCCCCCCCC*]

83. The compound, tautomer, stereoisomer, or a pharmaceutically acceptable salt thereof of claim 79, wherein L⁴ is heteroalkyl containing one or more oxygen or nitrogen atoms.

84. The compound, tautomer, stereoisomer, or a pharmaceutically acceptable salt thereof of claim 83, wherein L⁴ is C₁₋₁₂ heteroalkyl containing one or two oxygen atoms.

85. The compound, tautomer, stereoisomer, or a pharmaceutically acceptable salt thereof of claim 84, wherein L⁴ is C₃₋₁₂ heteroalkyl containing one or two oxygen atoms, and the oxygen atom is inserted into main chain of the C₃₋₁₂ heteroalkyl.

86. The compound, tautomer, stereoisomer, or a pharmaceutically acceptable salt thereof of claim 85, wherein L⁴ is

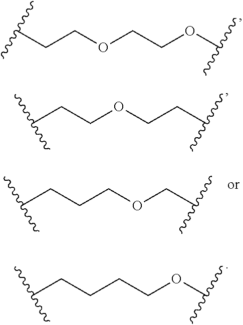 [SMILES: *CCOCCO* ; *CCOCC* ; *CCCOC* ; *CCCCO*]

87. The compound, tautomer, stereoisomer, or a pharmaceutically acceptable salt thereof of claim 83, wherein L⁴ is C₁₋₁₂ heteroalkyl containing one or two nitrogen atoms.

88. The compound, tautomer, stereoisomer, or a pharmaceutically acceptable salt thereof of claim 87, wherein L⁴ is C₃₋₁₂ heteroalkyl containing one nitrogen atom, and the nitrogen atom is inserted into main chain of the C₃₋₁₂ heteroalkyl.

89. The compound, tautomer, stereoisomer, or a pharmaceutically acceptable salt thereof of claim 88, wherein L⁴ is

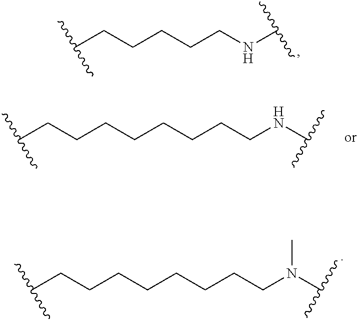 [SMILES: *CCCCCN* ; *CCCCCCCCN* ; *CCCCCCCCN(*)C]

90. The compound, tautomer, stereoisomer, or a pharmaceutically acceptable salt thereof of claim 83, wherein L⁴ is heteroalkenyl containing one or more oxygen atoms.

91. The compound, tautomer, stereoisomer, or a pharmaceutically acceptable salt thereof of claim 90, wherein L⁴ is C₂₋₁₂ heteroalkenyl containing one oxygen atom.

92. The compound, tautomer, stereoisomer, or a pharmaceutically acceptable salt thereof of claim 91, wherein L⁴ is C₂₋₁₂ heteroalkenyl containing one oxygen atom, and the oxygen atom is inserted into main chain of the C₂₋₁₂ heteroalkenyl.

93. The compound, tautomer, stereoisomer, or a pharmaceutically acceptable salt thereof of claim 92, wherein L⁴ is

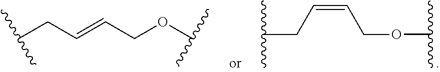 [SMILES: *C/C=C/CO* ; *C/C=C\CO* ; "or"]

94. The compound, tautomer, stereoisomer, or a pharmaceutically acceptable salt thereof of any one of claims 79 to 93, wherein R^{a5} is alkyl.

95. The compound, tautomer, stereoisomer, or a pharmaceutically acceptable salt thereof of claim 94, wherein R^{a5} is C₁₋₆ alkyl.

96. The compound, tautomer, stereoisomer, or a pharmaceutically acceptable salt thereof of claim 95, wherein R^{a5} is methyl.

97. The compound, tautomer, stereoisomer, or a pharmaceutically acceptable salt thereof of any one of the preceding claims, wherein R¹ is hydrogen or C₁₋₆ alkyl.

98. The compound, tautomer, stereoisomer, or a pharmaceutically acceptable salt thereof of any one of the preceding claims, wherein R² is hydrogen or C₁₋₆ alkyl.

99. The compound, tautomer, stereoisomer, or a pharmaceutically acceptable salt thereof of any one of the preceding claims, wherein q is 1.

100. The compound, tautomer, stereoisomer, or a pharmaceutically acceptable salt thereof of any one of the preceding claims, wherein R^{a3} is —OR^{c}.

101. The compound, tautomer, stereoisomer, or a pharmaceutically acceptable salt thereof of claim 100, wherein R^{c} is hydrogen or C₁₋₆ alkyl.

102. The compound, tautomer, stereoisomer, or a pharmaceutically acceptable salt thereof of claim 101, wherein R^{c} is hydrogen.

103. The compound, tautomer, stereoisomer, or a pharmaceutically acceptable salt thereof of claim 102, wherein n is 0 or 1.

104. The compound, tautomer, stereoisomer, or a pharmaceutically acceptable salt thereof of any one of the preceding claims, wherein R⁴ is alkyl.

105. The compound, tautomer, stereoisomer, or a pharmaceutically acceptable salt thereof of claim 104, wherein R⁴ is C₁₋₆ alkyl.

106. The compound, tautomer, stereoisomer, or a pharmaceutically acceptable salt thereof of claim 105, wherein R⁴ is methyl.

107. The compound, tautomer, stereoisomer, or a pharmaceutically acceptable salt thereof of claim 1 or 2, wherein the compound is represented by Formula (II), Formula (III) or Formula (IV) below: 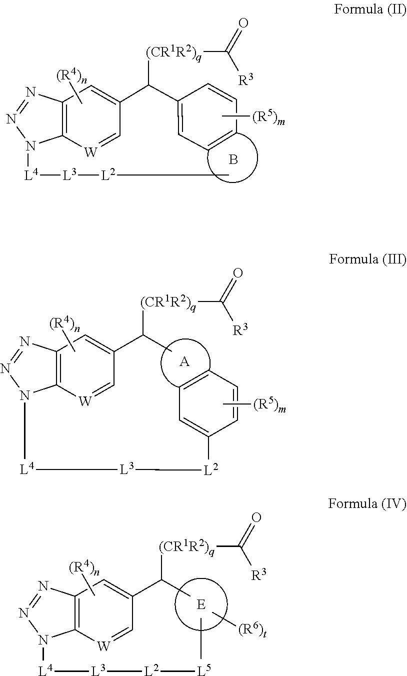
wherein,
each of R⁵ and R⁶ is independently selected from the group consisting of hydrogen, halogen, hydroxyl, sulfhydryl, cyano, —NH₂, —NO₂, alkoxyl, alkyl, alkenyl, alkynyl, heteroalkyl, heteroalkenyl, heteroalkynyl, haloalkyl, and alkylalkoxyl, wherein the alkyl, alkenyl and alkynyl, either alone or as part of another group, are independently optionally substituted with one or more R^{a7};
m is 0, 1, 2 or 3;
t is 0, 1, 2 or 3; and
each of W, R¹, R², R³, R⁴, L², L³, L⁴, L¹, Ring A, Ring B, Ring E, R^{a7}, n and q are as defined in claim 1.

108. The compound, tautomer, stereoisomer, or a pharmaceutically acceptable salt thereof of claim 107, wherein the compound is represented by Formula (IIa), Formula (IIb), Formula (IIIa), Formula (IIIb), Formula (IVa) or Formula (IVb) below: 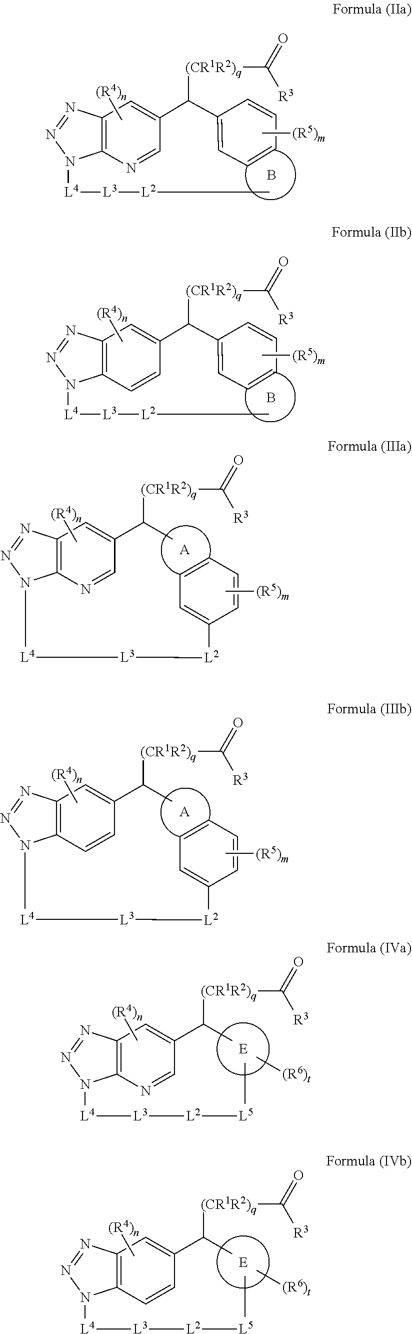
wherein, each of W, R¹, R², R³, R⁴, R⁵, R⁶, L², L³, L⁴, L⁵, Ring A, Ring B, Ring E, n, m, q and t are as defined in claim 107.

109. The compound, tautomer, stereoisomer, or a pharmaceutically acceptable salt thereof of claim 108, wherein the compound is represented by Formula (IIa1) or Formula (IIa2) below: 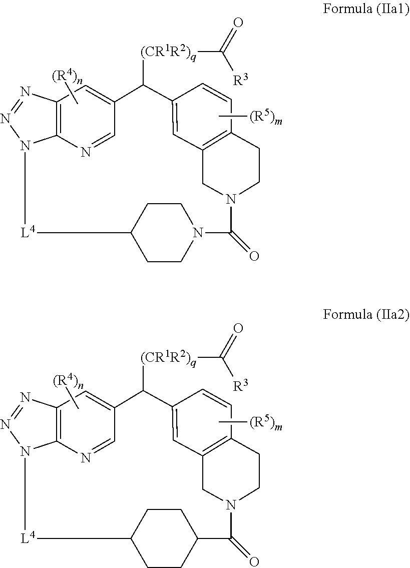
wherein,
L⁴ is C₃₋₈ alkyl or C₃₋₈ heteroalkyl;
R¹ is hydrogen, halogen or C₁₋₆ alkyl;
R² is hydrogen, halogen or C₁₋₆ alkyl;
R³ is —OR^{c}, R^{c} is hydrogen or C₁₋₆ alkyl;
R⁴ is hydrogen, halogen or C₁₋₆ alkyl;
R⁵ is hydrogen, halogen or C₁₋₆ alkyl;
n is 0 or 1;
mis 0 or 1; and
q is 1.

110. A compound, tautomer, stereoisomer, or a pharmaceutically acceptable salt thereof, selected from the group consisting of:

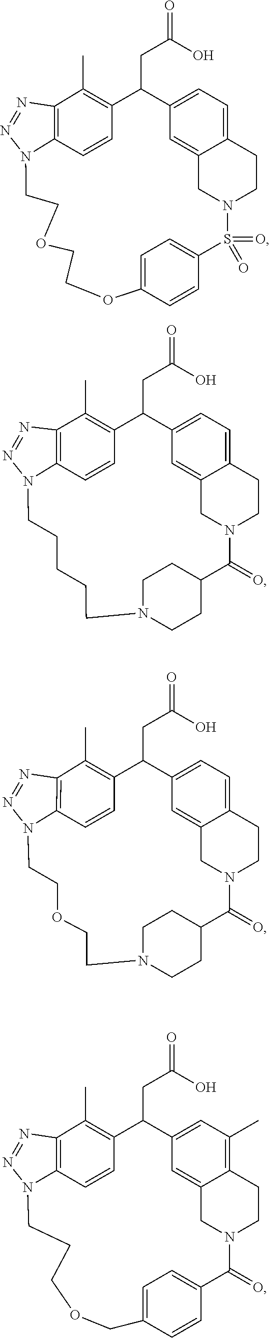

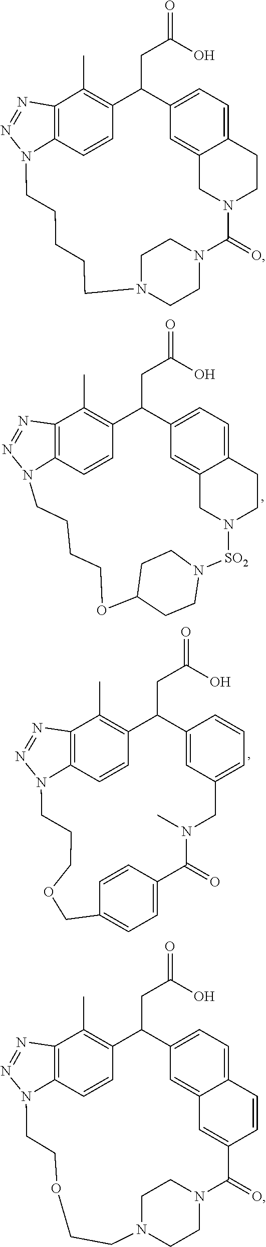

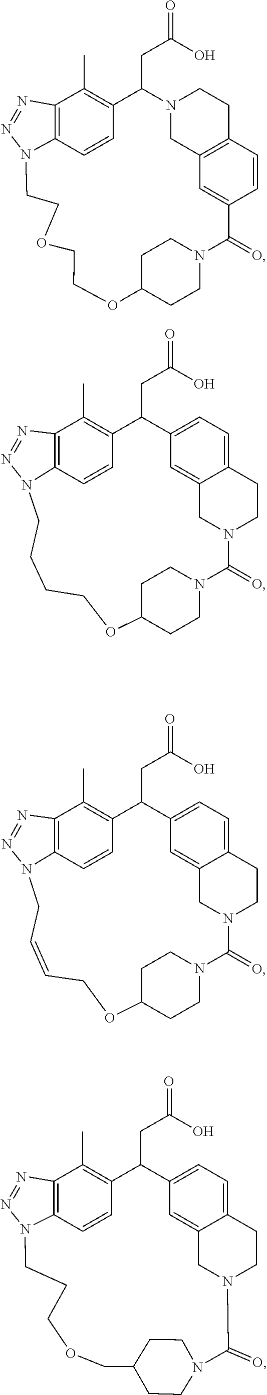

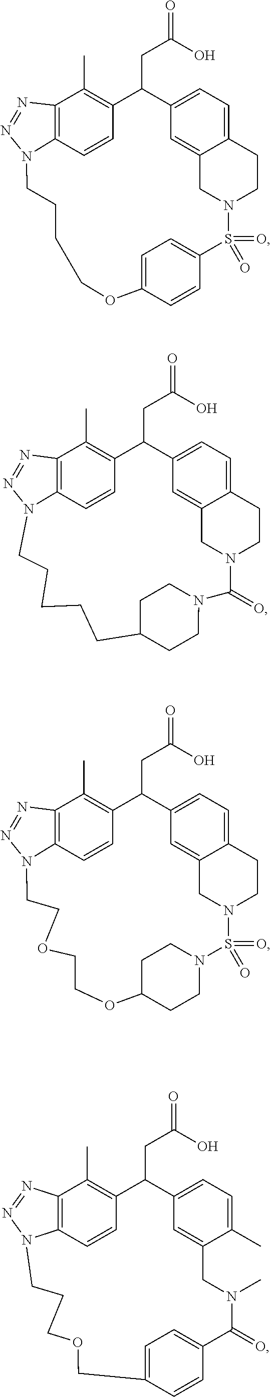 [SMILES: Cc1c2ccc3c1nnn3CCCCOc1ccc(cc1)S(=O)(=O)N1CCc3ccc(cc3C1)C2CC(=O)O ; Cc1c2ccc3c1nnn3CCCCCC1CCN(CC1)C(=O)N1CCc3ccc(cc3C1)C2CC(=O)O ; Cc1c2ccc3c1nnn3CCOCCOC1CCN(CC1)S(=O)(=O)N1CCc3ccc(cc3C1)C2CC(=O)O ; Cc1ccc2cc1CN(C)C(=O)c1ccc(cc1)COCCCn1nnc3c(C)c(ccc31)C2CC(=O)O]

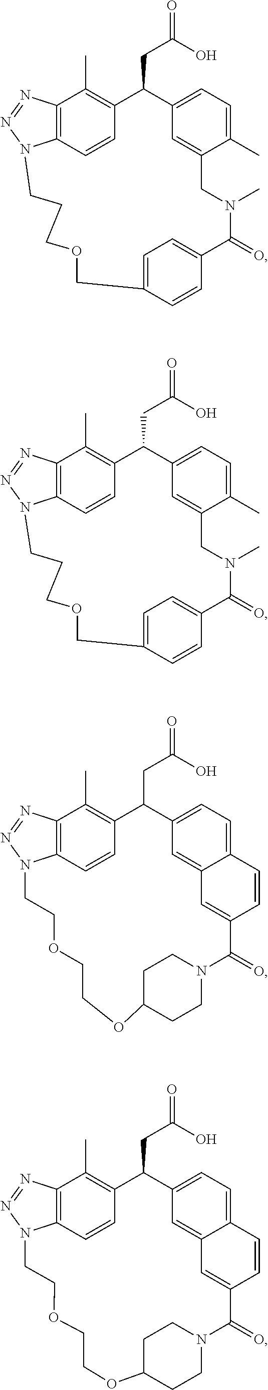 [SMILES: Cc1ccc2cc1CN(C)C(=O)c1ccc(cc1)COCCCn1nnc3c(C)c(ccc31)[C@H]2CC(=O)O ; Cc1ccc2cc1CN(C)C(=O)c1ccc(cc1)COCCCn1nnc3c(C)c(ccc31)[C@@H]2CC(=O)O ; Cc1c2ccc3c1nnn3CCOCCOC1CCN(CC1)C(=O)c1ccc3ccc(cc3c1)C2CC(=O)O ; Cc1c2ccc3c1nnn3CCOCCOC1CCN(CC1)C(=O)c1ccc3ccc(cc3c1)[C@@H]2CC(=O)O]

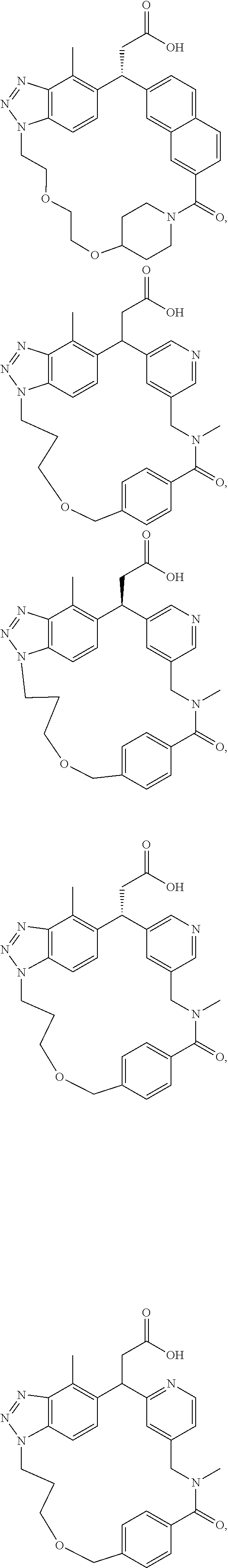

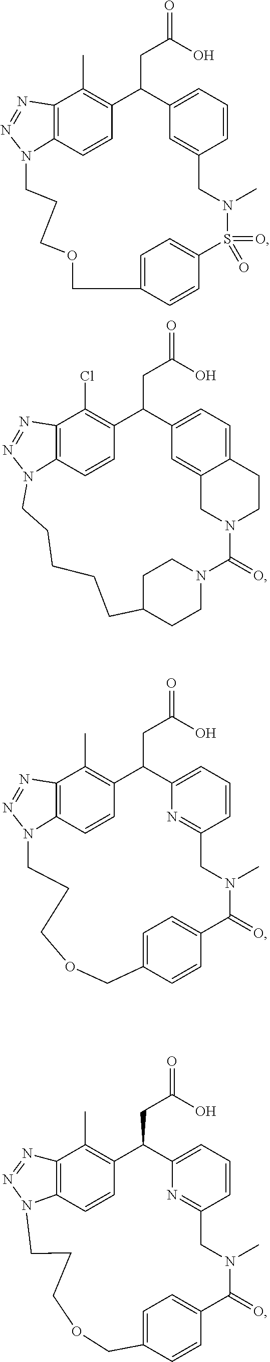

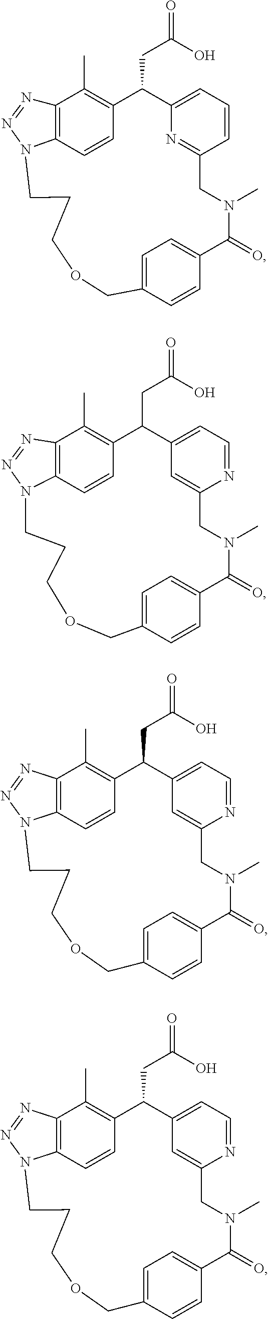

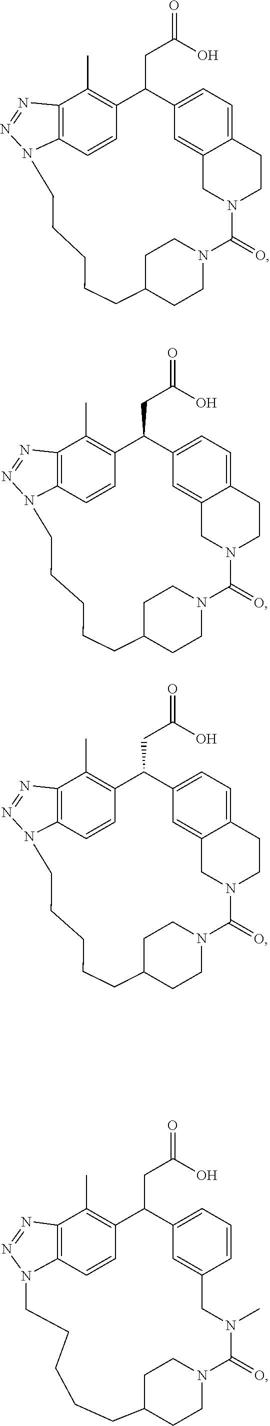

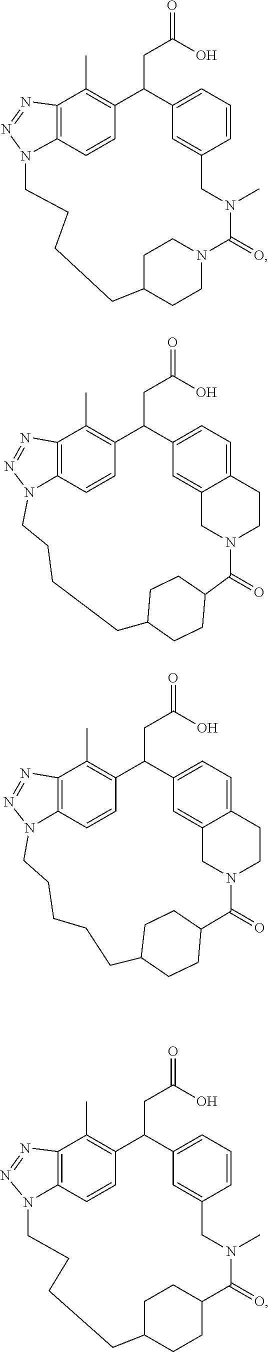

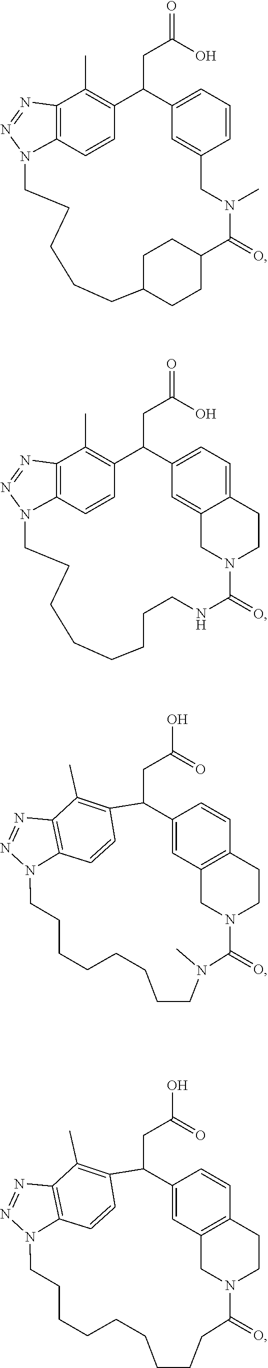 [SMILES: Cc1c2ccc3c1nnn3CCCCCC1CCC(CC1)C(=O)N(C)Cc1cccc(c1)C2CC(=O)O ; Cc1c2ccc3c1nnn3CCCCCCCCNC(=O)N1CCc3ccc(cc3C1)C2CC(=O)O ; Cc1c2ccc3c1nnn3CCCCCCCCN(C)C(=O)N1CCc3ccc(cc3C1)C2CC(=O)O ; Cc1c2ccc3c1nnn3CCCCCCCCCC(=O)N1CCc3ccc(cc3C1)C2CC(=O)O]

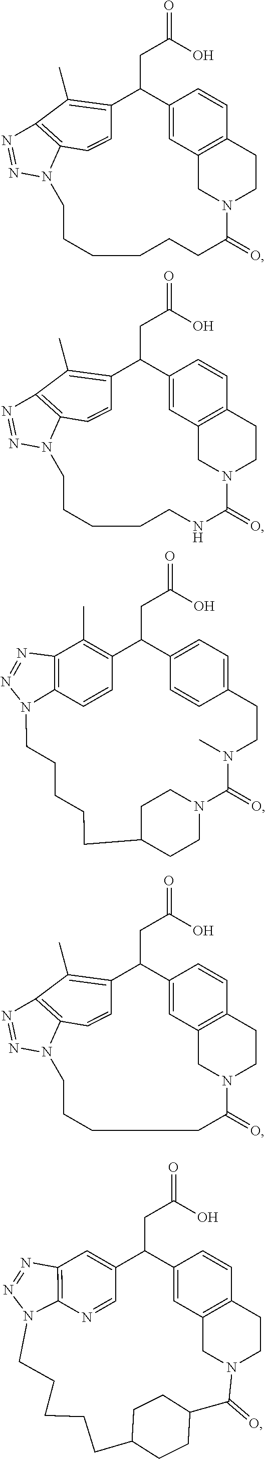

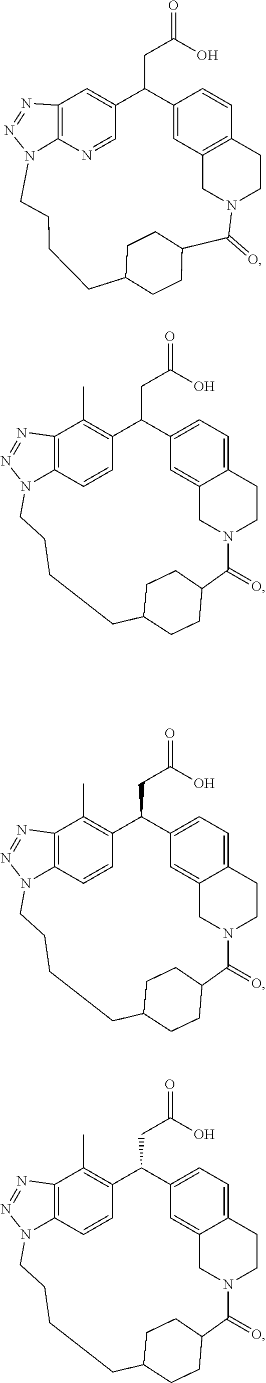

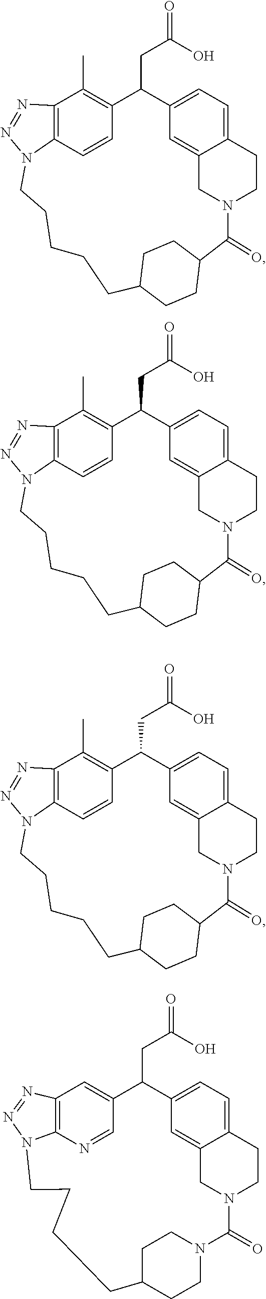

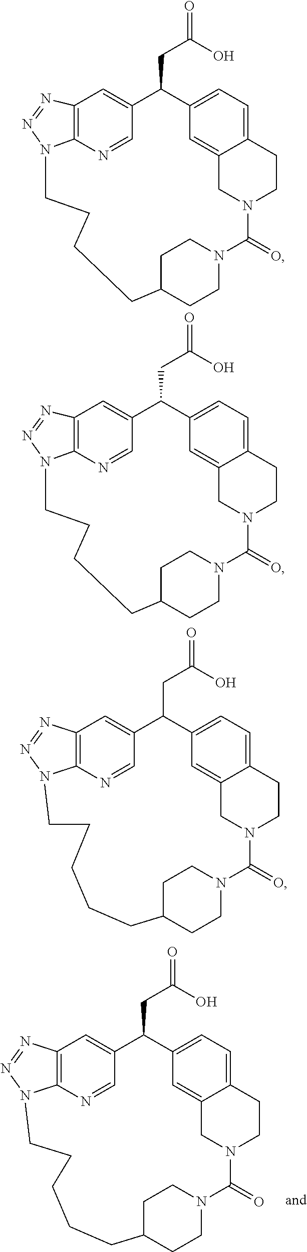

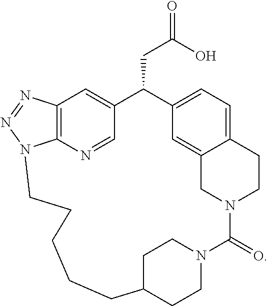 [SMILES: O=C(O)C[C@@H]1c2ccc3c(c2)CN(CC3)C(=O)N2CCC(CCCCCn3nnc4cc1cnc43)CC2]

111. A pharmaceutical composition comprising the compound, tautomer, stereoisomer, or a pharmaceutically acceptable salt thereof of any one of the preceding claims, and a pharmaceutically acceptable carrier or pharmaceutically acceptable excipient.

112. A method of increasing level or activity of Nrf2 in a cell, comprising exposing the cell to the compound, tautomer, stereoisomer, or a pharmaceutically acceptable salt thereof of any one of claims 1 to 110, or the pharmaceutical composition of claim 111.

113. A method of increasing level or activity of Nrf2 in a subject in need thereof, comprising administering to the subject a therapeutically effective amount of the compound, tautomer, stereoisomer, or a pharmaceutically acceptable salt thereof of any one of claims 1 to 110, or the pharmaceutical composition of claim 111.

114. A method of preventing, treating or alleviating a Nrf2-associated disease, disorder or condition in a subject in need thereof, comprising administering to the subject a therapeutically effective amount of the compound, tautomer, stereoisomer, or a pharmaceutically acceptable salt thereof of any one of claims 1 to 110, or the pharmaceutical composition of claim 111.

115. The method of claim 114, wherein the Nrf2-associated disease, disorder or condition is related to a decreased level or activity of Nrf2 protein.

116. The method of claim 114 or 115, wherein the Nrf2-associated disease, disorder or condition is related to increased oxidative stress, inflammation, impaired redox potential, impaired detoxification or deregulated metabolism.

117. The method of claim 114 or 115, wherein the disease, disorder or condition is selected from the group consisting of an ocular disease, a hepatic and bile duct disease, a cardiovascular disease, a lung disease, a kidney disease, a neurodegenerative disease, a neuropsychiatric disorder, a cancer, a sickle cell disease, a mitochondrial disease, an inflammatory disease, a respiratory disease, aging, an autoimmune disease, a brain disease, diabetes (e.g., Type I diabetes, Type II diabetes, maternal diabetes), metabolic syndrome and diabetic complications.

118. The method of claim 117, wherein the ocular disease is age related macular degeneration (AMD), retinitis pigmentosa (RP), geographic atrophy (GA), macular edema, macular edema following retinal vein occlusion (RVO), diabetic macular edema (DME), diabetic retinopathy (DR), retinal central vein occlusion, corneal neovascularization (CNV), ocular angiogenesis (ocular neovascularization affecting choroidal, corneal or retinal tissue), retinopathy of prematurity (ROP), pathological myopia, glaucoma (e.g., vascular glaucoma), retinoblastoma, retinal vein occlusion, uveitis, eye injury, Fuchs' endothelial corneal dystrophy (FECD), cataracts, ocular neurodegenerative diseases, optic neuropathy and neuromyelitis optica.

119. The method of claim 117, wherein the kidney disease is autosomal dominant polycystic kidney disease (ADPKD), acute kidney injury (AKI), diabetic nephrophaty, IgA nephropathy (IgAN), chronic kidney disease (CKD), Alstrom and Alport syndromes, renal fibrosis, focal segmental glomerulosclerosis, contrast-induced nephropathy, sepsis-induced acute kidney injury, and kidney disease or malfunction seen during kidney transplantation.

120. The method of claim 117, wherein the neurodegenerative disease is selected from the group consisting of Alzheimer's disease, Parkinson's disease, Huntington's disease, cognitive decline, amyloidosis, amyotrophic lateral sclerosis and multiple sclerosis.

121. The method of claim 117, wherein the neuropsychiatric disorder is selected from the group consisting of schizophrenia, bipolar disorder, depression, anxiety, Friedreich's ataxia, autism and attention deficit hyperactivity disorder.

122. The method of claim 117, wherein the diabetic complication is selected from the group consisting of diabetic cardiomyopathy, diabetic retinopathy, diabetic neuropathy, diabetic nephropathy and diabetic wound healing.

123. The method of claim 117, wherein the hepatic and bile duct disease is selected from the group consisting of nonalcoholic steatohepatitis (NASH), non-alcoholic fatty liver disease, toxin-induced liver disease (e.g., acetaminophen-induced hepatic disease), alcoholic liver disease (ALD), cholestasis, primary sclerosing cholangitis (PSC), viral hepatitis, cirrhosis, primary biliary cirrhosis (PBC), end stage liver disease, and liver fibrosis.

124. The method of claim 117, wherein the lung disease is selected from the group consisting of pulmonary fibrosis, idiopathic pulmonary fibrosis (IPF), cystic fibrosis, acute lung injury, lung infection, chronic obstructive pulmonary disease (COPD), emphysema, pulmonary arterial hypertension, lung disease secondary to environmental exposures, chronic and acute asthma, and acute respiratory distress syndrome.

125. The method of claim 117, wherein the cardiovascular disease is selected from the group consisting of atherosclerosis, hypertension, heart failure, stroke, cardiomyopathy, coronary heart disease, vascular endothelial dysfunction, blood-brain barrier dysfunction, reperfusion injury (brain, heart, kidney, liver, retina), and myocardial ischemia.

126. The method of claim 117, wherein the inflammatory disease is selected from the group consisting of inflammatory bowel disease, ulcerative colitis, Crohn's disease, pancreatitis, arthritis, osteoarthritis, dermatitis (e.g., radiation-induced dermatitis, allergic contact dermatitis), reflux-induced esophagitis and lupus nephritis.

127. The method of claim 117, wherein the autoimmune disease is selected from the group consisting of psoriasis, Sjogren syndrome, lupus, pemphigus, vitiligo and alopecia areata.

128. The method of claim 117, wherein the brain disease is selected from the group consisting of traumatic brain injury, brain edema, brain ischemia, encephalopathy (e.g., hepatic encephalopathy), and cerebral infarction.

129. The method of claim 117, wherein the Nrf2-associated disease, disorder or condition is selected from the group consisting of neuronal damage, epilepsy, spinal cord injury, immunosuppression due to radiation exposure, preeclampsia, high altitude sickness, wound healing, mitochondrial myopathies, malaria, ferroptosis/iron overload, alcohol dependence, anemia, asperger syndrome, eczema, chronic fatigue syndrome, Duchenne muscular dystrophy, edema, encephalitis, male/female fertility, fracture healing, gastroesophageal reflux disease, hearing loss, influenza infections, intestinal barrier dysfunction, osteoporosis, radiation-induced injury, seizures, skin ulcer and Down syndrome.

130. The method of claim 117, wherein the cancer is selected from the group consisting of colon cancer, lung cancer, esophageal cancer, breast cancer, bladder cancer, liver cancer, prostate cancer and colorectal cancer.

131. The method of any one of claims 114 to 130, wherein the compound, tautomer, stereoisomer, or a pharmaceutically acceptable salt thereof of any one of claims 1 to 110, or the pharmaceutical composition of claim 111 is administered simultaneously, separately or sequentially with a second therapy.

132. The method of claim 131, wherein the second therapy is chemotherapy or immunotherapy.

133. The method of claim 132, wherein the second therapy is selected from the group consisting of a chemotherapeutic agent, an anti-tumor agent, a radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesic, an antioxidant, a metal chelator, and cytokines.

134. The method of claim 131, wherein the second therapy is a Keap1 inhibitor.
